SEQUENCE LISTING (195 sequences)

SEQ ID NO: 1 - protein, 113 aa, synthetic construct
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGDYWGQGTTVTVSS

SEQ ID NO: 2 - protein, 113 aa, synthetic construct
QVQLQESGPGLVKPSQTLSLTCSVIGYSITSGYYWNWIRQPPGKGLEWIGSINYDGSNIYNPSLKDRVTISRDTSKNQFSLKLSSVTAADTAVYYCARGGDYWGQGTTVTVSS

SEQ ID NO: 3 - protein, 113 aa, synthetic construct; identical to SEQ ID NO: 2

SEQ ID NO: 4 - protein, 113 aa, synthetic construct
EVQLQESGPGLVKPSQTLSLTCTVIGYSITSGYYWNWIRQPPGKGLEWMGSINYDGSNIYNPSLKSRVTISRDTSKNQFSLKLSSVTAADTAVYYCARGGDYWGQGTTVTVSS

SEQ ID NO: 5 - protein, 113 aa, synthetic construct; identical to SEQ ID NO: 1

SEQ ID NO: 6 - protein, 113 aa, synthetic construct; identical to SEQ ID NO: 1

SEQ ID NO: 7 - protein, 113 aa, synthetic construct; identical to SEQ ID NO: 1

SEQ ID NO: 8 - protein, 113 aa, synthetic construct; identical to SEQ ID NO: 1

SEQ ID NO: 9 - protein, 113 aa, synthetic construct
EVQLKESGPALVKPTQTLTLTCTVIGYSITSGYYWNWIRQPPGKALEWMGSINYDGSNIYNPSLKTRLTISRDTSKNQFVLTMTNMDPVDTATYYCARGGDYWGQGTTVTVSS

SEQ ID NO: 10 - protein, 113 aa, synthetic construct; identical to SEQ ID NO: 9

SEQ ID NO: 11 - protein, 113 aa, synthetic construct; identical to SEQ ID NO: 9

SEQ ID NO: 12 - protein, 113 aa, synthetic construct; identical to SEQ ID NO: 9

SEQ ID NO: 13 - protein, 113 aa, synthetic construct
QVTLKESGPALVKPTQTLTLTCTVIGYSITSGYYWNWIRQPPGKALEWIGSINYDGSNIYNPSLKTRLTISRDTSKNQFVLTMTNMDPVDTATYYCARGGDYWGQGTTVTVSS

SEQ ID NO: 14 - protein, 113 aa, synthetic construct; identical to SEQ ID NO: 13

SEQ ID NO: 15 - protein, 113 aa, synthetic construct; identical to SEQ ID NO: 13

SEQ ID NO: 16 - protein, 113 aa, synthetic construct; identical to SEQ ID NO: 13

SEQ ID NO: 17 - protein, 113 aa, synthetic construct; identical to SEQ ID NO: 4

SEQ ID NO: 18 - protein, 113 aa, synthetic construct; identical to SEQ ID NO: 1

SEQ ID NO: 19 - protein, 113 aa, synthetic construct; identical to SEQ ID NO: 2

SEQ ID NO: 20 - protein, 113 aa, synthetic construct; identical to SEQ ID NO: 4

SEQ ID NO: 21 - protein, 113 aa, synthetic construct; identical to SEQ ID NO: 4

SEQ ID NO: 22 - protein, 113 aa, synthetic construct
EVQLQESGPGLVKPSQTLSLTCSVIGYSITSGYYWNWIRQPPGKGLEWMGSINYDGSNIYNPSLKDRVTISRDTSKNQFSLKLSSVTAADTAVYYCARGGDYWGQGTSVTVSS

SEQ ID NO: 23 - protein, 113 aa, synthetic construct; identical to SEQ ID NO: 22

SEQ ID NO: 24 - protein, 113 aa, synthetic construct; identical to SEQ ID NO: 22

SEQ ID NO: 25 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTFGGGTKLEIK

SEQ ID NO: 26 - protein, 107 aa, synthetic construct
DIVLTQSPAILSLSPGERATLSCSASSSVSYMHWFQQKSGQAPKRWIYDTSKLASGVPARFSGSGSGTDYTLTISSLEAEDFAYYCQQWSSDPPMLTFGGGTKLEIK

SEQ ID NO: 27 - protein, 107 aa, synthetic construct
EIVLTQSPATLSLSPGERATLSCSASSSVSYMHWFQQKPGQAPRRWIYDTSKRATGVPARFSGSGSGTDYTLTISSLEPEDFAYYCQQWSSDPPMLTFGGGTKLEIK

SEQ ID NO: 28 - protein, 108 aa, synthetic construct
DIQMTQSPSSLSASVGDRVTITCRASSSVSYMHWFQQKPGKAPKRLIYDTSKLASGVPSRFSGSGSGTDYTLTISSLQPEDFATYYCQQWSSDPPMLTFGGGTKLEIK

SEQ ID NO: 29 - protein, 108 aa, synthetic construct
DIVLTQSPATLSLSPGERATLSCRASSSVSYMHWFQQKPGQAPRRLIYDTSKLASGIPARFSGSGSGTDYTLTISSLEPEDFAVYYCQQWSSDPPMLTFGGGTKLEIK

SEQ ID NO: 30 - protein, 108 aa, synthetic construct
EIVLTQSPATLSLSPGERATLSCRASSSVSYMHWFQQKPGQAPRRLIYDTSKRATGIPARFSGSGSGTDYTLTISSLEPEDFAVYYCQQWSSDPPMLTFGGGTKLEIK

SEQ ID NO: 31 - protein, 108 aa, synthetic construct
DIQLTQSPSSLSASVGDRVTITCRASSSVSYMHWFQQKPGKAPKRLIYDTSKLASGVPSRFSGSGSGTDYTLTISSLQPEDFATYYCQQWSSDPPMLTFGGGTKLEIK

SEQ ID NO: 32 - protein, 108 aa, synthetic construct; identical to SEQ ID NO: 28

SEQ ID NO: 33 - protein, 108 aa, synthetic construct; identical to SEQ ID NO: 29

SEQ ID NO: 34 - protein, 108 aa, synthetic construct; identical to SEQ ID NO: 30

SEQ ID NO: 35 - protein, 108 aa, synthetic construct; identical to SEQ ID NO: 31

SEQ ID NO: 36 - protein, 108 aa, synthetic construct; identical to SEQ ID NO: 28

SEQ ID NO: 37 - protein, 108 aa, synthetic construct; identical to SEQ ID NO: 29

SEQ ID NO: 38 - protein, 108 aa, synthetic construct; identical to SEQ ID NO: 30

SEQ ID NO: 39 - protein, 108 aa, synthetic construct; identical to SEQ ID NO: 31

SEQ ID NO: 40 - protein, 108 aa, synthetic construct; identical to SEQ ID NO: 28

SEQ ID NO: 41 - protein, 108 aa, synthetic construct; identical to SEQ ID NO: 29

SEQ ID NO: 42 - protein, 107 aa, synthetic construct
DIVLTQSPAILSLSPGERATLSCSASSSVSYMHWFQQKSGQAPKRWIYDTSKLASGVPARFSGSGSGTDYTLTISSLEAEDFMYYCQQWSSDPPMLTFGGGTKLEIK

SEQ ID NO: 43 - protein, 108 aa, synthetic construct; identical to SEQ ID NO: 30

SEQ ID NO: 44 - protein, 108 aa, synthetic construct; identical to SEQ ID NO: 31

SEQ ID NO: 45 - protein, 108 aa, synthetic construct; identical to SEQ ID NO: 30

SEQ ID NO: 46 - protein, 108 aa, synthetic construct; identical to SEQ ID NO: 30

SEQ ID NO: 47 - protein, 107 aa, synthetic construct
EIVLTQSPATLSLSPGERATLSCSASSSVSYMHWFQQKPGQAPRRWIYDTSKRATGVPARFSGSGSGTDYTLTISSLEPEDFMYYCQQWSSDPPMLTFGGGTKLEIK

SEQ ID NO: 48 - protein, 107 aa, synthetic construct; identical to SEQ ID NO: 42

SEQ ID NO: 49 - protein, 113 aa, synthetic construct
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGDPWGQGTTVTVSS

SEQ ID NO: 50 - protein, 113 aa, synthetic construct
QVQLQESGPGLVKPSQTLSLTCTVIGYSITSGYYWNWIRQPPGKGLEWIGSINYDGTNIYNPSLKSRVTISRDTSKNQFSLKLSSVTAADTAVYYCARGGDPWGQGTTVTVSS

SEQ ID NO: 51 - protein, 113 aa, synthetic construct; identical to SEQ ID NO: 1

SEQ ID NO: 52 - protein, 113 aa, synthetic construct
QVQLQESGPGLVKPSQTLSLTCTVIGQSITAGYYWNWIRQPPGKGLEWIGSINYDGSNIYNPSLKSRVTISRDTSKNQFSLKLSSVTAADTAVYYCARGGHPWGQGTTVTVSS

SEQ ID NO: 53 - protein, 113 aa, synthetic construct
QVQLQESGPGLVKPSQTLSLTCTVIGQSITSGYNWNWIRQPPGKGLEWIGSINHDGSNFYNPSLKSRVTISRDTSKNQFSLKLSSVTAADTAVYYCARGGAYWGQGTTVTVSS

SEQ ID NO: 54 - protein, 113 aa, synthetic construct
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGRDWGQGTTVTVSS

SEQ ID NO: 55 - protein, 113 aa, synthetic construct
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGDPWGQGTTVTVSS

SEQ ID NO: 56 - protein, 113 aa, synthetic construct
QVQLQESGPGLVKPSQTLSLTCTVIGQSITSGYYWNWIRQPPGKGLEWIGSINFDGSNIYNPSLKSRVTISRDTSKNQFSLKLSSVTAADTAVYYCARGGDPWGQGTTVTVSS

SEQ ID NO: 57 - protein, 113 aa, synthetic construct
QVQLQESGPGLVKPSQTLSLTCTVIGYSITTGYYWNWIRQPPGKGLEWIGSINYDGSNIYNPSLKSRVTISRDTSKNQFSLKLSSVTAADTAVYYCARGGEPWGQGTTVTVSS

SEQ ID NO: 58 - protein, 113 aa, synthetic construct
QVQLQESGPGLVKPSQTLSLTCTVIGYSITQGYYWNWIRQPPGKGLEWIGSINYDGLNIYNPSLKSRVTISRDTSKNQFSLKLSSVTAADTAVYYCARGGRDWGQGTTVTVSS

SEQ ID NO: 59 - protein, 113 aa, synthetic construct
QVQLQESGPGLVKPSQTLSLTCTVIGQSITSGYNWNWIRQPPGKGLEWIGSINYDGSNFYNPSLKSRVTISRDTSKNQFSLKLSSVTAADTAVYYCARGGNLWGQGTTVTVSS

SEQ ID NO: 60 - protein, 113 aa, synthetic construct
QVQLQESGPGLVKPSQTLSLTCTVIGQSITAGYYWNWIRQPPGKGLEWIGSINYDGSNIYNPSLKSRVTISRDTSKNQFSLKLSSVTAADTAVYYCARGGDPWGQGTTVTVSS

SEQ ID NO: 61 - protein, 113 aa, synthetic construct
QVQLQESGPGLVKPSQTLSLTCTVIGYSITQGYYWNWIRQPPGKGLEWIGSINYDGSNIYNPSLKSRVTISRDTSKNQFSLKLSSVTAADTAVYYCARGGDYWGQGTTVTVSS

SEQ ID NO: 62 - protein, 113 aa, synthetic construct
QVQLQESGPGLVKPSQTLSLTCTVIGQSITSGYYWNWIRQPPGKGLEWIGSINYDGTNIYNPSLKSRVTISRDTSKNQFSLKLSSVTAADTAVYYCARGGDPWGQGTTVTVSS

SEQ ID NO: 63 - protein, 113 aa, synthetic construct
QVQLQESGPGLVKPSQTLSLTCTVIGYSITQGYYWNWIRQPPGKGLEWIGSINYNGSNIYNPSLKSRVTISRDTSKNQFSLKLSSVTAADTAVYYCARGGDYWGQGTTVTVSS

SEQ ID NO: 64 - protein, 113 aa, synthetic construct
QVQLQESGPGLVKPSQTLSLTCTVIGYSITSGYYWNWIRQTPGKGLEWIGSINYDGSNIYNPSLKSRVTISRDTSKNQFSLKLSSVTAADTAVYYCARGGDYWGQGTTVTVSS

SEQ ID NO: 65 - protein, 113 aa, synthetic construct
QVQLQESGPGLVKPSQTLSLTCTVIGQSITTGYYWNWIRQPPGKGLEWIGSINYDGSNIYNPSLKSRVTISRDTSKNQFSLKLSSVTAADTAVYYCARGGDPWGQGTTVTVSS

SEQ ID NO: 66 - protein, 113 aa, synthetic construct
QVQLQESGPGLVKPSQTLSLTCTVIGYSITQGYYWNWIRQPPGKGLEWIGSINYDGANIYNPSLKSRVTISRDTSKNQFSLKLSSVTAADTAVYYCARGGDYWGQGTTVTVSS

SEQ ID NO: 67 - protein, 113 aa, synthetic construct
QVQLQESGPGLVKPSQTLSLTCTVIGYSITSGYYWNWIRQSPGKGLEWIGSINYDGSNIYNPSLKSRVTISRDTSKNQFSLKLSSVTAADTAVYYCARGGDYWGQGTTVTVSS

SEQ ID NO: 68 - protein, 113 aa, synthetic construct
QVQLQESGPGLVKPSQTLSLTCTVIGYSITSSHYWNWIRQPPGKGLEWIGSINYDGSNIYNPSLKSRVTISRDTSKNQFSLKLSSVTAADTAVYYCARGGDYWGQGTTVTVSS

SEQ ID NO: 69 - protein, 113 aa, synthetic construct; identical to SEQ ID NO: 49

SEQ ID NO: 70 - protein, 113 aa, synthetic construct
QVQLQESGPGLVKPSQTLSLTCTVIGYSITSGYNWNWIRQPPGKGLEWIGSINPDGSNFYNPSLKSRVTISRDTSKNQFSLKLSSVTAADTAVYYCARGGDYWGQGTTVTVSS

SEQ ID NO: 71 - protein, 113 aa, synthetic construct; identical to SEQ ID NO: 1

SEQ ID NO: 72 - protein, 113 aa, synthetic construct; identical to SEQ ID NO: 1

SEQ ID NO: 73 - protein, 113 aa, synthetic construct
QVQLQESGPGLVKPSQTLSLTCTVIGQSITSGYYWNWIRQPPGKGLEWIGSINYNGSNIYNPSLKSRVTISRDTSKNQFSLKLSSVTAADTAVYYCARGGDPWGQGTTVTVSS

SEQ ID NO: 74 - protein, 113 aa, synthetic construct
QVQLQESGPGLVKPSQTLSLTCTVIGQSITSGYSWNWIRQPPGKGLEWIGSINHDGSNFYNPSLKSRVTISRDTSKNQFSLKLSSVTAADTAVYYCARGGNSWGQGTTVTVSS

SEQ ID NO: 75 - protein, 113 aa, synthetic construct; identical to SEQ ID NO: 1

SEQ ID NO: 76 - protein, 113 aa, synthetic construct
QVQLQESGPGLVKPSQTLSLTCTVIGYSITKGYYWNWIRQPPGKGLEWIGSINYDGSNIYNPSLKSRVTISRDTSKNQFSLKLSSVTAADTAVYYCARGGDYWGQGTTVTVSS

SEQ ID NO: 77 - protein, 113 aa, synthetic construct
QVQLQESGPGLVKPSQTLSLTCTVIGYSITSGYSWNWIRQPPGKGLEWIGSINSDGSNFYNPSLKSRVTISRDTSKNQFSLKLSSVTAADTAVYYCARGGDYWGQGTTVTVSS

SEQ ID NO: 78 - protein, 113 aa, synthetic construct; identical to SEQ ID NO: 1

SEQ ID NO: 79 - protein, 113 aa, synthetic construct
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGNSWGQGTTVTVSS

SEQ ID NO: 80 - protein, 113 aa, synthetic construct
QVQLQESGPGLVKPSQTLSLTCTVIGQSITSGYYWNWIRQPPGKGLEWIGSINYDGTNIYNPSLKSRVTISRDTSKNQFSLKLSSVTAADTAVYYCARGGRNWGQGTTVTVSS

SEQ ID NO: 81 - protein, 113 aa, synthetic construct; identical to SEQ ID NO: 49

SEQ ID NO: 82 - protein, 113 aa, synthetic construct
QVQLQESGPGLVKPSQTLSLTCTVIGQSITSGYYWNWIRQPPGKGLEWIGSISYDGSNIYNPSLKSRVTISRDTSKNQFSLKLSSVTAADTAVYYCARGGDPWGQGTTVTVSS

SEQ ID NO: 83 - protein, 113 aa, synthetic construct; identical to SEQ ID NO: 64

SEQ ID NO: 84 - protein, 113 aa, synthetic construct; identical to SEQ ID NO: 49

SEQ ID NO: 85 - protein, 113 aa, synthetic construct
QVQLQESGPGLVKPSQTLSLTCTVIGYSITQGYYWNWIRQPPGKGLEWIGSINYDGSNIYNPSLKSRVTISRDTSKNQFSLKLSSVTAADTAVYYCARGGDPWGQGTTVTVSS

SEQ ID NO: 86 - protein, 113 aa, synthetic construct
QVQLQESGPGLVKPSQTLSLTCTVIGYSITSGYYWNWIRQPPGKGLEWIGSINYDGTNIYNPSLKSRVTISRDTSKNQFSLKLSSVTAADTAVYYCARGGDYWGQGTTVTVSS

SEQ ID NO: 87 - protein, 113 aa, synthetic construct
QVQLQESGPGLVKPSQTLSLTCTVIGYSITNGYYWNWIRQPPGKGLEWIGSINYDGSNIYNPSLKSRVTISRDTSKNQFSLKLSSVTAADTAVYYCAMGGAYWGQGTTVTVSS

SEQ ID NO: 88 - protein, 113 aa, synthetic construct
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGHPWGQGTTVTVSS

SEQ ID NO: 89 - protein, 108 aa, synthetic construct; identical to SEQ ID NO: 25

SEQ ID NO: 90 - protein, 108 aa, synthetic construct; identical to SEQ ID NO: 25

SEQ ID NO: 91 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWTADPPMLTFGGGTKLEIK

SEQ ID NO: 92 - protein, 108 aa, synthetic construct; identical to SEQ ID NO: 25

SEQ ID NO: 93 - protein, 108 aa, synthetic construct; identical to SEQ ID NO: 25

SEQ ID NO: 94 - protein, 108 aa, synthetic construct; identical to SEQ ID NO: 25

SEQ ID NO: 95 - protein, 108 aa, synthetic construct; identical to SEQ ID NO: 25

SEQ ID NO: 96 - protein, 108 aa, synthetic construct; identical to SEQ ID NO: 25

SEQ ID NO: 97 - protein, 108 aa, synthetic construct; identical to SEQ ID NO: 25

SEQ ID NO: 98 - protein, 108 aa, synthetic construct; identical to SEQ ID NO: 25

SEQ ID NO: 99 - protein, 108 aa, synthetic construct; identical to SEQ ID NO: 25

SEQ ID NO: 100 - protein, 108 aa, synthetic construct; identical to SEQ ID NO: 25

SEQ ID NO: 101 - protein, 108 aa, synthetic construct; identical to SEQ ID NO: 25

SEQ ID NO: 102 - protein, 108 aa, synthetic construct; identical to SEQ ID NO: 25

SEQ ID NO: 103 - protein, 108 aa, synthetic construct; identical to SEQ ID NO: 25

SEQ ID NO: 104 - protein, 108 aa, synthetic construct
EIVLTQSPATLSLSPGERATLSCSASSSVSYMHWFQQKPGQAPRRWIYDTSIRATGVPARFSGSGSGTDYTLTISSLEPEDFAAYYCQQWTAHPPMLTFGGGTKLEIK

SEQ ID NO: 105 - protein, 108 aa, synthetic construct; identical to SEQ ID NO: 25

SEQ ID NO: 106 - protein, 108 aa, synthetic construct; identical to SEQ ID NO: 25

SEQ ID NO: 107 - protein, 108 aa, synthetic construct
EIVLTQSPATLSLSPGERATLSCSASSTVSYMHWFQQKPGQAPRRWIYDTSKRATGVPARFSGSGSGTDYTLTISSLEPEDFAAYYCQQWTDDPPMLTFGGGTKLEIK

SEQ ID NO: 108 - protein, 108 aa, synthetic construct; identical to SEQ ID NO: 25

SEQ ID NO: 109 - protein, 108 aa, synthetic construct
EIVLTQSPATLSLSPGERATLSCSASSSVSHMHWFQQKPGQAPRRWIYDTSKRATGVPARFSGSGSGTDYTLTISSLEPEDFAAYYCQQWTVDPPMLTFGGGTKLEIK

SEQ ID NO: 110 - protein, 108 aa, synthetic construct; identical to SEQ ID NO: 25

SEQ ID NO: 111 - protein, 108 aa, synthetic construct
EIVLTQSPATLSLSPGERATLSCSASSSVAYMHWFQQKPGQAPRRWIYDTSKRATGVPARFSGSGSGTDYTLTISSLEPEDFAAYYCQQWTADPPMLTFGGGTKLEIK

SEQ ID NO: 112 - protein, 108 aa, synthetic construct
EIVLTQSPATLSLSPGERATLSCSASSTVSYMHWFQQKPGQAPRRWIYDTSKRATGVPARFSGSGSGTDYTLTISSLEPEDFAAYYCQQWTADPPMLTFGGGTKLEIK

SEQ ID NO: 113 - protein, 108 aa, synthetic construct; identical to SEQ ID NO: 25

SEQ ID NO: 114 - protein, 108 aa, synthetic construct; identical to SEQ ID NO: 25

SEQ ID NO: 115 - protein, 108 aa, synthetic construct
EIVLTQSPATLSLSPGERATLSCSASQSVSYMHWFQQKPGQAPRRWIYDTSKRATGVPARFSGSGSGTDYTLTISSLEPEDFAAYYCQQWTADPPMLTFGGGTKLEIK

SEQ ID NO: 116 - protein, 108 aa, synthetic construct; identical to SEQ ID NO: 25

SEQ ID NO: 117 - protein, 108 aa, synthetic construct; identical to SEQ ID NO: 25

SEQ ID NO: 118 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWDADRRMLTFGGGTKLEIK

SEQ ID NO: 119 - protein, 108 aa, synthetic construct; identical to SEQ ID NO: 25

SEQ ID NO: 120 - protein, 108 aa, synthetic construct; identical to SEQ ID NO: 25

SEQ ID NO: 121 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTFGGGTQLEIK

SEQ ID NO: 122 - protein, 108 aa, synthetic construct; identical to SEQ ID NO: 25

SEQ ID NO: 123 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWTEDPPMLTFGGGTKLEIK

SEQ ID NO: 124 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTLGGGTKLEIK

SEQ ID NO: 125 - protein, 108 aa, synthetic construct; identical to SEQ ID NO: 25

SEQ ID NO: 126 - protein, 108 aa, synthetic construct; identical to SEQ ID NO: 25

SEQ ID NO: 127 - protein, 108 aa, synthetic construct; identical to SEQ ID NO: 25

SEQ ID NO: 128 - protein, 108 aa, synthetic construct; identical to SEQ ID NO: 25

SEQ ID NO: 129 - protein, 113 aa, synthetic construct
QVQLQESGPGLVKPSQTLSLTCTVIGYSITSGYYWNWIRQPPGKGLEWIGSINYDESNIYNPSLKSRVTISRDTSKNQFSLKLSSVTAADTAVYYCARGGDYWGQGTTVTVSS

SEQ ID NO: 130 - protein, 113 aa, synthetic construct
QVQLQESGPGLVKPSQTLSLTCTVIGYSITSGYYWNWIRQPPGKGLEWIGSINYDKSNIYNPSLKSRVTISRDTSKNQFSLKLSSVTAADTAVYYCARGGDYWGQGTTVTVSS

SEQ ID NO: 131 - protein, 113 aa, synthetic construct
QVQLQESGPGLVKPSQTLSLTCTVIGYSITSGYYWNWIRQPPGKGLEWIGSINYDASNIYNPSLKSRVTISRDTSKNQFSLKLSSVTAADTAVYYCARGGDYWGQGTTVTVSS

SEQ ID NO: 132 - protein, 113 aa, synthetic construct
QVQLQESGPGLVKPSQTLSLTCTVIGYSITSGYYWNWIRQPPGKGLEWIGSINYEGSNIYNPSLKSRVTISRDTSKNQFSLKLSSVTAADTAVYYCARGGDYWGQGTTVTVSS

SEQ ID NO: 133 - protein, 113 aa, synthetic construct
QVQLQESGPGLVKPSQTLSLTCTVIGYSITSGYYWNWIRQPPGKGLEWIGSINYQGSNIYNPSLKSRVTISRDTSKNQFSLKLSSVTAADTAVYYCARGGDYWGQGTTVTVSS

SEQ ID NO: 134 - protein, 113 aa, synthetic construct
QVQLQESGPGLVKPSQTLSLTCTVIGYSITSGYYWNWIRQPPGKGLEWIGSINYRGSNIYNPSLKSRVTISRDTSKNQFSLKLSSVTAADTAVYYCARGGDYWGQGTTVTVSS

SEQ ID NO: 135 - protein, 113 aa, synthetic construct
QVQLQESGPGLVKPSQTLSLTCTVIGYSITSGYYWNWIRQPPGKGLEWIGSINYAASNIYNPSLKSRVTISRDTSKNQFSLKLSSVTAADTAVYYCARGGDYWGQGTTVTVSS

SEQ ID NO: 136 - protein, 113 aa, synthetic construct
QVQLQESGPGLVKPSQTLSLTCTVIGYSITSGYYWNWIRQPPGKGLEWIGSINYEESNIYNPSLKSRVTISRDTSKNQFSLKLSSVTAADTAVYYCARGGDYWGQGTTVTVSS

SEQ ID NO: 137 - protein, 113 aa, synthetic construct
QVQLQESGPGLVKPSQTLSLTCTVIGYSITSGYYWNWIRQPPGKGLEWIGSINYFFSNIYNPSLKSRVTISRDTSKNQFSLKLSSVTAADTAVYYCARGGDYWGQGTTVTVSS

SEQ ID NO: 138 - protein, 113 aa, synthetic construct
QVQLQESGPGLVKPSQTLSLTCTVIGYSITSGYYWNWIRQPPGKGLEWIGSINYGGSNIYNPSLKSRVTISRDTSKNQFSLKLSSVTAADTAVYYCARGGDYWGQGTTVTVSS

SEQ ID NO: 139 - protein, 113 aa, synthetic construct
QVQLQESGPGLVKPSQTLSLTCTVIGYSITSGYYWNWIRQPPGKGLEWIGSINYHHSNIYNPSLKSRVTISRDTSKNQFSLKLSSVTAADTAVYYCARGGDYWGQGTTVTVSS

SEQ ID NO: 140 - protein, 113 aa, synthetic construct
QVQLQESGPGLVKPSQTLSLTCTVIGYSITSGYYWNWIRQPPGKGLEWIGSINYKKSNIYNPSLKSRVTISRDTSKNQFSLKLSSVTAADTAVYYCARGGDYWGQGTTVTVSS

SEQ ID NO: 141 - protein, 113 aa, synthetic construct
QVQLQESGPGLVKPSQTLSLTCTVIGYSITSGYYWNWIRQPPGKGLEWIGSINYLLSNIYNPSLKSRVTISRDTSKNQFSLKLSSVTAADTAVYYCARGGDYWGQGTTVTVSS

SEQ ID NO: 142 - protein, 113 aa, synthetic construct
QVQLQESGPGLVKPSQTLSLTCTVIGYSITSGYYWNWIRQPPGKGLEWIGSINYDNSNIYNPSLKSRVTISRDTSKNQFSLKLSSVTAADTAVYYCARGGDYWGQGTTVTVSS

SEQ ID NO: 143 - protein, 113 aa, synthetic construct
QVQLQESGPGLVKPSQTLSLTCTVIGYSITSGYYWNWIRQPPGKGLEWIGSINYPGSNIYNPSLKSRVTISRDTSKNQFSLKLSSVTAADTAVYYCARGGDYWGQGTTVTVSS

SEQ ID NO: 144 - protein, 113 aa, synthetic construct
QVQLQESGPGLVKPSQTLSLTCTVIGYSITSGYYWNWIRQPPGKGLEWIGSINYQQSNIYNPSLKSRVTISRDTSKNQFSLKLSSVTAADTAVYYCARGGDYWGQGTTVTVSS

SEQ ID NO: 145 - protein, 113 aa, synthetic construct
QVQLQESGPGLVKPSQTLSLTCTVIGYSITSGYYWNWIRQPPGKGLEWIGSINYRRSNIYNPSLKSRVTISRDTSKNQFSLKLSSVTAADTAVYYCARGGDYWGQGTTVTVSS

SEQ ID NO: 146 - protein, 113 aa, synthetic construct
QVQLQESGPGLVKPSQTLSLTCTVIGYSITSGYYWNWIRQPPGKGLEWIGSINYDTSNIYNPSLKSRVTISRDTSKNQFSLKLSSVTAADTAVYYCARGGDYWGQGTTVTVSS

SEQ ID NO: 147 - protein, 113 aa, synthetic construct
QVQLQESGPGLVKPSQTLSLTCTVIGYSITSGYYWNWIRQPPGKGLEWIGSINYVVSNIYNPSLKSRVTISRDTSKNQFSLKLSSVTAADTAVYYCARGGDYWGQGTTVTVSS

SEQ ID NO: 148 - protein, 113 aa, synthetic construct
QVQLQESGPGLVKPSQTLSLTCTVIGYSITSGYYWNWIRQPPGKGLEWIGSINYWWSNIYNPSLKSRVTISRDTSKNQFSLKLSSVTAADTAVYYCARGGDYWGQGTTVTVSS

SEQ ID NO: 149 - protein, 113 aa, synthetic construct
QVQLQESGPGLVKPSQTLSLTCTVIGYSITSGYYWNWIRQPPGKGLEWIGSINYYYSNIYNPSLKSRVTISRDTSKNQFSLKLSSVTAADTAVYYCARGGDYWGQGTTVTVSS

SEQ ID NO: 150 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDAPMLTFGGGTKLEIK

SEQ ID NO: 151 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDKPMLTFGGGTKLEIK

SEQ ID NO: 152 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSDNPMLTFGGGTKLEIK

SEQ ID NO: 153 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSEPPMLTFGGGTKLEIK

SEQ ID NO: 154 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSKPPMLTFGGGTKLEIK

SEQ ID NO: 155 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSNPPMLTFGGGTKLEIK

SEQ ID NO: 156 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSQPPMLTFGGGTKLEIK

SEQ ID NO: 157 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSRPPMLTFGGGTKLEIK

SEQ ID NO: 158 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSAAPMLTFGGGTKLEIK

SEQ ID NO: 159 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSEEPMLTFGGGTKLEIK

SEQ ID NO: 160 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSFFPMLTFGGGTKLEIK

SEQ ID NO: 161 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSGPPMLTFGGGTKLEIK

SEQ ID NO: 162 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSHHPMLTFGGGTKLEIK

SEQ ID NO: 163 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSIIPMLTFGGGTKLEIK

SEQ ID NO: 164 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSKKPMLTFGGGTKLEIK

SEQ ID NO: 165 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSLLPMLTFGGGTKLEIK

SEQ ID NO: 166 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSNNPMLTFGGGTKLEIK

SEQ ID NO: 167 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSQQPMLTFGGGTKLEIK

SEQ ID NO: 168 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSRRPMLTFGGGTKLEIK

SEQ ID NO: 169 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSSPPMLTFGGGTKLEIK

SEQ ID NO: 170 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSTTPMLTFGGGTKLEIK

SEQ ID NO: 171 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSVVPMLTFGGGTKLEIK

SEQ ID NO: 172 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSWNPMLTFGGGTKLEIK

SEQ ID NO: 173 - protein, 108 aa, synthetic construct
EIVLTQSPATLSLSPGERATLSCSASSSVSYMHWFQQKPGQAPRRWIYDTSKRATGVPARFSGSGSGTDYTLTISSLEPEDFAAYYCQQWSSYYPMLTFGGGTKLEIK

SEQ ID NO: 174 - DNA, 339 bp, synthetic construct
  source 1..339: mol_type = other DNA
gaagtgcagctgcaggagtccggacctggcctcgtgaaaccttctcagtctctgtctctcacctgctctgtcattggctactccatcaccagtggttattactggaactggatccggcagtttccaggaaacaaactggaatggatgggctccataaactacgacggtagcaatatctacaatccatctctcaaagatcgaatctccatcactcgtgacacatctaagaaccagtttttcctgaagttgaattctgtgactactgaggacacagctacatattactgtgcaagagggggggactactggggtcaaggaacctcagtcaccgtctcctca

SEQ ID NO: 175 - DNA, 351 bp, synthetic construct
  source 1..351: mol_type = other DNA
gatattgtgctgacccagactccagcaatcatgtctgcatctccaggggagaaggtcaccttgacctgcagtgccagctcaagtgtaagttacatgcactggttccagcaaaagtcaggcacctcccccaaaagatggatttatgacacatccaaactggcttctggagtccctgctcgcttcagtggcagtgggtctgggacctcttactctctcacaatcagcagcatggaggctgaggatgctgccgcttattactgccagcagtggagtagtgacccacccatgctcacgttcggtgctgggaccaagctggagctgaaacgggctgatgctgcaccaactgtatcc

SEQ ID NO: 176 - protein, 113 aa, synthetic construct
EVQLQESGPGLVKPSQSLSLTCSVIGYSITSGYYWNWIRQFPGNKLEWMGSINYDGSNIYNPSLKDRISITRDTSKNQFFLKLNSVTTEDTATYYCARGGDYWGQGTSVTVSS

SEQ ID NO: 177 - protein, 107 aa, synthetic construct
IVLTQTPAIMSASPGEKVTLTCSASSSVSYMHWFQQKSGTSPKRWIYDTSKLASGVPARFSGSGSGTSYSLTISSMEAEDAAAYYCQQWSSDPPMLTFGAGTKLELK

SEQ ID NO: 178 - protein, 25 aa, synthetic construct
IVLTQTPAIMSASPGEKVTLTCSAS

SEQ ID NO: 179 - protein, 5 aa, synthetic construct
SSVSY

SEQ ID NO: 180 - protein, 17 aa, synthetic construct
MHWFQQKSGTSPKRWIY

SEQ ID NO: 181 - skipped

SEQ ID NO: 182 - protein, 36 aa, synthetic construct
KLASGVPARFSGSGSGTSYSLTISSMEAEDAAAYYC

SEQ ID NO: 183 - protein, 11 aa, synthetic construct
QQWSSDPPMLT

SEQ ID NO: 184 - protein, 25 aa, synthetic construct
EVQLQESGPGLVKPSQSLSLTCSVI

SEQ ID NO: 185 - protein, 9 aa, synthetic construct
GYSITSGYY

SEQ ID NO: 186 - protein, 17 aa, synthetic construct
WNWIRQFPGNKLEWMGS

SEQ ID NO: 187 - protein, 7 aa, synthetic construct
INYDGSN

SEQ ID NO: 188 - protein, 38 aa, synthetic construct
IYNPSLKDRISITRDTSKNQFFLKLNSVTTEDTATYYC

SEQ ID NO: 189 - protein, 6 aa, synthetic construct
ARGGDY

SEQ ID NO: 190 - DNA, 15 bp, synthetic construct
  source 1..15: mol_type = other DNA
tcaagtgtaagttac

SEQ ID NO: 191 - DNA, 33 bp, synthetic construct
  source 1..33: mol_type = other DNA
cagcagtggagtagtgacccacccatgctcacg

SEQ ID NO: 192 - DNA, 27 bp, synthetic construct
  source 1..27: mol_type = other DNA
ggctactccatcaccagtggttattac

SEQ ID NO: 193 - DNA, 20 bp, synthetic construct
  source 1..20: mol_type = other DNA
ataaactacgacggtagcaa

SEQ ID NO: 194 - DNA, 18 bp, synthetic construct
  source 1..18: mol_type = other DNA
gcaagagggggggactac

SEQ ID NO: 195 - protein, 108 aa, synthetic construct
DIVLTQTPAIMSASPGEKVTLTCSASSSVSYMHWFQQKSGTSPKRWIYDTSKLASGVPARFSGSGSGTSYSLTISSMEAEDAAAYYCQQWSSDPPMLTFGAGTKLELK

(19) United States
(12) Patent Application Publication
(10) Pub. No.: US 2026/0306655 A1
(43) Pub. Date: Oct. 8, 2026

(54) ANTIBODY-DRUG CONJUCATES EMPLOYING NOVEL LINKER-PAYLOAD SYSTEMS FOR ENHANCED TARGETING OF CANCER-ASSOCIATED GLYCAN ANTIGENS

(71) Applicant: BioNTech SE, Mainz (DE)

(72) Inventors: Gonçalo Bernardes, Torres Vedras (PT); Nuno Prego Ramos, Ramada, Odivelas (PT); Mariangela Natale, Lisboa (PT)

(21) Appl. No.: 19/481,518

(22) PCT Filed: May 2, 2024
(86) PCT No.: PCT/EP2024/062120

§ 371 Date: Nov. 4, 2025

(30) Foreign Application Priority Data
May 5, 2023 (GB) 2306706.9
May 6, 2023 (GB) 2306730.9

(51) Int. Cl. A61K 47/68; A61P 35/00

(52) U.S. Cl. CPC A61K 47/6889; A61K 47/68037; A61K 47/6843; A61P 35/00

(57) ABSTRACT

An antibody-drug conjugate (ADC) is provided and comprises an antibody which binds to sialyl Tn (STn) or a glycan terminated by an alpha 2,6-linked sialic acid, conjugated to a drug via a linker, wherein the linker comprises a cleavable linker, and the drug comprises a growth inhibitory agent which is exatecan, deruxtecan, or a derivative thereof. The antibody-drug conjugate (ADC) of the invention comprises a linker cleavable by glucuronidase, for examples the linker comprises a β-glucuronide moiety, such as shown in formula I: Formula (I). The antibody-drug conjugate (ADC) comprises a linker which is linked to, or is modified to link to, the drug via a carbamate linkage, or a linker which is linked to, or is modified to link to, the drug via a quaternary ammonium salt linkage. The antibody-drug conjugate (ADC) may comprise a linker which is PEGylated with a group comprising polyethylene glycol (PEG). Novel linkers for use in ADCs are also described.

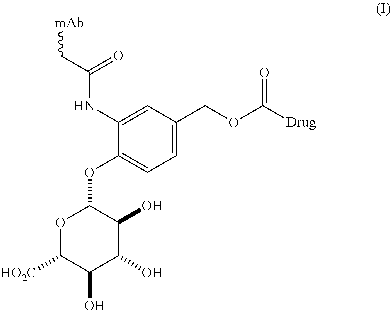

### TECHNICAL FIELD

The present invention pertains to the development of novel Antibody-Drug Conjugates (ADCs) employing monoclonal antibodies, functional antibody fragments, or probes thereof, that are specifically directed against a group of antigens, including but not limited to sialyl Tn (STn) antigens, which are highly prevalent in various types of cancer. These ADCs leverage the use of exatecan based constructs, with particular emphasis on beta-glucuronide linkers in two distinct versions, to target tumor antigens and exploit the bystander effect observed with other exatecan-based ADCs, such as trastuzumab deruxtecan, for example as described in WO2022/048883. Additionally, this invention encompasses potential applications in both human and animal health, expanding the scope of its therapeutic utility.

### STATE OF THE ART

The sialyl Tn (STn) antigen is a truncated O-glycan structure that plays a significant role in various types of carcinomas. As a disaccharide, STn consists of sialic acid (Neu5Acα) linked to N-acetylgalactosamine (GalNAc) in a 2,6 configuration, with the O-glycosidic bond formed between the GalNAc residue and either serine or threonine amino acid residues within a polypeptide chain. The presence of this truncated glycan has been detected in various carcinoma types with differing frequencies, as reported by Julian, Videira, & Delannoy (2012). Notably, STn is not found in normal healthy tissues, which underscores its relevance as a target in cancer therapy.

STn has been identified as a crucial factor in metastatic, drug-resistant, and highly malignant tumors, exhibiting several distinctive characteristics that make it a promising target for cancer treatments:
1. Association with early and metastatic cancer cells: STn expression has been linked to the early stages of cancer and metastatic cancer cells, indicating its role in tumor progression and dissemination to other sites within the body (Okasaki et al., 2012). This association highlights the potential of STn-targeting therapies in disrupting cancer progression and metastasis.
2. Correlation with poor prognosis and reduced overall survival: Increased STn expression in patients has been correlated with a poorer prognosis, reduced overall survival, and a lack of response to chemotherapy (Choi et al., 2000). This correlation suggests that STn may play a role in promoting tumor aggressiveness and therapy resistance, making it an attractive target for the development of novel cancer treatments.
3. Evasion of immune surveillance: STn has been implicated in the evasion of immune-cell surveillance, which contributes to the tumor's ability to avoid detection and elimination by the immune system (Carrascal et al., 2014). By targeting STn, novel cancer therapies may be able to overcome this immune evasion mechanism, thus enhancing the immune system's ability to recognize and eliminate tumor cells.
4. Antibody-drug conjugates (ADCs) in cancer therapy: ADCs are a class of therapeutic agents that combine the targeting specificity of antibodies with the cytotoxic potency of small molecule drugs. This targeted approach helps to minimize the impact on healthy tissues while maximizing the destruction of cancer cells. ADCs have emerged as a promising strategy for cancer treatment, with several ADCs already approved for clinical use and many more in clinical development (Chari et al., 2014; Sievers & Senter, 2013).
5. ADCs targeting STn: Some efforts have been made to develop ADCs targeting STn for cancer therapy, including the development of an ADC using a humanized anti-STn antibody conjugated to a cytotoxic drug, such as maytansinoid DM1 (SYL-001) (Li et al., 2018). However, there is still room for improvement in terms of ADCs targeting STn, including the development of new linker-payload systems that offer better stability, more efficient drug release, and improved therapeutic efficacy.
6. Exatecan linker-payload systems: Exatecan is a water-soluble, topoisomerase I inhibitor, which has shown potent antitumor activity in preclinical models and clinical trials (Kummar et al., 2006). Recently, exatecan derivatives have been explored as payloads for ADCs (e.g., DS-8201, a HER2-targeting ADC with an exatecan derivative as the payload) (Doi et al., 2017). The exatecan linker-payload systems have demonstrated favourable characteristics, such as high potency, improved stability, and efficient drug release in the tumor microenvironment, which make them attractive for ADC development.

Prendergast et al. mAbs 2017, 9 (4), 615-627, describes novel anti-sialyl-Tn monoclonal antibodies and ADCs containing them. The ADC-linker technology used is the MC-vc-PAB-MMAE. The MMAE moiety is a monomethyl auristatin, which is the growth inhibitory agent, and the MC-vc-PAB cleavable linker contains a maleimidocaproyl moiety, a valine-citrulline dipeptide moiety, and a p-aminobenzyloxycarbonyl moiety. The document also discloses a further ADC containing an MMAF moiety, which also contains a maleimidocaproyl moiety. However, none of the above linkers are cleavable by glucuronidase.

Starbuck et al. Oncotarget, 2018, 9 (33), 23289-23305, describes an ADC containing an anti-STn antibody wherein the growth inhibitory agent is a monomethyl auristatin (MMAE), and the cleavable linker is also maleimidocaproyl-valine-citrulline-p-aminobenzyloxycarbonyl. These linkers are also not cleavable by glucuronidase.

Burke et al. Molecular Cancer Therapeutics, 2016, 15 (5), 938-945, describe a glucuronide drug-linker system wherein the drug release mechanism can either be a carbamate or a quaternary ammonium). ADCs containing these linkers are also described. However, this document does not describe a linker structure including a PEG moiety.

WO2022/253035 describes ADCs wherein the linker contains a carbamate moiety and a PEG moiety, and some of the compounds also include a glucuronide moiety. However, none of these structures terminate in a maleimide moiety. Moreover, the document also does not describe a linker moiety including a quaternary ammonium moiety.

WO2022/237884 describes linker and linker-payload structures containing a quaternary ammonium moiety and glucuronide moiety, and ADCs containing this linker structure. However, this linker structure does not include a PEG moiety.

WO2018/103739 describes compounds wherein the linker contains a carbamate moiety and a glucuronide moiety, and ADCs containing this linker structure. This linker structure includes a PEG moiety, and terminates in a maleimide group. However, the maleimide-triethylene glycol moiety in the compounds described in this document is connected to the amide linker by a methylene group. It does not disclose compounds where the PEGylated moiety is of formula II as these contain an ethylene group at this position. Moreover, the document also does not describe a linker moiety including a quaternary ammonium moiety.

The present invention aims to address the need for improved antibody-drug conjugates (ADCs) targeting cancer biomarkers, by employing novel exatecan linker-payload systems in combination with novel humanized antibodies or functional antibody fragments. By fusing the specificity of these antibodies with the cytotoxic activity and bystander effect of exatecan derivatives with, in one particular aspect, two different linkers, the invention seeks to provide enhanced therapeutic efficacy and higher tumour uptake while reducing the impact of cancer therapies on healthy tissues.

ADC constructs using cancer-specific antibodies offer several advantages, contributing to their potential as effective cancer therapies and cancer diagnostics:
1. Targeted delivery: By leveraging the specificity of antibodies, ADCs can selectively bind to antigens present on the surface of cancer cells, minimizing off-target effects and sparing healthy tissues from the cytotoxic payload.
2. Enhanced potency: ADCs enable the delivery of highly potent cytotoxic agents that would be too toxic for systemic administration as free drugs. The targeted delivery to tumor cells allows for higher local concentrations of the cytotoxic payload, improving the therapeutic index and enhancing the overall potency of the treatment.
3. Reduced systemic toxicity: Due to the selective delivery of the cytotoxic payload to cancer cells, ADCs can reduce systemic exposure to the cytotoxic agent, which may lower the risk of adverse side effects commonly associated with traditional chemotherapy.
4. Synergistic effects: The combination of targeted antibody binding and potent cytotoxic payload in ADCs can lead to synergistic effects, wherein the antibody not only delivers the payload but also interferes with the recognition of ligands by host cell receptors implicated in tumor progression and immune evasion mechanisms.
5. Customizable properties: ADCs can be designed with various linker chemistries and payloads, allowing for the optimization of properties such as stability, drug release, and cytotoxic potency. This flexibility enables the development of ADCs for the treatment and detection of cancer, including theragnostics, tailored to specific cancer types, stages, or patient populations.

The invention also contemplates the use of the linkers described, in combination with other monoclonal antibodies directed to cancer antigens overexpressed in cancer cells. However, by concentrating on specific cancer biomarkers and merging the targeting capabilities of antibodies with the cytotoxic properties of exatecan linker-payload systems, the invention aims to develop more effective and targeted cancer approaches. These strategies have the potential to improve therapeutic efficacy while minimizing adverse effects on healthy tissues, enabling new detection methods and thus addressing the ongoing demand for enhanced cancer therapies.

The invention also describes a novel exatecan derivative payload with the potential of increasing safety while maintaining its efficacy profile.

### SUMMARY OF THE INVENTION

The present invention introduces an innovative Antibody-Drug Conjugate (ADC) construct designed to address the challenges in treating cancer in humans and potentially expand into animal health applications. These ADCs construct combine the specificity of antibodies or functional antibody fragments targeting Sialyl Tn (STn) and other alpha-2, 6-linked sialic acid-terminated glycans, which are known cancer biomarkers, with the potent cytotoxic activity of, for example, exatecan derivatives. These biomarkers are characterized by their overexpression in cancer cells while being absent in healthy cells.

These ADC constructs employ, in a preferred aspect, a beta-glucuronide linker in two distinct versions, connecting the antibody or functional antibody fragment to a payload such as an Exatecan payload. The antibody features protein sequences that have been engineered to optimize their performance in therapeutic applications, taking into consideration factors such as immunogenicity, pharmacokinetic profile, and binding specificity to STn. This enables the precise detection of tumor cells and the potential to interfere with the recognition of ligands by host cell receptors that contribute to tumor progression and immune evasion mechanisms.

By employing an affinity maturation process, the invention further develops high-affinity antibodies or functional antibody fragments targeting STn, thereby improving the antitumor response. This process results in a series of new and distinct antibodies or functional antibody fragments, including a clone denoted as mAb_v1, which exhibit increased affinity and binding to the target STn.

The ADCs with a payload such as exatecan targeting STn exploit the bystander effect, as observed in other exatecan-based ADCs such as trastuzumab deruxtecan, to enhance therapeutic efficacy. The bystander effect allows the exatecan payload to diffuse into neighbouring cells, effectively targeting not only the cells expressing the cancer biomarkers but also those in the surrounding tumor microenvironment. This leads to improved tumor cell killing, while minimizing the impact on healthy tissues.

In the present invention, in one aspect, an alternative embodiment of the ADC construct is disclosed, which involves replacing the carbamate handle/linker with a quaternary amine as the handle of the beta-glucuronide linker to a payload such as exatecan. This modification brings additional advantages and benefits to the ADC's properties and efficacy.

It has been found that using a quaternary amine as the handle of the beta-glucuronide linker provides an extra layer of safety by ensuring that the cytotoxic payload maintains a positive charge when cleaved outside the tumor cells. This positive charge effectively inhibits the internalization of the circulating payload by healthy cells, thereby reducing the risk of off-target toxicity and improving the ADC's overall safety profile.

Additionally, the quaternary amine handle/linker can enhance the ADC's stability in circulation, ensuring that the payload remains securely attached to the antibody until it reaches the tumor microenvironment. Once inside the tumor cells, the linker is cleaved by the action of intracellular enzymes, releasing the active payload specifically within the cancer cells. This selective release mechanism ensures that the cytotoxic payload is delivered primarily to the intended target cells while minimizing exposure to healthy tissues and reducing the risk of adverse effects.

Furthermore, the quaternary amine handle/linker can potentially improve the ADC's pharmacokinetic properties, as the positive charge may facilitate the interaction with negatively charged components in the tumor microenvironment. This could potentially lead to enhanced tumor penetration and retention of the ADC, resulting in improved therapeutic efficacy.

In summary, in one aspect, disclosed herein is the introduction of a quaternary amine handle in place of the carbamate handle/linker in the ADC construct, which offers several advantages, including improved safety, enhanced stability in circulation, and potentially better pharmacokinetics. These features contribute to the overall efficacy and safety of the ADC, further supporting its potential as a promising cancer therapy for both human and animal health applications. Other aspects, including further improved linker design, and a novel exatecan derivative are disclosed herein.

Furthermore, the present invention's scope extends to potential applications in animal health, where the optimized antibodies or functional antibody fragments can be adapted for use in the treatment of cancer in animals, subject to necessary modifications and validations. This broadens the potential impact of this novel ADC construct in addressing the challenges faced in both human and animal cancer treatment.

In summary, the present invention provides versatile ADC constructs that combine antibodies or functional antibody fragments targeting STn with, in a preferred aspect, Exatecan payloads or derivatives thereof using beta-glucuronide linkers, capitalizing on the bystander effect to enhance therapeutic efficacy and specificity in cancer treatment. The invention's potential to extend into animal health applications further underscores its significance in addressing the challenges faced in cancer therapy. Certain novel linkers are also provided, and these have the potential to be used with a range of different antibodies and payloads in addition to the antibodies and payloads specifically described herein.

### BRIEF DESCRIPTION OF FIGURES

FIG. 1a shows synthetic route of linker-payload Version1 (ExV1) synthesis for the compounds of the invention containing a carbamic linkage-based glucuronide drug linker.

FIG. 1b shows synthetic route of linker-payload Version2 (ExV2) synthesis. Quaternary ammonium linkage-based glucuronide drug linker.

FIG. 2a shows ADCs features listed with relative ID, scale (mg), BCA Conc. (mg/ml), Yield (mg), Recovery (%), MS-DAR, HIC-DAR, SEC monomer %, free drug level % and Endo (EU/mg). Final ADCs produced were ADC-AFI-ExV1, ADC-IgG1-ExV1 and ADC-AFI-DXd.

FIG. 2b shows SEC profile represented as response [mAU] y-axis and retention time [min] x-axis for final ADC-AFI-ExV1, ADC-IgG1-ExV1 and ADC-AFI-DXd.

FIG. 2c shows reduced MS data of second batch of final product ADCs including ADC-AFI-ExV1, ADC-IgG1-ExV1 and ADC-AFI-DXd.

FIG. 3 shows summary of endotoxin levels in ADC-AFI-ExV1, ADC-IgG1-ExV1 and ADC-AFI-DXd.

FIGS. 4a-b show the efficacy study using CDX-SNU16 (Gastric Cancer cell model). A Tumor Growth curves of different treatment groups including Vehicle treated mice; ADC-IgG1-ExV1 (Isotype control); ADC-AFI-DXd and ADC-AFI-ExV1 (novel linker payload Version1). All ADCs used were at DAR4. Female BALB/c Nude mice bearing SNU-16 established tumors were used. I.V., PG-D0, D18; Mice per group used, n=7. B Head to head comparison of ADC-AFI-ExV1 (novel linker payload Version1) versus ADC-AFI-DXd (known linker payload) using same dosing [8 mg/Kg] dose and same DAR=4. Unpaired T-test analysis. Bars represent mean±S.E.M. *P<0.01.

FIG. 4c-d shows D Body Weight Change (%) and D Body Weight gain or loss relative to efficacy study using mice bearing SNU-16 established tumors. I.V., PG-DO, D18; Mice per group used, n=7. Error bars represent standard error of the mean (SEM).

FIG. 5a-b shows A the biodistribution analysis over time and B relative mAb uptake detected as % ID/g at 96 hs of tumor breast cancer cell line 4T1-STn and relative parental WT line as control (4T1-WT). A PET images with the same heat scale. B Distribution in all groups; One-way ANOVA followed by Bonferroni post-hoc test. Bars represent mean±S.E.M. ***P<0.0001.

FIG. 6a-c summarize different developability analysis of first and second generation humanized clones compared to benchmark mAbs. A Absorbance detected at A450-A620 using ELISA for DNA, LPS, Lysozyme and Cell lysate, using CBS 2nd and 1st Generation antibody, together with control Palivizumab and Trastuzumab. Data normalized to Palivizumab. B Mean of Stickiness analysis compared to Palivizumab. C Antibody signal [mV] vs retention time [min] of Palivizumab, mAb_v64 and mAb_v1, using pH3 and temperature stress (48 h at 45° C.).

FIG. 7a-d present antibody internalization assay using several cancer cell lines expressing different level of STn. MDA-MB-231-STn+ (High); MDA-MB-231-WT (null; control); SNU16 (Intermediate/High) and COLO205 (Low) cell line. Antibody internalization was reported as internalization factor normalized to IgG1 control mAb Palivizumab. Different antibody anti-STn were employed including positive control mAb_PC (anti-STn); mAb_v57; mAb_v48; mAb_v46; mAb_v53; mAb_v25; mAb_v64; mAb_v1 and parental L2A5 mAb.

FIG. 8 shows EC50 assay using cancer cell lines expressing different level of STn. COLO205 (Low); SNU16 (Intermediate/High) and OV90 (High) cell line. Different antibody anti-STn were used including positive control mAb_PC (anti-STn); mAb_v1; mAb_v46; mAb_v53; mAb_v25 and mAb_v64.

FIG. 9 shows the sequence ID numbers assigned to the variants disclosed herein.

FIG. 10a shows the amino acid sequence of the VH heavy chain of humanised variants disclosed herein. The H-CDR1, H-CDR2 and H-CDR3 sequences are highlighted in bold.

FIG. 10b shows the amino acid sequence of the VL light chain of humanised antibody variants disclosed herein. The L-CDR1, L-CDR2 and L-CDR3 sequences are highlighted in bold.

FIG. 11a shows the amino acid sequence of the VH heavy chain of affinity-matured variants disclosed herein. The H-CDR1, H-CDR2 and H-CDR3 sequences are highlighted in bold.

FIG. 11b shows the amino acid sequence of the VL light chain of affinity-matured variants disclosed herein. The L-CDR1, L-CDR2 and L-CDR3 sequences are highlighted in bold.

FIG. 12 shows the amino acid sequences of the variable chains (VH and VL) of the affinity-matured variants disclosed herein, together with the clone variant name and their corresponding sequence identity no.

FIG. 13a shows the sequence ID No and amino acid sequence of the VH heavy chain of additional humanised V1 variants disclosed herein. The H-CDR1, H-CDR2 and H-CDR3 sequences are highlighted in bold.

FIG. 13b shows the sequence ID No and amino acid sequence of the VL light chain of additional humanised V1 variants disclosed herein. The L-CDR1, L-CDR2 and L-CDR3 sequences are highlighted in bold.

FIG. 14 shows sequence ID No and amino acid and polynucleotide sequences for certain mouse antibodies disclosed herein.

FIG. 15a shows HPLC relative to Compound 2

FIG. 15b shows LCMS relative to Compound 2

FIG. 16a shows HPLC relative to Compound 3

FIG. 16b shows LCMS relative to Compound 3

FIG. 17a shows HPLC relative to Compound 4

FIG. 17b shows LCMS relative to Compound 4

FIG. 18a shows HPLC relative to Compound 5

FIG. 18b shows LCMS relative to Compound 5

FIG. 19a shows HPLC relative to Compound 6

FIG. 19b shows LCMS relative to Compound 6

FIG. 20a shows HPLC relative to Compound 7

FIG. 20b shows LCMS relative to Compound 7

FIG. 21a shows HPLC relative to Compound 8

FIG. 21b shows LCMS relative to Compound 8

FIG. 22a shows HPLC relative to Compound 8 and 1a-1

FIG. 22b shows LCMS relative to Compound 8 and 1a-1

FIG. 23a shows HPLC relative to Compound 6a-1

FIG. 23b shows LCMS relative to Compound 6a-1

FIG. 24a shows HPLC relative to Compound 9

FIG. 24b shows LCMS relative to Compound 9

FIG. 25a shows HPLC relative to Compound 10

FIG. 25b shows LCMS relative to Compound 10

FIG. 26a shows HPLC relative to Compound 11

FIG. 26b shows LCMS relative to Compound 11

FIG. 27a shows HPLC relative to Compound 11 and 1a-1

FIG. 27b shows LCMS relative to Compound 11 and 1a-1

FIG. 28 shows a list of abbreviations relative to linker-payload analysis.

FIG. 29a presents the summary of pilot conjugations including ADC-AFI-ExV1 (1-01/02 and 03) with relative TCEP/mAb ratio; Drug/mAb ratio, monomer %; HIC-DAR and reduced MS-DAR data.

FIG. 29b shows HIC Antibody response [mAU] y-axis and retention time [min] x-axis with relative SEC profile for pilot conjugations showed as response [mAU] y-axis and retention time [min] x-axis for ADC-AFI-ExV1 (1-01/02 and 03).

FIG. 30a shows reduced LC-MS data for pilot conjugations in ADC-AFI-ExV1 (1-01).

FIG. 30b shows reduced LC-MS data for pilot conjugations in ADC-AFI-ExV1 (1-02).

FIG. 30c shows reduced LC-MS data for pilot conjugations in ADC-AFI-ExV1 (1-03).

FIG. 31 shows Curve of TCEP/mAb ration vs reduced MS-DAR.

FIG. 32a present ADC-AFI-ExV1 (Test1) sample features listed in table.

FIG. 32b shows SEC profile for ADC-AFI-ExV1 (Test1) conjugations represented as response [mAU] y-axis and retention time [min] x-axis.

FIG. 32c shows reduced MS data of the confirming conjugation of ADC-AFI-ExV1 (Test1).

FIG. 33a presents the ADC-AFI-ExV1 with relative ID, scale (mg), TCEP/mAb ratio, Drug/mAb ratio, monomer and MS-DAR.

FIG. 33b presents the SEC profile for final ADC-AFI-ExV1 conjugations represented as response [mAU] y-axis and retention time [min] x-axis.

FIG. 33c shows reduced MS data of the confirming conjugation of final ADC-AFI-ExV1.

FIG. 34a presents ADC-AFI-ExV1 with relative ID, scale (mg), TCEP/mAb ratio, Drug/mAb ratio, monomer and MS-DAR.

FIG. 34b presents SEC profile for final ADC-AFI-ExV1 conjugations represented as response [mAU] y-axis and retention time [min] x-axis.

FIG. 34c shows reduced MS data of the confirming conjugation of final ADC-AFI-ExV1.

FIG. 35a shows ADC-AFI-ExV1 bulk conjugation product (15 mg) with relative ID, scale (mg), TCEP/mAb ratio, OD at 280 nm, UV Con. (mg/ml), MS-DAR and SEC monomer %.

FIG. 35b shows SEC profile for final ADC-AFI-ExV1 conjugations represented as response [mAU] y-axis and retention time [min] x-axis.

FIG. 35c presents reduced MS data of second batch of bulk conjugation for ADC-AFI-ExV1.

FIG. 36 shows RP data of final ADC products (370 nm).

FIG. 37 shows HIC relative to ADC-AFI-DXd

FIG. 38 presents FACS staining of SNU16 using mAb_V64 versus IgG1 (isotype control) before cell injection into Nude BALB/C mice. SNU16 present 99.5% STn staining using mAb_V64.

FIGS. 39a-b show A CDX-SNU16 (Gastric Cancer cell model) Efficacy treatment using clinical validate vedotin platform (Linker: Val-Cit-PAB; Payload: MMAE; Conjugation: Maleimide random Cys). Antibodies used were AFI (mAb_v64); human positive control anti STn (PC). Tumor Growth curves of different treatment groups including Vehicle treated mice; ADC-IgG1-MMAE (Isotype control); ADC-AFI-MMAE and ADC-PC-MMAE. AII ADCs used were at DAR=4. B Body Weight of mice relative to same efficacy treatment. Female BALB/c Nude mice bearing SNU-16 established tumors were used. Dosing schedule [2 mg/Kg] PG-D0, D7, D14 18; [3.5 mg/Kg] PG-D18, D25). I.P. weekly; mice per group used, n=7.

FIG. 40 shows the biodistribution analysis of different other organs in same experimental 4T1 syngeneic mouse-mice. Data represented as relative % ID/g at 96 hs.

### BRIEF DESCRIPTION OF TABLES

Table 1. ADC-AFI-ExV1, ADC-IgG1-ExV1 and ADC-AFI-DXd with relative amount (mg), antibody, linker payload and target DAR.

Table 2. SEC-HPLC Method.

Table 3. HIC-HPLC Method.

Table 4. LC-MS Method.

Table 5. Linker-payload Standard Curve Preparation.

Table 6. RP-HPLC method for free drug test.

Table 7. Conjugation and formulation buffer.

Table 8 Description of experimental design. N: number of animals per group; Dose volume: adjust dosing volume based on body weight 10 μL/g.

### OVERVIEW OF THE INVENTION

In a broad aspect, the present invention provides an antibody-drug conjugate (ADC) comprising an antibody which binds to sialyl Tn (STn) or a glycan terminated by an alpha 2,6-linked sialic acid, conjugated to a drug via a linker, wherein the linker comprises a cleavable linker, and the drug comprises a growth inhibitory agent. The growth inhibitory agent may for example be an anti-cancer agent, such as a chemotherapeutic agent or cytotoxic agent. The growth inhibitory agent may for example be exatecan or a derivative thereof, or deruxtecan or a derivative thereof.

In one aspect, the invention provides an antibody-drug conjugate (ADC) comprising an antibody which binds to sialyl Tn (STn) or a glycan terminated by an alpha 2,6-linked sialic acid, conjugated to a drug via a linker, wherein the linker comprises a linker cleavable by glucuronidase, and the drug comprises a growth inhibitory agent.

In one aspect, the linker used in the ADC comprises a linker cleavable by glucuronidase. The linker may for example be cleavable by a glucuronidase from a human or animal source, that is, a human or animal enzyme.

In one aspect, the linker comprises a β-glucuronide moiety.

The antibody-drug conjugate (ADC) may for example comprise a linker comprising a β-glucuronide moiety as shown in formula l′:

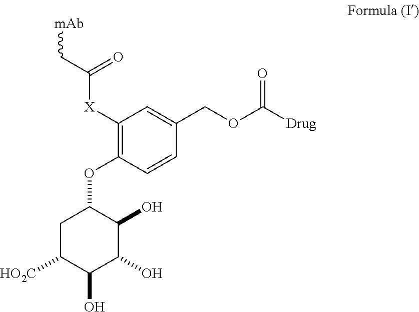

wherein X is NH, N—CH₃ or CF₂.

In one embodiment of formula (I′), X is NH. In one embodiment of formula (I′), X is N—CH₃. In one embodiment of formula (I′), X is CF₂.

The antibody-drug conjugate (ADC) may for example comprise a linker comprising a β-glucuronide moiety as shown in formula I:

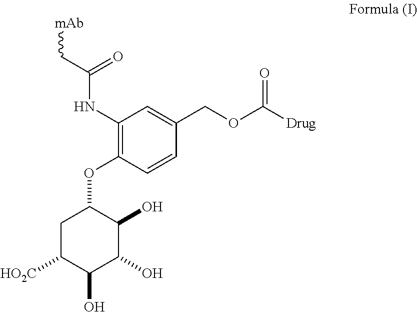

Although it will be understood that the specific formula shown above may be modified so long as it remains cleavable by glucuronidase.

In one aspect, therefore, the antibody-drug conjugate (ADC) according to the invention may comprise a linker which is linked to the drug via a carbamate linkage, as for example shown in formula I′ or I.

In a further aspect, the carbamate linkage may be modified or replaced by another chemical moiety.

In a preferred aspect, the antibody-drug conjugate (ADC) provided herein comprises a linker wherein the linker is linked to, or is modified to link to, the drug via a quaternary ammonium salt linkage. Thus, for example, in formula I above the carbamate linkage or moiety may be replaced with a quaternary ammonium salt linkage or moiety. One suitable quaternary ammonium salt linkage or moiety is shown illustrated in formula IV or VI below, it being understood that this linkage or moiety could be employed in linker structures other than those specifically shown in formula IV or VI.

According to a further aspect of the present invention, the linker used in the antibody-drug conjugate (ADC) may be PEGylated with a group or moiety comprising polyethylene glycol (PEG), that is one or more polyethylene glycol (PEG) units. The group may comprise one or more PEG units alone, or one of more PEG units may be incorporated within a larger chemical group.

In one preferred aspect, the ADC comprises a linker which comprises a β-glucuronide moiety PEGylated with a group comprising polyethylene glycol (PEG).

In a particularly preferred aspect, the ADC comprises a linker which comprises a β-glucuronide moiety PEGylated with a group comprising polyethylene glycol (PEG), wherein the linker is also linked to the drug via a quaternary ammonium salt linkage or moiety.

In one aspect, the linker may be PEGylated with a group comprising polyethylene glycol (PEG) based on the structure:

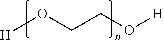

wherein n is from 1 to 5,
   although if desired one or both of the terminal H substituents in the above structure may be replaced by another suitable chemical group, provided that the functioning of the linker is substantially unaffected.

In a preferred aspect, in the structure above n is 2 to 4. In a more preferred aspect, in the structure above n is 3, that is, the PEG comprises three individual monomer units.

In one preferred aspect, the invention provides an antibody-drug conjugate (ADC) comprising an antibody as defined herein, conjugated to a drug via a linker, wherein the linker comprises a group of formula IIA:

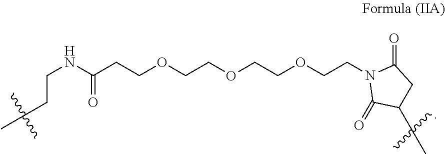 [SMILES: *CCNC(=O)CCOCCOCCOCCN1C(=O)CC(*)C1=O]

In one preferred aspect, the antibody-drug conjugate (ADC) comprises a linker which is PEGylated with a group of formula II:

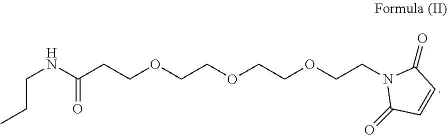 [SMILES: CCCNC(=O)CCOCCOCCOCCN1C(=O)C=CC1=O]

The PEGylation may for example be on the amide group of the linker, for example on the amide group of the structure shown in formula I. In a preferred aspect, a linker comprising a group of formula II is also linked to the drug via a quaternary ammonium salt linkage or moiety.

In one preferred aspect, the invention provides an antibody-drug conjugate (ADC) comprising an antibody as defined herein, conjugated to a drug via a linker, wherein the linker is a group as shown in formula IIIA′:

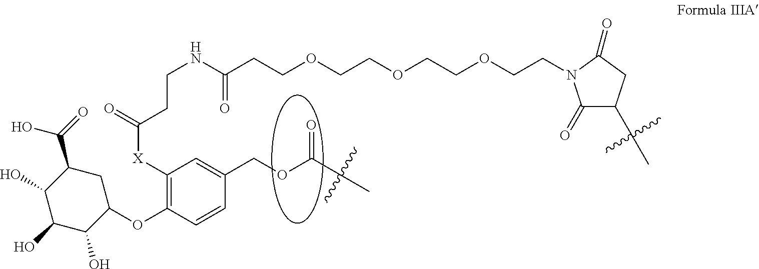

wherein X is NH, N—CH₃ or CF₂.

In one embodiment of formula IIIA′, X is NH. In one embodiment of formula IIIA′, X is N—CH₃. In one embodiment of formula IIIA′, X is CF₂.

In one preferred aspect, the invention provides an antibody-drug conjugate (ADC) comprising an antibody as defined herein, conjugated to a drug via a linker, wherein the linker is a group as shown in formula IIIA:

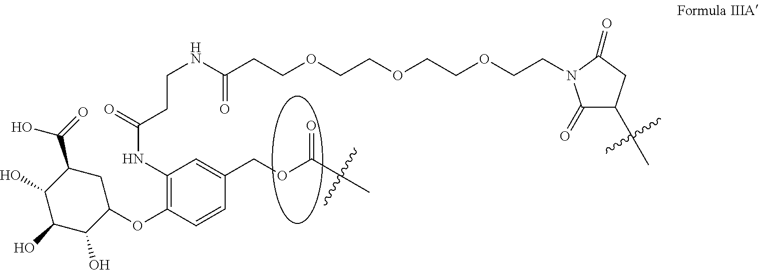 [SMILES: *C(=O)OCc1ccc(OC2C[C@H](C(=O)O)[C@@H](O)[C@H](O)[C@H]2O)c(NC(=O)CCNC(=O)CCOCCOCCOCCN2C(=O)CC(*)C2=O)c1]

Thus, in one preferred example, the antibody-drug conjugate (ADC) according to the invention may comprise a linker which is a PEGylated linker as shown in formula III:

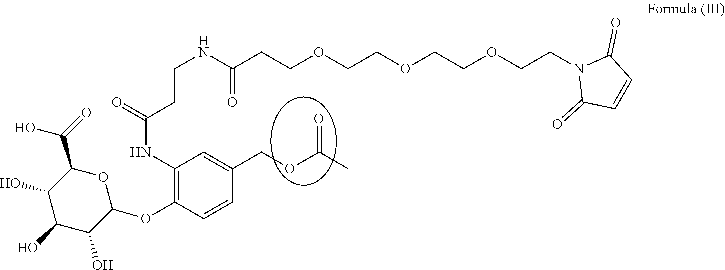 [SMILES: CC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

In one preferred embodiment, the invention provides an antibody-drug conjugate (ADC) comprising an antibody as defined herein conjugated to a drug via a linker, wherein the linker is as shown in formula IVA′:

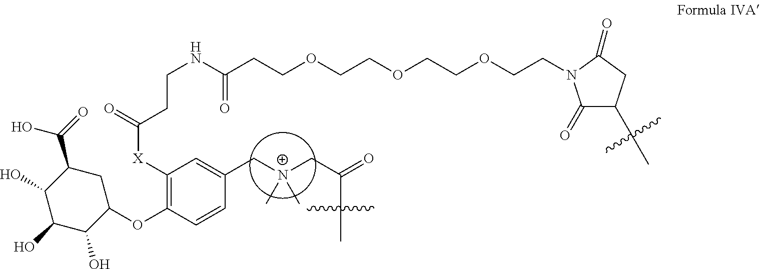

wherein X is NH, N—CH₃ or CF₂.

In one embodiment of formula IVA′, X is NH. In one embodiment of formula IVA′, X is N—CH₃. In one embodiment of formula IVA′, X is CF₂.

In one preferred embodiment, the invention provides an antibody-drug conjugate (ADC) comprising an antibody as defined herein conjugated to a drug via a linker, wherein the linker is as shown in formula IVA:

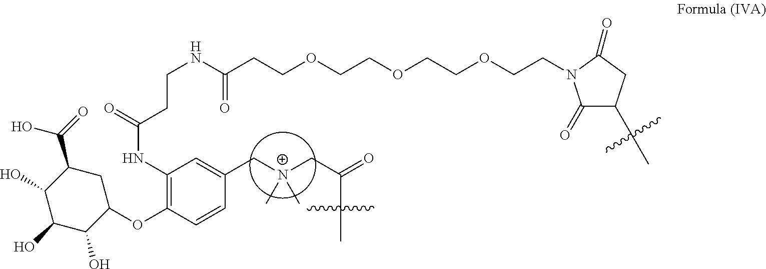 [SMILES: *C(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)CC(*)C2=O)c1]

In another preferred example, the antibody-drug conjugate (ADC) according to the invention may comprise a linker which is a PEGylated linker as shown in formula IV:

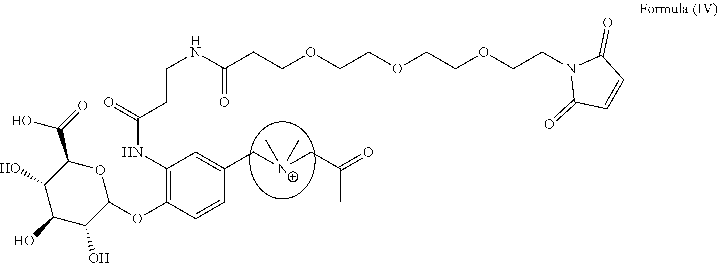 [SMILES: CC(=O)C[N+](C)(C)Cc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

In one preferred embodiment, the antibody-drug conjugate (ADC) of the invention comprises an antibody as defined herein, conjugated to a drug via a linker, wherein the drug-linker moiety of the ADC is as shown in formula VA′:

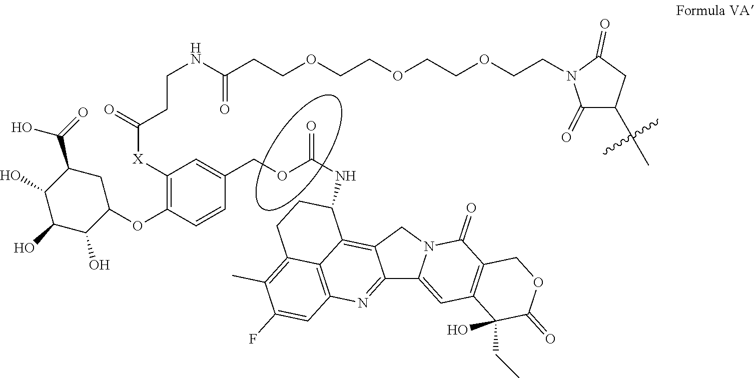

wherein X is NH, N—CH₃ or CF₂.

In one embodiment of formula VA′, X is NH. In one embodiment of formula VA′, X is N—CH₃. In one embodiment of formula VA′, X is CF₂.

In one preferred embodiment, the antibody-drug conjugate (ADC) of the invention comprises an antibody as defined herein conjugated to a drug via a linker, wherein the drug-linker moiety of the ADC is as shown in formula VA:

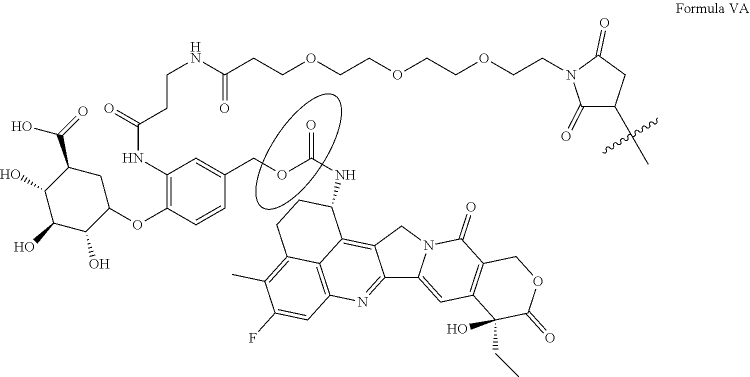 [SMILES: *C1CC(=O)N(CCOCCOCCOCCC(=O)NCCC(=O)Nc2cc(COC(=O)N[C@H]3CCc4c(C)c(F)cc5nc6c(c3c45)Cn3c-6cc4c(c3=O)COC(=O)[C@]4(O)CC)ccc2OC2C[C@H](C(=O)O)[C@@H](O)[C@H](O)[C@H]2O)C1=O]

In one preferred embodiment, the antibody-drug conjugate (ADC) of the invention comprises an antibody as defined herein conjugated to a drug via a linker, wherein the drug-linker moiety of the ADC is as shown in formula VIA′:

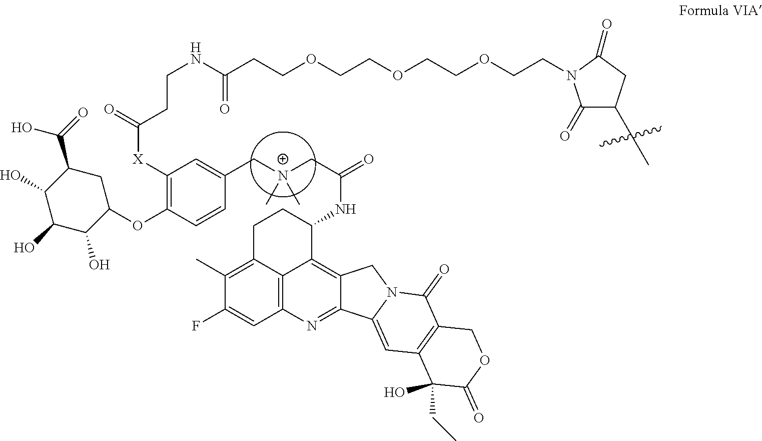

wherein X is NH, N—CH₃ or CF₂.

In one embodiment of formula VIA′, X is NH. In one embodiment of formula VIA′, X is N—CH₃. In one embodiment of formula VIA′, X is CF₂.

In one preferred embodiment, the antibody-drug conjugate (ADC) of the invention comprises an antibody as defined herein conjugated to a drug via a linker, wherein the drug-linker moiety of the ADC is as shown in formula VIA:

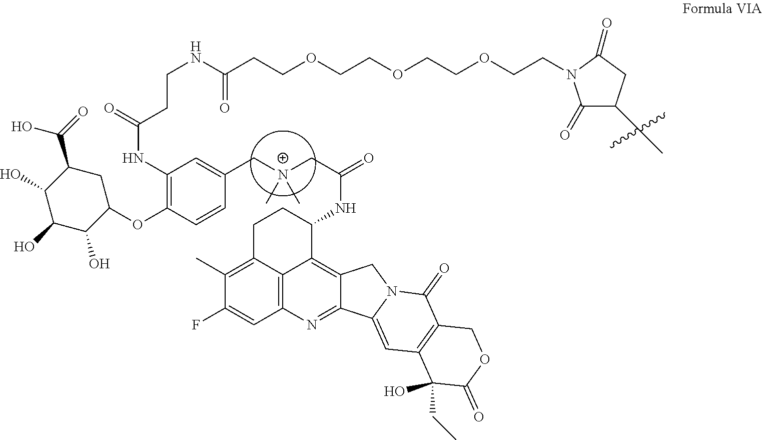 [SMILES: *C1CC(=O)N(CCOCCOCCOCCC(=O)NCCC(=O)Nc2cc(C[N+](C)(C)CC(=O)N[C@H]3CCc4c(C)c(F)cc5nc6c(c3c45)Cn3c-6cc4c(c3=O)COC(=O)[C@]4(O)CC)ccc2OC2C[C@H](C(=O)O)[C@@H](O)[C@H](O)[C@H]2O)C1=O]

In one preferred aspect, the antibody-drug conjugate (ADC) according to the invention comprises a drug-linker moiety or payload comprising the structure as shown in formula V or VI:

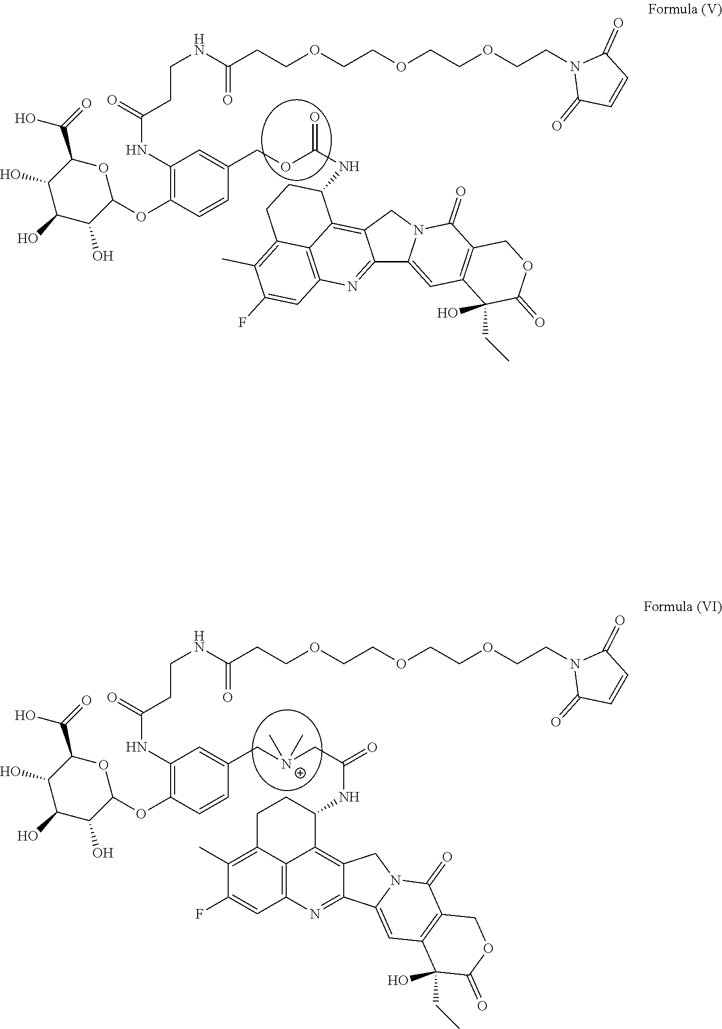

According to the present invention, the antibody-drug conjugate (ADC) as described herein the sialyl Tn (STn) or a glycan terminated by an alpha 2,6-linked sialic acid to which the ADC binds is a human or animal protein.

In a preferred aspect, the drug or payload employed in the antibody-drug conjugate (ADC) as described herein is exatecan.

### Drug-Antibody Ratio

In one embodiment, the ADC according to the invention has a drug-antibody ratio (DAR) which is an integer from 1 to 10, such as 1, 2, 3, 4, 5, 6, 7, 8, 9 or 10. In one embodiment, the ADC according to the invention has a drug-antibody ratio (DAR) which is an integer from 1 to 8. In one embodiment, the ADC according to the invention has a drug-antibody ratio (DAR) which is an integer from 2 to 6. In one embodiment, the ADC according to the invention has a drug-antibody ratio (DAR) which is an integer from 3 to 5. In one embodiment, the ADC according to the invention has a drug-antibody ratio (DAR) which is 2. In one embodiment, the ADC according to the invention has a drug-antibody ratio (DAR) which is 3. In one embodiment, the ADC according to the invention has a drug-antibody ratio (DAR) which is 4. In one embodiment, the ADC according to the invention has a drug-antibody ratio (DAR) which is 5.

In another aspect, there is provided a composition comprising an ADC according to the invention or a mixture thereof. As is apparent to the person skilled in the art, such a composition may contain a mixture of ADCs having different DARs, such that the DAR of the composition is expressed as an average DAR which may be non-integral. In one embodiment, the composition according to the invention has an average drug-antibody ratio (DAR) which is an integer or decimal from 1 to 10. In one embodiment, the composition according to the invention has an average drug-antibody ratio (DAR) which is an integer or decimal from 1 to 8. In one embodiment, the composition according to the invention has an average drug-antibody ratio (DAR) which is an integer or decimal from 2 to 6. In one embodiment, the composition according to the invention has an average drug-antibody ratio (DAR) which is an integer or decimal from 3 to 5. In one embodiment, the composition according to the invention has an average drug-antibody ratio (DAR) which is about 2.0. In one embodiment, the composition according to the invention has an average drug-antibody ratio (DAR) which is about 2.1. In one embodiment, the composition according to the invention has an average drug-antibody ratio (DAR) which is about 2.2. In one embodiment, the composition according to the invention has an average drug-antibody ratio (DAR) which is about 2.3. In one embodiment, the composition according to the invention has an average drug-antibody ratio (DAR) which is about 2.4. In one embodiment, the composition according to the invention has an average drug-antibody ratio (DAR) which is about 2.5. In one embodiment, the composition according to the invention has an average drug-antibody ratio (DAR) which is about 2.6. In one embodiment, the composition according to the invention has an average drug-antibody ratio (DAR) which is about 2.7. In one embodiment, the composition according to the invention has an average drug-antibody ratio (DAR) which is about 2.8. In one embodiment, the composition according to the invention has an average drug-antibody ratio (DAR) which is about 2.9. In one embodiment, the ADC according to the invention has an average drug-antibody ratio (DAR) which is about 3 . . . . In one embodiment, the ADC according to the invention has an average drug-antibody ratio (DAR) which is about 3.1. In one embodiment, the ADC according to the invention has an average drug-antibody ratio (DAR) which is about 3.2. In one embodiment, the ADC according to the invention has an average drug-antibody ratio (DAR) which is about 3.3. In one embodiment, the ADC according to the invention has an average drug-antibody ratio (DAR) which is about 3.4. In one embodiment, the ADC according to the invention has an average drug-antibody ratio (DAR) which is about 3.5. In one embodiment, the ADC according to the invention has an average drug-antibody ratio (DAR) which is about 3.6. In one embodiment, the ADC according to the invention has an average drug-antibody ratio (DAR) which is about 3.7. In one embodiment, the ADC according to the invention has an average drug-antibody ratio (DAR) which is about 3.8. In one embodiment, the ADC according to the invention has an average drug-antibody ratio (DAR) which is about 3.9. In one embodiment, the ADC according to the invention has an average drug-antibody ratio (DAR) which is about 4 . . . . In one embodiment, the ADC according to the invention has an average drug-antibody ratio (DAR) which is about 4.1. In one embodiment, the ADC according to the invention has an average drug-antibody ratio (DAR) which is about 4.2. In one embodiment, the ADC according to the invention has an average drug-antibody ratio (DAR) which is about 4.3. In one embodiment, the ADC according to the invention has an average drug-antibody ratio (DAR) which is about 4.4. In one embodiment, the ADC according to the invention has an average drug-antibody ratio (DAR) which is about 4.5. In one embodiment, the ADC according to the invention has an average drug-antibody ratio (DAR) which is about 4.6. In one embodiment, the ADC according to the invention has an average drug-antibody ratio (DAR) which is about 4.7. In one embodiment, the ADC according to the invention has an average drug-antibody ratio (DAR) which is about 4.8. In one embodiment, the ADC according to the invention has an average drug-antibody ratio (DAR) which is about 4.9. In one embodiment, the ADC according to the invention has an average drug-antibody ratio (DAR) which is about 5.0.

### Antibody

In one aspect, the antibody employed in the ADC is preferably a monoclonal antibody. The antibody may for example be an antibody fragment, for example a functional antibody fragment. In one aspect, the antibody fragment may be selected from the group consisting of Fv, Fab, F(ab′)2, Fab′, dsFv, (dsFv)2, scFv, sc(Fv)2, and diabodies.

The antibody employed in the antibody-drug conjugate (ADC) as described herein may for example be a human or animal antibody, or fragment thereof. The present invention is envisaged to be of utility in both human and animal applications. Thus both medical and veterinary treatments are within the scope of this invention.

In one aspect, in the antibody-drug conjugate (ADC) as described herein, the glycans terminated by alpha 2,6-linked sialic acids comprise STn, 2,6-sialyl T, di-sialyl T, or 2,6-sialolactosamine.

In another aspect, in the antibody-drug conjugate (ADC) as described herein, the antibody is subject to glycan changes at glycosylation sites.

In another aspect, in the antibody-drug conjugate (ADC) as described herein, the antibody is a monoclonal antibody, a chimeric antibody, or a humanized antibody.

In another aspect, in the antibody-drug conjugate (ADC) as described herein, the antibody is a functional antibody fragment that binds STn and a group of glycans terminated by alpha 2,6-linked sialic acids.

In a further aspect of the invention, one advantage of the present antibody-drug conjugates (ADCs) as described herein is that they enable the drug antibody ratio (DAR) to be kept low, whilst still maintaining high efficacy. Thus, in one aspect, there is provided an antibody-drug conjugate (ADC) as described herein wherein the DAR is about 4 or less. Preferably, the antibody component is coupled to the linker-payload via cysteine conjugation, although in principle any suitable coupling may be used.

We have found certain antibodies to be of particular interest and utility in the ADC of the present invention, and these antibodies specifically bind to either human or animal antigenic protein which is, or comprises, sialyl Tn (STn) or a glycan terminated by an alpha 2,6-linked sialic acid. Certain preferred antibodies of this type are described further below. These may for example be specifically coupled to the drug-linker moieties or payloads described above.

Preferred antibodies include those described in our co-pending patent application WO2023/249502. This application describes new antibodies which have improved antibody affinity and binding to the target, Sialyl Tn (STn). These are related to the clone described in patent WO2019/147152A1. In particular, the present inventors have provided a number of different humanised antibody clones, including the humanized clone referred to herein with the acronym of V1. Using in particular this humanized V1 clone, the present inventors have also now provided a series of new and different antibodies resulting from an affinity maturation process. These new clones or variants possess differences in the amino acid sequences and an increase in affinity and binding to the target Sialyl Tn (STn), thus substantially improving over known antibodies.

The present application thus includes the use of these high affinity humanized anti-STn antibodies obtained through a process of affinity maturation, in an ADC. For certain applications, it is highly desirable to have antibodies with high affinity to the target antigen, improving the antitumor response. For instance, this may allow for:
antibody-drug conjugates or radioimmunoconjugates with increased tumour uptake and antitumor function.
fine-tuning of bispecific T cell engagers and CAR receptors to induce the desired T cell activation level.
improved blocking of STn in vivo, leading to antitumor responses by restoring the function of immune cells.

Thus, in a preferred aspect, an antibody-drug conjugate (ADC) according to the present invention may comprise an antibody which comprises:
(a) a heavy chain variable region (VH) wherein the VH comprises complementarity determining regions (CDRs) selected from the group consisting of:
(i) H-CDR1, H-CDR2 and H-CDR3 as shown in any one SEQ ID NOs. 1 to 24 or 49 to 88 respectively and/or,
(b) a light chain variable region (VL) wherein the VL comprises complementarity determining regions (CDRs) selected from the group consisting of:
(i) L-CDR1, L-CDR2, and L-CDR3 as shown in any one of SEQ ID NOs. 25 to 48 or 89 to 128 respectively. Optionally, at least one CDR may comprise one or two amino acid substitutions compared to the recited sequence.

The fragment may be a functional antibody fragment of the antibody disclosed—that is, retain the ability to bind antigen.

In one aspect, the antibody or fragment thereof may comprise a heavy chain variable region (VH) and a light chain variable region (VL). Constant regions may also be provided, as will be understood.

It will be understood that monoclonal antibodies (mAbs) refers to an antibody that is produced by a single B cell clone. MAbs can be also produced by an hybridoma, which is a hybrid between a B cell and myeloma cell, or cell lines that express recombinant DNA coding for the immunoglobulin heavy and light chain, and therefore will produce a single and specific antibody.

The antibodies may be expressed to the extracellular milieu and then purified from there.

The specificity of an antibody is its ability to react with one antigen or a group of antigens that share a certain epitope. An epitope, also known as antigenic determinant, is the part of an antigen that is recognised by the antibody.

An antibody belongs to the immunoglobulin class of proteins and it is typically an assembling of two identical heavy chains (around 50-70 kDa) and too identical light chains (around 25 kDa). In the amino-terminal of each heavy or light chain there is a sequence: of 100-130 amino acids that code for the variable region. In the carboxyl-terminal of each heavy or light chain there is a sequence that codes the constant region. Typically, each antibody binds the same antigen, i.e. is bivalent.

The antigen-binding fragment (Fab) is the antibody fragment that binds to antigens. Each Fab is composed of one constant and one variable domain from each heavy and light chain of the antibody. The Fragment crystallisable (Fc) region is composed of 2 or 3 domains of the carboxy-terminal of the two heavy chains. While the Fab ensures binding to the antigen, the Fc region ensures that each antibody generates an effector immune response. The Fc region binds to various cell receptors, such as Fc receptors, and other molecules, such as complement proteins, mediating different physiological effects including opsonization to facilitate phagocytosis by phagocytes, cell lysis by natural killer cells, and degranulation of mast cells, basophils and eosinophils.

The term “variable domain” or “variable region” is the amino-terminal part of the light or heavy chains of an antibody that interacts with the antigen. It typically has a length of about 120 to 130 amino acids in the heavy chain and typically about 100 to 110 amino acids in the light chain. The sequences of each of the variable regions are substantially varied, particularly in the complementary determining regions (CDRs) responsible for the interaction with the specific antigen. The CDRs are flanked by less varied framework regions (FR). There are typically three CDRs in each of the light and heavy chains. Thus, for example, CDRs L1, L2, and L3 are within the light chain, and CDRs H1, H2 and H3 are within the heavy chain.

The expression “functional antibody fragment or probe” suitably refers to a part of the antibody that includes the variable region of the heavy and the light chain of the antibody or includes either the variable region of the heavy or the variable region of the light chain of the antibody. For example, a functional antibody fragment or probe retains most or all the binding activity of the initial antibody from which the fragment or probe is derived. Such functional antibody fragments or probes can for example include the single chain Fv (scFv), diabody, triabody, tetra-body and mini-body.

It will be appreciated that the term fragment as used herein in particular relates to fragments of antibodies specifically as described and these form an important aspect of the present disclosure. In this way, a monoclonal or recombinant antibody as provided by the present disclosure may for example be provided as any of the following fragments: (i) the Fab fragment consisting of VL, VH, CL and CH1 domains; (ii) the Fd fragment consisting of the VH and CH1 domains; (iii) the Fv fragment consisting of the VL and VH domains; (iv) the dAb fragment which consists of a VH domain; (v) the isolated CDR regions; (vi) F(ab′)2 fragments, a bivalent fragment comprising two linked Fab fragments; and (vii) single chain Fv molecules (scFv), wherein a VH domain and a VL domain are linked by a peptide linker which allows the two domains to associate to form an antigen binding site.

Alternatively, as will be understood, an antibody according to the present disclosure may comprise a whole IgG antibody, whereby the antibody includes variable and constant regions.

The term “nucleotide sequences” refers to a sequence of nucleotides of any length, either deoxy ribonucleotides or ribonucleotides or their analogues thereof.

As will be understood, nucleotide sequences can be transcribed to produce mRNA, which is then translated into a polypeptide and/or a fragment thereof.

Further aspects of the invention will now be described. In one preferred aspect, the antibody-drug conjugate (ADC) comprises an antibody or functional antibody fragment or probe thereof, wherein the antibody or fragment or probe comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 2 paired with L-CDR1, L-CDR2, and L-CDR3 as shown in SEQ ID NO. 26;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 4 paired with L-CDR1, L-CDR2, and L-CDR3 as shown in SEQ ID NO. 28;
H-CDR1, H-CDR2 and H-CDR3 as shown in SEQ ID NO. 5 paired with L-CDR1, L-CDR2, and L-CDR3 as shown in SEQ ID NO. 29;
H-CDR1, H-CDR2 and H-CDR3 as shown in SEQ ID NO. 6 paired with L-CDR1, L-CDR2, and L-CDR3 as shown in SEQ ID NO. 30;
H-CDR1, H-CDR2 and H-CDR3 as shown in SEQ ID NO. 7 paired with L-CDR1, L-CDR2, and L-CDR3 as shown in SEQ ID NO. 31;
H-CDR1, H-CDR2 and H-CDR3 as shown in SEQ ID NO. 8 paired with L-CDR1, L-CDR2, and L-CDR3 as shown in SEQ ID NO. 32;
H-CDR1, H-CDR2 and H-CDR3 as shown in SEQ ID NO. 9 paired with L-CDR1, L-CDR2, and L-CDR3 as shown in SEQ ID NO. 33;
H-CDR1, H-CDR2 and H-CDR3 as shown in SEQ ID NO. 10 paired with L-CDR1, L-CDR2, and L-CDR3 as shown in SEQ ID NO. 34;
H-CDR1, H-CDR2 and H-CDR3 as shown in SEQ ID NO. 11 paired with L-CDR1, L-CDR2, and L-CDR3 as shown in SEQ ID NO. 35;
H-CDR1, H-CDR2 and H-CDR3 as shown in SEQ ID NO. 12 paired with L-CDR1, L-CDR2, and L-CDR3 as shown in SEQ ID NO. 36;
H-CDR1, H-CDR2 and H-CDR3 as shown in SEQ ID NO. 13 paired with L-CDR1, L-CDR2, and L-CDR3 as shown in SEQ ID NO. 37;
H-CDR1, H-CDR2 and H-CDR3 as shown in SEQ ID NO. 14 paired with L-CDR1, L-CDR2, and L-CDR3 as shown in SEQ ID NO. 38;
H-CDR1, H-CDR2 and H-CDR3 as shown in SEQ ID NO. 15 paired with L-CDR1, L-CDR2, and L-CDR3 as shown in SEQ ID NO. 39;
H-CDR1, H-CDR2 and H-CDR3 as shown in SEQ ID NO. 16 paired with L-CDR1, L-CDR2, and L-CDR3 as shown in SEQ ID NO. 40;
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 18 paired with L-CDR1, L-CDR2, and L-CDR3 as shown in SEQ ID NO. 42;
H-CDR1, H-CDR2 and H-CDR3 as shown in SEQ ID NO. 19 paired with L-CDR1, L-CDR2, and L-CDR3 as shown in SEQ ID NO. 43;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45;
H-CDR1, H-CDR2 and H-CDR3 as shown in SEQ ID NO. 22 paired with L-CDR1, L-CDR2, and L-CDR3 as shown in SEQ ID NO. 46;
H-CDR1, H-CDR2 and H-CDR3 as shown in SEQ ID NO. 23 paired with L-CDR1, L-CDR2, and L-CDR3 as shown in SEQ ID NO. 47; or
H-CDR1, H-CDR2 and H-CDR3 as shown in SEQ ID NO. 24 paired with L-CDR1, L-CDR2, and L-CDR3 as shown in SEQ ID NO. 48.

Optionally, at least one CDR may comprise one or two amino acid substitutions compared to the recited sequence.

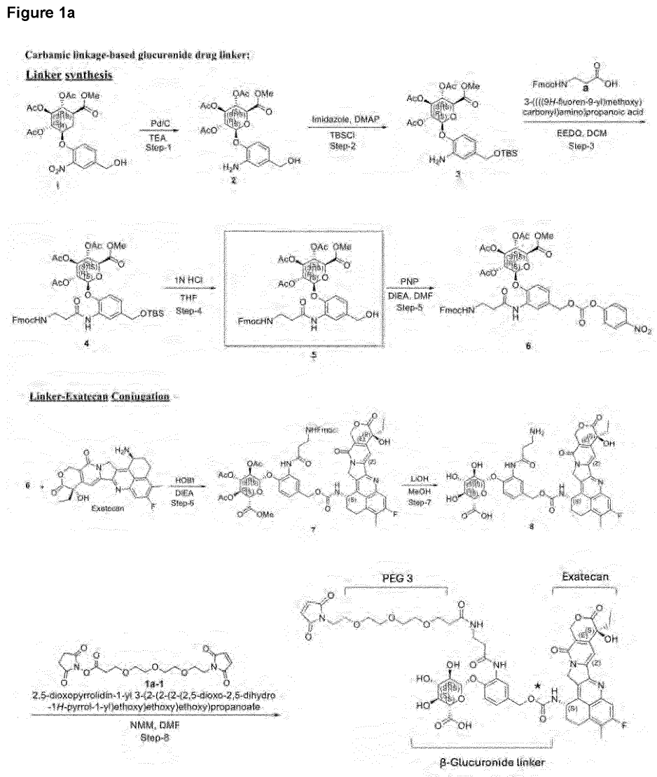

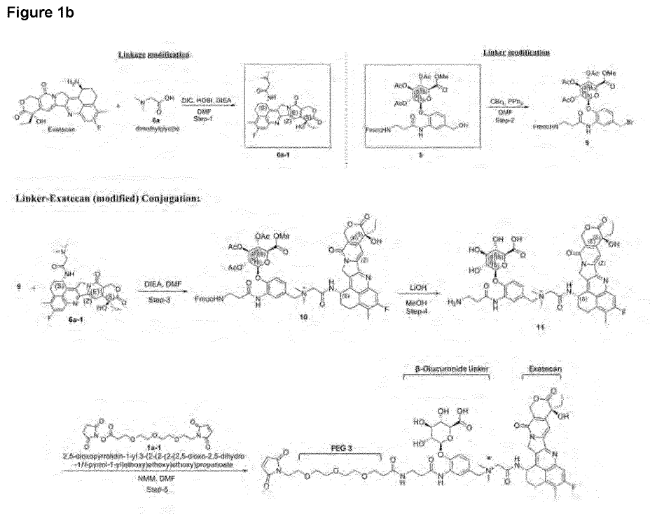

Sequence identity information identifying the humanised and affinity-matured variants provided by the present invention, including the full VH and VL sequences and the CDR regions for each variant is given in FIGS. 1 to 6.

The antibody-drug conjugate (ADC) of the present invention thus preferably includes, as the antibody part, humanised and affinity-matured antibody variants which show excellent binding affinity and specificity to the antigen STn, whilst also showing decreased immunogenicity. Humanised variants are disclosed herein by way of SEQ ID Nos 1-24 (variable VH region) and SEQ ID Nos 25-48 (variable VL region), and affinity-matured antibody variants are disclosed herein by way of SEQ ID Nos 49-88 (variable VH region) and SEQ ID Nos 89-128 (variable VL region),

In one preferred aspect, the antibody-drug conjugate (ADC) comprises an antibody or functional antibody fragment or probe thereof, wherein the antibody or fragment or probe comprises one of the following pairs of heavy chain CDRs and light chain CDRs:
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45.

Optionally, at least one CDR may comprise one or two amino acid substitutions compared to the recited sequence.

In a further aspect, the antibody-drug conjugate (ADC) comprises an antibody or functional fragment or probe thereof, wherein the antibody or fragment or probe comprises one of the following pairs of light chain CDRs and heavy chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 50 paired with L-CDR1, L-CDR2, and L-CDR3 as shown in SEQ ID NO. 90;
H-CDR1, H-CDR2 and H-CDR3 as shown in SEQ ID NO. 51 paired with L-CDR1, L-CDR2, and L-CDR3 as shown in SEQ ID NO. 91;
H-CDR1, H-CDR2 and H-CDR3 as shown in SEQ ID NO. 52 paired with L-CDR1, L-CDR2, and L-CDR3 as shown in SEQ ID NO. 92;
H-CDR1, H-CDR2 and H-CDR3 as shown in SEQ ID NO. 53 paired with L-CDR1, L-CDR2, and L-CDR3 as shown in SEQ ID NO. 93;
H-CDR1, H-CDR2 and H-CDR3 as shown in SEQ ID NO. 54 paired with L-CDR1, L-CDR2, and L-CDR3 as shown in SEQ ID NO. 94;
H-CDR1, H-CDR2 and H-CDR3 as shown in SEQ ID NO. 55 paired with L-CDR1, L-CDR2, and L-CDR3 as shown in SEQ ID NO. 95;
H-CDR1, H-CDR2 and H-CDR3 as shown in SEQ ID NO. 56 paired with L-CDR1, L-CDR2, and L-CDR3 as shown in SEQ ID NO. 96;
H-CDR1, H-CDR2 and H-CDR3 as shown in SEQ ID NO. 57 paired with L-CDR1, L-CDR2, and L-CDR3 as shown in SEQ ID NO. 97;
H-CDR1, H-CDR2 and H-CDR3 as shown in SEQ ID NO. 58 paired with L-CDR1, L-CDR2, and L-CDR3 as shown in SEQ ID NO. 98;
H-CDR1, H-CDR2 and H-CDR3 as shown in SEQ ID NO. 59 paired with L-CDR1, L-CDR2, and L-CDR3 as shown in SEQ ID NO. 99;
H-CDR1, H-CDR2 and H-CDR3 as shown in SEQ ID NO. 60 paired with L-CDR1, L-CDR2, and L-CDR3 as shown in SEQ ID NO. 100;
H-CDR1, H-CDR2 and H-CDR3 as shown in SEQ ID NO. 61 paired with L-CDR1, L-CDR2, and L-CDR3 as shown in SEQ ID NO. 101;
H-CDR1, H-CDR2 and H-CDR3 as shown in SEQ ID NO. 62 paired with L-CDR1, L-CDR2, and L-CDR3 as shown in SEQ ID NO. 102;
H-CDR1, H-CDR2 and H-CDR3 as shown in SEQ ID NO. 63 paired with L-CDR1, L-CDR2, and L-CDR3 as shown in SEQ ID NO. 103;
H-CDR1, H-CDR2 and H-CDR3 as shown in SEQ ID NO. 64 paired with L-CDR1, L-CDR2, and L-CDR3 as shown in SEQ ID NO. 104;
H-CDR1, H-CDR2 and H-CDR3 as shown in SEQ ID NO. 65 paired with L-CDR1, L-CDR2, and L-CDR3 as shown in SEQ ID NO. 105;
H-CDR1, H-CDR2 and H-CDR3 as shown in SEQ ID NO. 66 paired with L-CDR1, L-CDR2, and L-CDR3 as shown in SEQ ID NO. 106;
H-CDR1, H-CDR2 and H-CDR3 as shown in SEQ ID NO. 67 paired with L-CDR1, L-CDR2, and L-CDR3 as shown in SEQ ID NO. 107;
H-CDR1, H-CDR2 and H-CDR3 as shown in SEQ ID NO. 68 paired with L-CDR1, L-CDR2, and L-CDR3 as shown in SEQ ID NO. 108;
H-CDR1, H-CDR2 and H-CDR3 as shown in SEQ ID NO. 69 paired with L-CDR1, L-CDR2, and L-CDR3 as shown in SEQ ID NO. 109;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 71 paired with L-CDR1, L-CDR2, and L-CDR3 as shown in SEQ ID NO. 111;
H-CDR1, H-CDR2 and H-CDR3 as shown in SEQ ID NO. 72 paired with L-CDR1, L-CDR2, and L-CDR3 as shown in SEQ ID NO. 112;
H-CDR1, H-CDR2 and H-CDR3 as shown in SEQ ID NO. 73 paired with L-CDR1, L-CDR2, and L-CDR3 as shown in SEQ ID NO. 113;
H-CDR1, H-CDR2 and H-CDR3 as shown in SEQ ID NO. 74 paired with L-CDR1, L-CDR2, and L-CDR3 as shown in SEQ ID NO. 114;
H-CDR1, H-CDR2 and H-CDR3 as shown in SEQ ID NO. 75 paired with L-CDR1, L-CDR2, and L-CDR3 as shown in SEQ ID NO. 115;
H-CDR1, H-CDR2 and H-CDR3 as shown in SEQ ID NO. 76 paired with L-CDR1, L-CDR2, and L-CDR3 as shown in SEQ ID NO. 116;
H-CDR1, H-CDR2 and H-CDR3 as shown in SEQ ID NO. 77 paired with L-CDR1, L-CDR2, and L-CDR3 as shown in SEQ ID NO. 117;
H-CDR1, H-CDR2 and H-CDR3 as shown in SEQ ID NO. 78 paired with L-CDR1, L-CDR2, and L-CDR3 as shown in SEQ ID NO. 118;
H-CDR1, H-CDR2 and H-CDR3 as shown in SEQ ID NO. 79 paired with L-CDR1, L-CDR2, and L-CDR3 as shown in SEQ ID NO. 119;
H-CDR1, H-CDR2 and H-CDR3 as shown in SEQ ID NO. 80 paired with L-CDR1, L-CDR2, and L-CDR3 as shown in SEQ ID NO. 120;
H-CDR1, H-CDR2 and H-CDR3 as shown in SEQ ID NO. 81 paired with L-CDR1, L-CDR2, and L-CDR3 as shown in SEQ ID NO. 121;
H-CDR1, H-CDR2 and H-CDR3 as shown in SEQ ID NO. 82 paired with L-CDR1, L-CDR2, and L-CDR3 as shown in SEQ ID NO. 122;
H-CDR1, H-CDR2 and H-CDR3 as shown in SEQ ID NO. 83 paired with L-CDR1, L-CDR2, and L-CDR3 as shown in SEQ ID NO. 123;
H-CDR1, H-CDR2 and H-CDR3 as shown in SEQ ID NO. 84 paired with L-CDR1, L-CDR2, and L-CDR3 as shown in SEQ ID NO. 124;
H-CDR1, H-CDR2 and H-CDR3 as shown in SEQ ID NO. 85 paired with L-CDR1, L-CDR2, and L-CDR3 as shown in SEQ ID NO. 125;
H-CDR1, H-CDR2 and H-CDR3 as shown in SEQ ID NO. 86 paired with L-CDR1, L-CDR2, and L-CDR3 as shown in SEQ ID NO. 126;
H-CDR1, H-CDR2 and H-CDR3 as shown in SEQ ID NO. 87 paired with L-CDR1, L-CDR2, and L-CDR3 as shown in SEQ ID NO. 127; or
H-CDR1, H-CDR2 and H-CDR3 as shown in SEQ ID NO. 88 paired with L-CDR1, L-CDR2, and L-CDR3 as shown in SEQ ID NO. 128.

Optionally, at least one CDR may comprise one or two amino acid substitutions compared to the recited sequence.

In a preferred aspect, the antibody-drug conjugate (ADC) comprises an antibody or fragment thereof or probe wherein the antibody or fragment or probe comprises one of the following pairs of heavy chain CDRs and light chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 81 paired with L-CDR1, L-CDR2, and L-CDR3 as shown in SEQ ID NO. 121; or
H-CDR1, H-CDR2 and H-CDR3 as shown in SEQ ID NO. 88 paired with L-CDR1, L-CDR2, and L-CDR3 as shown in SEQ ID NO. 128.

Optionally, at least one CDR may comprise one or two amino acid substitutions compared to the recited sequence.

In a further aspect of the invention, the antibody-drug conjugate (ADC) comprises an antibody or functional antibody fragment or probe thereof wherein the antibody or fragment or probe comprises in addition to the CDR regions described herein:
(a) a heavy chain variable region (VH) wherein the VH comprises a humanised heavy chain framework region and/or,
(b) a light chain variable region (VL) wherein the VL comprises a humanised light chain framework region.

The humanised framework regions may, for example, be as further described below. It will be understood that the variable framework regions refer to the sequences surrounding the CDR regions. Thus different combinations of the CDR regions and variable framework regions described herein may be made if desired.

Thus, in one aspect of the present invention, the antibody-drug conjugate (ADC) comprises an antibody or fragment thereof, wherein the antibody or fragment comprises:
(a) a heavy chain variable region (VH) wherein the VH comprises a humanised heavy chain framework region as shown in the heavy chain variable region (VH) sequences selected from the group consisting of:
(i) any one SEQ ID NOs. 1 to 24 or 49 to 88 respectively or a sequence with at least 80% sequence identity to the sequences recited
and/or,
(b) a light chain variable region (VL) wherein the VL comprises a humanised light chain framework region as shown in the light chain variable region (VL) sequences selected from the group consisting of:
(ii) any one of SEQ ID NOs. 25 to 48 or 89 to 128 respectively or a sequence with at least 80% sequence identity to the sequences recited.

Thus, in one aspect of the present invention, the antibody-drug conjugate (ADC) comprises an antibody or fragment thereof, wherein the antibody or fragment comprises:
(a) a heavy chain variable region (VH) wherein the VH comprises a humanised heavy chain framework region as shown in the heavy chain variable region (VH) sequences selected from the group consisting of:
(i) any one SEQ ID NOs. 1 to 24 or 49 to 88 respectively
and/or,
(b) a light chain variable region (VL) wherein the VL comprises a humanised light chain framework region as shown in the light chain variable region (VL) sequences selected from the group consisting of:
(ii) any one of SEQ ID NOs. 25 to 48 or 89 to 128 respectively.

One of the above described humanised heavy chain framework regions may be paired with any one of the humanised light chain framework regions, as desired.

Certain pairings of the framework regions are preferred. In one aspect of the invention, there is provided an antibody-drug conjugate (ADC) comprising an antibody or fragment thereof or probe thereof wherein the antibody or fragment comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 1 paired with light chain framework region from SEQ ID NO. 25;
heavy chain framework region of SEQ ID NO. 2 paired with light chain framework region from SEQ ID NO. 26;
heavy chain framework region of SEQ ID NO. 3 paired with light chain framework region from SEQ ID NO. 27;
heavy chain framework region of SEQ ID NO. 4 paired with light chain framework region from SEQ ID NO. 28;
heavy chain framework region of SEQ ID NO. 5 paired with light chain framework region from SEQ ID NO. 29;
heavy chain framework region of SEQ ID NO. 6 paired with light chain framework region from SEQ ID NO. 30;
heavy chain framework region of SEQ ID NO. 7 paired with light chain framework region from SEQ ID NO. 31;
heavy chain framework region of SEQ ID NO. 8 paired with light chain framework region from SEQ ID NO. 32;
heavy chain framework region of SEQ ID NO. 9 paired with light chain framework region from SEQ ID NO. 33;
heavy chain framework region of SEQ ID NO. 10 paired with light chain framework region from SEQ ID NO. 34;
heavy chain framework region of SEQ ID NO. 11 paired with light chain framework region from SEQ ID NO. 35;
heavy chain framework region of SEQ ID NO. 12 paired with light chain framework region from SEQ ID NO. 36;
heavy chain framework region of SEQ ID NO. 13 paired with light chain framework region from SEQ ID NO. 37;
heavy chain framework region of SEQ ID NO. 14 paired with light chain framework region from SEQ ID NO. 38;
heavy chain framework region of SEQ ID NO. 15 paired with light chain framework region from SEQ ID NO. 39;
heavy chain framework region of SEQ ID NO. 16 paired with light chain framework region from SEQ ID NO. 40;
heavy chain framework region of SEQ ID NO. 17 paired with light chain framework region from SEQ ID NO. 41;
heavy chain framework region of SEQ ID NO. 18 paired with light chain framework region from SEQ ID NO. 42;
heavy chain framework region of SEQ ID NO. 19 paired with light chain framework region from SEQ ID NO. 43;
heavy chain framework region of SEQ ID NO. 20 paired with light chain framework region from SEQ ID NO. 44;
heavy chain framework region of SEQ ID NO. 21 paired with light chain framework region from SEQ ID NO. 45;
heavy chain framework region of SEQ ID NO. 22 paired with light chain framework region from SEQ ID NO. 46;
heavy chain framework region of SEQ ID NO. 23 paired with light chain framework region from SEQ ID NO. 47; or
heavy chain framework region of SEQ ID NO. 24 paired with light chain framework region from SEQ ID NO. 48.

Optionally, the heavy chain framework region and/or the light chain framework region have at least 80% sequence identity to the sequences recited.

In a further aspect of the invention, there is provided an antibody-drug conjugate (ADC) comprising an antibody or fragment thereof or probe thereof wherein the antibody or fragment or probe comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 49 paired with light chain framework region from SEQ ID NO. 89;
heavy chain framework region of SEQ ID NO. 50 paired with light chain framework region from SEQ ID NO. 90;
heavy chain framework region of SEQ ID NO. 51 paired with light chain framework region from SEQ ID NO. 91;
heavy chain framework region of SEQ ID NO. 52 paired with light chain framework region from SEQ ID NO. 92;
heavy chain framework region of SEQ ID NO. 53 paired with light chain framework region from SEQ ID NO. 93;
heavy chain framework region of SEQ ID NO. 54 paired with light chain framework region from SEQ ID NO. 94;
heavy chain framework region of SEQ ID NO. 55 paired with light chain framework region from SEQ ID NO. 95;
heavy chain framework region of SEQ ID NO. 56 paired with light chain framework region from SEQ ID NO. 96;
heavy chain framework region of SEQ ID NO. 57 paired with light chain framework region from SEQ ID NO. 97;
heavy chain framework region of SEQ ID NO. 58 paired with light chain framework region from SEQ ID NO. 98;
heavy chain framework region of SEQ ID NO. 59 paired with light chain framework region from SEQ ID NO. 99;
heavy chain framework region of SEQ ID NO. 60 paired with light chain framework region from SEQ ID NO. 100;
heavy chain framework region of SEQ ID NO. 61 paired with light chain framework region from SEQ ID NO. 101;
heavy chain framework region of SEQ ID NO. 62 paired with light chain framework region from SEQ ID NO. 102;
heavy chain framework region of SEQ ID NO. 63 paired with light chain framework region from SEQ ID NO. 103;
heavy chain framework region of SEQ ID NO. 64 paired with light chain framework region from SEQ ID NO. 104;
heavy chain framework region of SEQ ID NO. 65 paired with light chain framework region from SEQ ID NO. 105;
heavy chain framework region of SEQ ID NO. 66 paired with light chain framework region from SEQ ID NO. 106;
heavy chain framework region of SEQ ID NO. 67 paired with light chain framework region from SEQ ID NO. 107;
heavy chain framework region of SEQ ID NO. 68 paired with light chain framework region from SEQ ID NO. 108;
heavy chain framework region of SEQ ID NO. 69 paired with light chain framework region from SEQ ID NO. 109;
heavy chain framework region of SEQ ID NO. 70 paired with light chain framework region from SEQ ID NO. 110;
heavy chain framework region of SEQ ID NO. 71 paired with light chain framework region from SEQ ID NO. 111;
heavy chain framework region of SEQ ID NO. 72 paired with light chain framework region from SEQ ID NO. 112;
heavy chain framework region of SEQ ID NO. 73 paired with light chain framework region from SEQ ID NO. 113;
heavy chain framework region of SEQ ID NO. 74 paired with light chain framework region from SEQ ID NO. 114;
heavy chain framework region of SEQ ID NO. 75 paired with light chain framework region from SEQ ID NO. 115;
heavy chain framework region of SEQ ID NO. 76 paired with light chain framework region from SEQ ID NO. 116;
heavy chain framework region of SEQ ID NO. 77 paired with light chain framework region from SEQ ID NO. 117;
heavy chain framework region of SEQ ID NO. 78 paired with light chain framework region from SEQ ID NO. 118;
heavy chain framework region of SEQ ID NO. 79 paired with light chain framework region from SEQ ID NO. 119;
heavy chain framework region of SEQ ID NO. 80 paired with light chain framework region from SEQ ID NO. 120;
heavy chain framework region of SEQ ID NO. 81 paired with light chain framework region from SEQ ID NO. 121;
heavy chain framework region of SEQ ID NO. 82 paired with light chain framework region from SEQ ID NO. 122;
heavy chain framework region of SEQ ID NO. 83 paired with light chain framework region from SEQ ID NO. 123;
heavy chain framework region of SEQ ID NO. 84 paired with light chain framework region from SEQ ID NO. 124;
heavy chain framework region of SEQ ID NO. 85 paired with light chain framework region from SEQ ID NO. 125;
heavy chain framework region of SEQ ID NO. 86 paired with light chain framework region from SEQ ID NO. 126;
heavy chain framework region of SEQ ID NO. 87 paired with light chain framework region from SEQ ID NO. 127; or
heavy chain framework region of SEQ ID NO. 88 paired with light chain framework region from SEQ ID NO. 128.

Optionally, the heavy chain framework region and/or the light chain framework region have at least 80% sequence identity to the sequences recited.

It is the case that the framework regions allow a certain degree of variability in the exact sequence, whilst still allowing for maintenance of function, including binding affinity and specificity. Accordingly, the invention also provides an antibody-drug conjugate (ADC) comprising an antibody or fragment or probe thereof as described, wherein the heavy chain framework region and/or the light chain framework region may have 80% or more, preferably 85% or more, more preferably 90% or more, even more preferably 95% or more, or even more preferably 99% or more sequence identity to the specific sequences recited herein. For example, this may be by way of substitution, addition, or deletion of amino acid residues, with substitution often being preferred. Substitutions may, for example, be conservative amino acid substitutions.

In a preferred aspect of the invention, the antibody-drug conjugate (ADC) may comprise the antibody or fragment or probe thereof as described herein such that the heavy chain framework region and/or the light chain framework region have 90% or more, preferably 95% or more, or 99% or more sequence identity to the specific sequences recited herein. For example, this may be by way of substitution, addition, or deletion of amino acid residues, with substitution often being preferred. Substitutions may, for example, be conservative amino acid substitutions.

In a further aspect of the present invention, there is provided an antibody-drug conjugate (ADC) comprising an antibody or fragment thereof or probe thereof wherein the antibody or fragment or probe comprises:
(a) a heavy chain variable region (VH) selected from the group consisting of:
(i) any one SEQ ID NOs. 1 to 24 or 49 to 88 respectively or a sequence with at least 80% sequence identity to the sequences recited and/or,
(a) a light chain variable region (VL) selected from the group consisting of:
(ii) any one of SEQ ID NOs. 25 to 48 or 89 to 128 respectively or a sequence with at least 80% sequence identity to the sequences recited.

In a further aspect of the present invention, there is provided an antibody-drug conjugate (ADC) comprising an antibody or fragment thereof or probe thereof wherein the antibody or fragment or probe comprises:
(a) a heavy chain variable region (VH) selected from the group consisting of:
(i) any one SEQ ID NOs. 1 to 24 or 49 to 88 respectively
and/or,
(a) a light chain variable region (VL) selected from the group consisting of:
(ii) any one of SEQ ID NOs. 25 to 48 or 89 to 128 respectively.

Any one of the VH regions may be paired with any one of the VL regions, although certain pairings are preferred.

Thus, in a further aspect, the present invention provides an antibody-drug conjugate (ADC) comprising an antibody or fragment thereof or probe thereof as described herein, wherein the antibody or fragment or probe comprises one of the following pairs of heavy chain variable regions (VH) and light chain variable regions (VL):
SEQ ID NO. 1 paired with SEQ ID NO. 25;
SEQ ID NO. 2 paired with SEQ ID NO. 26;
SEQ ID NO. 3 paired with SEQ ID NO. 27;
SEQ ID NO. 4 paired with SEQ ID NO. 28;
SEQ ID NO. 5 paired with SEQ ID NO. 29;
SEQ ID NO. 6 paired with SEQ ID NO. 30;
SEQ ID NO. 7 paired with SEQ ID NO. 31;
SEQ ID NO. 8 paired with SEQ ID NO. 32;
SEQ ID NO. 9 paired with SEQ ID NO. 33;
SEQ ID NO. 10 paired with SEQ ID NO. 34;
SEQ ID NO. 11 paired with SEQ ID NO. 35;
SEQ ID NO. 12 paired with SEQ ID NO. 36;
SEQ ID NO. 13 paired with SEQ ID NO. 37;
SEQ ID NO. 14 paired with SEQ ID NO. 38;
SEQ ID NO. 15 paired with SEQ ID NO. 39;
SEQ ID NO. 16 paired with SEQ ID NO. 40;
SEQ ID NO. 17 paired with SEQ ID NO. 41;
SEQ ID NO. 18 paired with SEQ ID NO. 42;
SEQ ID NO. 19 paired with SEQ ID NO. 43;
SEQ ID NO. 20 paired with SEQ ID NO. 44;
SEQ ID NO. 21 paired with SEQ ID NO. 45;
SEQ ID NO. 22 paired with SEQ ID NO. 46;
SEQ ID NO. 23 paired with SEQ ID NO. 47; or
SEQ ID NO. 24 paired with SEQ ID NO. 48.

Optionally, the heavy chain variable region (VH) and/or the light chain variable region (VL) may have at least 80% sequence identity to the sequences recited.

In a further aspect of the invention, the antibody-drug conjugate (ADC) comprises an antibody or fragment thereof or probe thereof wherein the antibody or fragment or probe comprises one of the following pairs of heavy chain variable regions (VH) and light chain variable regions (VL):
SEQ ID NO. 49 paired with SEQ ID NO. 89;
SEQ ID NO. 50 paired with SEQ ID NO. 90;
SEQ ID NO. 51 paired with SEQ ID NO. 91;
SEQ ID NO. 52 paired with SEQ ID NO. 92;
SEQ ID NO. 53 paired with SEQ ID NO. 93;
SEQ ID NO. 54 paired with SEQ ID NO. 94;
SEQ ID NO. 55 paired with SEQ ID NO. 95;
SEQ ID NO. 56 paired with SEQ ID NO. 96;
SEQ ID NO. 57 paired with SEQ ID NO. 97;
SEQ ID NO. 58 paired with SEQ ID NO. 98;
SEQ ID NO. 59 paired with SEQ ID NO. 99;
SEQ ID NO. 60 paired with SEQ ID NO. 100;
SEQ ID NO. 61 paired with SEQ ID NO. 101;
SEQ ID NO. 62 paired with SEQ ID NO. 102;
SEQ ID NO. 63 paired with SEQ ID NO. 103;
SEQ ID NO. 64 paired with SEQ ID NO. 104;
SEQ ID NO. 65 paired with SEQ ID NO. 105;
SEQ ID NO. 66 paired with SEQ ID NO. 106;
SEQ ID NO. 67 paired with SEQ ID NO. 107;
SEQ ID NO. 68 paired with SEQ ID NO. 108;
SEQ ID NO. 69 paired with SEQ ID NO. 109;
SEQ ID NO. 70 paired with SEQ ID NO. 110;
SEQ ID NO. 71 paired with SEQ ID NO. 111;
SEQ ID NO. 72 paired with SEQ ID NO. 112;
SEQ ID NO. 73 paired with SEQ ID NO. 113;
SEQ ID NO. 74 paired with SEQ ID NO. 114;
SEQ ID NO. 75 paired with SEQ ID NO. 115;
SEQ ID NO. 76 paired with SEQ ID NO. 116;
SEQ ID NO. 77 paired with SEQ ID NO. 117;
SEQ ID NO. 78 paired with SEQ ID NO. 118;
SEQ ID NO. 79 paired with SEQ ID NO. 119;
SEQ ID NO. 80 paired with SEQ ID NO. 120;
SEQ ID NO. 81 paired with SEQ ID NO. 121;
SEQ ID NO. 82 paired with SEQ ID NO. 122;
SEQ ID NO. 83 paired with SEQ ID NO. 123;
SEQ ID NO. 84 paired with SEQ ID NO. 124;
SEQ ID NO. 85 paired with SEQ ID NO. 125;
SEQ ID NO. 86 paired with SEQ ID NO. 126
SEQ ID NO. 87 paired with SEQ ID NO. 127; or
SEQ ID NO. 88 paired with SEQ ID NO. 128.

Optionally, the heavy chain variable region (VH) and/or the light chain variable region (VL) may have at least 80% sequence identity to the sequences recited.

In the same way as described above, the invention also provides an antibody-drug conjugate (ADC) comprising an antibody or fragment thereof or probe thereof as described, wherein the heavy chain variable region (VH) and/or the light chain variable region (VL) may have 80% or more, preferably 85% or more, more preferably 90% or more, even more preferably 95% or more, or even more preferably 99% or more sequence identity to the specific VH and VL sequences recited herein. For example, this may be by way of substitution, addition, or deletion of amino acid residues, with substitution often being preferred. Substitutions may, for example, be conservative amino acid substitutions.

In a preferred aspect of the invention, the antibody-drug conjugate (ADC) comprises an antibody or fragment or probe thereof as described herein such that the heavy chain variable region (VH) and/or the light chain variable region have 90% or more, preferably 95% or more, even more preferably 99% or more sequence identity to the specific sequences recited herein. For example, this may be by way of substitution, addition, or deletion of amino acid residues, with substitution often being preferred. Substitutions may, for example, be conservative amino acid substitutions.

In terms of functionality, the invention in particular provides an antibody-drug conjugate (ADC) comprising an antibody or fragment thereof, or a probe thereof, as described herein, that binds to STn and a group of glycans terminated by alpha 2,6-linked sialic acids. The glycans terminated by alpha 2,6-linked sialic acids may comprise for example STn, 2,6-sialyl T, di-sialyl T, or 2,6-sialolactosamine.

An overall group of glycans recognized by the antibody or fragment thereof, or a probe thereof, as described herein includes:
1. Sialyl Tn:

### NeuAca--6GalNAcα/β1-

2. 2,6-sialyl T:

### Gal β1-3GalNAcα1-NeuAcα2-6

3. di sialyl T:

### NeuAcα2-3Galβ1-3GalNAcα1 - - - NeuAcα2-6

4. 2,6-sialo-N-acetyllactosamine:

### NeuAcα2-6Galβ1-4Glcβ1-

The sialyl Tn, aka, STn, sialosyl Tn, sialylated Tn, Neu5Ac-α2, 6GalNAcα-O-Ser/Thr, or also referred to as CD175s by the “cluster of differentiation’ nomenclature, is the simplest sialylated mucin-type O-glycan. The STn is a truncated O-glycan containing a sialic acid (Neu5Ac) α-2,6 linked (via carbon 6} to N-acetyl-galactosamine (GalNAc) alpha-O-linked to a serine/threonine (Ser/Thr) (Neu5Ac-α2, 6GalNAcα-O-Ser/Thr). The sialylation prevents the formation of various core structures otherwise found in mucin-type O-glycans.

STn is expressed by more than 80% of human carcinomas and is associated with poor prognosis and decreased overall survival in different cancer patients. The biosynthesis of the STn antigen has been linked to the expression of the sialyltransferase ST6GalNAc1, and to mutations or loss of heterozygosity of the COSMC gene.

Antibodies that bind to STn with such specificity are particularly interesting because of their high tumour specificity and low or absent reactivity to normal cells, in contrast to many current antibody therapies.

The antibody-drug conjugate (ADC) described herein, comprising an antibody or fragment thereof or probe thereof as described herein, is able to specifically bind to STn or a group of alpha-2,6 sialylated glycans, as described herein.

The antibodies or fragments thereof described herein, may be subject to glycan changes at glycosylation sites.

In one aspect of the invention, an antibody or fragment thereof or probe thereof as described herein, as part of the antibody-drug conjugate (ADC), may be provided in any suitable form.

For example, the antibody or fragment or probe may be provided as a ScFv, monoclonal antibody, chimeric antibody, humanized antibody, bispecific antibody, or CAR-T-cell, or other format, as will be understood by the skilled person.

Thus, for example, an antibody or fragment or probe thereof, as part of the ADC, may be provided as a single-chain fragment variable antibody (scFv). This refers to a functional antibody fragment containing only the VL and VH regions, which are joined by a linker, forming a monovalent antigen binding site. Diabodies, tribodies and tetrabodies are antibodies including dimers, trimers or tetramers of scFv, i. e. containing two, three and four polypeptide chains respectively, and forming two, three and four antigen binding sites respectively, which can be the same or different. Such may also be used.

The antibody, functional antibody fragment or probes thereof, component of the ADC of the present invention, may have one or more binding sites. If containing more than one binding site, these sites can be identical to one another or can be different. In the case of two different binding sites, the antibody, functional antibody fragment or probe thereof, is named a “bispecific” antibody.

The invention also provides a pharmaceutical composition comprising an antibody-drug conjugate (ADC) as described herein comprising an antibody or functional antibody fragment or probe thereof as described herein, and a pharmaceutically acceptable carrier.

In another aspect, there is provided a method of detecting a tumour biomarker in a patient sample using an antibody-drug conjugate (ADC) as described herein comprising an antibody or functional antibody fragment or probe thereof as described herein, or using a pharmaceutical composition as described herein. The methodology involves the staining of biological samples obtained from a subject with the nucleotide sequences encoding an antibody or functional antibody fragment or probe thereof, or an antibody drug conjugate (ADC), as described herein, under suitable conditions for specific binding to the said antibody. The presence or absence of binding of the said antibody is indicative of tumour cells expressing cell surface STn, 2,6-sialyl T, di-sialyl T, or 2,6-sialolactosamine. For example, the biological samples analysed, may include isolated cells, or tissue, or tumour derived proteins.

The invention also provides comprising an antibody-drug conjugate (ADC) comprising an antibody or functional antibody fragment or probe thereof, or a pharmaceutical composition for medical use, all of them as herein described. In particular, an antibody-drug conjugate (ADC) comprising an antibody (as described herein) as a pharmaceutical composition for medical use aims at being used for treating cancer patients. It is envisaged that various types of tumours may be treated with the variants disclosed herein.

Thus, in some aspects, the antibody, functional antibody fragment or probe thereof part of the an antibody-drug conjugate (ADC) of the invention, may be conjugated or fused to one or more diagnostic or therapeutic agents, or any other desired molecules. The resulting conjugated antibody, functional antibody fragment or probe thereof, can be useful to monitor or diagnose the onset development, progression and/or severity of a disease associated with the expression of STn or alpha-2, 6 sialylated glycans.

An antibody-drug conjugate (ADC) comprising an antibody or functional fragment or probe thereof as described herein may also be used to detect the expression of STn or alpha-2,6 sialylated glycans in any biological sample using classical immunohistological methods (IHC or immunoassays, such as the enzyme linked immunosorbent assay (ELISA) and the radioimmunoassay (RIA), flow cytometry, and immunoblotting.

The antibody-drug conjugate (ADC) of the invention comprising an antibody, functional antibody fragment or probe thereof as described herein may be included alone, conjugated, or in combination with a pharmaceutical composition, provided in an effective concentration, to wield a therapeutically useful effect, with minimal side effects.

In a further aspect, the present disclosure includes an isolated polynucleotide comprising a nucleic acid sequence, wherein the nucleic acid sequence encodes an antibody or functional antibody fragment or probe thereof, as described herein, in particular the variable heavy chain region of the antibody, the variable light chain region domain of the antibody, or functional antibody fragments or probes thereof.

In a further aspect, the present disclosure provides an expression vector comprising a polynucleotide encoding an antibody or fragment or probe as described herein. As will be understood, a suitable host cell comprising such an expression vector may be provided.

In accordance with one aspect of the present disclosure, a method of producing an antibody or functional antibody fragment or probe thereof, as described herein may comprise using such a suitable host cell.

In a further aspect, the invention also provides a method of making an antibody-drug conjugate (ADC) as described herein, which method comprises the steps of providing a drug which is a growth inhibitory agent coupled to an antibody which binds to sialyl Tn (STn) or a glycan terminated by an alpha 2,6-linked sialic acid at a suitable DAR; providing a linker as described herein, wherein the linker comprises a cleavable linker; and conjugating the coupled drug-antibody to the linker. The growth inhibitory agent may for example be an anti-cancer agent, such as a chemotherapeutic agent or cytotoxic agent. The growth inhibitory agent may for example be exatecan or a derivative thereof, or deruxtecan or a derivative thereof. The resulting ADC is cleavable to release the drug (or payload) into the microenvironment of the tumour or tumour cells.

The linker may be any one of the linkers as described herein. Preferably, the linker is one of the linkers described in the novel linker section below, especially wherein the linker comprises one of the structures shown in Formulas I to VI below.

The antibody may for example be any one of the antibodies as described herein.

Preferably, the DAR is about 4 or less.

The presently disclosed ADCs targeting cancer-specific biomarkers comprise an innovative design. The present invention provides novel Antibody-Drug-Conjugate (ADC) constructs that combine the specificity of cancer-specific antibodies with the potent cytotoxic activity of for example exatecan derivatives, using in particular beta-glucuronide linkers with improved solubility (for example, the use of 3 PEGs).

The present invention targets the STn glycan, a short O-glycan antigen that is truncated and overexpressed in different types of carcinomas, while being absent in normal healthy tissues. The targeting of this unique biomarker is expected to have a significant impact on the treatment of different types of cancer, particularly those that are metastatic, drug-resistant, and highly malignant.

The present invention demonstrates superior efficacy in inhibiting tumor growth in vitro and in vivo, compared to controls. The use, for example, of a quaternary amine as a handle in the beta-glucuronide linker has shown increased tumor uptake and decreased payload internalization when cleaved outside the tumor, leading to better safety profiles.

The presently disclosed ADCs, and other aspects of the invention, also have potential for use in animal health, as the invention targets a cancer-specific biomarker that is present in various types of animal cancer. This novel approach could lead to the development of new therapies for cancer in animals, providing a significant benefit to the veterinary industry.

The presently disclosed ADCs, in particular, combining different elements in a unique way. The present invention combines different elements, including humanized antibodies, an affinity maturation process, beta-glucuronide linkers, PEGs and carbamate/quaternary amine handles, and for example exatecan derivatives, in a novel way that results in a more effective and specific cancer treatment. This unique combination of elements is expected to have a significant impact on the field of cancer therapy, particularly in the treatment of different types of cancer that have proven difficult to treat.

We have found improved internalization for the presently disclosed ADCs, compared to Trastuzumab-based ADCs, and this provides a significant advantage in terms of efficacy. This is due to the fact that internalization is a key factor in the delivery of the cytotoxic payload to the tumor cells. By improving internalization, we are able to deliver a higher amount of cytotoxic payload to the target cells, resulting in a more potent and effective treatment. Additionally, the range of internalization capabilities offered by the cancer-specific antibodies disclosed herein allows fine-tuning of the design of the ADCs to specific types of tumors and/or patient populations, providing further customization and improved treatment outcomes.

The improved uptake of the presently disclosed ADCs in the tumor microenvironment is a result of the cancer-specific antibodies disclosed herein (see the bio distribution data). By selectively targeting cancer-specific antigens such as STn, the presently disclosed ADCs can accumulate more efficiently within the tumor microenvironment, improving treatment outcomes. This specificity also leads to a decreased likelihood of off-target effects, further improving the safety of the ADCs.

### Sequence Liabilities

As described in our pending patent application WO2023/249502, we have also looked at identifying sequence liabilities (i.e. post-translational modification-PTM-sites) in the CDRs of the humanized V1 and affinity-matured clones described herein. We have identified at least one PTM site in the heavy chain and another other in the light chain. To remove the liabilities, it is proposed to introduce a single amino acid alteration in each PTM, therefore changing the CDR.

The highest risk positions have been assessed to be:

In the VL:
CDR3 position 93/94 “DP” Aspartate Fragmentation site

In the VH:
CDR2 position 53/54 “NS” Deamidation site.

This risk appears to be specific to the affinity-matured variant mAb-v53.
CDR2 position 55/56 “DG” Aspartate Isomerisation site.

Accordingly, in one aspect of the invention, in an ADC as described herein, any one of the L-CDR3 sequences or VL variable light chain sequences disclosed herein (either humanised or affinity-matured) may be further mutated to replace “D” (aspartic acid/aspartate) at position 93 with any one of the following amino acid residues:
A, E, F, G, H, I, K, L, N, P, Q, R, S, T, V, W, or Y.

In a further aspect, any one of the L-CDR3 sequences or VL variable light chain sequences disclosed herein (either humanised or affinity-matured) may be further mutated to replace “P” (proline) at position 94 with any one of the following amino acid residues:
A, E, F, H, I, K, L, N, Q, R, T, V, W, or Y.

In one of the changes indicated for position 93 may be paired with any one of the changes indicated for position 94.

In one preferred aspect, any one of the L-CDR3 sequences or VL variable light chain sequences disclosed herein (either humanised or affinity-matured) may be further mutated to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY.

Thus, the VL CDR3 sequence in any of the sequences disclosed herein may be modified in the above way. Mutations DA, DK, DN, EP, KP, NP, QP, or RP may be preferred.

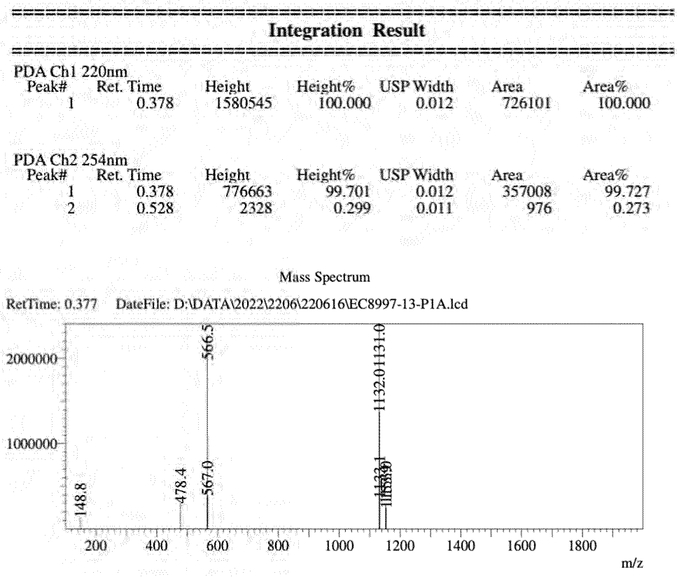

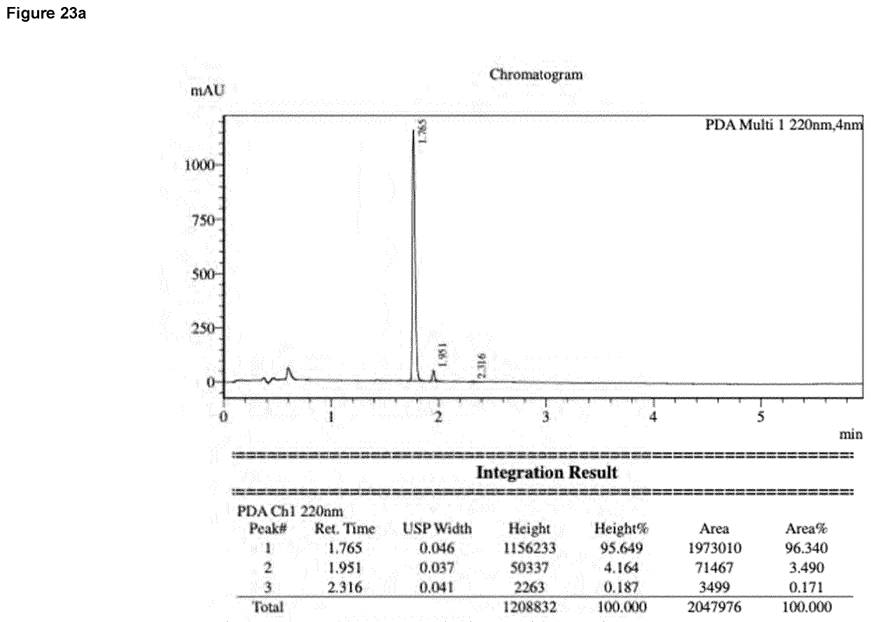

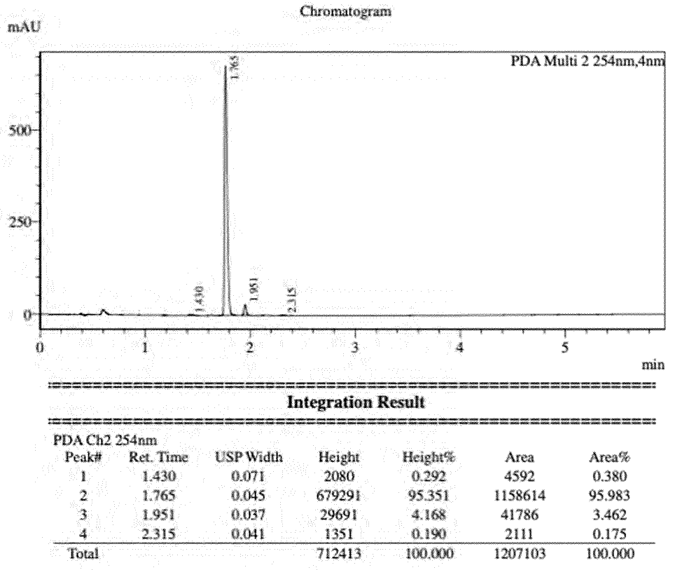

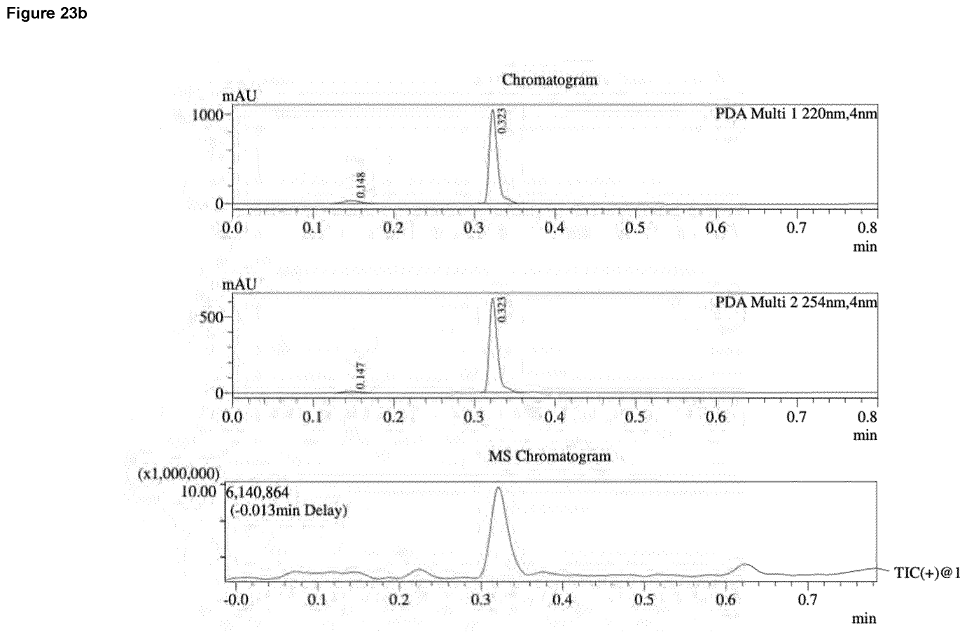

Certain preferred VL sequences incorporating the above sequence liability modifications are shown in FIG. 23b, which discloses the variable light chains of humanised variants (v65 to v88) based on V1. These are shown as SEQUENCE ID Nos 150 to 173.

Accordingly to a further aspect of the invention, in an ADC as described herein, any one of the H-CDR2 sequences or VH variable heavy chain sequences disclosed herein (either humanised or affinity-matured) may be further mutated to replace “D” (aspartic acid/aspartate) at position 55 with any one of the following amino acid residues:
A, E, F, G, H, I, K, L, P, Q, R, V, W or Y.

In a further aspect, any one of the H-CDR2 sequences or VH variable heavy chain sequences disclosed herein (either humanised or affinity-matured) may be further mutated to replace “G” (glycine) at position 56 with any one of the following amino acid residues:
A, E, F, H, I, K, L, N, Q, R, T, V, W or Y.

In one of the changes indicated for position 55 may be paired with any one of the changes indicated for position 56.

In one preferred aspect, any one of the H-CDR2 sequences or VH variable heavy chain sequences disclosed herein (either humanised or affinity-matured) may be further mutated to replace the “DG” at positions 55 and 56 with any one of the following pairs of amino acid residues:
DE, DK, DA, EG, QG, RG, AA, EE, FF, GG, HH, KK, LL, DN, PQ, QQ, RR, DT, VV, WW, or YY.

Thus, the VH CDR2 sequence in any of the sequences disclosed herein may be modified in the above way. Mutations DE, DK, DA, EG, QG, or RG may be preferred.

Certain preferred VH sequences incorporating the above sequence liability modifications are shown in FIG. 23a, which discloses the variable heavy chains of humanised variants (v65 to v85) based on V1. These are shown as SEQUENCE ID Nos 129 to 149.

In a preferred aspect, in an ADC as described herein, any one of the L-CDR3 sequences or VL variable light chain sequences disclosed herein (either humanised or affinity-matured) and containing a mutation to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY
may be paired with any one of the H-CDR2 sequences or VH variable heavy chain sequences disclosed herein (either humanised or affinity-matured) and containing a mutation to replace the “DG” at positions 55 and 56 with any one of the following pairs of amino acid residues:
DE, DK, DA, EG, QG, RG, AA, EE, FF, GG, HH, KK, LL, DN, PQ, QQ, RR, DT, VV, WW, or YY.

In a further aspect of the invention, in an ADC as described herein, in the affinity-matured variant mAb-v53, it may be advantageous to further mutate this variant to replace “NS” (asparagine/serine) at position 53 and 54 of CDR2 in the variable heavy chain with alternative amino acids. For example, the N may be replaced with one of A, E, F, G, H, I, K, L, P, Q, R, V, W or Y, whilst keeping S at position 54. Alternatively, the S may be replaced with A, E, F, G, H, I, K, L, P, Q, R, V, W or Y whilst keeping N at position 54. Or both could N and S could be changed, such that a combination of the above variations may be used.

In an alternative aspect, the antibody component of the ADC as described herein may be based on, or incorporate, an antibody as described in WO2019/147152A1, to which further reference can be made for details.

Thus, in a further aspect of the present invention, an antibody-drug conjugate (ADC) is provided, wherein the ADC comprises an antibody comprising a combination of a light chain variable region (VL) and a heavy chain variable region (VH), wherein:
the VL comprises complementarity determining regions (CDRs) L-CDR1, L-CDR2, and L-CDR3 as set forth in SEQ ID NOs. 179, 181, and 183, respectively; and,
the VH comprising CDRs H-CDR1, H-CDR2 and H-CDR3 as set forth in SEQ ID NOs. 185, 187 and 189, respectively.

The antibody-drug conjugate (ADC) may comprise an antibody wherein the VL comprises SEQ ID NOs. 178, 180, and 182; and the VH comprises SEQ ID NOs. 184, 186 and 188. In one preferred aspect, the antibody-drug conjugate (ADC) comprises an antibody as described above wherein the VL comprises SEQ ID No. 177 and the VH comprises SEQ ID No. 176.

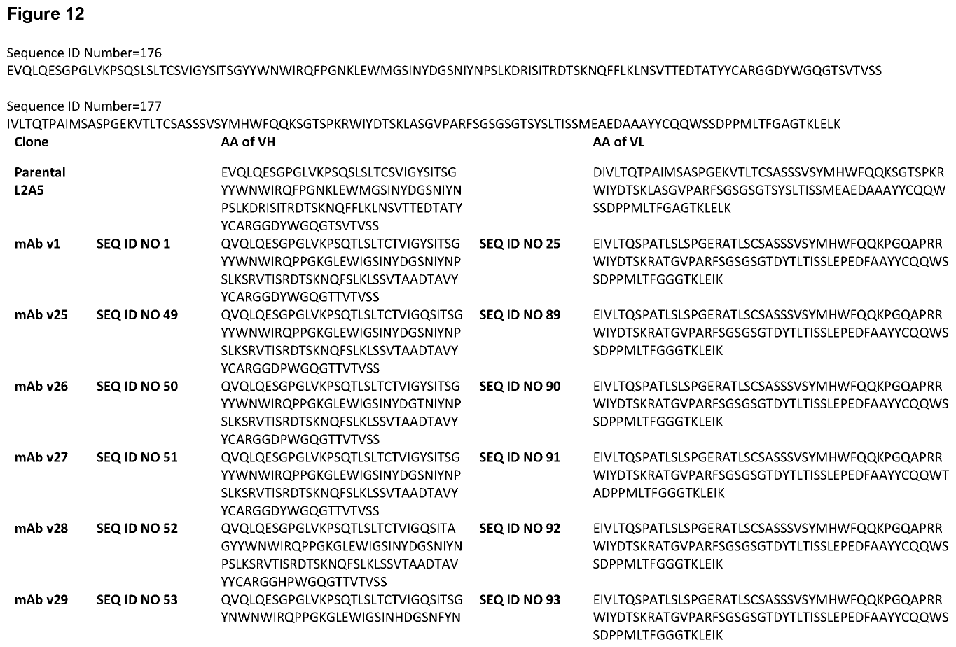

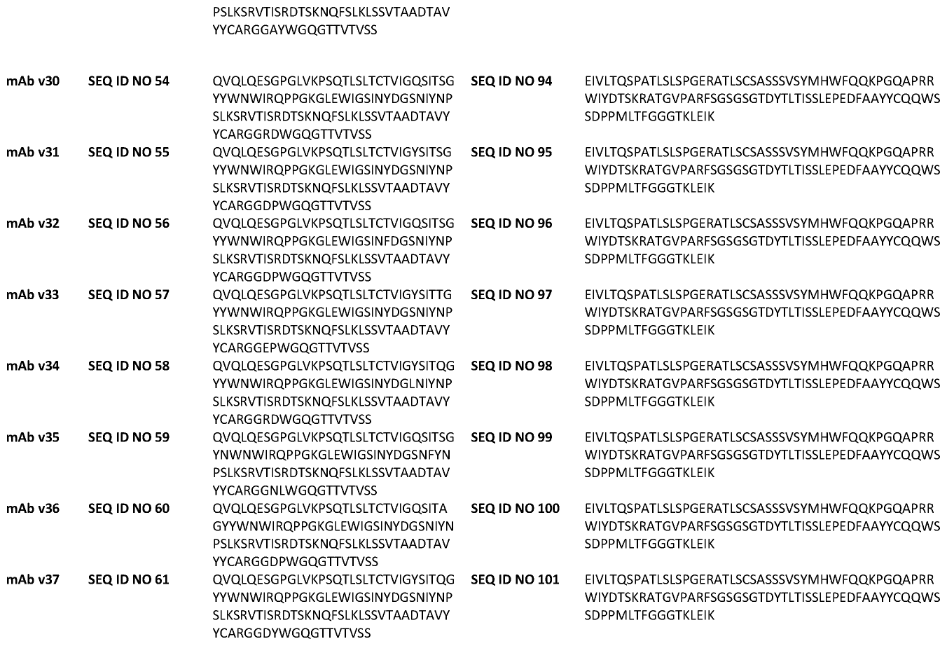

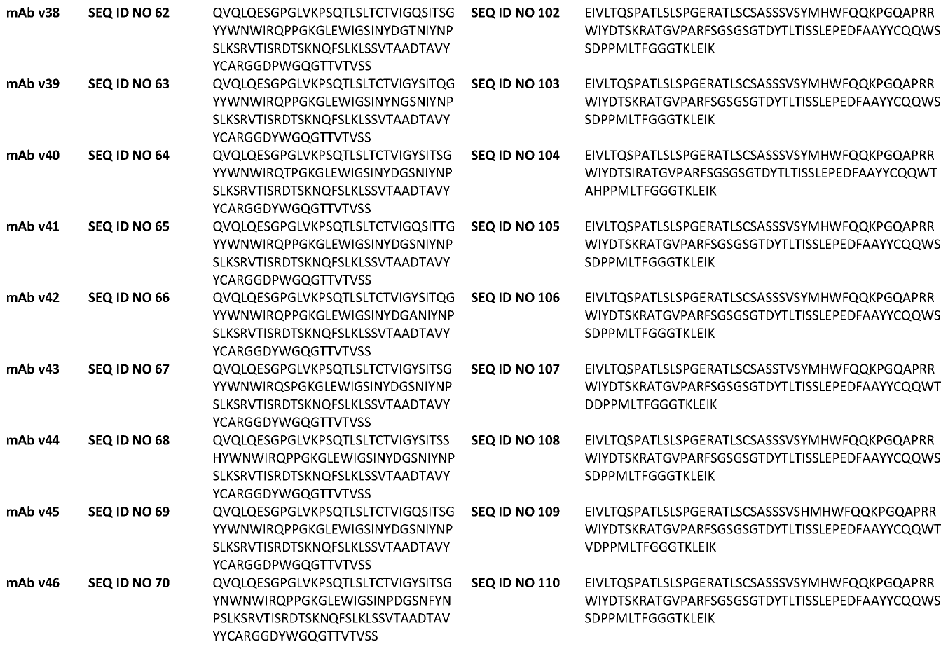

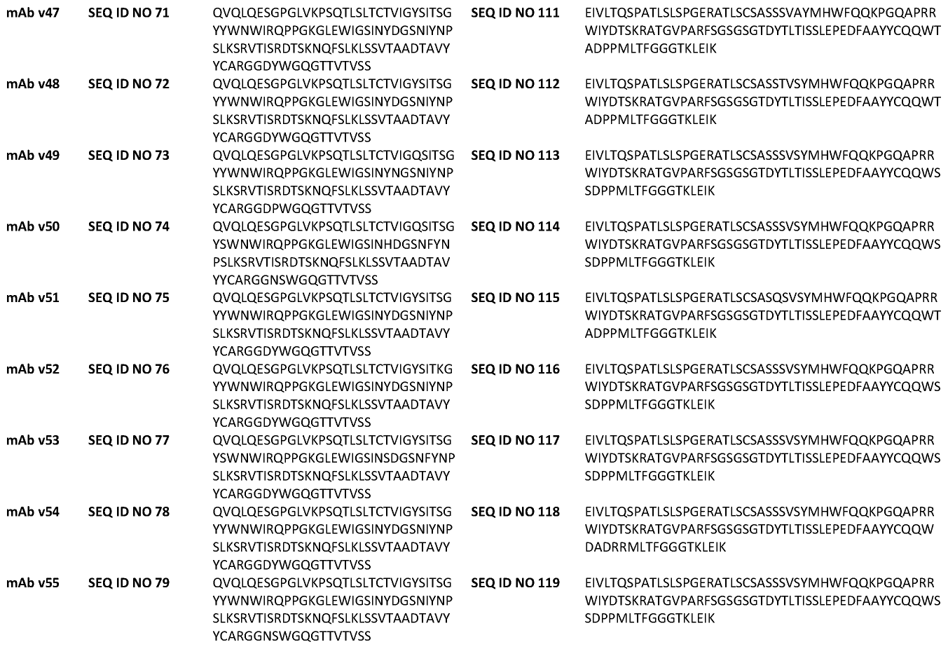

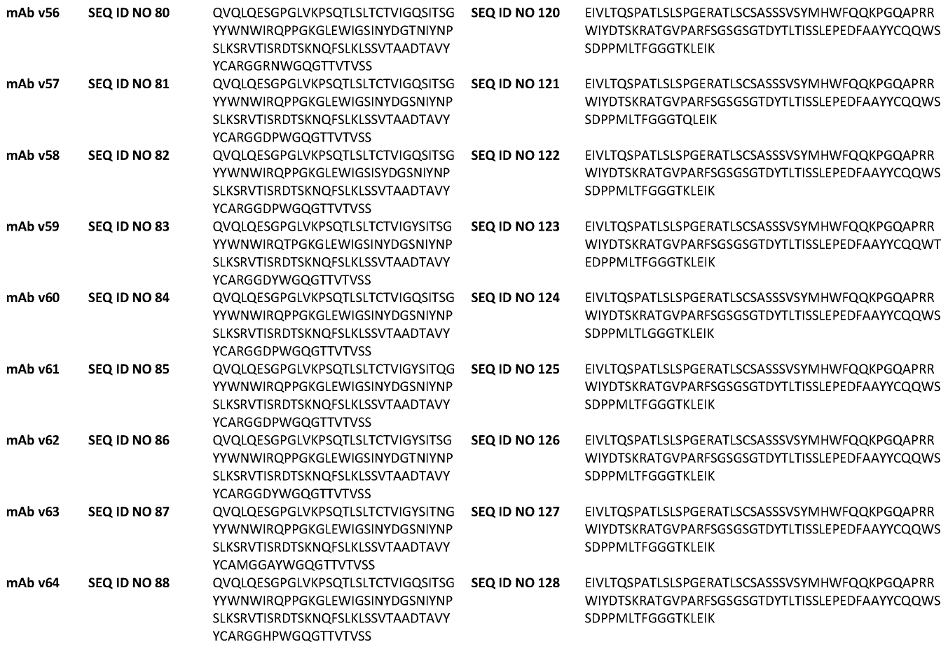

In one preferred aspect, the antibody-drug conjugate (ADC) comprises an antibody as described above wherein the VL and VH amino acid sequences comprise those shown for L2A5 in FIG. 12.

The antibody described preferably binds STn and a group of glycans terminated by alpha 2,6-linked sialic acids. Preferably, the glycans terminated by alpha 2,6-linked sialic acids comprise STn, 2,6-sialyl T, di-sialyl T, or 2,6-sialolactosamine. The antibody described may be subject to glycan changes at glycosylation sites.

The antibody described above may be a monoclonal antibody, chimeric antibody, or a humanized antibody.

The antibody described above may further be in the form of a functional antibody fragment thereof, that binds STn and a group of glycans terminated by alpha 2,6-linked sialic acids. In an aspect of the present disclosure, there is also provided an expression vector comprising a polynucleotide encoding an antibody as described above, optionally wherein the polynucleotide comprises:
SEQ ID NO. 174 and/or SEQ ID NO. 175; SEQ ID NO. 190, GACACATCC and SEQ ID NO. 191; or SEQ ID NO. 192, SEQ ID NO. 193, and SEQ ID NO. 194.

Also disclosed herein in a host cell comprising an expression vector as described above.

A method of producing an antibody as described above may for example comprise using a host cell as described above.

### Applications of the Novel ADC

The present invention also provides a pharmaceutical composition comprising an antibody-drug conjugate (ADC) as described herein, and a pharmaceutically acceptable carrier.

In a further aspect, there is also provided an antibody-drug conjugate (ADC) as described herein for use in medicine. “Medicine” is used herein in its broadest aspect, to include treatment of both the human and animal body. Thus, veterinary applications, for treatment of animals, are specifically contemplated, in addition to the treatment of human patients.

In a preferred aspect, there is also provided an antibody-drug conjugate (ADC) as described herein for use in treating cancer.

The novel ADC described presents innovative features, including the use of antibodies targeting cancer specific biomarker (absent in normal tissues) in conjugation with novel linker payload with increasing stability in circulation.

The incorporation of extra PEG units, for example preferably three extra PEG units, in the linker structure provides several advantages, including increased solubility and stability of the ADC, as well as enhanced pharmacokinetics. The carbamate or quaternary ammonium handle/linker ensures a stable bond between the antibody and the cytotoxic payload, while also allowing for the precise release of the payload upon internalization into cancer cells.

Finally, the beta glucuronide linker together with PEG3 increases the hydrophilicity, reducing aggregation during conjugation, compared to other linkers and increasing stability in circulation.

### Novel Linkers

As a result of their work, the present inventors have in fact now devised certain linkers which have hitherto been undisclosed, that is, they are novel linkers. These novel linkers have been found to have particular utility, including possessing certain advantages as described herein, when used in an ADC with antibodies which bind to sialyl Tn (STn) or a glycan terminated by an alpha 2,6-linked sialic acid, including particularly the specific antibodies described above; especially when the drug or payload is a chemotherapeutic agent or cytotoxic agent, such as for example exatecan or a derivative thereof, or deruxtecan or a derivative thereof. But these linkers are also of more general utility and can, in principle, be used with other types of antibody and other drugs or payloads, other than those specifically described herein—where they are expected to confer the same or similar advantages to those described herein.

Thus, in accordance with a further aspect of the present invention, there is provided a linker for use in an antibody-drug conjugate (ADC), which ADC is suitable for treating cancer, wherein the linker comprises a linker cleavable by glucuronidase, wherein the linker is configured to be coupled to a drug via a quaternary ammonium salt linkage.

There is also provided, in another aspect, a linker for use in an antibody-drug conjugate (ADC), which ADC, is suitable for treating cancer, wherein the linker comprises a linker cleavable by glucuronidase, wherein the linker is PEGylated with a group comprising polyethylene glycol (PEG).

In a further aspect, the invention provides a linker cleavable by glucuronidase, wherein the linker is configured to be coupled to a drug via a quaternary ammonium salt linkage, and is PEGylated with a group comprising polyethylene glycol (PEG).

In a further aspect, the invention provides a linker cleavable by glucuronidase, wherein the linker is configured to be coupled to a drug via a carbamate linkage, and does not include an alkyne moiety.

In a further aspect, the invention provides a linker cleavable by glucuronidase, wherein the linker is configured to be coupled to a drug via a carbamate linkage and terminates in a maleimide moiety.

In a preferred aspect, a linker is provided wherein the linker is configured to be coupled to a drug via a quaternary ammonium salt linkage and wherein the linker is also PEGylated with a group comprising polyethylene glycol (PEG).

In one aspect, the linker may comprise a β-glucuronide moiety.

In one aspect, the linker comprises a β-glucuronide moiety as shown in formula I′:

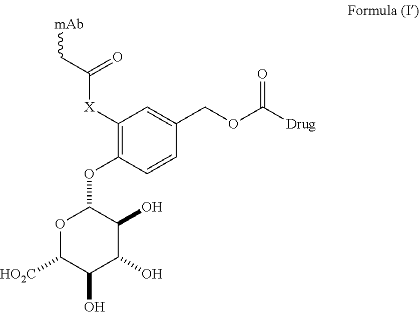

wherein X is NH, N—CH₃ or CF₂.

In one embodiment of formula (I′), X is NH. In one embodiment of formula (I′), X is N—CH₃. In one embodiment of formula (I′), X is CF₂.

In a preferred aspect, the linker may comprise a β-glucuronide moiety as shown in formula I:

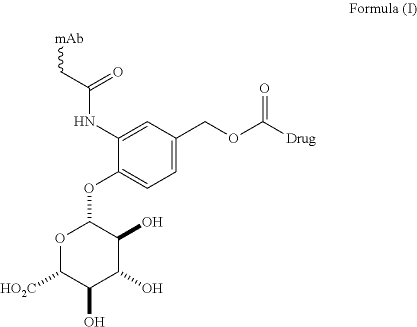

wherein formula (I) is either further modified to be configured to be coupled to a drug via a quaternary ammonium salt linkage, or wherein the linker is PEGylated with a group comprising polyethylene glycol (PEG). In a preferred aspect, where PEGylation is employed, the linker is PEGylated with a group comprising polyethylene glycol (PEG) on the amide moiety in formula (I′) or (I).

In one preferred aspect, formula (I′) or (I) is both further modified to be configured to be coupled to a drug via a quaternary ammonium salt linkage, and the linker is PEGylated with a group comprising polyethylene glycol (PEG). Preferably, the linker is PEGylated with a group comprising polyethylene glycol (PEG) on the amide moiety in formula (I′) or (I).

Preferred examples of linkers in provided according to the present invention include those where the linker is configured to be coupled to a drug via a quaternary ammonium salt moiety or linkage.

Thus in a preferred aspect, there is provided a linker wherein the linker is configured to be linked to, or is modified to link to, a drug via a quaternary ammonium salt linkage. Thus, for example, in formula I above the carbamate linkage or moiety may be replaced with a quaternary ammonium salt linkage or moiety. One suitable quaternary ammonium salt linkage or moiety is shown illustrated in formula IV or VI below, it being understood that this linkage or moiety could be employed in linker structures other than those specifically shown in formula IV or VI.

Where PEGylation of the linker is employed, the linker may for example be PEGylated with a group comprising polyethylene glycol (PEG) based on the structure:

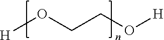

wherein n is from 1 to 5, preferably 2 to 4,
   although if desired one or both of the terminal H substituents in the above structure may be replaced by another suitable chemical group, provided that the functioning of the linker is substantially unaffected. In a preferred aspect, in the structure above n is 3, that is, the PEG comprises three individual monomer units.

In a preferred aspect, the linker may be PEGylated with a group of formula II:

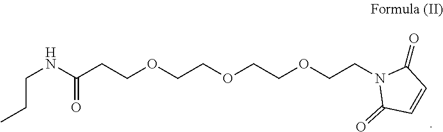 [SMILES: CCCNC(=O)CCOCCOCCOCCN1C(=O)C=CC1=O]

The PEGylation may for example be on the amide group of the linker, for example on the amide group of the structure shown in formula I. In a preferred aspect, a linker comprising a group of formula II is also configured to be coupled to a drug via a quaternary ammonium salt linkage or moiety.

In one preferred aspect, the linker is as shown in formula III′:

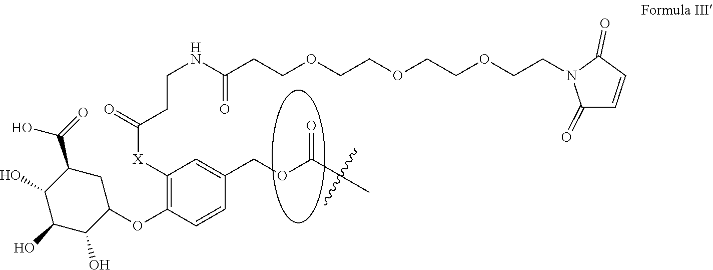

wherein X is NH, N—CH₃ or CF₂.

In one embodiment of formula III′, X is NH. In one embodiment of formula III′, X is N—CH₃. In one embodiment of formula III′, X is CF₂.

In one aspect, a preferred linker according to the invention is a PEGylated linker as shown in formula III:

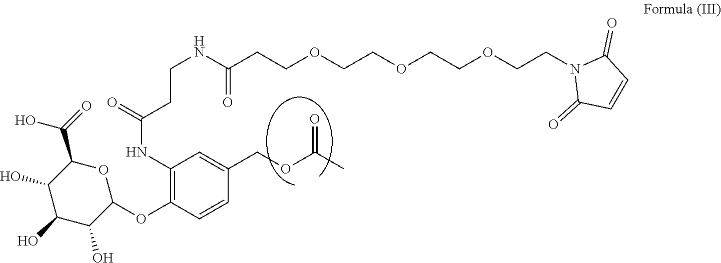 [SMILES: CC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

In one preferred aspect, the linker is as shown in formula IV′:

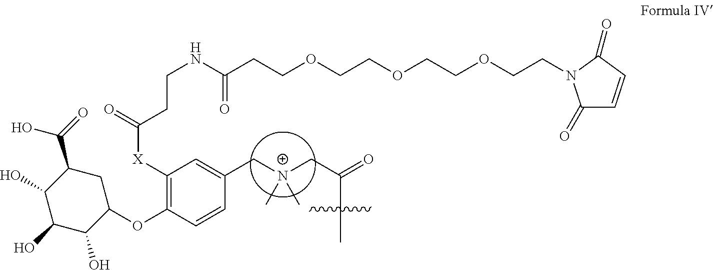

wherein X is NH, N—CH₃ or CF₂.

In one embodiment of formula IV′, X is NH. In one embodiment of formula IV′, X is N—CH₃. In one embodiment of formula IV′, X is CF₂.

In another aspect, a preferred linker according to the invention is a PEGylated linker as shown in formula IV:

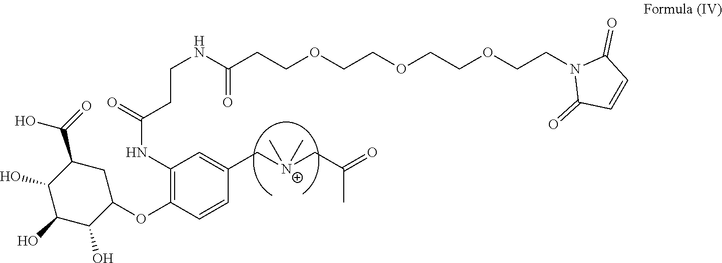 [SMILES: CC(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)C=CC2=O)c1]

In a further preferred aspect, a linker is provided wherein the linker is coupled to a drug to form a drug-linker payload, as shown in formula V′:

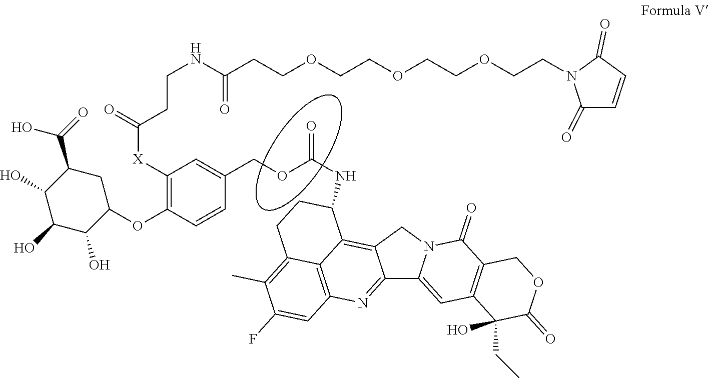

wherein X is NH, N—CH₃ or CF₂.

In one embodiment of formula V′, X is NH. In one embodiment of formula V′, X is N—CH₃. In one embodiment of formula V′, X is CF₂.

In a further preferred aspect, a linker is provided wherein the linker is coupled to a drug to form a drug-linker payload, wherein the linker is as shown in formula VI′:

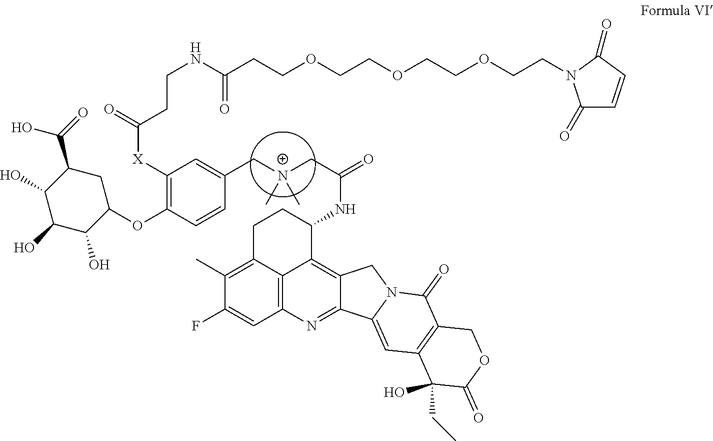

wherein X is NH, N—CH₃ or CF₂.

In one embodiment of formula VI′, X is NH. In one embodiment of formula VI′, X is N—CH₃. In one embodiment of formula VI′, X is CF₂.

In a further preferred aspect, a linker is provided wherein the linker is coupled to a drug to form a drug-linker payload, wherein the linker is as shown in formula V or VI:

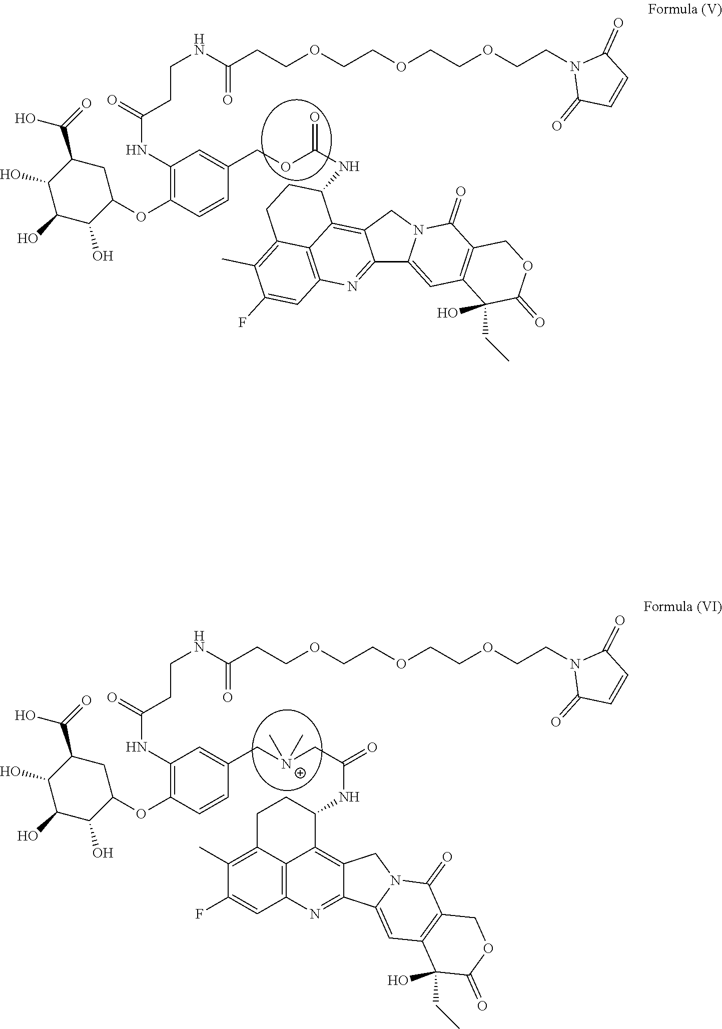

The invention also provides an exatecan derivative compound with a handle moiety covalently bonded to said exatecan parent molecule at a position that does not significantly affect the therapeutic or pharmacokinetic properties of the parent molecule, wherein said handle moiety comprises a quaternary ammonium salt of the formula:

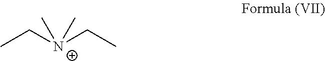

In a preferred aspect, there is also provided an exatecan derivative compound having the following general structure:

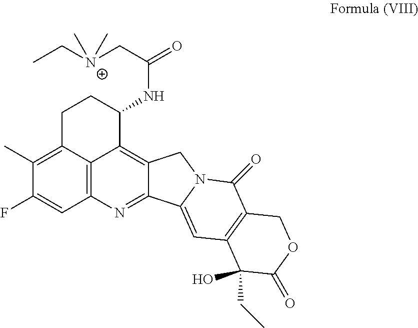 [SMILES: CC[C@@]1(O)C(=O)OCc2c1cc1n(c2=O)Cc2c-1nc1cc(F)c(C)c3c1c2[C@@H](NC(=O)C[N+](C)(C)CC)CC3]

The exatecan derivative compounds provided as described above exhibit an improved safety profile compared to the exatecan parent molecule and retain therapeutic activity against cancer cells.

Thus, it will be appreciated that the invention also provides an antibody drug conjugate (ADC) comprising an antibody conjugated to a drug via a linker as described above. Preferably, the ADC is for treating cancer, but it will be understood that, in principle, the linkers described may be employed in other types of ADCs, not only those directed at the treatment of cancer.

However, in a preferred aspect, an antibody-drug conjugate (ADC) employing a linker as described herein will comprise an antibody which binds to sialyl Tn (STn) or a glycan terminated by an alpha 2,6-linked sialic acid.

Also, in a preferred example, an antibody-drug conjugate (ADC) employing a linker as described herein will comprise a drug which is a growth inhibitory agent suitable for treating cancer, such as exatecan or a similar compound.

Preferably, an antibody-drug conjugate (ADC) employing a linker as described herein will comprise an antibody which binds to sialyl Tn (STn) or a glycan terminated by an alpha 2,6-linked sialic acid, and also a drug which is a growth inhibitory agent suitable for treating cancer, such as exatecan or a similar compound.

### Results

In the current invention, we describe cancer-specific antibody drug conjugates, suitable for precision medicine with the intention to improve target specificity, bystander effect and safety profile.

### Synthesis and Conjugation of Novel Linker-Payload:

The linker-payload used in the present invention may for example be synthesized through a multi-step process that ensures the formation of a stable and cleavable bond between the cytotoxic payload and the linker as shown in FIG. 1a-b. The synthesis process involves the following steps:
1. Preparation of the cytotoxic payload, exatecan, using well-established synthetic procedures at DAR4. The selected DAR offers an optimal balance between therapeutic efficacy and safety, ensuring a sufficient number of cytotoxic molecules per antibody to effectively target and kill tumor cells, while minimizing the risk of toxicity to healthy tissues.
2. Synthesis of the linker, which comprises a beta-glucuronide moiety, 3 polyethylene glycol (PEG) chains, and a handle (carbamate or quaternary amine) for attachment to the antibody. Incorporation of, for example, three extra PEG units in the linker structure provides several advantages, including increased solubility and stability of the ADC, as well as enhanced pharmacokinetics. The carbamate handle/linker ensures a stable bond between the antibody and the cytotoxic payload, while also allowing for the precise release of the payload upon internalization into cancer cells. The two final linker-payloads, underlined in FIG. 1a-b (final construct) were designed to provide stability, solubility, and cleavability in the context of an ADC.
3. Conjugation of the beta-glucuronide linker to the exatecan payload. This step includes the formation of a cleavable bond between the cytotoxic payload and the beta-glucuronide linker.

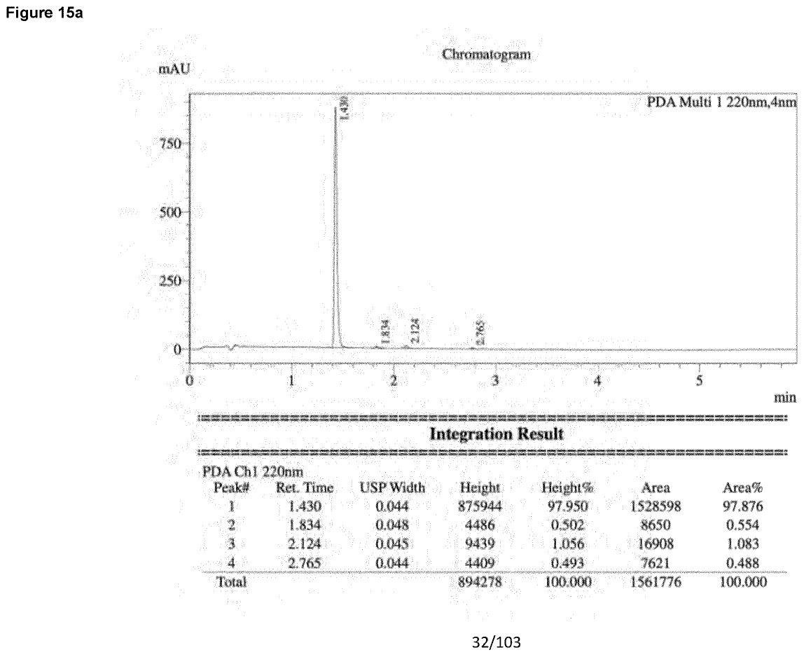

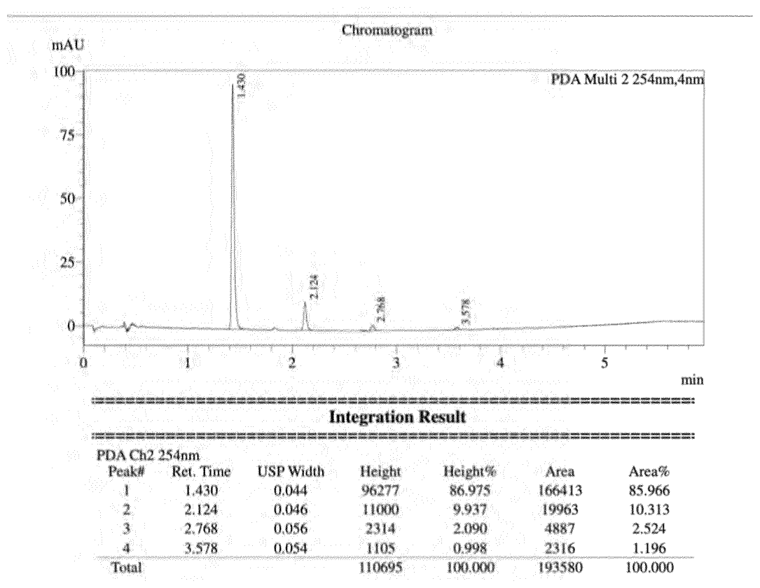

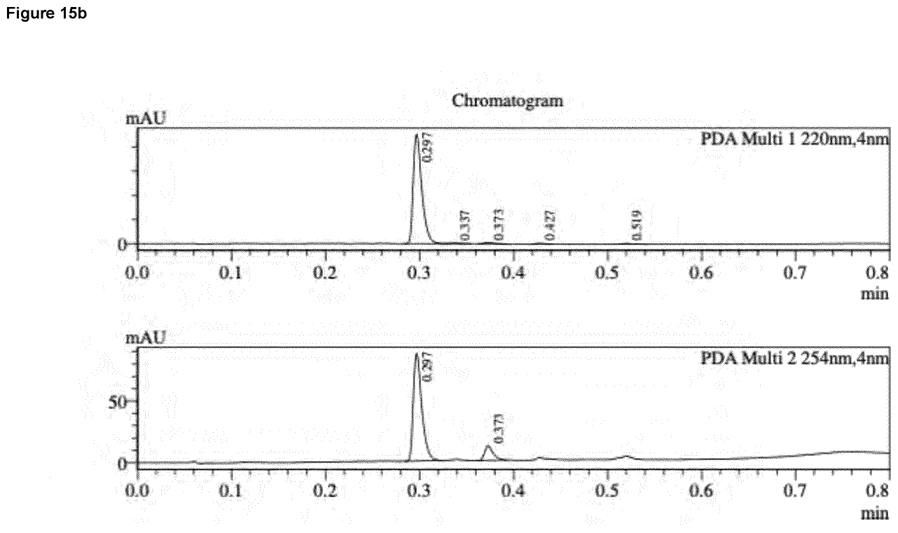

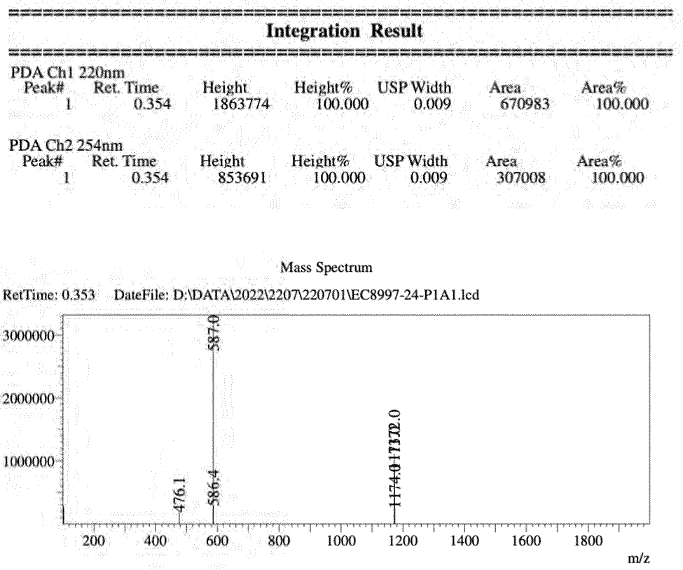

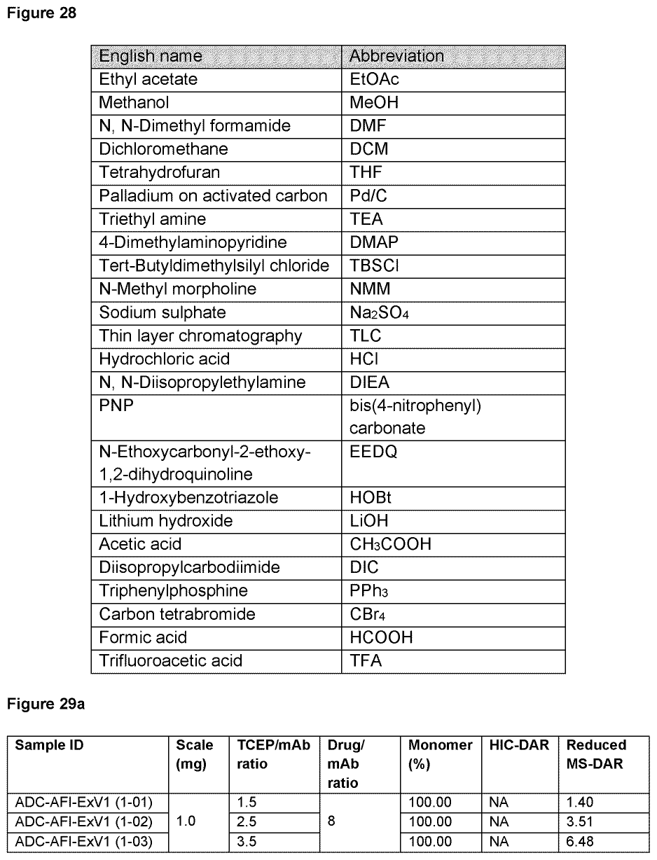

Step by step synthesis for linker-payload version 1 and version 2 are listed in Detailed description section, under the Carbamic linkage-based glucuronide drug linker (for Version 1) and Quaternary ammonium linkage-based glucuronide drug linker (for Version 2). HPLC and LCMS relative to each compound were listed in FIGS. 15-27b. Relative acronym legend was listed in FIG. 28. The numbering of the compounds referred to is given in the Supplementary Figures.

### Bioconjugation Process:

The bioconjugation process involves the attachment of the linker-payload to the antibody, yielding a homogeneous and well-defined ADC with a specific drug-to-antibody ratio (DAR).

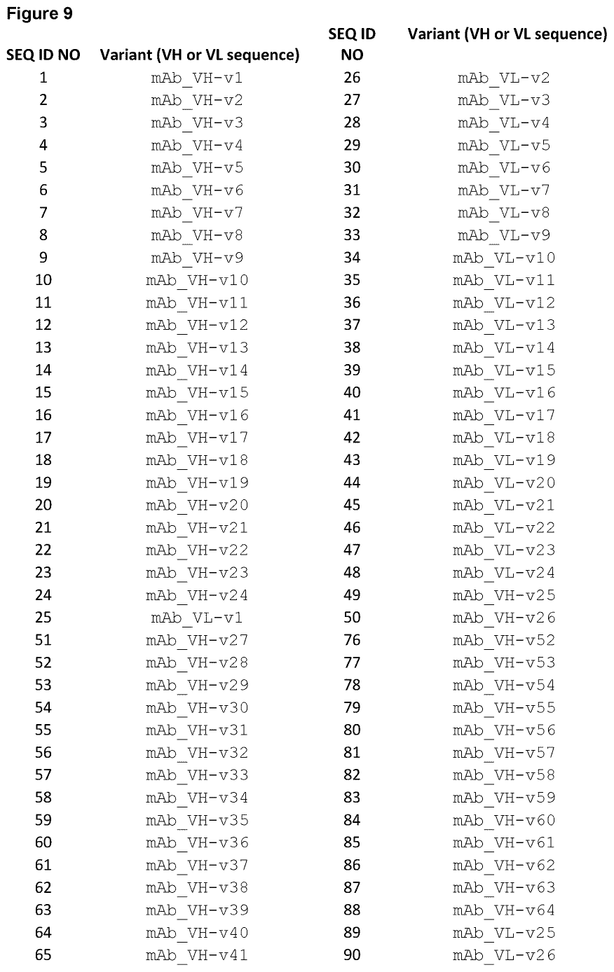

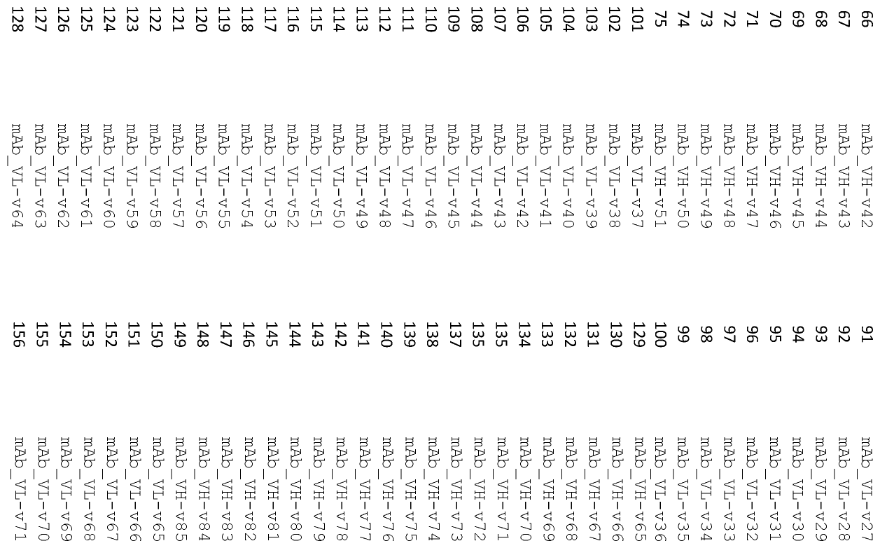

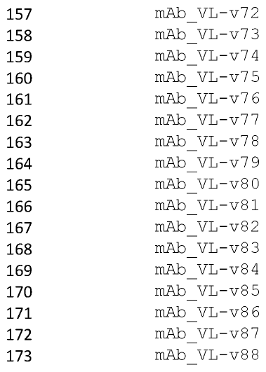

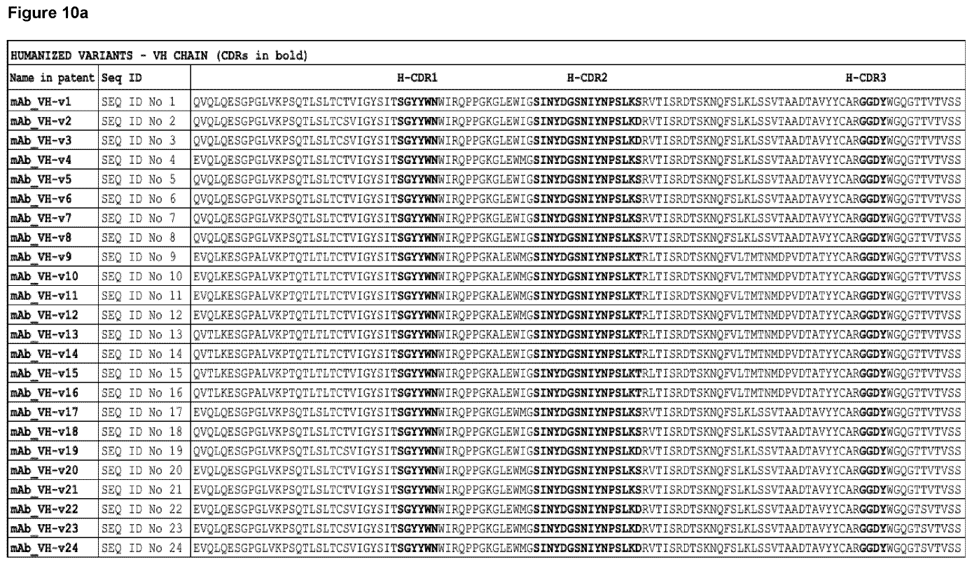

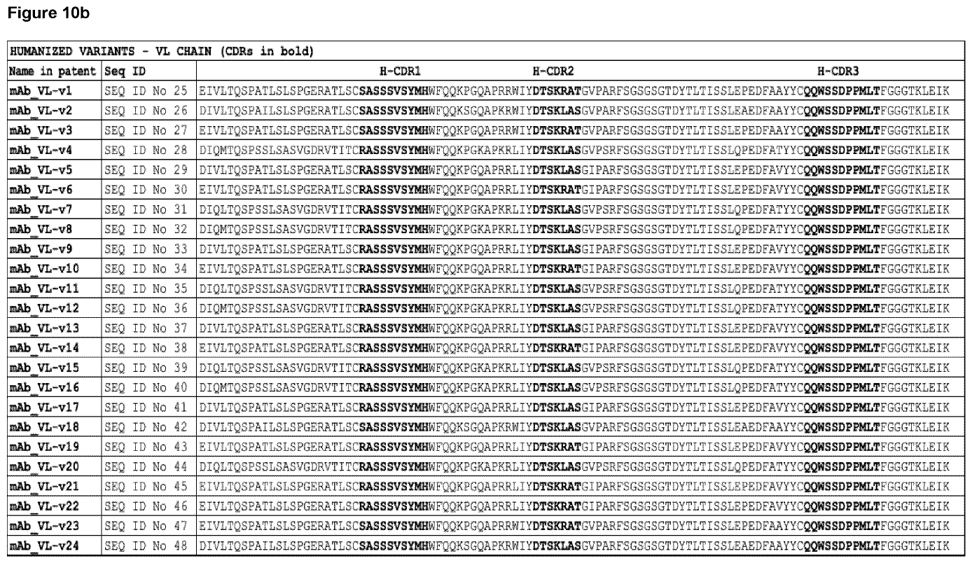

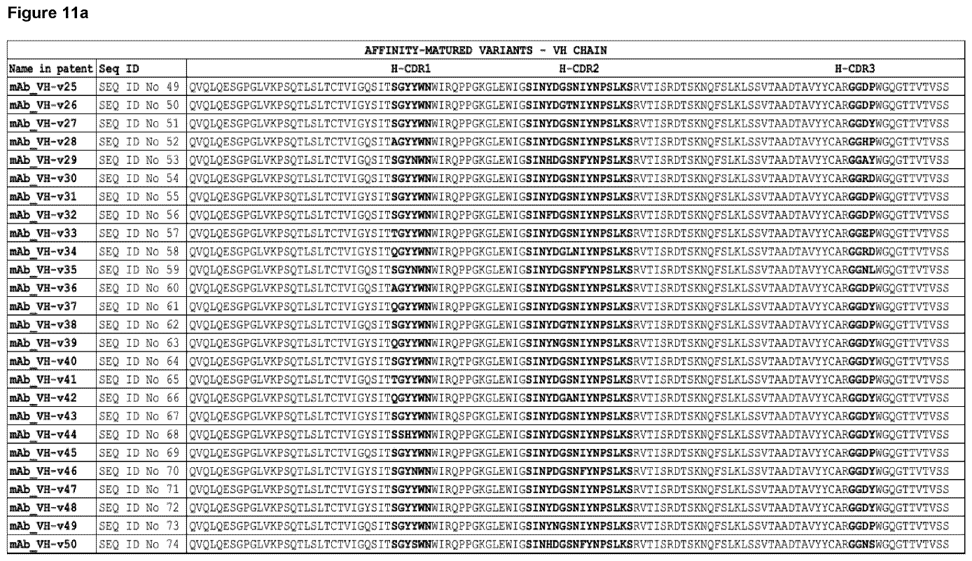

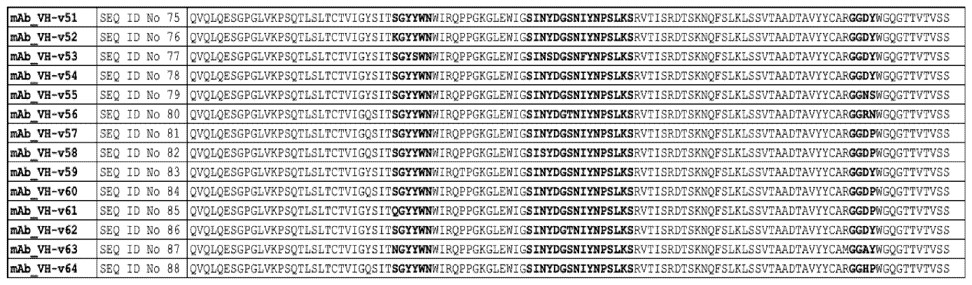

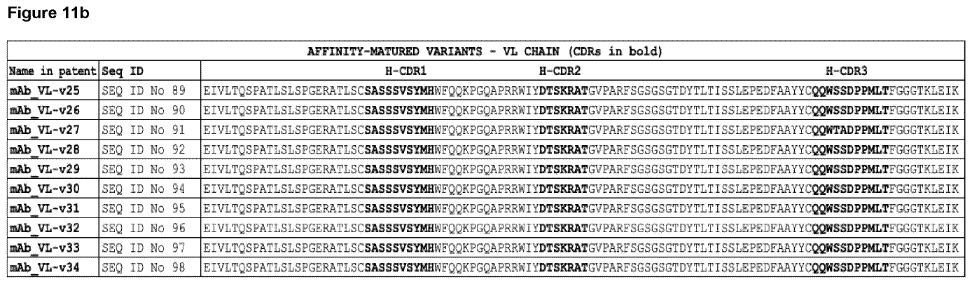

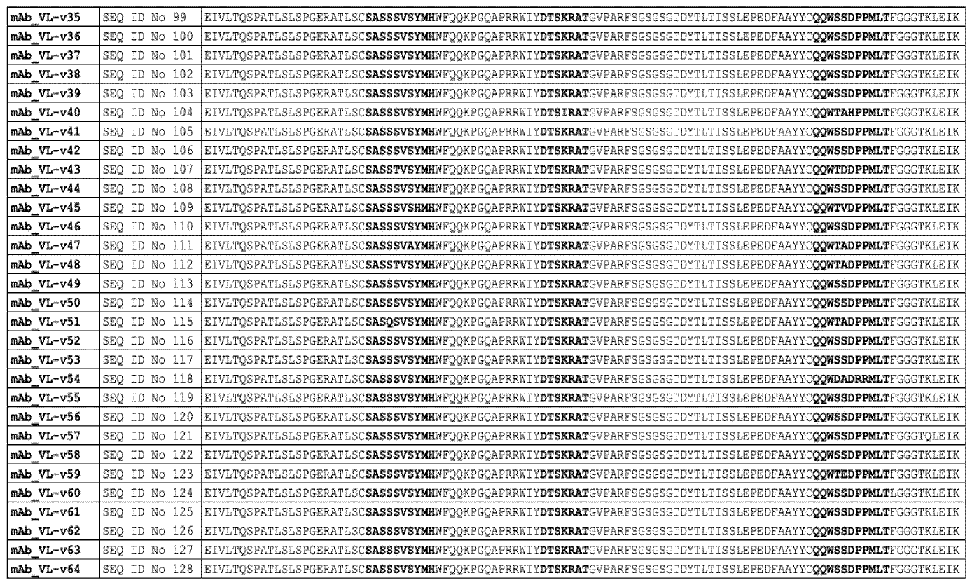

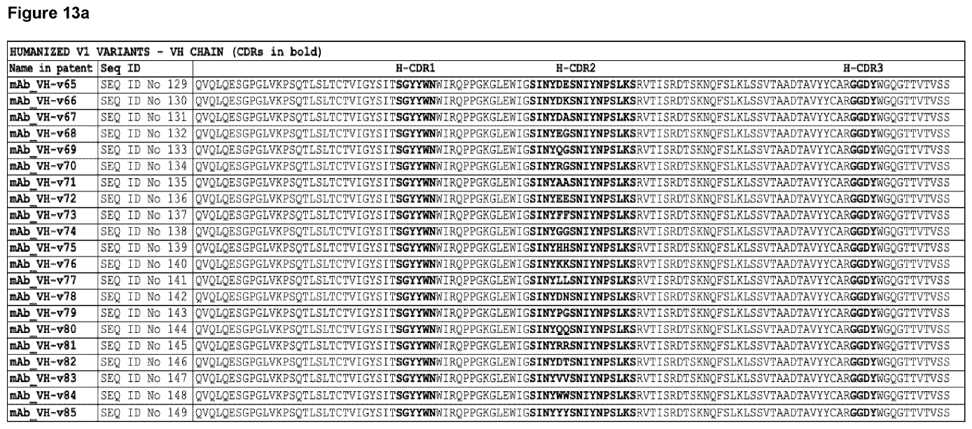

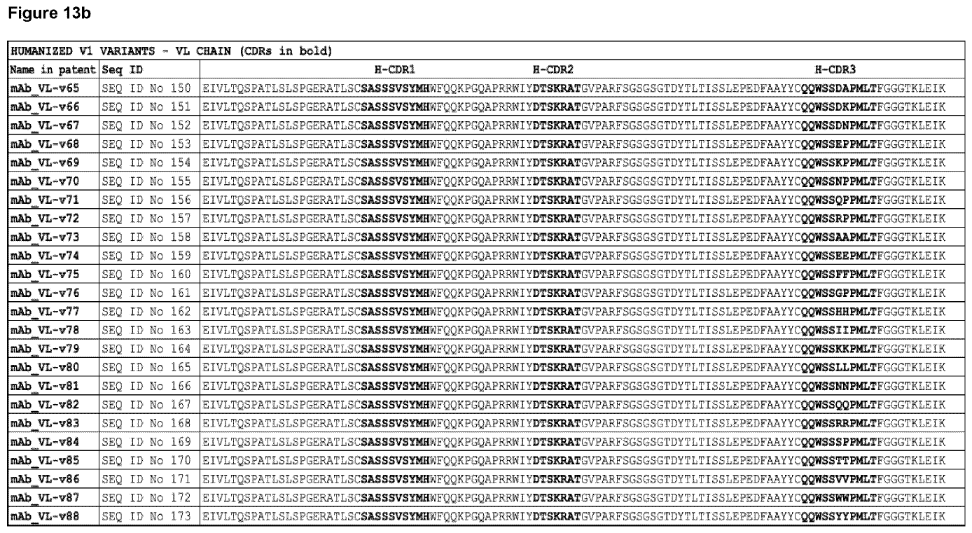

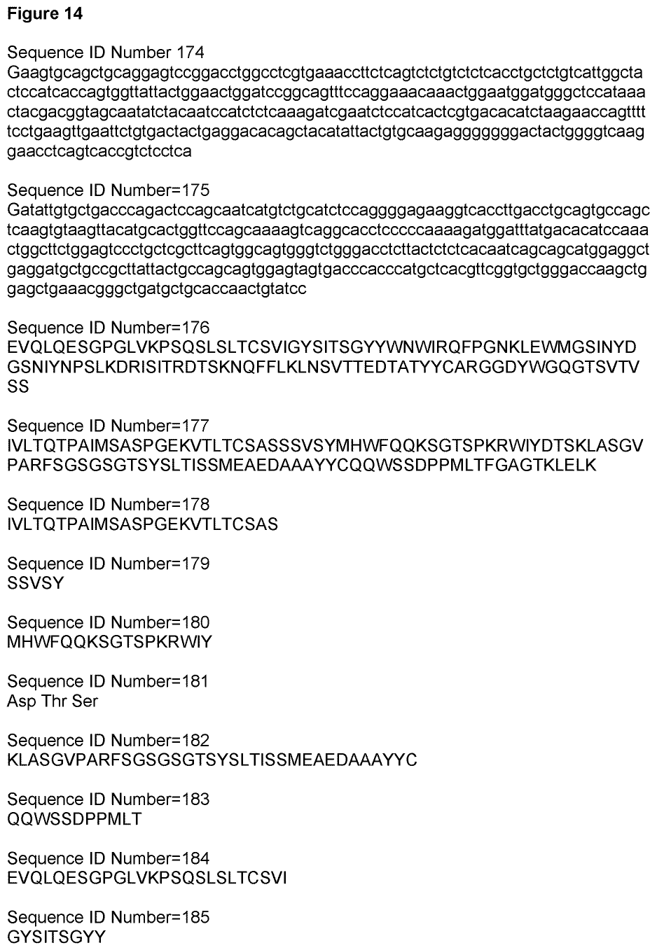

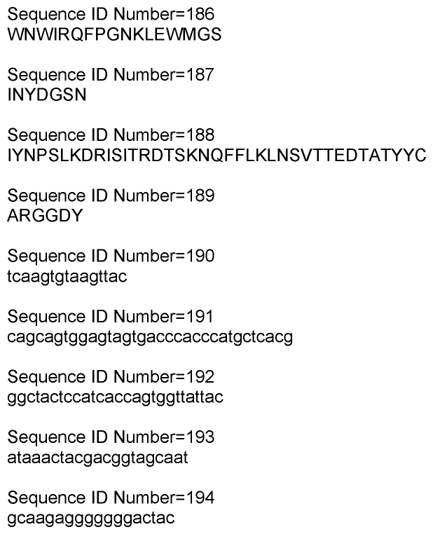

The bioconjugation process may be carried out using the following steps:
1. Selection and preparation of the antibody or antibody fragment, which specifically recognizes the target antigen, STn. This may involve the production of recombinant antibodies in suitable expression systems. A list of different mAb clones candidate is reported in FIGS. 9 to 14.
2. Identification and selection of suitable conjugation sites on the antibody, such as cysteine residues or lysine residues, ensuring that the conjugation does not compromise the antibody's binding affinity or specificity.
3. Conjugation of the linker-payload to the selected conjugation site(s) on the antibody using a site-specific coupling chemistry. This step may involve the formation of a stable bond, such as a thioether bond or an amide bond, between the antibody and the linker-payload.
4. Purification of the resulting ADC to remove any unreacted linker-payload, unconjugated antibodies, and other impurities. This step may involve techniques such as size-exclusion chromatography, ion-exchange chromatography, or other appropriate methods as listed in Detailed Description section.
5. Characterization of the purified ADC to confirm the drug-to-antibody ratio (DAR), the integrity of the antibody, the stability of the linker-payload, and the retention of antigen-binding properties. This step may involve various analytical techniques, such as mass spectrometry, SDS-PAGE, and surface plasmon resonance (SPR).

As disclosed herein one humanized affinity matured clone antibody (mAb_v64, comprising SEQ ID Nos 88 (VH) and 128 (VL)) was used for conjugation with the novel linker-payload version1 (carbamic linkage-based) resulting in the named ADC-AFI-ExV1, as used herein. Sequence relative to mAb_v64 were reported in Supplementary Sequence antibody list (FIG. 12)

### Pilot Small Scale (1 mg) ADC-AFI-V1 Production:

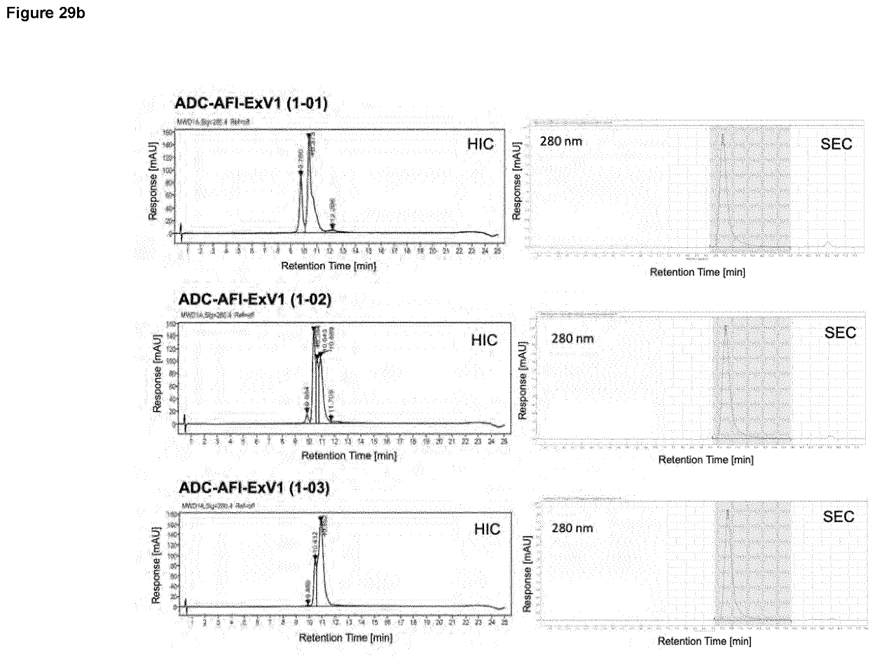

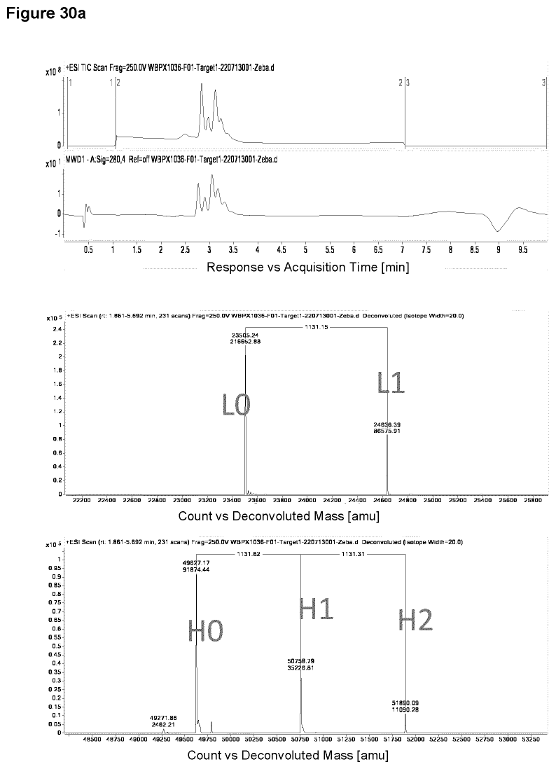

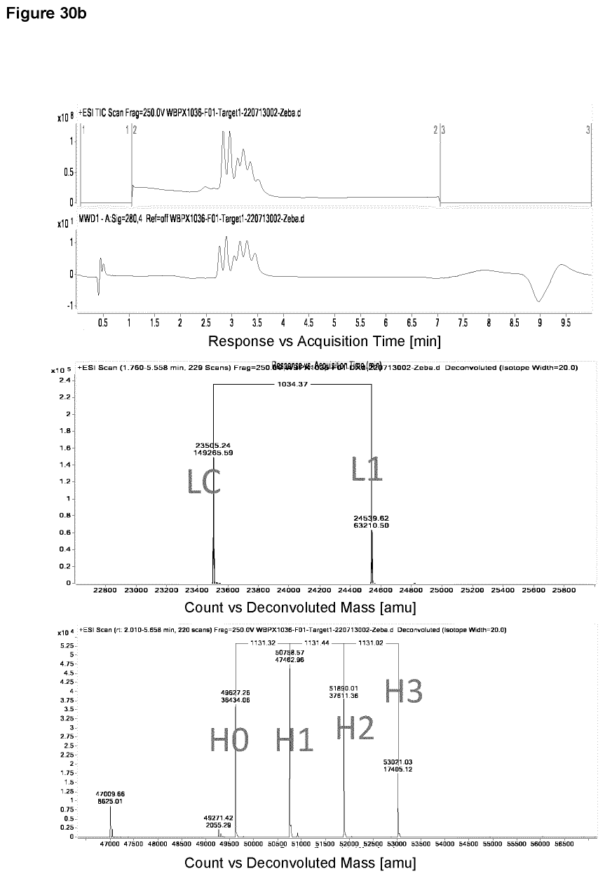

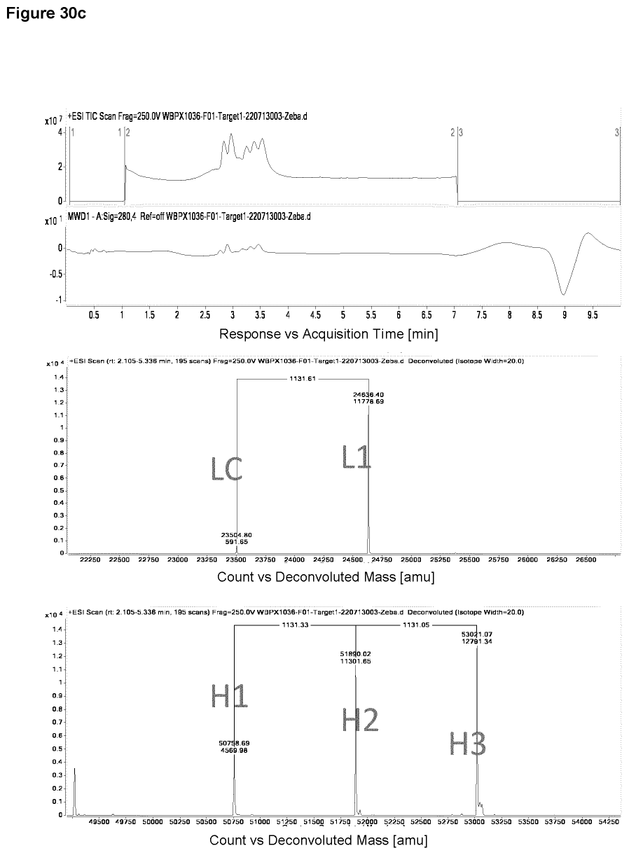

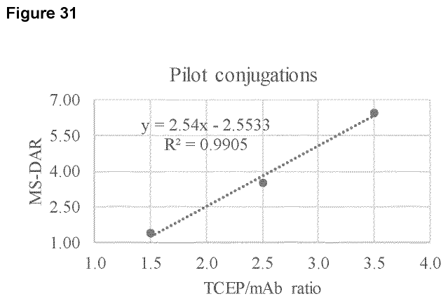

Process involved an initial pilot small-scale ADC-AFI-ExV1 production at 1 mg scale, and TCEP/mAb molar eq. was set as 1.5, 2.5 and 3.5, respectively. The results were summarized in FIG. 29a. To determine the DAR, HIC-HPLC was firstly used as shown in FIG. 29b (left graph). Using this technique different DAR species did not separate well in HIC-HPLC which reflect the high hydrophilicity of linker payload Version 1. This data confirms and validate the high hydrophilicity of ADC-AFI-ExV1. The reduced MS-DAR was then used to determine the DAR of ADC-AFI-ExV1. Raw data relative to the SEC-HPLC was shown in FIG. 29b (right graphs) and data relative to LC-MS was reported in FIG. 30a-c. The curve that represents the relationship between TCEP/mAb eq. and reduced MS-DAR is shown in FIG. 31. According to the relationship between TCEP/mAb ratio and reduced MS-DAR, the optimum TCEP/mAb ratio for ADC-AFI-ExV1 with target DAR of 4.0 was 2.58. All ADC products had good purity under all conditions.

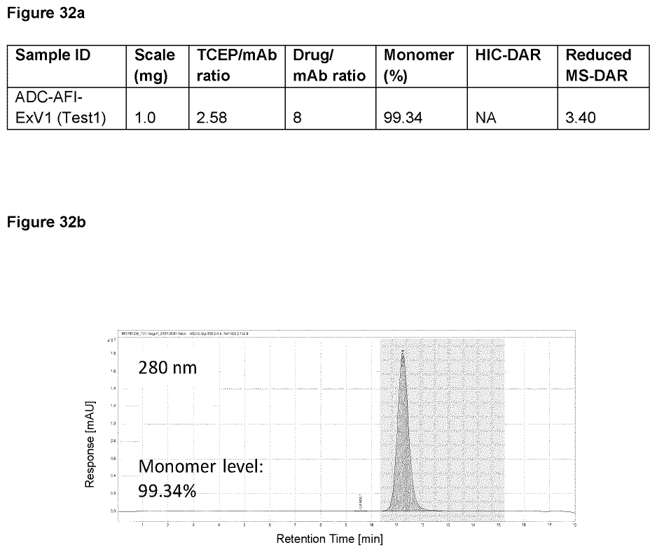

Since the optimum condition was found in pilot conjugation, confirming conjugation (1 mg scale) was performed to verify the condition. The results were summarized in FIG. 32a, and SEC and LC-MS data were shown in FIG. 32b-c, respectively. As shown in FIG. 32b, the product showed high monomer levels, however as shown in FIGS. 32a and 32c, the reduced MS-DAR is lower than expected DAR of 4.0.

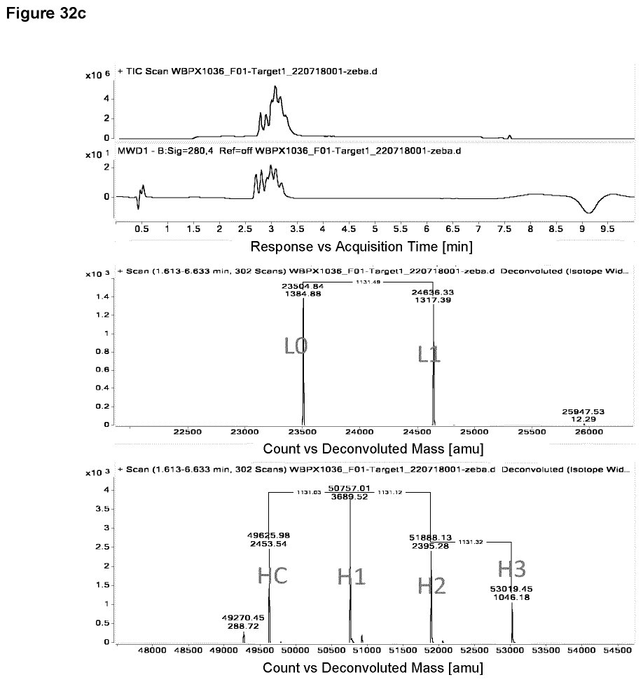

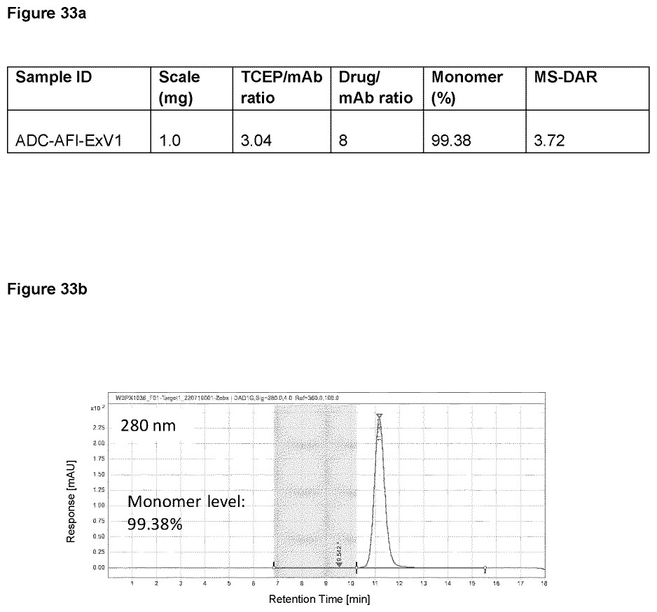

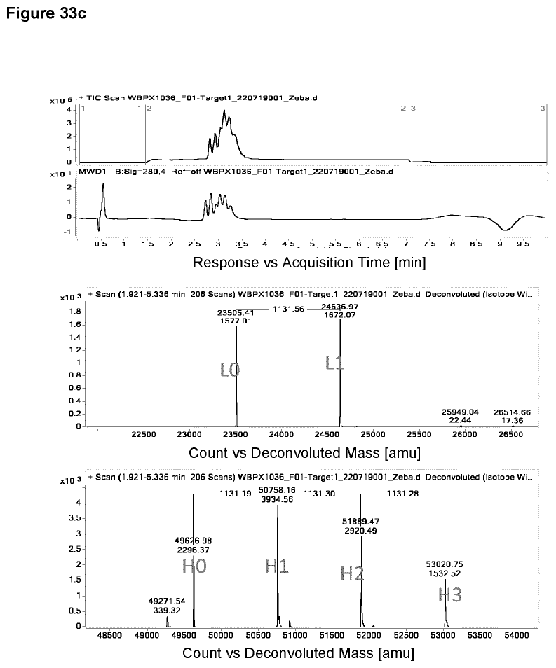

In order to obtained ADC product with target DAR of 4.0, the TCEP/mAb molar eq. was increased to 3.04 in the 2^{nd} confirming conjugation. The result of new confirming conjugation is summarized in FIG. 33a and the SEC data and LC-MS data are shown in FIG. 33b-c, respectively. As shown in FIG. 33b, the product showed high monomer levels. As shown in FIGS. 33a and 33c, the optimized TCEP/mAb ratio of 3.04 is suitable for bulk conjugation since the target DAR is 4±0.4.

### Final (30 mg) ADC-AFI-V1 Production (Bulk Conjugation):

For decreasing the deviation caused by scale-up, the 30 mg bulk conjugation is divided into two batches (15 mg each).

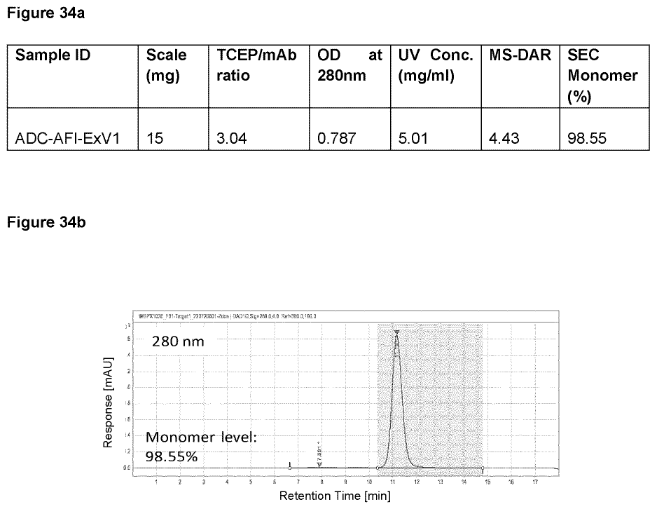

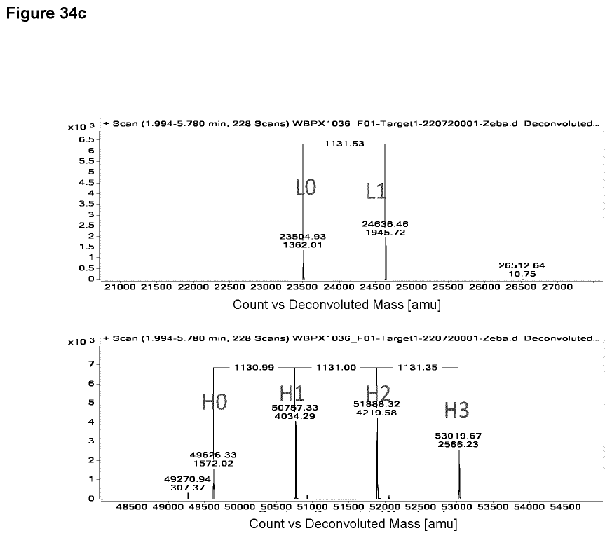

Since the confirming conjugation was successful, the first bulk conjugation was performed with the confirmed optimum condition with TCEP/mAb of 3.04. The result is summarized in FIG. 34a, and SEC and LC-MS data are shown in FIG. 34b-c respectively. As shown in FIG. 34b, the product has high monomer level. As shown in FIGS. 34a and 34c, the reduced MS-DAR was a slightly higher than target of 4.0.

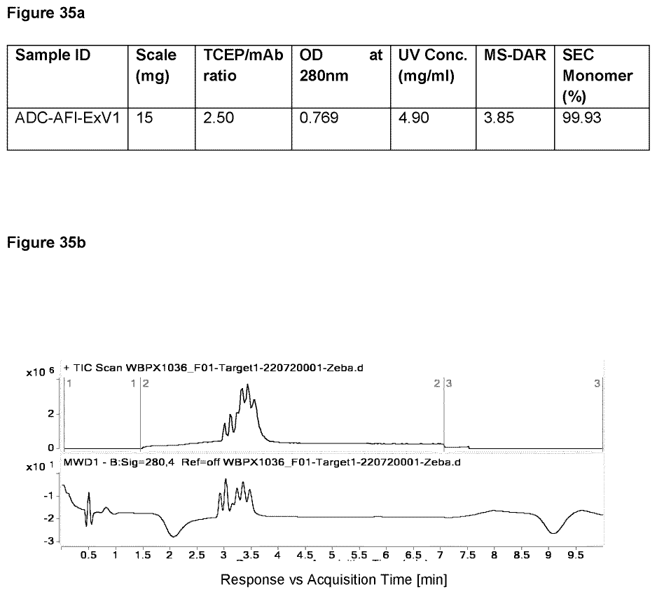

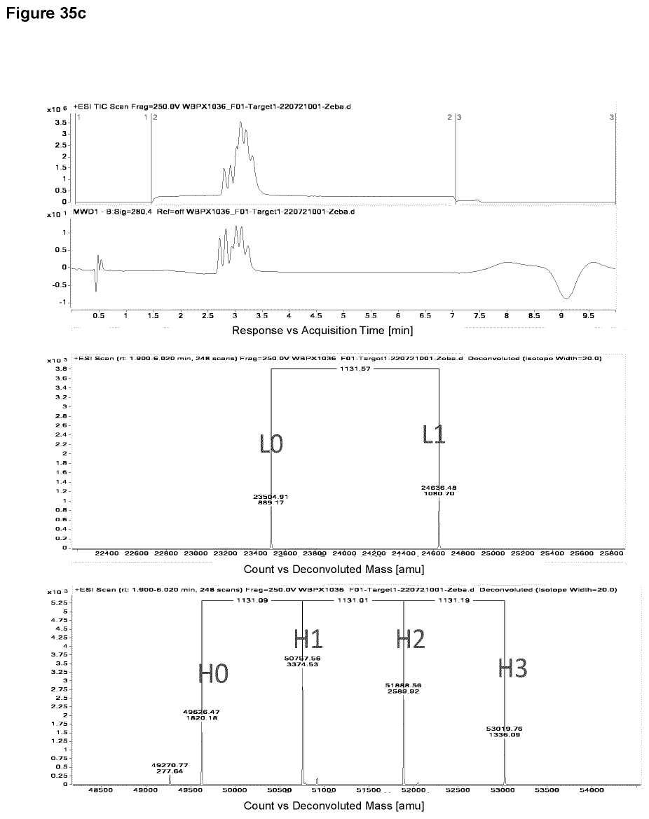

In the second 15 mg batch conjugation, the TCEP/mAb ratio is decreased to 2.50. The result is summarized in FIG. 35a, and SEC data and LC-MS data are shown in FIG. 35b-c, respectively. As shown in FIG. 35b, the product has high monomer level. As shown in FIGS. 35a and 35c, the reduced MS-DAR meets the requirement.

Finally, the first and the second batch of ADC-AFI-ExV1 was combined for dialysis against formulation buffer. After dialysis, the product was applied for dextran coated charcoal treatment to remove residual free drug. ADC was filtered by 0.22 μm membrane as final product and submitted for characterization.

### Final Conjugation of Three Different ADC Products:

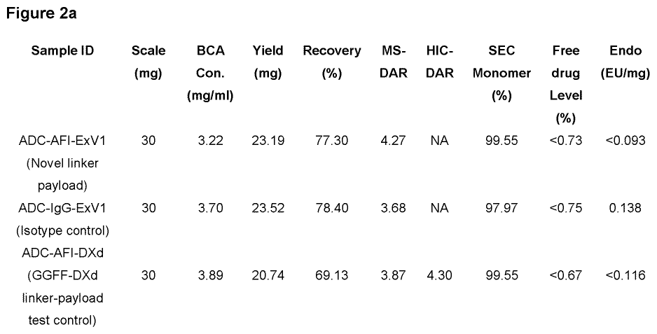

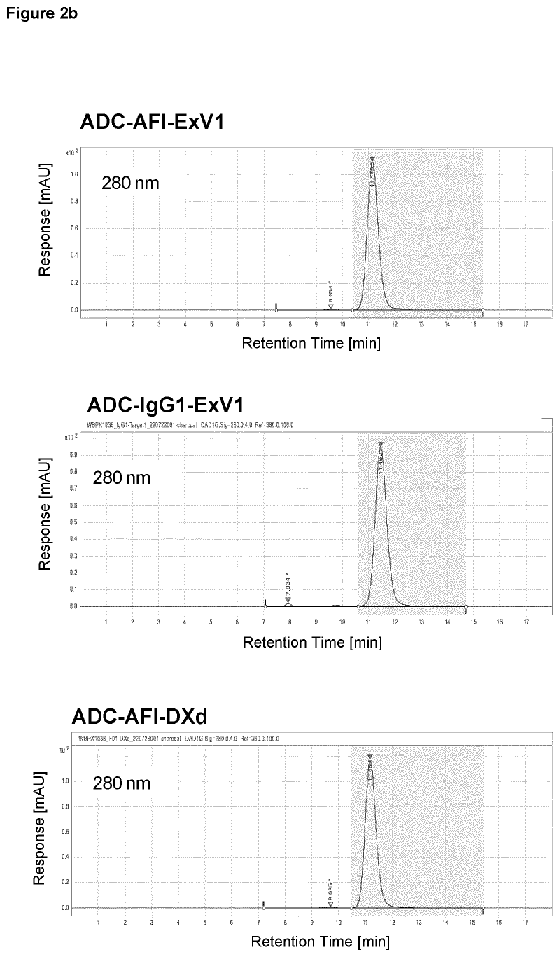

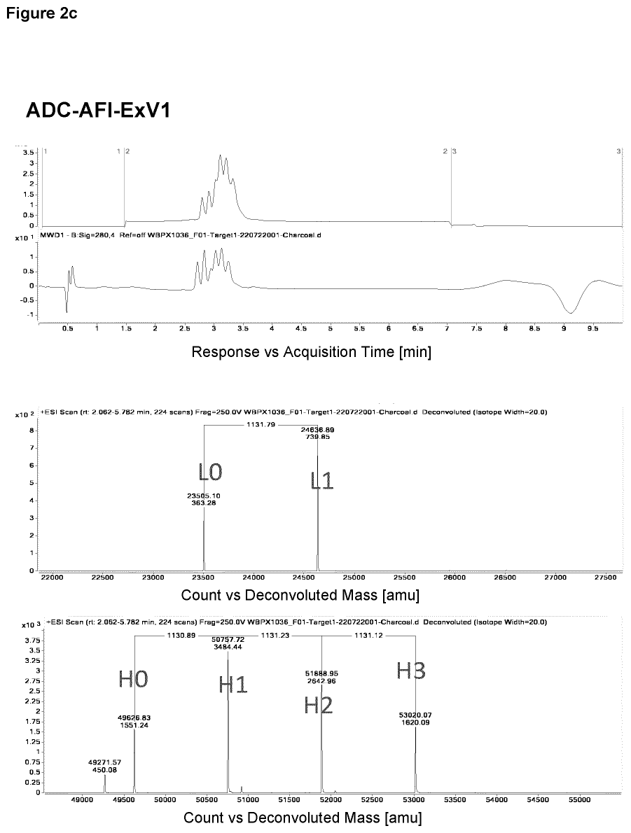

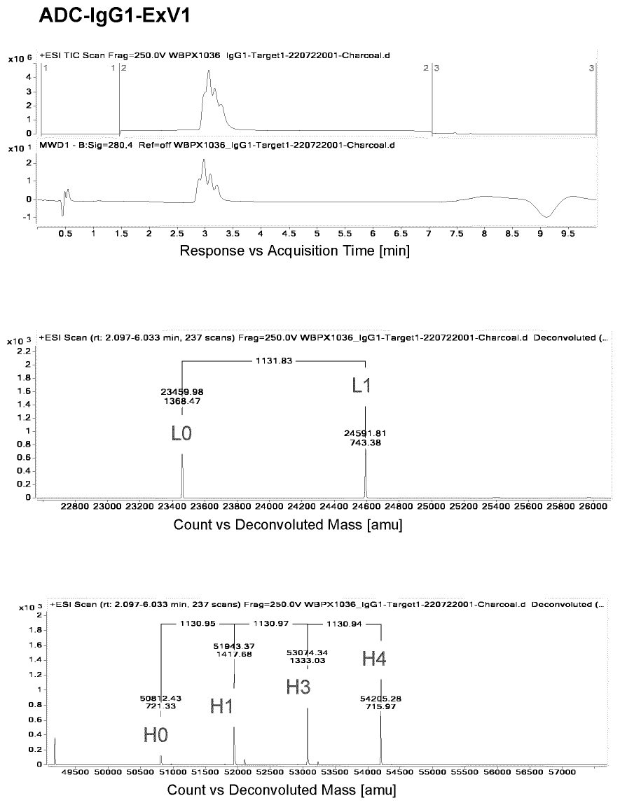

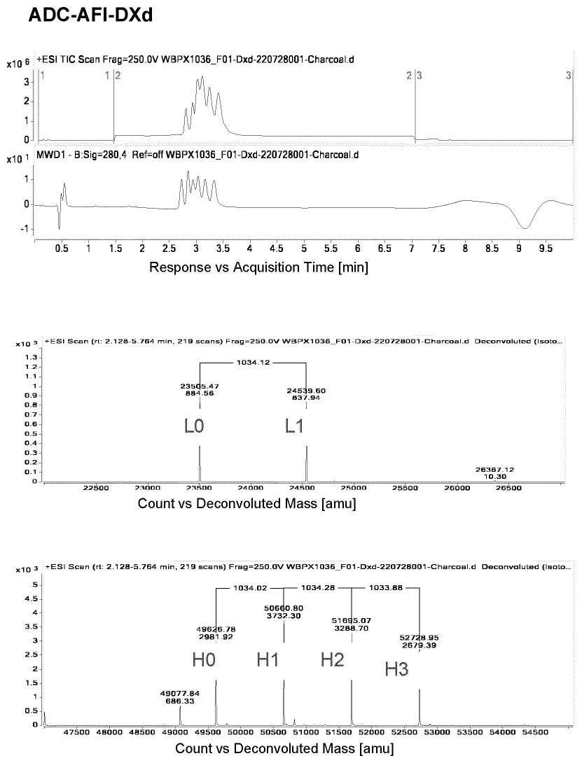

With the intention to evaluate the efficacy of ADC-AFI-ExV1, the same naked antibody (mAb_v64) was conjugated as well with available Deruxtecan (GGFG-DXd) linker payload. In addition a IgG1 isotype control was included as well as control in conjugation with the same novel linker-payload Version 1 (V1) and named ADC-IgG1-ExV1. Results for all three ADCs are listed in FIG. 2a including three antibodies included in the scale-up production (30 mg):
ADC-AFI-ExV1 (mAb v64; Exatecan carbamic linkage-based “version1”)
ADC-AFI-DXd (mAb v64; GGFG-DXd “Deruxtecan”)
ADC-IgG1-ExV1 (IgG1 isotype control; Exatecan carbamic linkage-based “version1”)

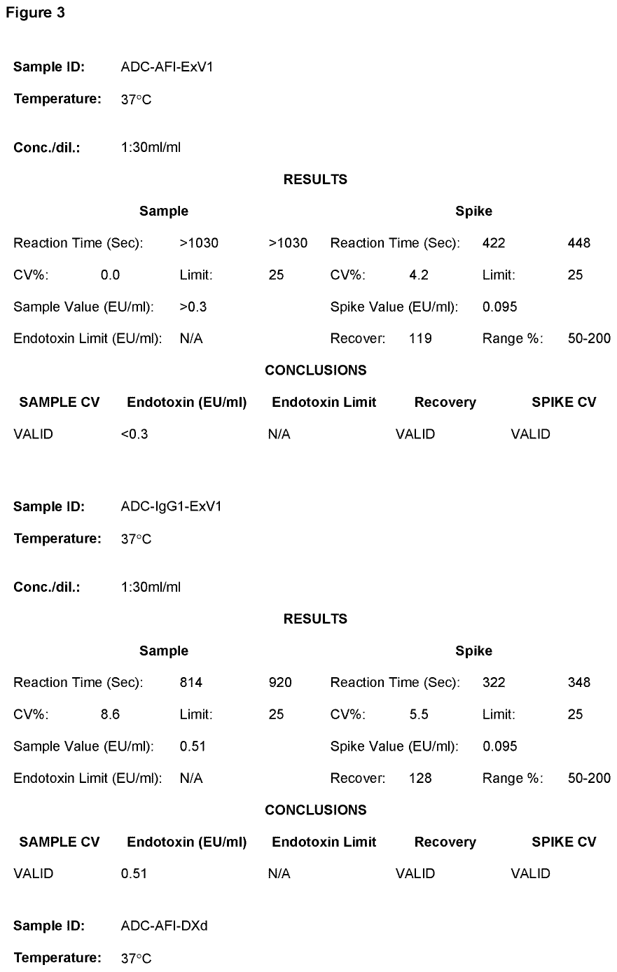

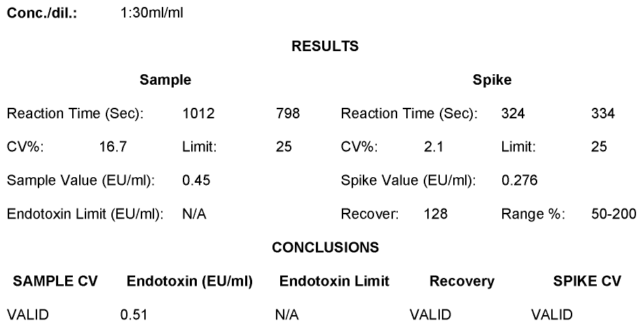

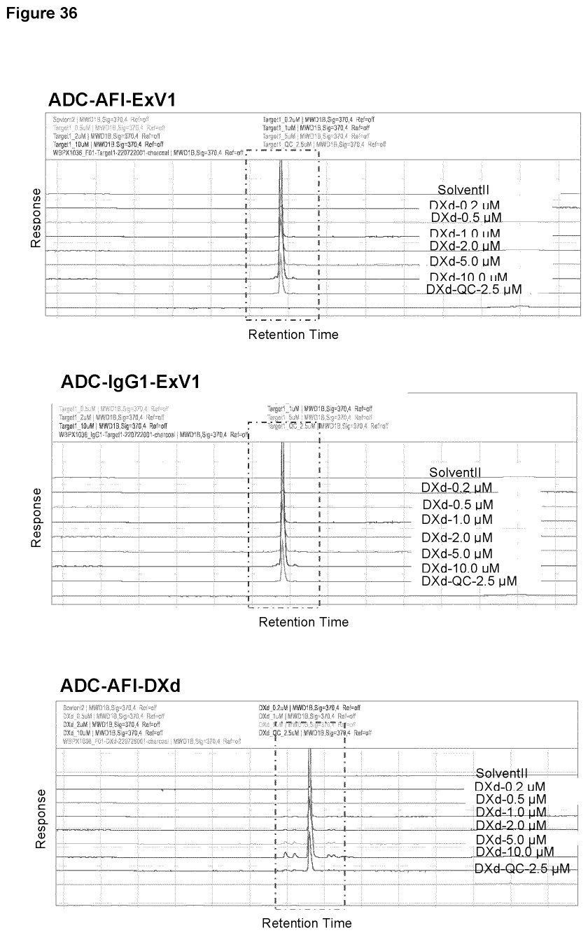

SEC and reduced MS data relative to the three ADCs were listed respectively in FIG. 2b-c. Additional RP data relative to same ADCs was listed in FIG. 36. Finally, data reported in FIG. 3 showed minimal endotoxin levels for all ADCs listed above, indicating safety of testing products.

Differently than ADC-AFI-ExV1 (FIG. 29b; HIC graph), DAR evaluation using Hydrophobic Interaction Chromatography (HIC) was possible only when analyzing ADC-AFI-DXd (FIG. 37). Together these data underline a higher hydrophilicity profile of the new ADC-AFI-ExV1 compared to the ADC-AFI-DXd (antibody linker-payload control). Additional data on RP relative to all ADCs produced were listed in FIG. 37.

The present invention thus discloses a method for producing a stable and efficacious ADC, comprising a linker-payload and a specific antibody, and a bioconjugation process that ensures the formation of a well-defined ADC with a specific drug-to-antibody ratio (DAR)—preferably a DAR of 4 or less. The production and bioconjugation methods described herein are amenable to scale-up and can be adapted for the manufacturing of ADCs for human and animal health applications.

### SNU16-CDX In Vivo Efficacy Data

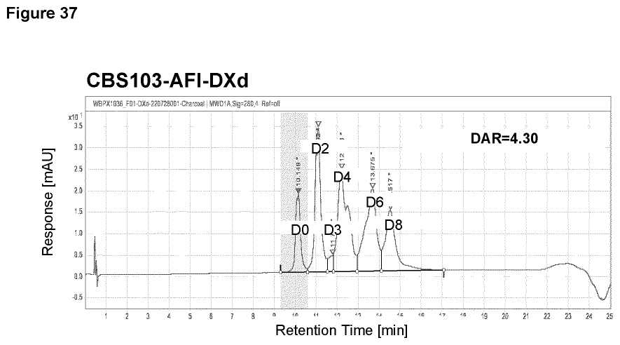

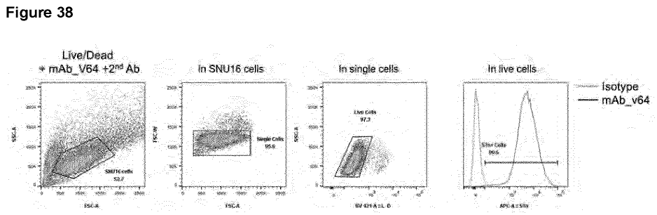

The objective of this study was to evaluate the in vivo anti-tumor efficacy of Exatecan-V1 linker-payload as described in ADC-AFI-ExV1 compared to well-known linker payload: Deruxtecan (GGFG-DXd). The model used in the efficacy evaluation is the human gastric SNU-16 subcutaneous xenograft model (CDX) in female BALB/c Nude mice. SNU16, express high level of the target STn, as shown in FIG. 38, representing a good cell-line candidate for testing efficacy.

Mice were intravenously (i.v.) administered with two doses, following the experimental plan listed in Table 8.

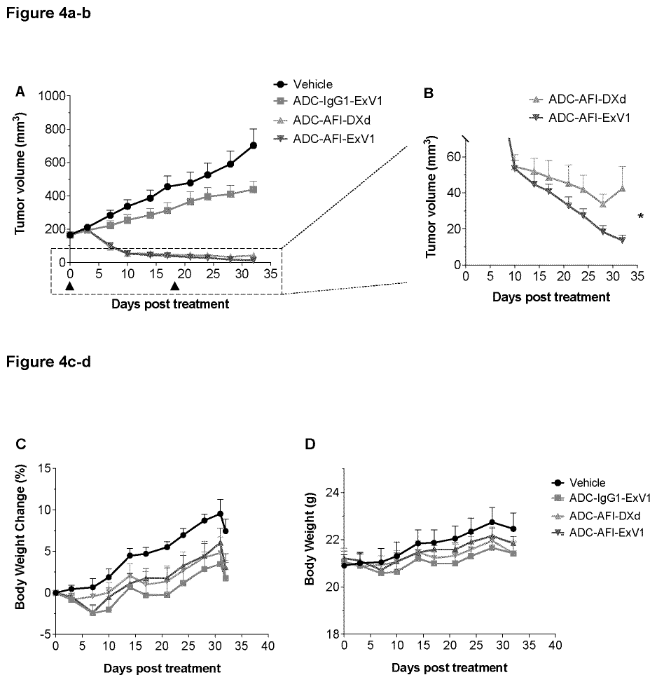

Analysis of tumor growth (FIG. 4a) demonstrates the potential of this ADC construct in selectively targeting cancer cells expressing the STn antigen. This increased specificity is achieved through the use of a cancer-specific antibody, which ensures greater tumor uptake and reduced off-target effects.

The beta-glucuronide linker, with its inherent stability and favorable release characteristics, further contributes to the ADC's potency and selectivity.

In the available data, animal models treated with the ADC exhibited significant tumor growth inhibition compared to control groups, demonstrating the advantages of targeting the STn antigen with this specific ADC composition.

In addition, head-to-head comparison of ADC-AFI-ExV1 (Exatecan, carbamic linkage-based “version1”) to ADC-AFI-DXd (Deruxtecan, linker-payload control), shows increased efficacy of our novel linker-payload as shown in magnification reported in FIG. 4b. ADC-AFI-ExV1 and ADC-AFI-DXd presented DAR=4 and were administered using same schedule and dosing [8 mg/Kg]. Finally, the body weight analysis present increase overtime (FIG. 4c-d), underlining good tolerability of treatment.

Though the safety data is limited, the features of this ADC construct, such as the cancer-specific antibody and the well-designed linker, suggest a favorable safety profile with minimal adverse effects on normal tissues. The advantages of this ADC in terms of tumor targeting, increased potency, and potential safety compared to traditional cancer therapies warrant further investigation into its safety and efficacy in both in vitro and in vivo models.

In summary, the present invention discloses, in a preferred aspect, a novel ADC with a unique combination of features, including a cancer-specific anti-STn antibody, a beta-glucuronide linker with three additional PEG units, and a carbamate handle/linker at a DAR of 4. The available tumor growth data demonstrates the potential of this ADC for improved cancer treatment. The ADC's design suggests promising safety characteristics, which should be further explored in subsequent studies to validate its potential for human and animal health applications.

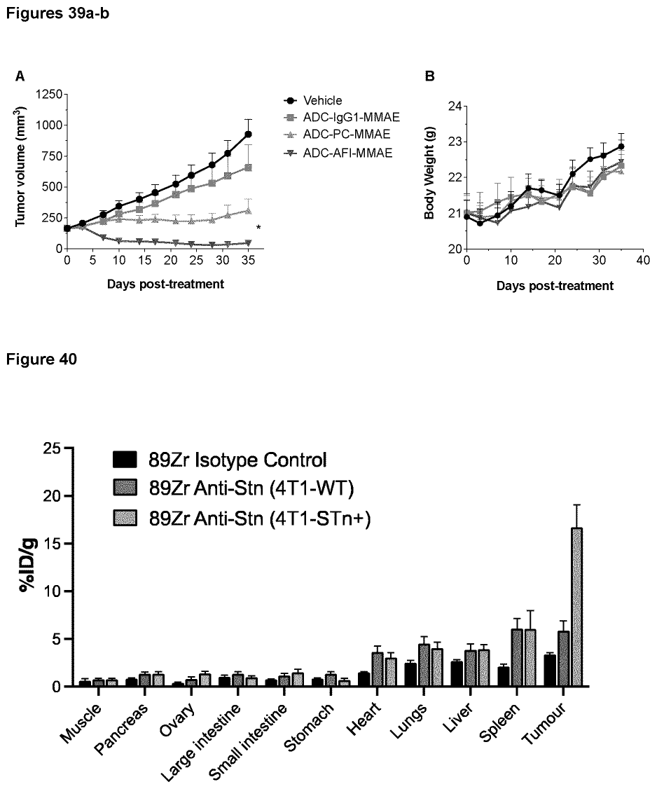

In addition to the novelty properties of newly generated ADC-AFI-ExV1, we generated data using the same antibody (mAb_v64) in conjugation with a clinically validate linker-payload (Vedotinin-platform). In this case the linker used was a protease cleavable linker (Val-Cit-PAB) and Payload monomethyl auristatin E (MMAE) using a maleimide conjugation (random Cys). With the intention to validate the efficacy of our antibody, a control anti-STn human mAb was used as positive control (mAb_PC). Interestingly, we noted increased efficacy in tumor reduction when using our novel mAb in a different ADC configuration, named as ADC-AFI-MMAE when compared to control ADC-PC-MMAE, as shown in FIG. 39a. Further body weight analysis showed efficacy of treatment with increased body weight overtime as shown in FIG. 39b.

Together these data indicate the superiority of our novel antibodies and the possibility of using different antibody clones (as listed in FIGS. 9 to 13 Supplementary Antibody List) in the development of different ADCs technology.

### Biodistribution Analysis

The aim of this study was to longitudinally evaluate the accumulation and distribution of 89Zr-Ab in a murine model of breast cancer expressing 4T1 parental cancer cell line and 4T1 STn cancer cell line.

Radiolabeled parental L2A5 antibody biodistribution was evaluated using Syngeneic murine mouse model inoculated with 4T1-STn triple negative breast cancer cell line engineered to overexpress STn.

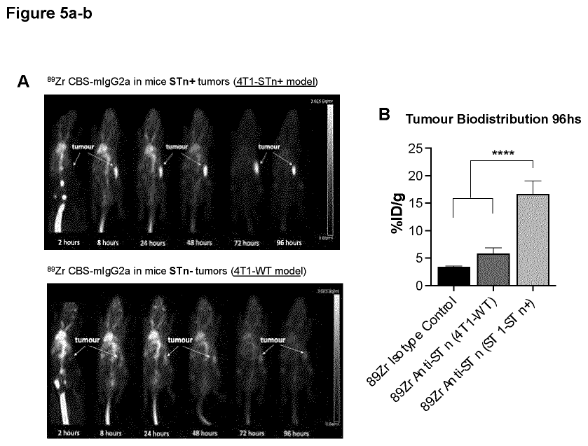

Data obtained from this analysis showed specificity of antibody binding only in presence of STn antigen expression in 4T1-STn+ group compared to control parental cell line (WT) along different time of exposure detected by PET and shown in FIG. 5a. High tumor uptake was detected after 96 hs of antibody injection-delivery as shown in FIG. 5b, underlying the specificity of the antibody delivery system. Finally, safety profile was denoted in several other organs (FIG. 40), with a relatively minor uptake in well-known highly vascularized organs, further underling safety of antibody treatment.

### Antibody Developability Analysis

To characterize affinity-matured mAb_v64 (affinity matured clone) and humanized mAb_v1 (humanized clone) in terms of stability, aggregation, temperature stress, stickiness, different tests were used, and data were compared and normalization to therapeutic antibody (Palivizumab and Trastuzumab).

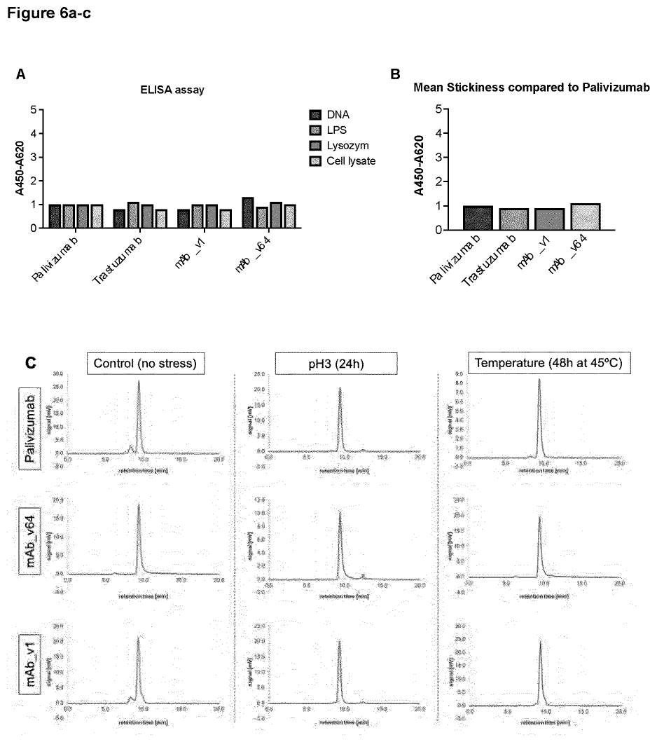

Interestingly, mAb_v64 and mAb_v1 did not present poli-reactivity or stickiness to DNA, LPS, Lysozyme and cell lysate, showing binding comparable to therapeutic antibody controls as Palivizumab and Trastuzumab as shown in FIG. 6a. Mean of stickiness analysis as well shows comparable and safety profile compared to control mAbs Palivizumab and Trastuzumab as shown in FIG. 6b.

Additionally, different stress tests such as temperature stress (48 h at 45° C.) and pH stress (24 h at pH3) showed no aggregation or antibody degradation, and no relevant increase in high or low MW fractions, revealing that those antibodies are stable without presenting any degradation (FIG. 6c).

Overall, this data underlines safety of developability of certain humanized antibodies.

### Antibody Internalization Profile in Different Cancer Cell Lines

To evaluate the internalization kinetics of certain antibodies including the parental antibody, first generation humanized clones and affinity matured antibody clones listed in FIGS. 9 to 14 were tested using different cancer cell lines.

In this assay, the antibodies were labelled with a Zenon pHrodo fluorophore that it is activated only in low pH conditions found in the early endosome. Antibody internalization was indirectly evaluated by measuring pHrodo fluorescence (MFI) by flow cytometry. All data were normalized to a control IgG1 mAb, Palivizumab.

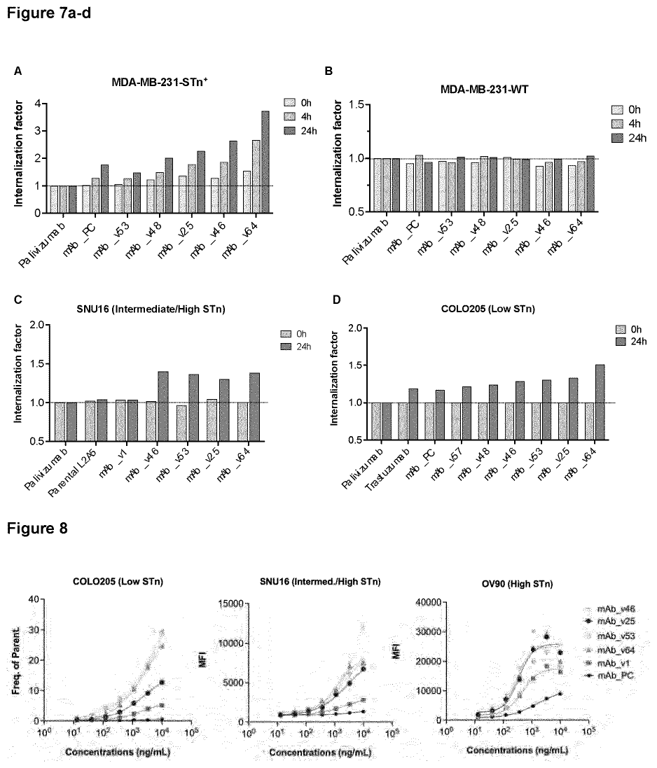

Interestingly, certain antibodies used presented different internalization profile (FIG. 7a-d) based on the STn expression level, thus suitable for different antibody-based therapy treatment.

### EC50 Analysis of mAb Clones in Different Cancer Cell Lines

In support of previously generated data, and as disclosed in our co-pending patent application WO2023/249502, we have generated proof of concept data using different mAb clones (as listed in Supplementary antibody sequence, FIGS. 9 to 14) and binding affinity measurements using FACS.

This data showed increased binding of different mAb clones, including mAb_v1, mAb_v64, mAb_v53, mAb_v25, mAb_v46, compared to anti-STn human positive control mAb_PC using different cancer cell lines, including colon cancer (COLO205), gastric cancer (SNU16) and ovarian cancer (OV90). Interestingly, different antibody clones presented different binding properties using cancer cell lines naturally expressing STn at different level (FIG. 8). Together these data underline the binding specificity of the antibodies disclosed herein against the target STn, suitable for the treatment of different type of cancers.

### DETAILED DESCRIPTION

### Materials and Methods for Antibody Component

In general, where applicable, a method for the production of the antibodies as described herein can include fusion between two cells producing an hybridoma, introducing a nucleotide sequence of the invention into a host cell, culturing the host cell under suitable conditions and for a sufficient time for the production of the encoded heavy and/or light chain of the antibody or functional fragment or probe of the invention, following purification of the heavy and/light chain of an antibody or functional fragment or probe thereof.

Recombinant expression of an antibody or functional antibody fragment or probe thereof of the invention, that binds to STn or a group of alpha-2,6 sialylated antigens, can include the construction of an expression vector containing a nucleotide sequence that encodes the heavy and/or light chain of an antibody or functional antibody fragment or probe thereof of the invention.

The vector can be produced by recombinant DNA technology. Such vectors can also include other coding nucleotide sequences, originating a chimeric antibody sequence. For instance, they may include the nucleotide sequence encoding the constant region of the antibody molecule (see WO 86/05807 and WO 89701036) enabling the expression of a chimera protein, containing the amino acid sequence of the antibody, functional antibody fragment or probe thereof, of the present invention followed by the entire heavy, or light chain, or both the entire heavy and light chains of the antibody

The expression vector can be transferred to a host cell by Transfection/Transduction techniques and the resulting cells produce the antibody or functional antibody fragment thereof of the invention. Thus, the invention includes host cells containing nucleotide sequences encoding the antibody or functional antibody fragment or probe thereof of the invention.

The host cell can be chosen to modify the characteristics of the product derived from the inserted nucleotide sequences.

In one aspect, these host cells can add glycosylation or phosphorylation sites, or other modifications to the coded proteins. For example, the host cells can provide the correct processing and cell trafficking/secretion of the proteins.

Finally, in order to increase the similarity to antibodies normally produced in humans and seek to decrease immunogenicity effects, the present inventors have provided new and improved useful antibody variants, including the humanization of the parental L2A5 antibody, and throughout antibody affinity maturation of a selected humanized variant, named with the acronym of mAb_v1, leading to the generation of antibody variants with increased binding and affinity to the target STn.

Antibody clones/variants obtained were characterised in terms of binding to different cancer cell lines, specificity to the target, and immunogenicity as further described below. In general terms, the methods employed by the inventors, and the specific processes described, in terms of their technical details, will be well understood by those skilled in this field.

### Humanization

### Variable Domain Analysis and CDR Identification

For the purpose of identifying complementarity determining regions (CDRs) and analysing the closest matching germline sequences the IMGT Domain Gap Align tool was used: http://www.imgt.org/3Dstructure-DB/cgi/DomainGapAlign.cgi

### Molecular Modelling

Molecular models were built for VH and VL domains based on homology to previously published antibody crystal structures using in-house software. PDB files allow for viewing in any molecular visualization software. Images were generated using PyMol.

### Sequence Liability Analysis

Antibody sequences were analysed for specific liabilities based on published protein motifs. Analysis was performed using an in-house system built in Microsoft Excel. The software used the following motifs where X represents any amino acid apart from Proline:

| Risk | Liability | Motif |
|---|---|---|
| High | Glycosylation | NXS or NXT |
| | Free Cysteine | C |
| | Deamidation | NG, NS or QG |
| | Isomerization | DG, DP or DS |
| Medium | Deamidation | NH |
| | Hydrolysis | NP |
| | Cleavage | TS, KK, RK or KR |
| | Transglutaminase | LLQG |
| | Integrin binding | RGD or KGD |
| | Plastic binding | FHENSP or WXXW |

### Gene Synthesis and Cloning

Variable heavy and variable light domains were designed with appropriate restriction sites at the 5′ and 3′ ends to enable cloning into Absolute Antibody cloning and expression vectors. Variable domain sequences were codon optimized for expression in human cells. Following gene synthesis the variable domains were cloned into Absolute Antibody vectors of the appropriate species and type. The correct sequence was verified by Sanger sequencing with raw data analysed using DNASTAR Lasergene software. Once confirmed plasmid DNA preparations of the appropriate size were performed to generate a sufficient quantity of high quality DNA for transfection.

### Expression and Purification

HEK 293 (human embryonic kidney 293) mammalian cells were expanded to the optimum stage for transient transfection. Cells were transiently transfected with heavy and light chain expression vectors and cultured for additional 6 days. Cultures were harvested by centrifugation at 4000 rpm, and filtered through a 0.22 M filter. A first step of purification was performed by Protein A affinity chromatography with elution using citrate pH3.0 buffer, followed by neutralization with 0.5M Tris, pH 9.0. The obtained eluted protein was then buffer exchanged into PBS, using a desalting column. Antibody concentration was determined by UV spectroscopy and the antibodies concentrated as necessary.

### Antibody Analytics

Antibody purity was determined by SDS-PAGE (sodium dodecyl sulphate polyacrylamide gel electrophoresis) and HPLC (high performance liquid chromatography). SEC-HPLC was performed on an Agilent 1100 series instrument using an appropriate size exclusion column (SEC). Antibody expression titre was determined by Protein A HPLC.

### Humanized Antibody Characterization

Different assays were performed to demonstrate whether the humanized variants maintained biophysical properties (specificity, affinity, and internalization) similar to the parental clone. These assays included:
evaluation of binding to BSM mucin (STn carrier) by ELISA
evaluation of binding to different STn+ cell lines (flow cytometry)
NMR studies to understand the antibody interaction with STn-serine glycan
in silico Immunogenicity analysis
Glycan array to determine antibody specificity
Affinity measurement by SPR
TMAs to evaluate mAb binding to patient-derived cancer tissue

The results of these studies (reported in WO2023/249502) demonstrated that the humanized variants maintained good biophysical properties in terms of specificity, affinity, and internalization.

### Affinity Maturation

### Library Generation

A bioinformatic analysis of the parental antibody was performed to generate a site directed CDR-mutation library. After homology modelling of the antibody Fv regions, and CDR grafting onto the template, CDR residues possibly involved in antigen binding were identified. For the heavy chain, 16 positions and for the light chain, 14 positions were identified. By analysing a NGS database, commonly used amino acids for the specific germline were identified. Based on this, degenerated codons were designed, introducing mutations at the identified position possibly involved in antigen binding. Amino acids with unfavourable characteristics were generally avoided. The introduction of mutations can be described by a gaussian distribution with an average of four mutations for each antibody chain. Primers were designed based on the degenerated codons and used for introduction of mutations into the antibody sequence. The mutated antibody genes were cloned into Yumab's scFv phage display vector and three libraries were generated and packaged into antibody-phage particles. A library with a total functional diversity greater than 5×10⁸ cfu was generated. Antibody clones with a functional open reading frame were determined by DNA sequence analysis. Packaging and purification of antibody-phage particles resulted in at least 3×10¹¹ cfu/ml for each library.

### Affinity Maturation by In Vitro Selection

The generated antibody-phage library was used for the affinity maturation by in vitro selection. The same overall excess of antibody-phage particles to functional size was used for each individual library. The specific amount was pooled into one library for in-vitro selection.

For the first panning round, a biotinylated BSM was used. The antibody phage output of panning round one, generated on the biotinylated protein, was used for a second round on decreasing numbers of STn+ cells to increase the stringency and drive the output towards antibodies with increased affinity. In both panning rounds a negative selection against several negative antigens was performed. Four different strategies were used for affinity maturation by in-vitro selection. By increasing the stringency from strategy one to four, a decreasing amount of eluted antibody-phage particles is expected.

### Antibody Screening

Eluted antibody-phage particles after panning round two were used for infection of E. Coli. 384 clones from each strategy were selected randomly for antibody screening. In total, 1536 antibody clones were used for production of monoclonal scFv antibodies in the bacterial system. The produced antibody clones were tested for binding activity on the positive and negative cell line. The provided control antibody (IgG) as well as the parental scFv antibody were used as positive control. The parental scFv antibody was identified with a signal to noise ratio of 20, therefore clones with a signal to noise ratio greater than 20 were identified as hits.

### Antibody Sequencing

210 clones were identified as hits and selected for DNA sequence analysis. Sequence analysis revealed 40 uniquely mutated antibodies. These antibodies showed between one and six mutations in the CDR. Several hotspot mutations were identified indicating preferable mutations at different positions.

In addition, all 40 uniquely mutated antibodies were selected and soluble scFv were produced. The production was used for an ELISA screening on two positive antigens (biotinylated BSM and non-biotinylated BSM), as well as two negative antigens (streptavidin and BSA). The signal to noise ratio between positive and negative antigens was calculated for analysis. Most antibodies showed potent binding to both positive antigens and no binding to the negative antigens. Based on the generated results antibodies were selected for conversion into the final format and production in mammalian cell culture.

### Conversion to Final Format (Human IgG1):

Based on the obtained results 20 antibodies were selected for conversion into human IgG1. The antibodies were cloned into Yumab's mammalian expression vector and produced in mammalian cell culture. The antibodies were purified using protein A affinity chromatography and buffer exchanged to phosphate buffered saline. A quality control was performed by UV/VIS spectrometry and reducing SDS-PAGE. 18 antibodies were successfully produced and showed high purity and integrity. In parallel the parental antibody was cloned in the same format and produced simultaneously.

### Affinity Ranking

For validation of antibody binding, a titration on the provided positive (MDA-MB-231 STn) and negative cell lines (MDA-MB-231 WT), and also to Bovine Submaxillary Mucin (BSM), was performed. Additional titration experiments were performed with cell lines that naturally express STn (COLO205, SNU16, OV90). All antibodies showed potent and specific binding to the target cells. An EC50 value was calculated. The best antibodies showed an EC50 value around 0.6 nM, whereas the parental antibody was calculated with an EC50 value of 2 nM. The specificity of the affinity-matured antibody clones was evaluated by glycan arrays.

### EXAMPLES

The following describes the preparation of certain specific ADCs. It will be understood by the skilled person that other ADCs, as disclosed herein, may be prepared in a similar manner, with appropriate modification depending upon the identity of the antibody, linker or drug component.

### Carbamic Linkage-Based Glucuronide Drug Linker—Version 1 (V1):

### General Procedure for Preparation of Compound 2—Reduction of —NO₂ Group:

The round-bottom flask was purged with Ar for 3 times, and added dry Pd/C (4.0 g, 10% purity) carefully. EtOAc (20 mL) was then added to infiltrate the dry Pd/C. The solution of compound 1 (20.0 g, 41.37 mmol) in EtOAc (200 mL) and added followed by the slow addition of TEA (627.95 mg, 6.21 mmol, 863.76 μL) under Ar atmosphere. The resulting mixture was degassed and purged with Ar for 3 times, and then H2 for 3 times. The mixture was stirred at 25° C. for 16 hrs. under H₂ atmosphere (15 psi). LCMS indicated the reaction was completed. The mixture was filtered and concentrated under reduced pressure. Compound 2 (18 g, 38.66 mmol, 93.43% yield, 97.8% purity) was obtained as a white solid. HPLC and LC-MS analysis relative to Compound 2 were listed respectively in FIG. 15a-b.

### General Procedure for Preparation of Compound 3—Protection of Primary Alcohol:

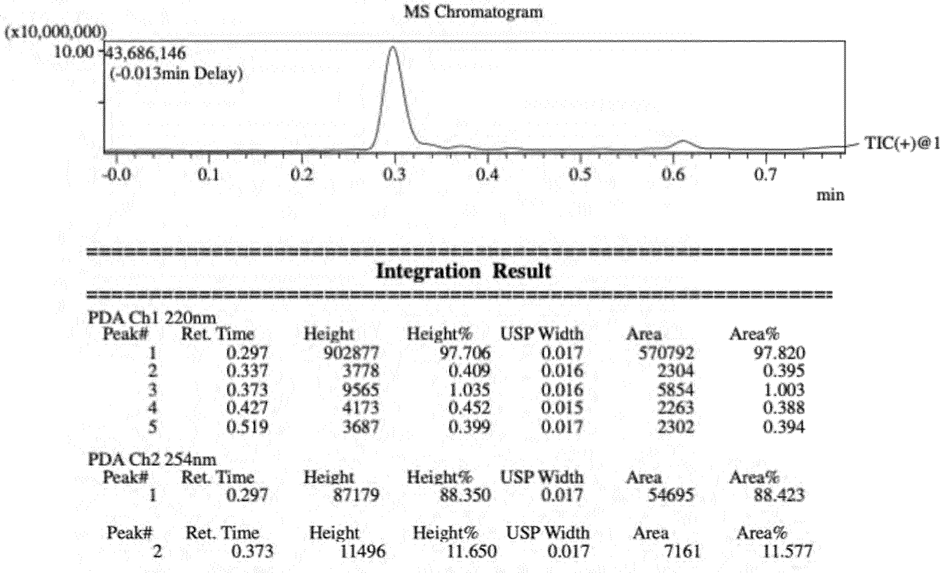

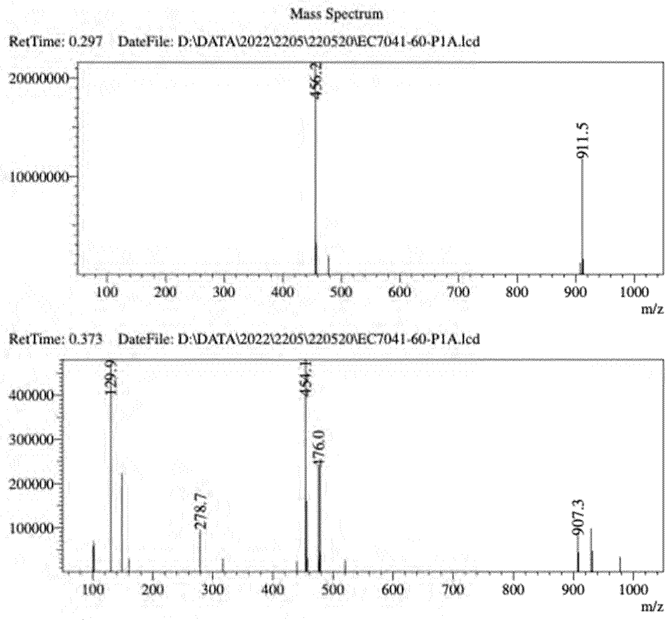

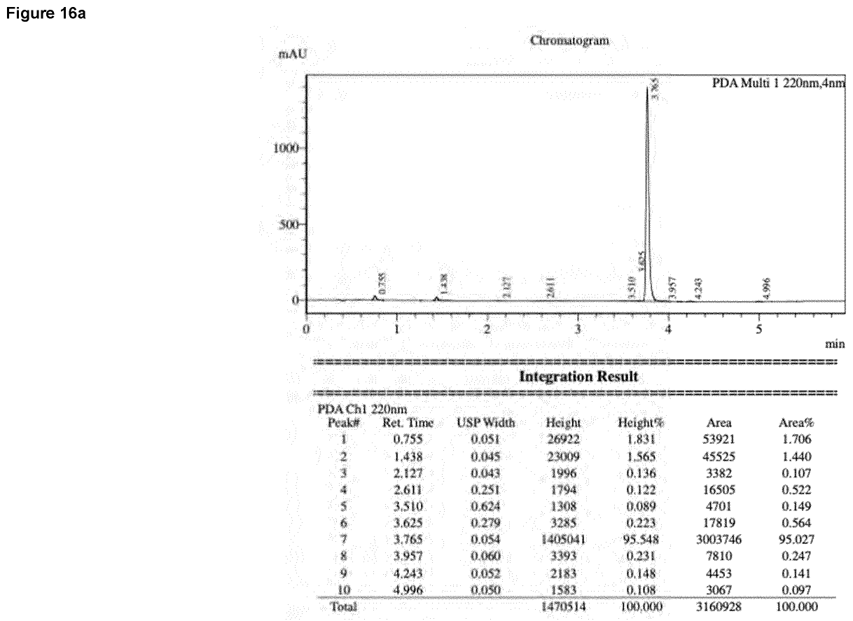

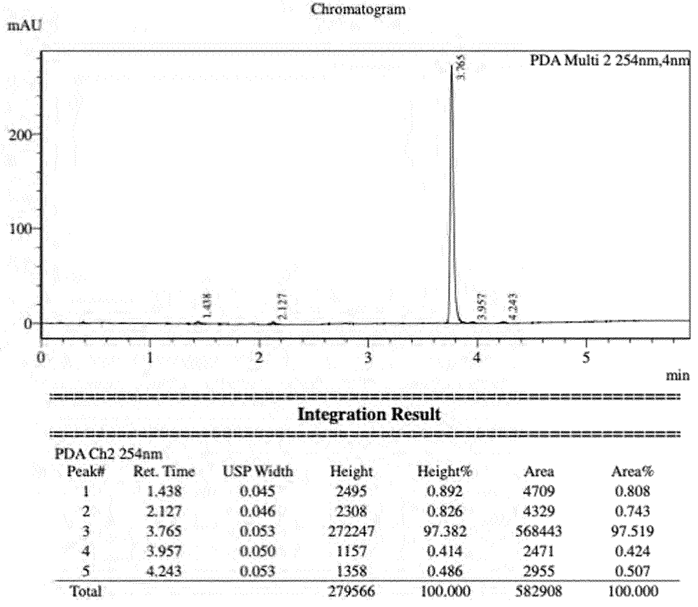

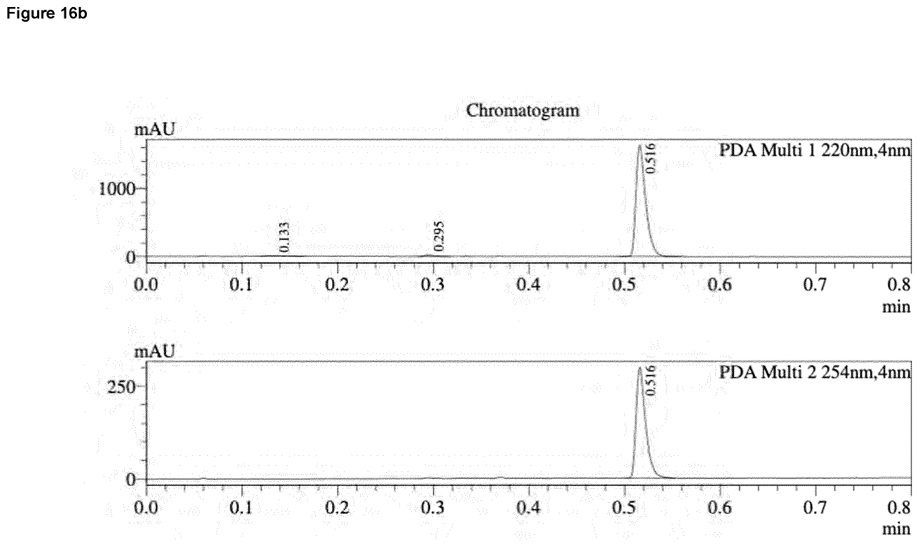

To a solution of compound 2 (18 g, 39.52 mmol, 1 eq.) were added imidazole (16.14 g, 237.15 mmol, 6 eq.), DMAP (1.21 g, 9.88 mmol, 0.25 eq.) in DMF (200 mL), and TBSCl (35.74 g, 237.15 mmol, 29.06 mL, 6 eq) successively. The mixture was stirred at 25° C. for 1 hr. TLC indicated the desired compound (Petroleum ether:Ethyl acetate=2:1, R_{f}=0.60). The reaction mixture was then diluted with water (400 mL) and extracted with EtOAc (400 mL) for two times. The combined organic layers were washed with brine (200 mL) and dried over Na₂SO₄. Filtered the organic part and concentrated under reduced pressure. The residue was purified by column chromatography (SiO₂, Petroleum ether/Ethyl acetate=50/1 to 2/1) to afford the compound 3 (14.4. g, 25.28 mmol, 63.95% yield) as a white solid. HPLC and LC-MS analysis relative to Compound 3 were listed respectively in FIG. 16a-b.

### General Procedure for Preparation of Compound 4—Coupling Reaction:

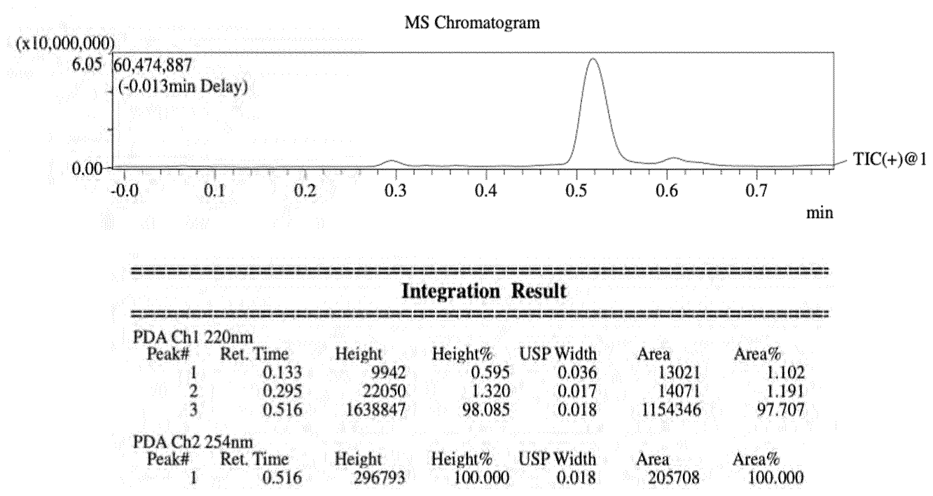

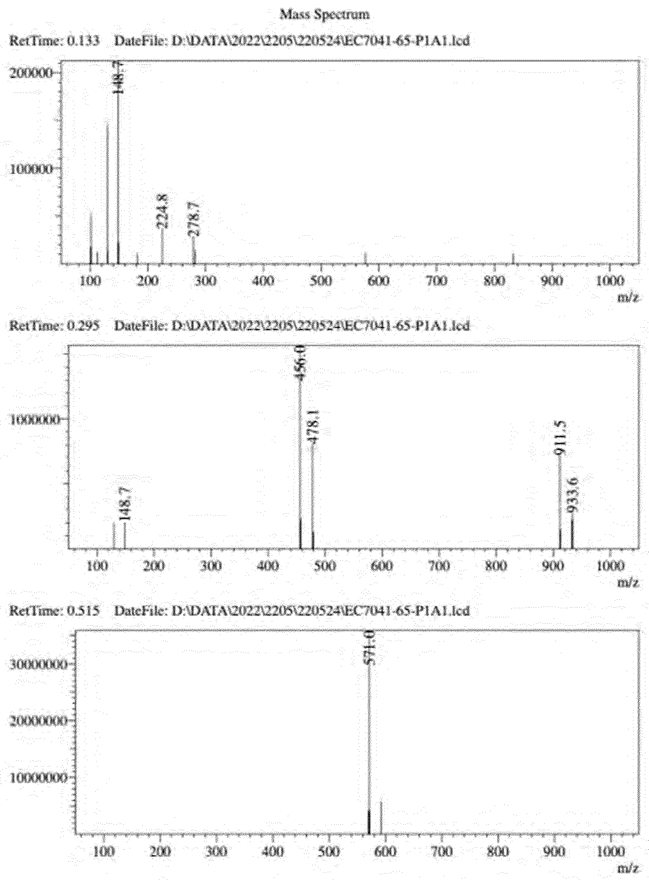

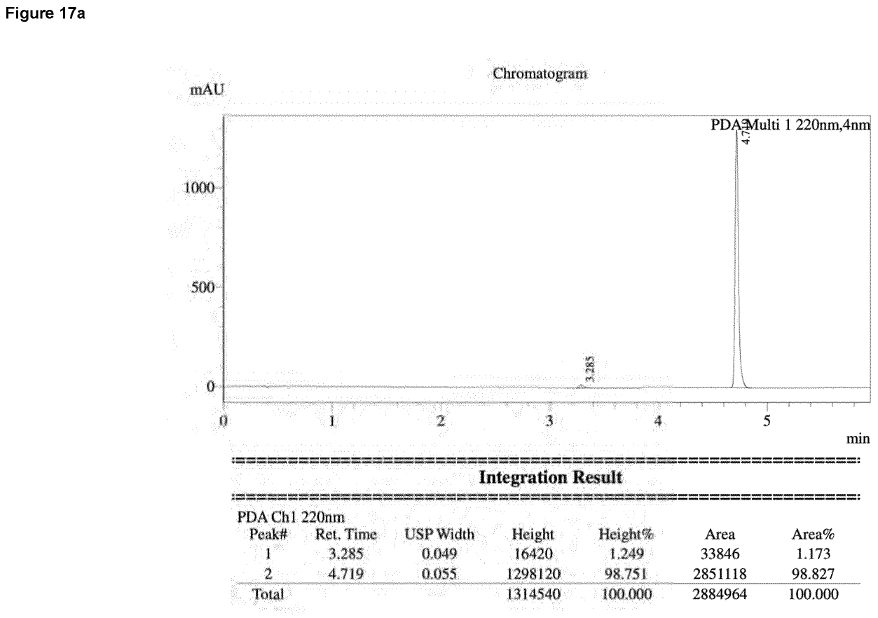

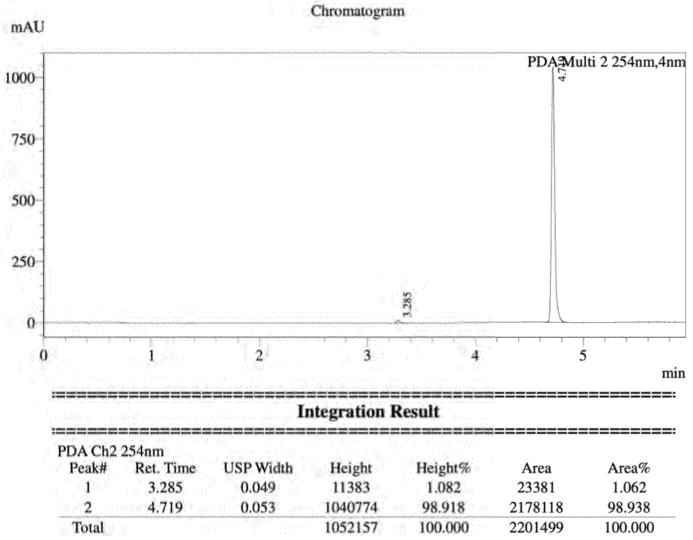

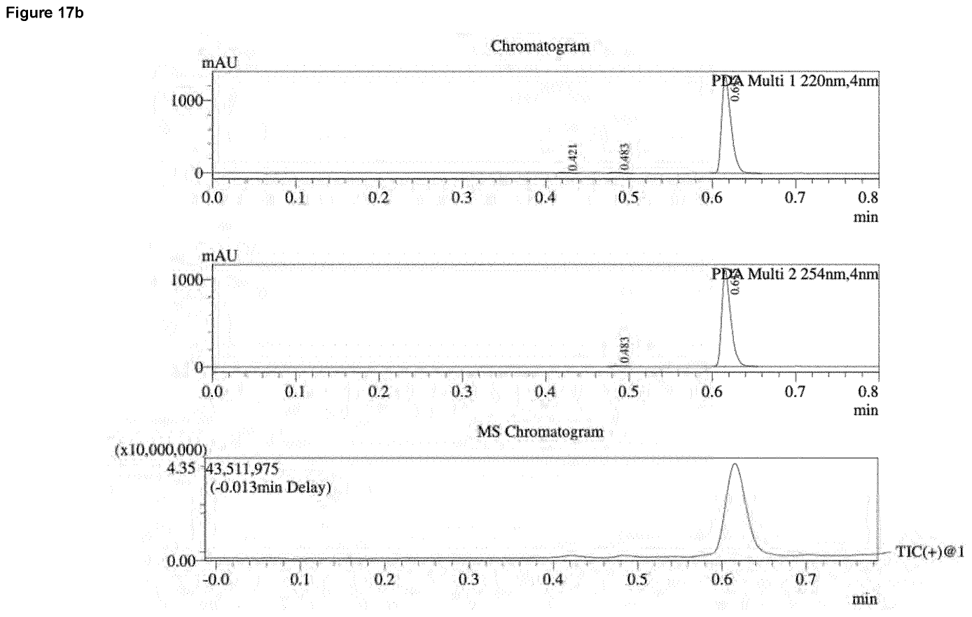

To a solution of compound 3 (5 g, 8.78 mmol) in DCM (50 mL) was added EEDQ (4.34 g, 17.55 mmol), and compound a (3.28 g, 10.53 mmol). The mixture was stirred at 25° C. for 16 hrs. LCMS showed the main peak with desired mass as well as the consumption of starting material. The solvent was removed under reduced pressure. The residue was purified by column chromatography (SiO₂, Petroleum ether/Ethyl acetate=100/1 to 2/1) to afford compound 4 (7.00 g, 8.01 mmol, 91.31% yield, 98.8% purity) as a white solid. HPLC and LC-MS analysis relative to Compound 4 were listed respectively in FIG. 17a-b.

### General Procedure for Preparation of Compound 5—Deprotection of TBS Group:

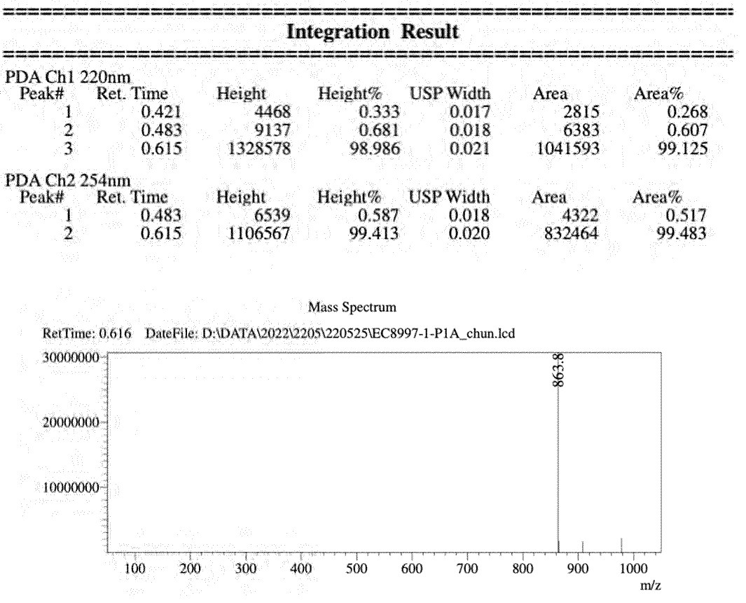

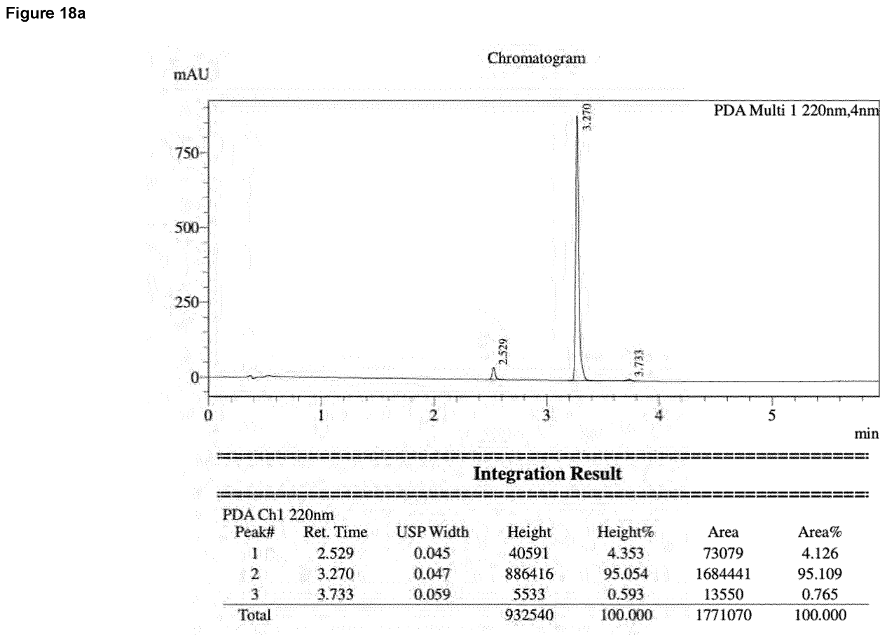

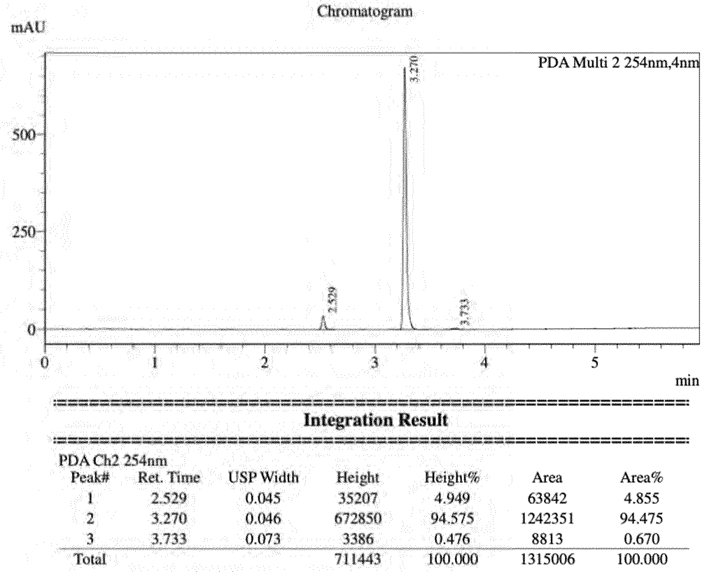

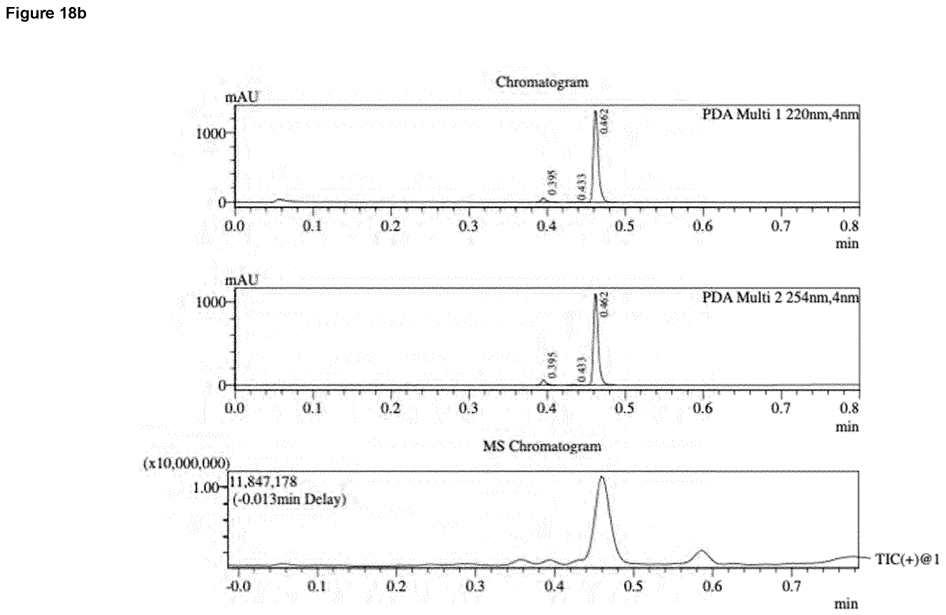

To a solution of compound 4 (5 g, 5.79 mmol) in THF (50 mL) was added HCl (1 M, 11.59 mL). The mixture was stirred at 25° C. for 1 hr. LCMS indicated the main peak with desired mass as well as the consumption of starting material. The reaction mixture was then diluted with H₂O (100 mL) and extracted with EtOAc (100 mL*2). The combined organic layers were washed with brine (100 mL) and dried over Na₂SO₄. Filtered the organic portion and concentrated under reduced pressure. Crude compound 5 (4.00 g, 5.08 mmol, 87.69% yield, 95.1% purity) was obtained as a white solid, and directly used for the next step without purification. HPLC and LC-MS analysis relative to Compound 5 were listed respectively in FIG. 18a-b.

### General Procedure for Preparation of Compound 6—PNP Activation:

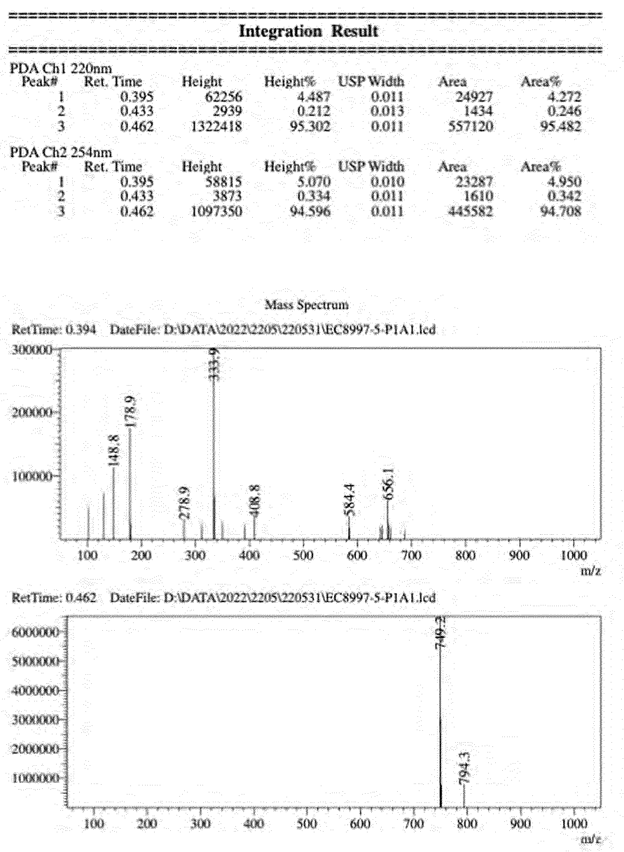

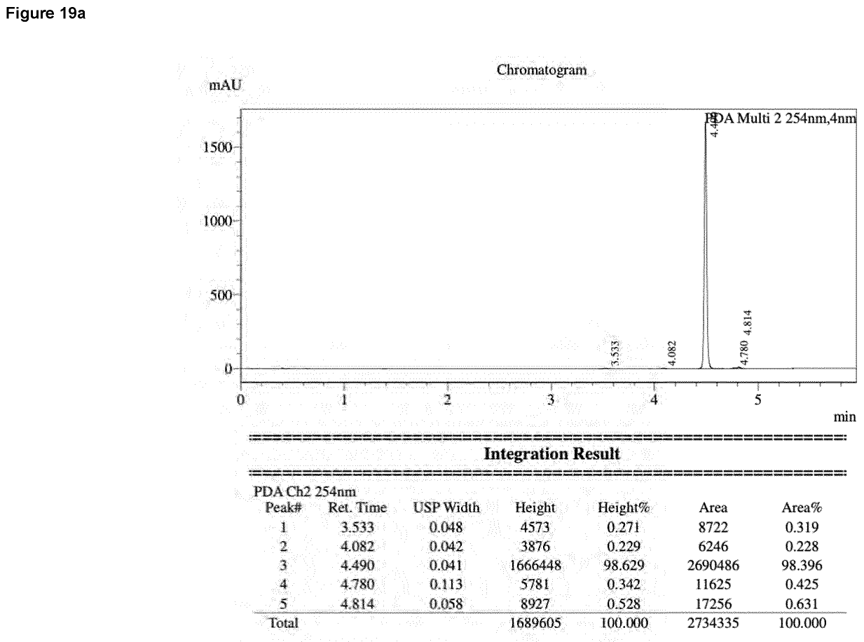

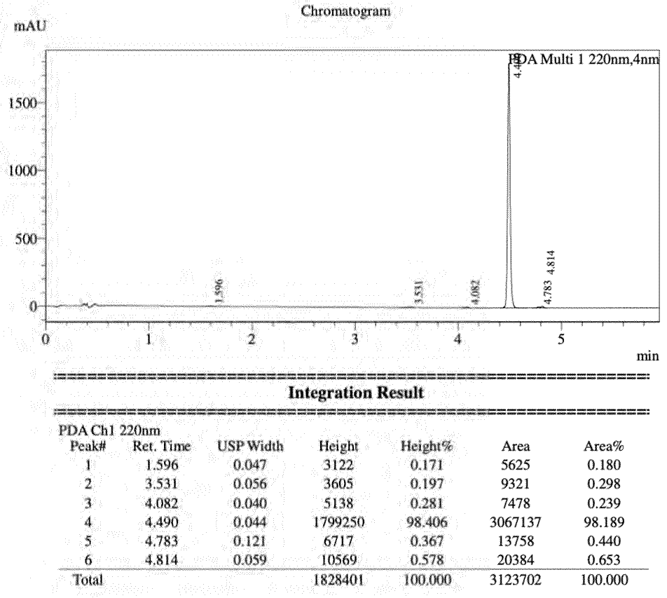

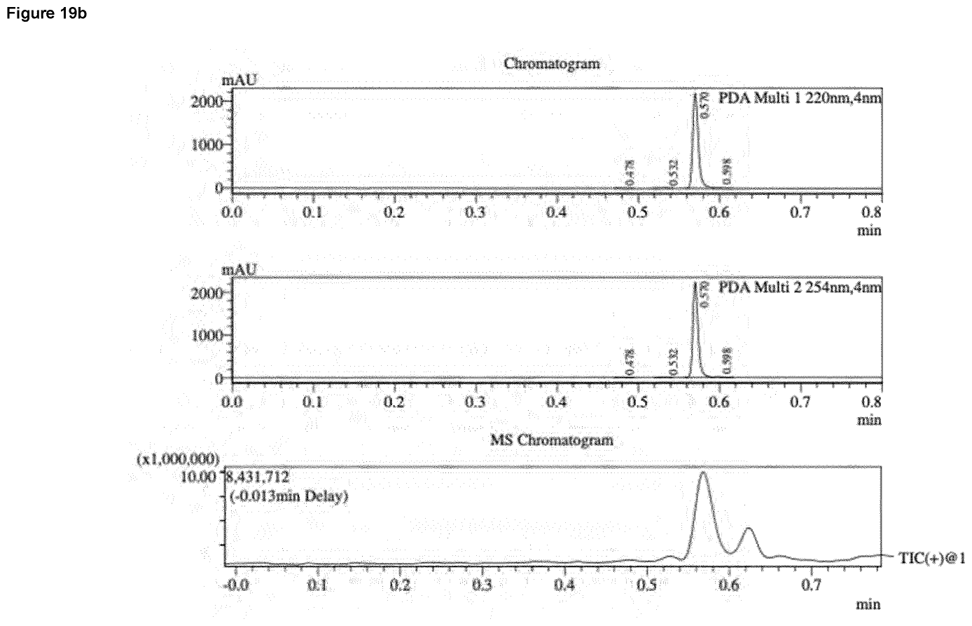

To a solution of compound 5 (4.00 g, 5.34 mmol, 1 eq) in DMF (40 mL) was added DIEA (4.14 g, 32.05 mmol, 5.58 mL, 6 eq) and PNP (4.88 g, 16.03 mmol, 3 eq). The mixture was stirred at 25° C. for 3 hr. LCMS showed the reaction was complete. Then the reaction mixture was washed with 1 M HCl (20 mL) and extracted with EtAOc (50 mL*2). The combined organic layers were washed with brine (50 mL), dried over Na₂SO₄, filtered and concentrated under reduced pressure to give a residue. The residue was purified by column chromatography (SiO₂, Petroleum ether/Ethyl acetate=50/1 to 1/2, TLC: Petroleum ether/Ethyl acetate=1:1, R_{f}=0.240) to give compound 6 (4.00 g, 4.29 mmol, 80.38% yield, 98.1% purity) as a white solid. HPLC and LC-MS analysis relative to Compound 6 were listed respectively in FIG. 19a-b.

General Procedure for Preparation of Compound 7—Conjugation with Exatecan:

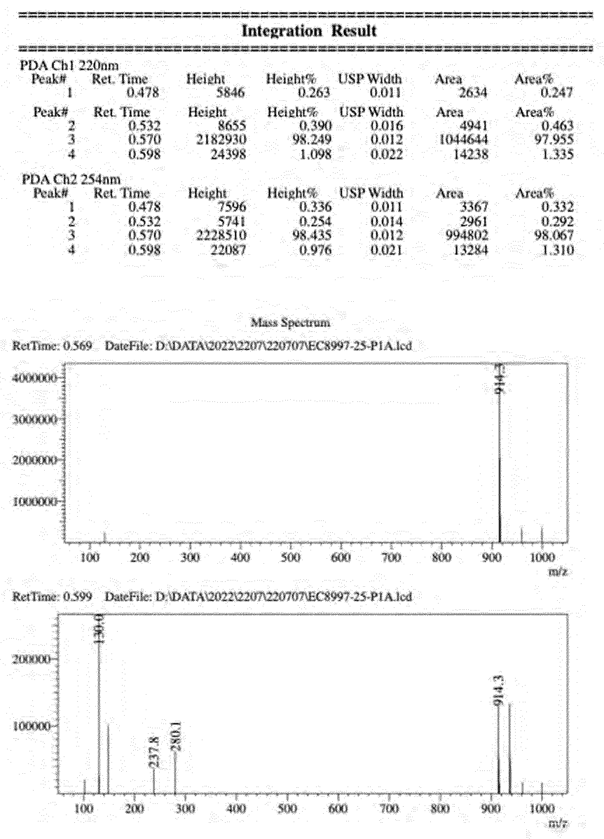

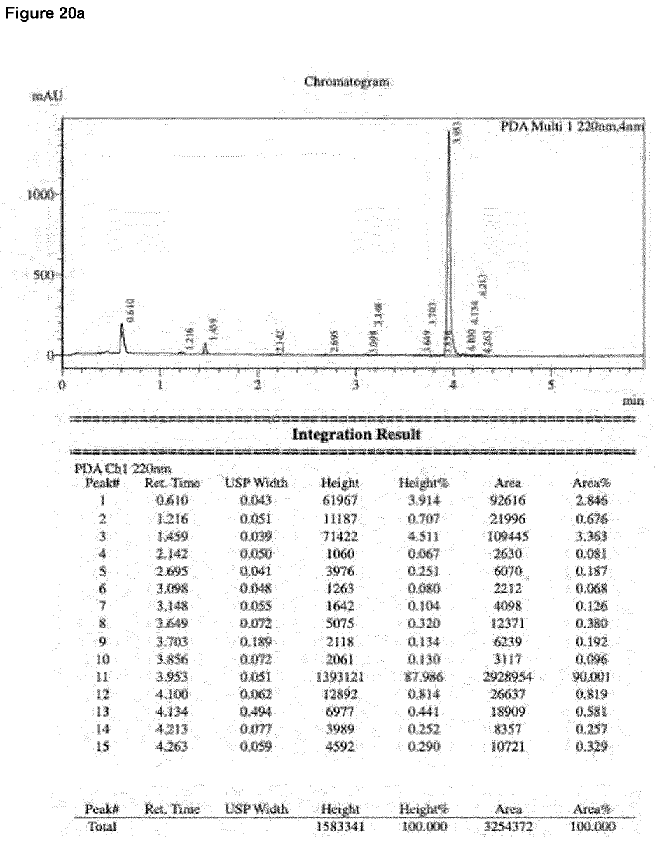

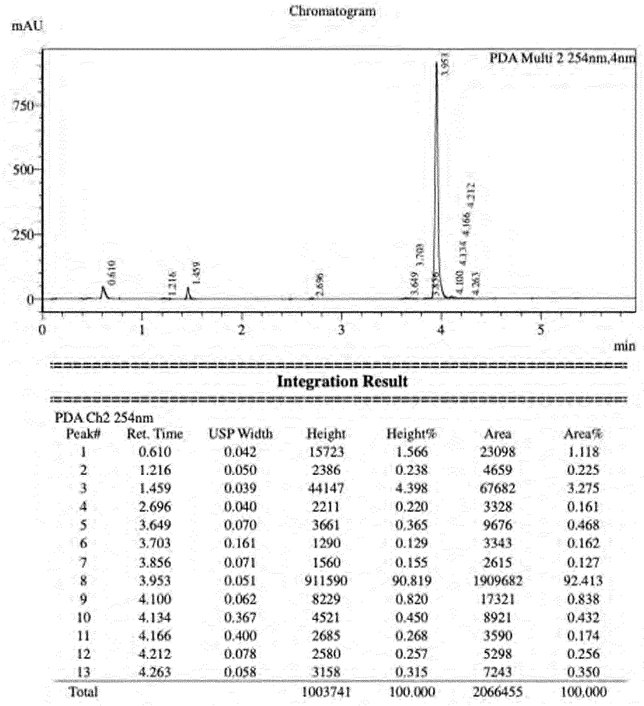

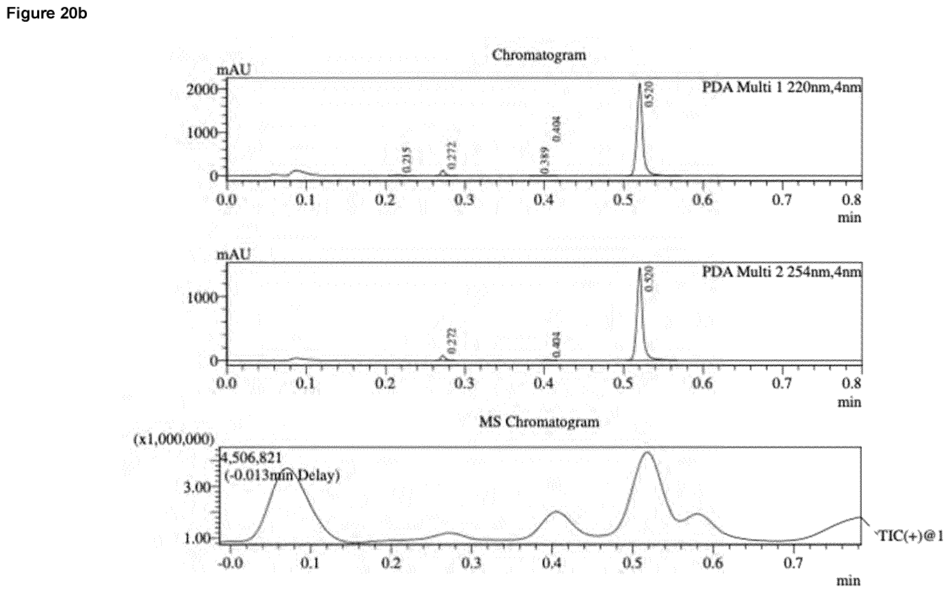

To a solution of compound 6 (1 g, 1.09 mmol, 1 eq.), exatecan (610.76 mg, 1.15 mmol, 1.05 eq.) in DMF (10 mL) was added HOBt (295.73 mg, 2.19 mmol, 2 eq.), and DIEA (424.29 mg, 3.28 mmol, 571.82 μL, 3 eq.). The mixture was stirred at 25° C. for 2 hrs. LCMS indicated the main peak with desired mass as well as the consumption of compound 6. The crude product was triturated with isopropyl ether (200 mL*2) at 25° C. for 10 mins. Then filtered and concentrated under reduced pressure to afford crude compound 7 (1.3 g, 1.02 mmol, 93.26% yield, 95% purity) as a yellow solid. HPLC and LC-MS analysis relative to Compound 7 were listed respectively in FIG. 20a-b.

### General Procedure for Preparation of Compound 8—Deprotection of Fmoc Group:

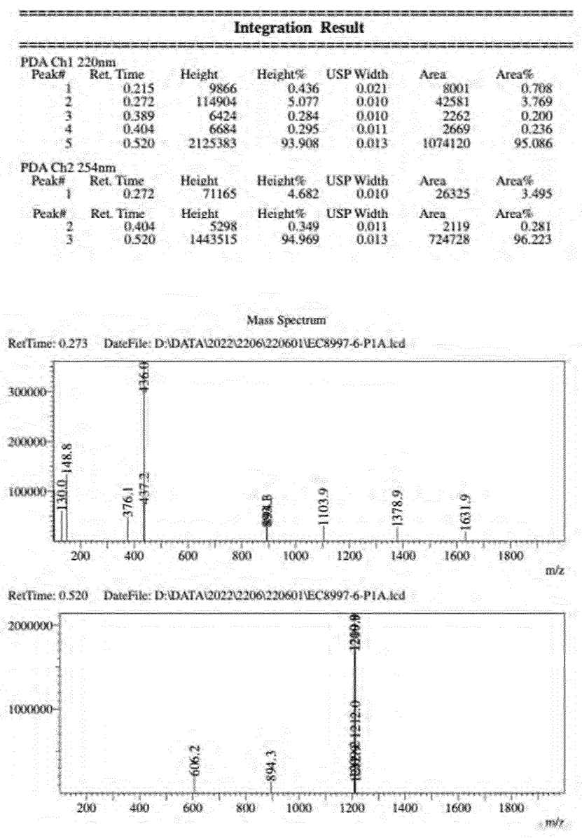

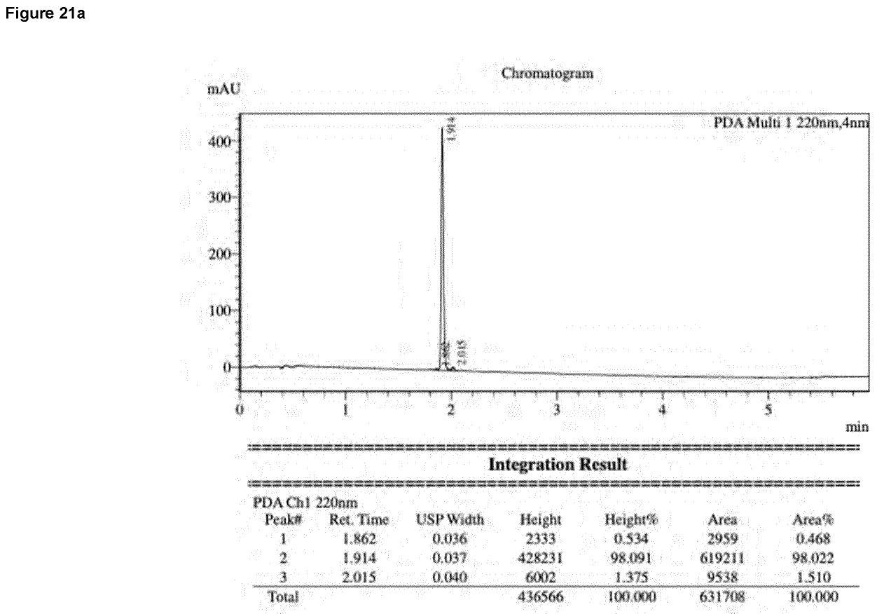

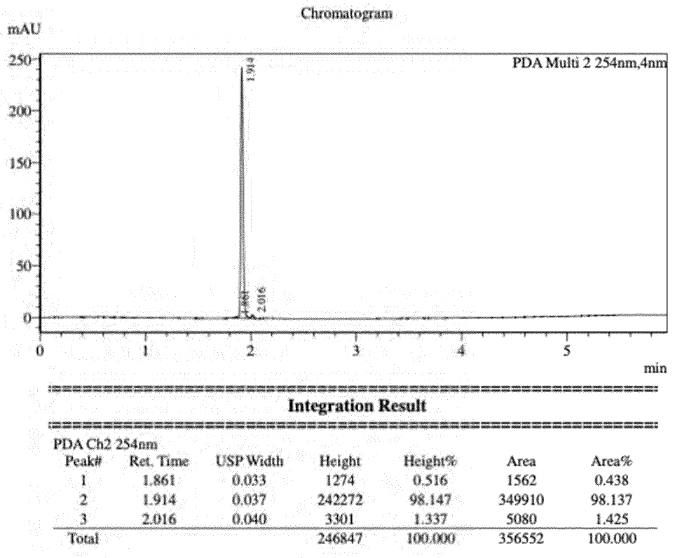

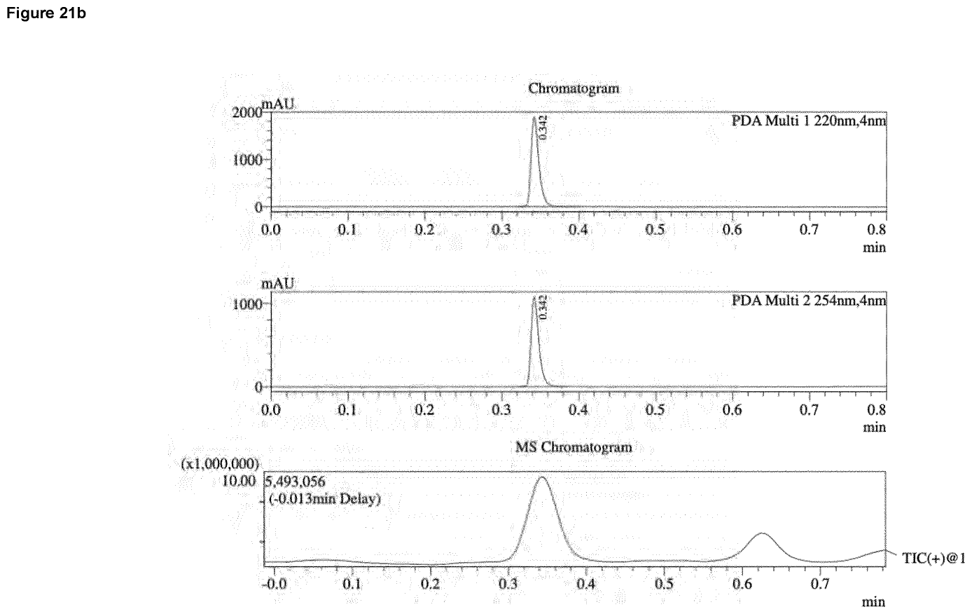

To a solution of compound 7 (1 g, 826.33 μmol, 1 eq.) in MeOH (10 mL) was added LiOH·H₂O (69.35 mg, 1.65 mmol, 2 eq.) in H₂O (10 mL). The reaction mixture was stirred at 0° C. for 12 hrs. Two reactions were carried out parallel. The reaction was completed indicated by LCMS. Then two reactions mixture were combined and quenched by addition of CH₃COOH (6 mL) at 0° C. (pH=5). The residue was purified by prep-HPLC (TFA condition) to afford compound 8 (780 mg, 901.63 μmol, 54.56% yield, 98.0% purity) as a faint yellow solid. HPLC and LC-MS analysis relative to Compound 8 were listed respectively in FIG. 21a-b.

### General Procedure for Preparation of Compound Carbamic Linkage-Based Glucuronide Drug Linker (Target 1)—Coupling Between 8 and 1a-1:

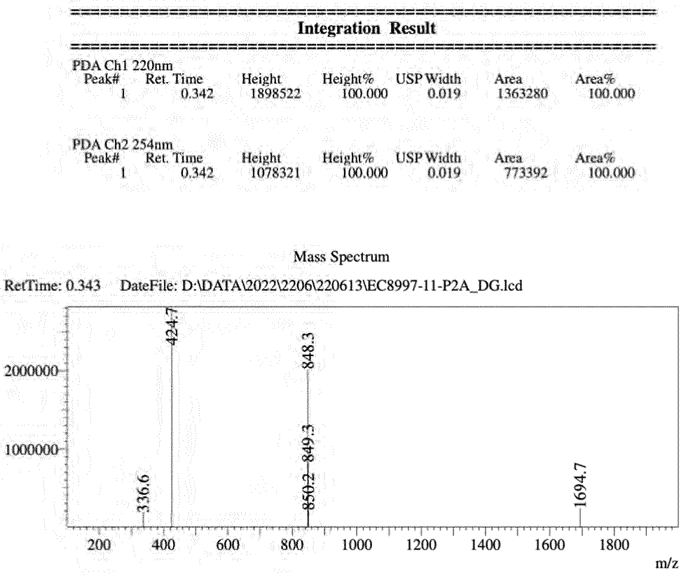

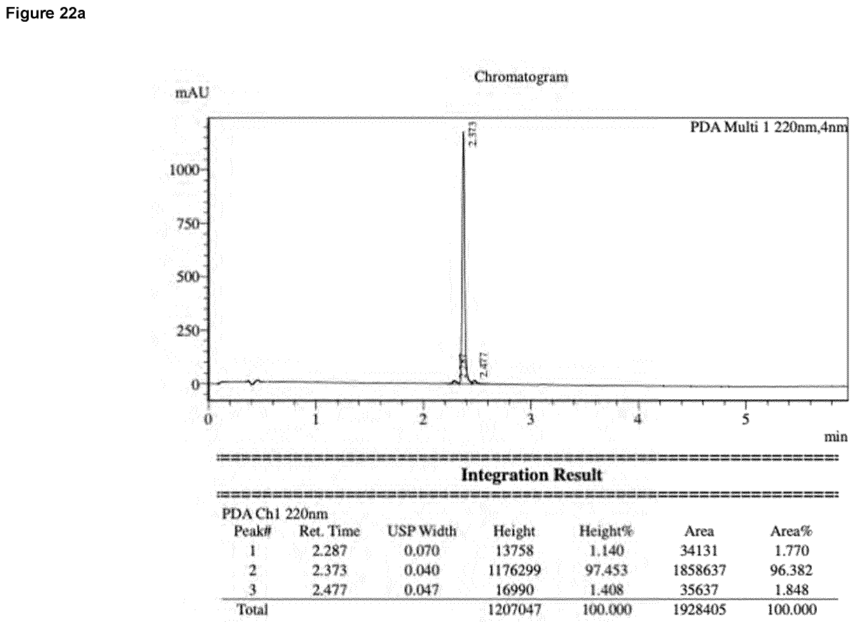

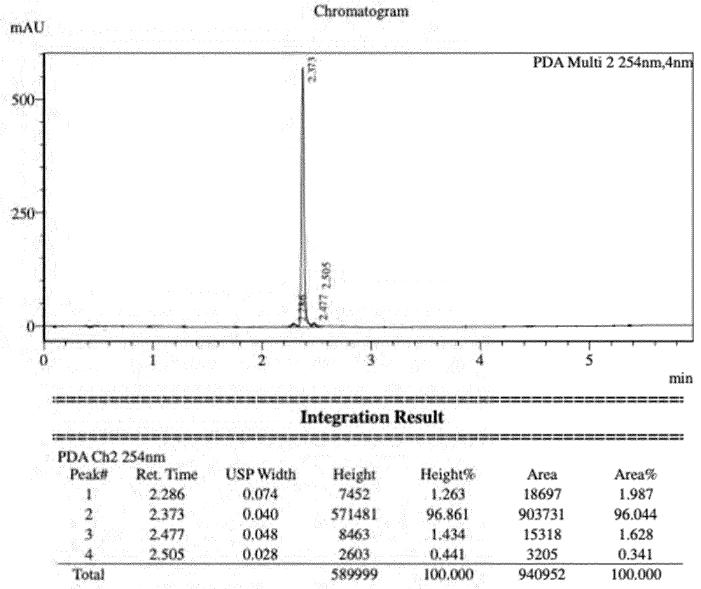

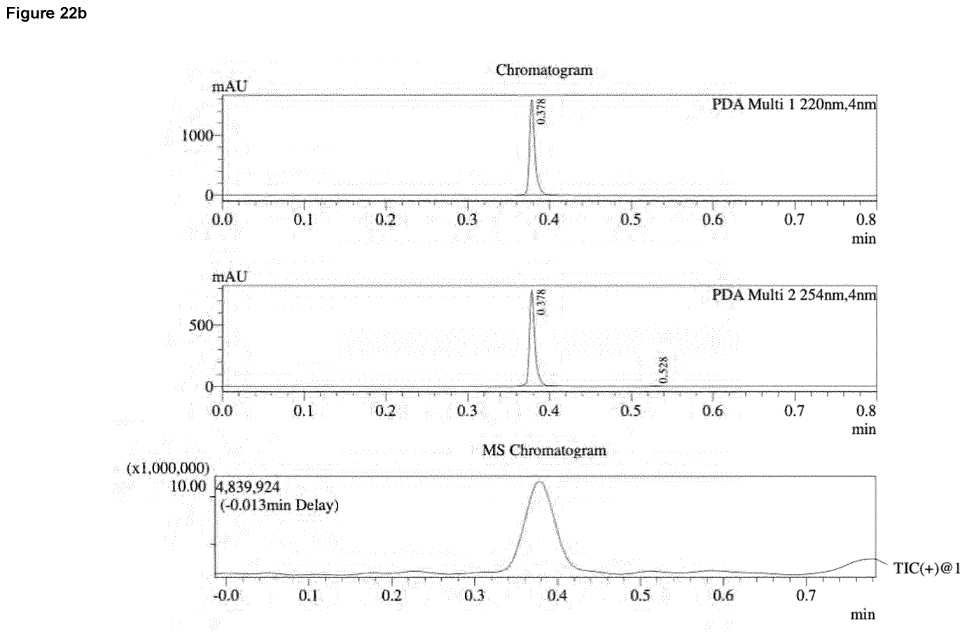

To a solution of compound 8 (400 mg, 471.81 μmol, 1 eq.) in DMF (4 mL) was added NMM (95.44 mg, 943.63 μmol, 103.74 μL, 2 eq.), and compound 1a-1 (206.75 mg, 518.99 μmol, 1.1 eq.). The mixture was stirred at 25° C. for 3 hrs. LCMS indicated the main peak with desired mass as well as the consumption of compound 8. Then quenched reaction mixture with 0.1 mL of formic acid. The resultant was directly purified by prep-HPLC (FA condition). Finally, carbamic linkage-based glucuronide drug linker (220 mg, 194.51 umol, 41.23% yield) was obtained as a faint yellow solid. HPLC and LC-MS analysis relative to Compound 8 and 1a-1 were listed respectively in FIG. 22a-b.

### Quaternary Ammonium Linkage-Based Glucuronide Drug Linker:

### General Procedure for Preparation of Compound 6a-1

To a solution of compound 6a (500 mg, 940.64 μmol, 1 eq.) in DMF (20 mL) were added DIC (474.83 mg, 3.76 mmol, 582.62 μL, 4 eq.), DIEA (243.14 mg, 1.88 mmol, 327.69 μL, 2 eq.), and HOBt (254.20 mg, 1.88 mmol, 2 eq.). The reaction mixture was then stirred at 25° C. for 16 hrs. LCMS showed the desired compound as a main peak. The reaction mixture was then triturated with isopropyl ether (100 mL*2) at 25° C. for 10 mins. It was filtered, and the solid was concentrated under reduced pressure to afford compound 6a-1 (500 mg, 924.98 μmol, 98.34% yield, 96.3% purity) as a white solid. HPLC and LC-MS analysis relative to Compound 6a-1 were listed respectively in FIG. 23a-b.

### General Procedure for Preparation of Compound 9-Linker Modification:

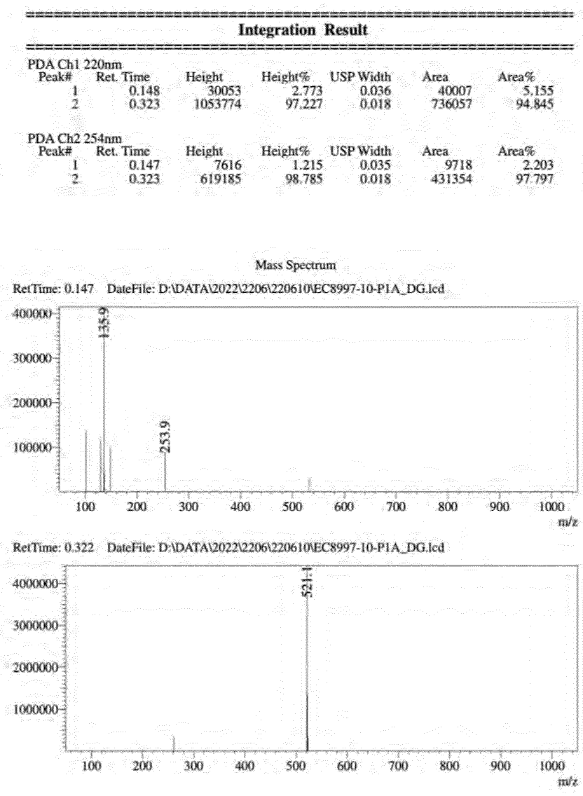

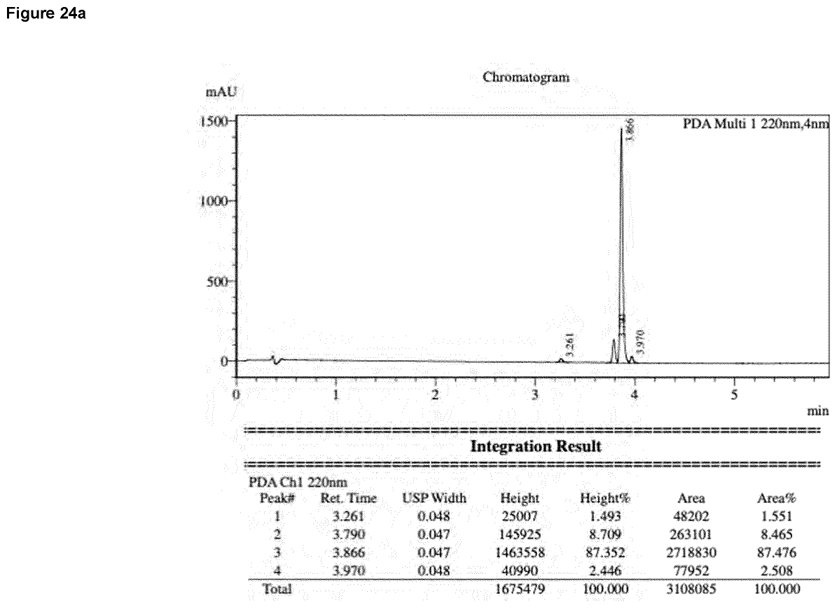

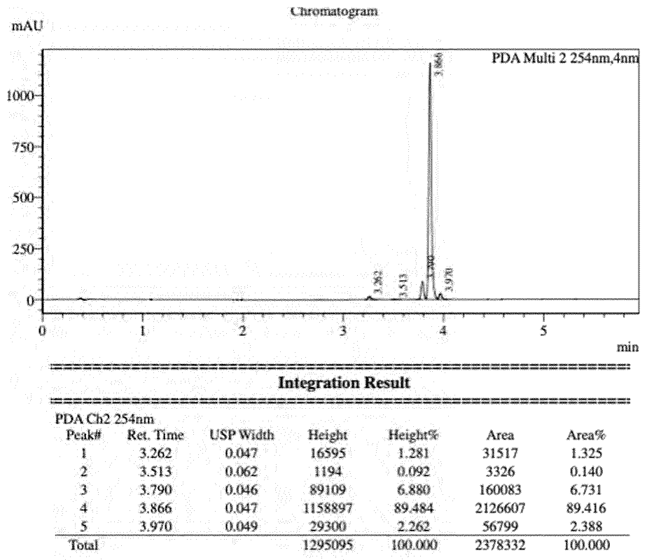

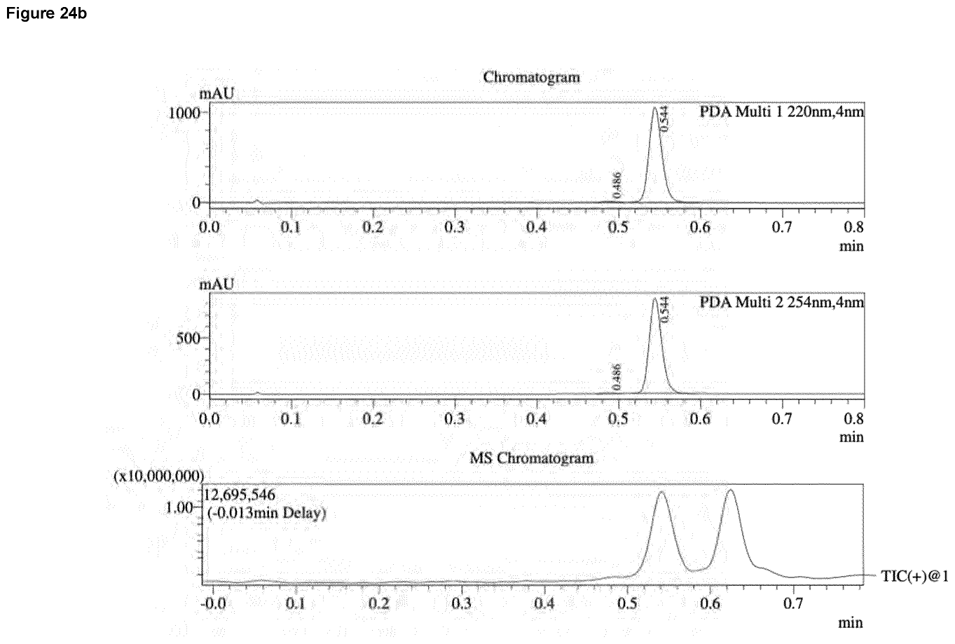

To a solution of compound 5 (2.8 g, 3.74 mmol, 1 eq.) in DMF (28 mL) was added PPh₃ (2.94 g, 11.22 mmol, 3 eq.), and CBr₄ (1.24 g, 3.74 mmol, 1.00 eq.). The reaction mixture was stirred at 25° C. for 2 hrs. LCMS detected the desired compound as a main peak. The reaction mixture was concentrated under reduced pressure to afford a residue which was purified by prep-HPLC (TFA condition). Compound 9 (1.7 g, 1.83 mmol, 48.95% yield, 87.4% purity) was obtained as a white solid. HPLC and LC-MS analysis relative to Compound 9 were listed respectively in FIG. 24a-b.

General Procedure for Preparation of Compound 10—Conjugation with Modified Exatecan:

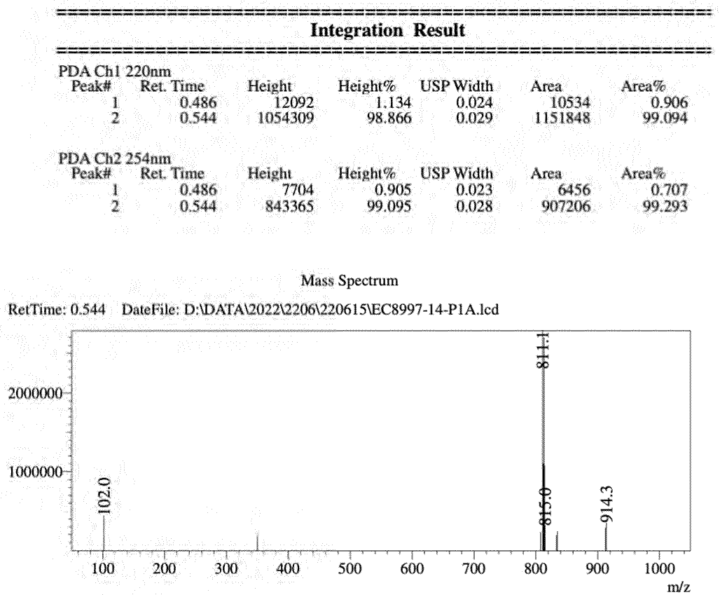

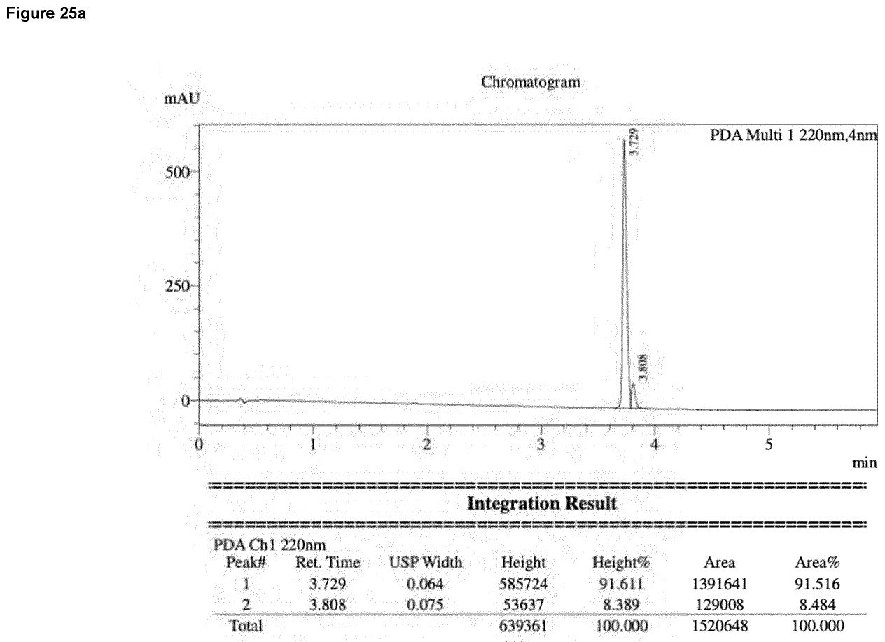

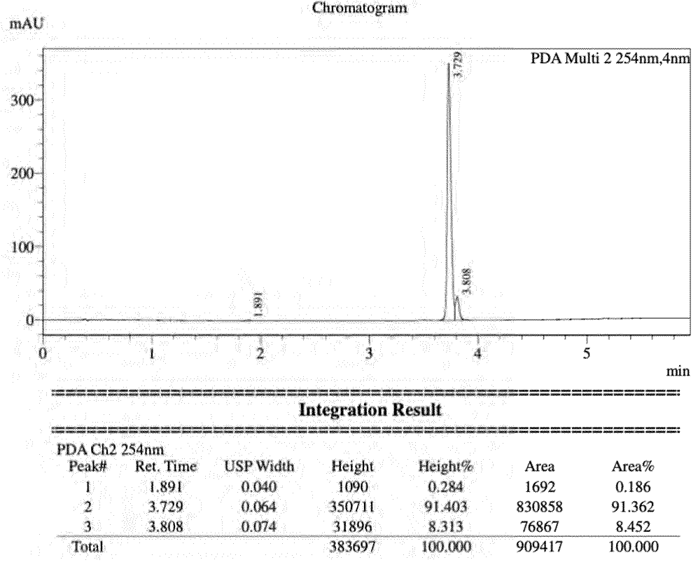

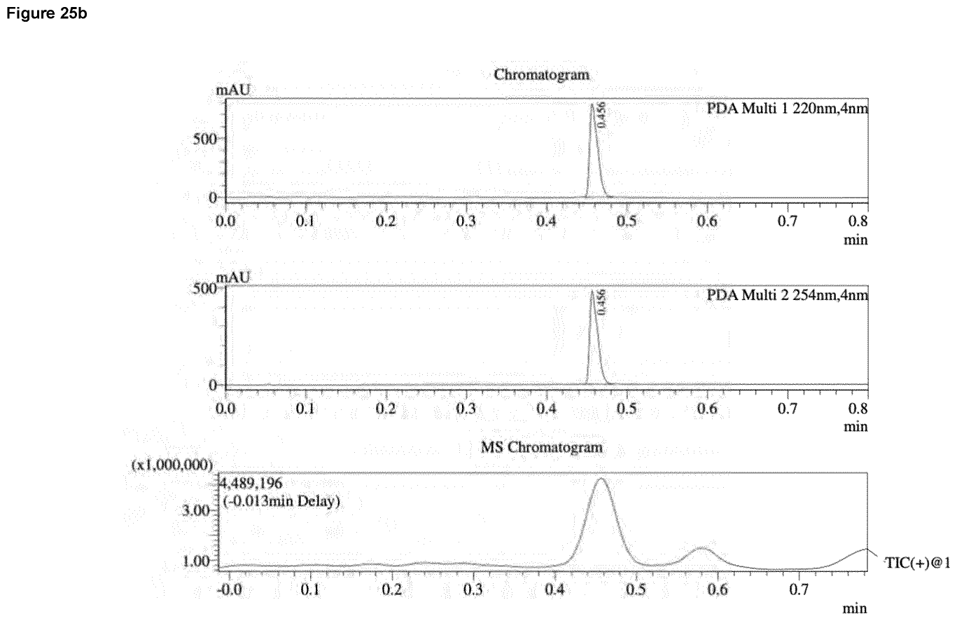

To a solution of compound 6a-1 (560.56 mg, 1.08 mmol, 1 eq.) in DMF (10 mL) were added DIEA (139.18 mg, 1.08 mmol, 187.57 μL, 1 eq.), and compound 9 (1 g, 1.08 mmol, 87.4% purity, 1 eq.). The mixture was stirred at 25° C. for 3 hrs. LCMS detected the desired compound as a main peak. The crude product was triturated with isopropyl ether (300 mL*2) at 25° C. for 5 min. The residue was purified by prep-HPLC (TFA condition) to afford compound 10 (900 mg, 657.61 μmol, 61.07% yield, 91.5% purity) as a yellow solid. HPLC and LC-MS analysis relative to Compound 10 were listed respectively in FIG. 25a-b.

### General Procedure for Preparation of Compound 11—Deprotection of Fmoc Group:

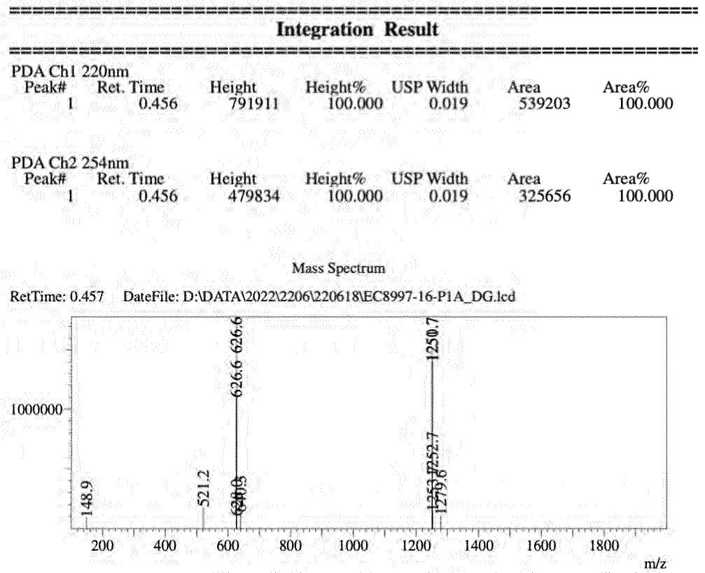

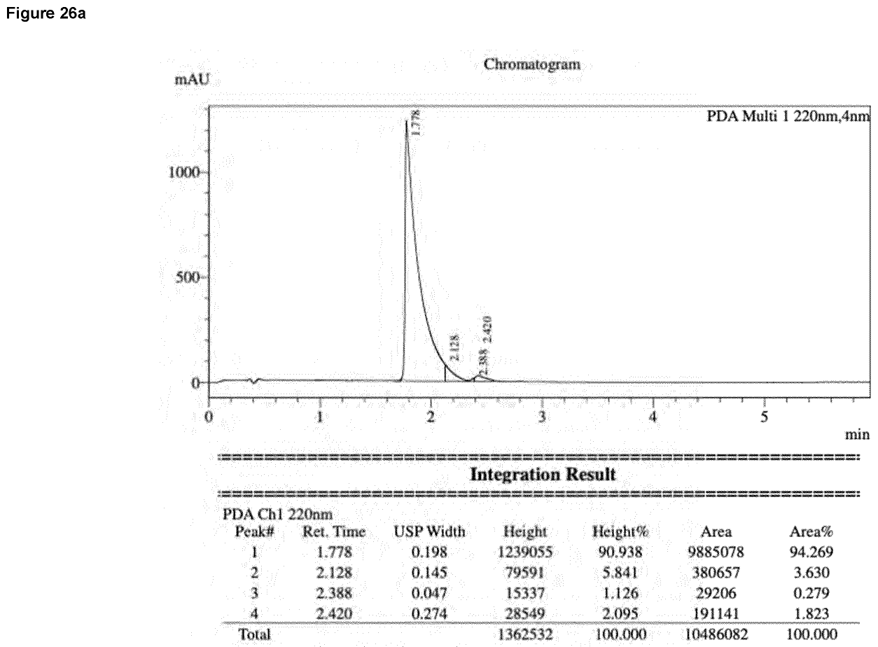

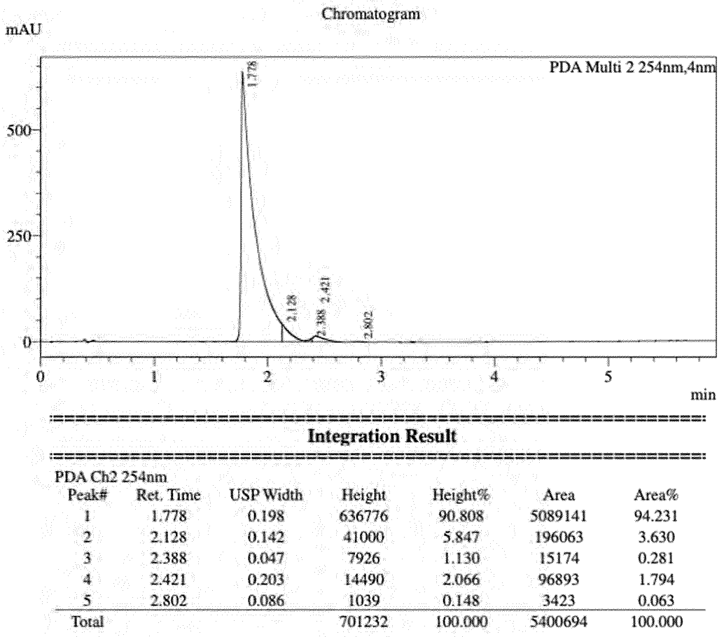

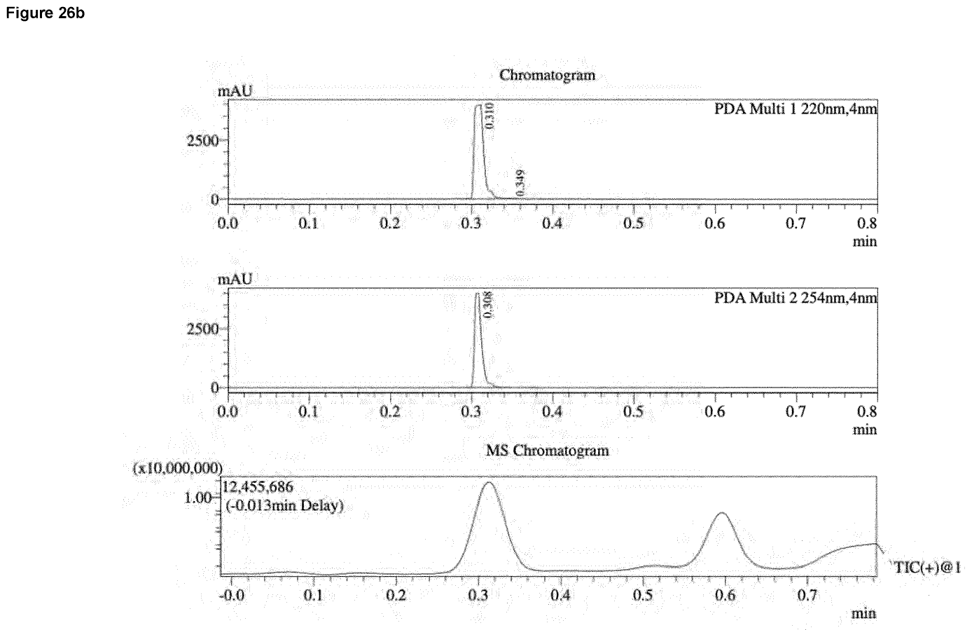

LiOH·H₂O (338.90 mg, 8.08 mmol, 10 eq.) in MeOH (15 mL) was added to a solution of compound 10 (1.0 g, 807.59 μmol, 1 eq.) in H₂O (5 mL). The mixture was stirred at 0° C. for 16 hrs, and at 25° C. for 1 hr. LCMS detected the desired compound as a main peak. Then quenched the reaction mixture with 1 mL of CH₃COOH and purified the crude by prep-HPLC (TFA condition) to afford compound 11 (430 mg, 455.18 μmol, 56.36% yield, 94.2% purity) as a yellow solid. HPLC and LC-MS analysis relative to Compound 11 were listed respectively in FIG. 26a-b.

### General Procedure for Preparation of Compound Quaternary Ammonium Linkage-Based Glucuronide Drug Linker (Target 2)—Coupling Between 11 and 1a-1:

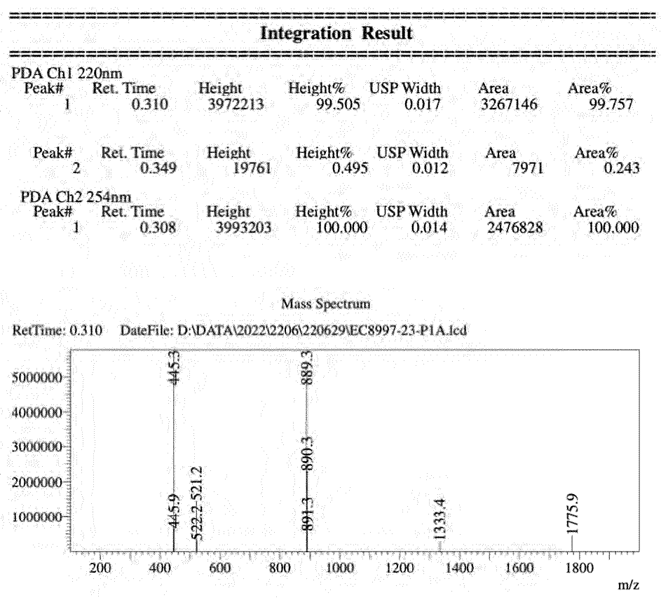

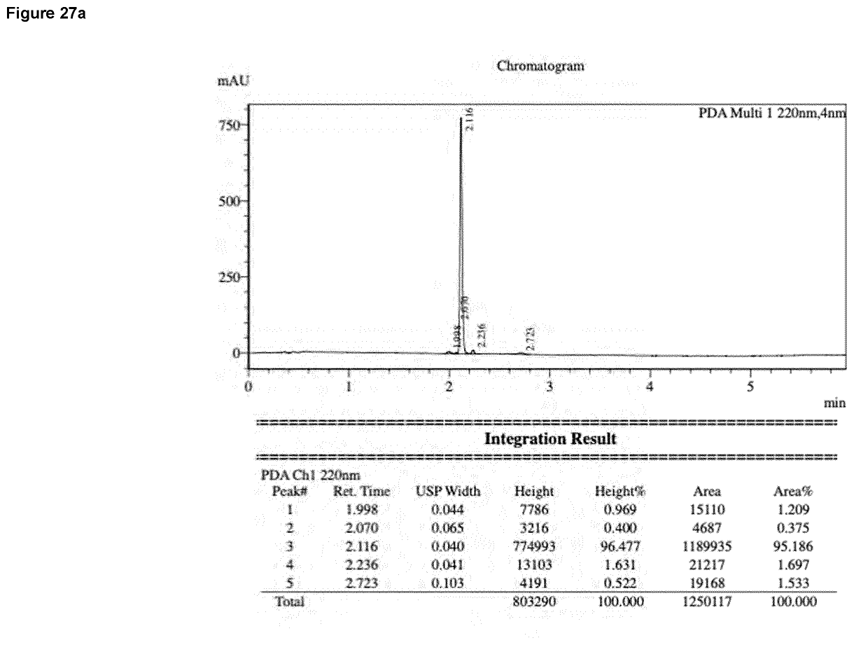

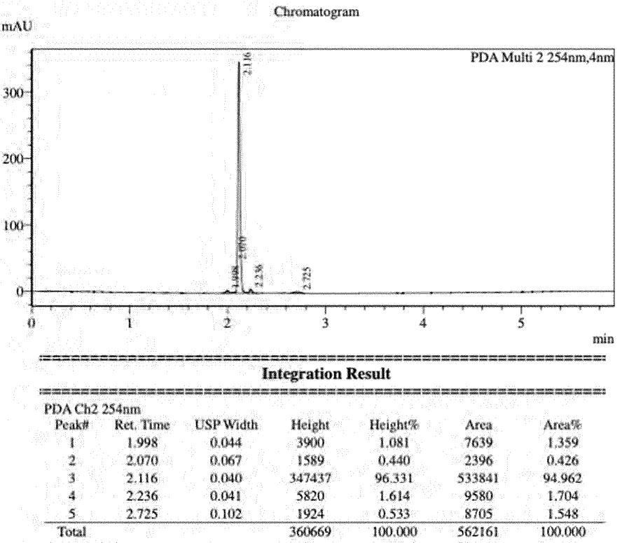

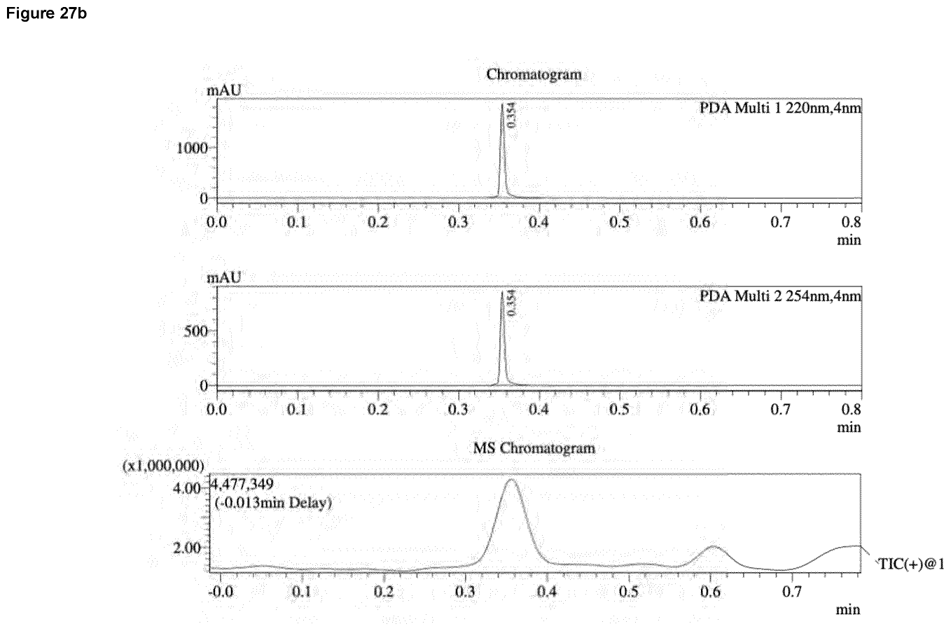

To a solution of compound 11 (500 mg, 561.86 μmol, 1 eq.) in DMF (4.3 mL) were added NMM (113.66 mg, 1.12 mmol, 123.55 μL, 2 eq.), and compound 1a-1 (246.21 mg, 618.05 μmol, 1.1 eq.). The mixture was stirred at 25° C. for 3 hrs. LCMS detected the desired compound as a main peak. The reaction mixture was quenched by the addition of HCOOH (0.1 mL) at 0° C. The resultant was purified by prep-HPLC (FA condition) to afford quaternary ammonium linkage-based glucuronide drug linker (255 mg, 298.34 μmol 95.1% purity) as a white solid. HPLC and LC-MS analysis relative to Compound 12 were listed respectively in FIG. 27a-b.

Final ADCS Produced with Relative Analysis.

### ADC Samples List

ADC-AFI-ExV1 (mAb_v64) and ADC-IgG1-ExV1 (IgG1 control) were produced from linker-payload and mAb via cysteine conjugation, targeting DAR4. Additional ADC-AFI-DXd (mAb_v64) was produced using GGFG-DXd linker payload. Deruxtecan (GGFG-DXd) used in this analysis (Cat. HY-13631E; Vendor MedChemExpress). Detailed information can be seen in Table 1.

TABLE 1
ADC-AFI-ExV1, ADC-IgG1-ExV1 and ADC-AFI-DXd with relative
amount (mg), antibody, linker payload and target DAR.

| | Amount | | | |
|---|---|---|---|---|
| Product Name | (mg) | Antibody | Linker-payload | Target DAR |
| ADC-AFI-ExV1 | >15 | mAb_v64 | Carbamic linkage-based | DAR4.0 +/− 0.4 |
| | | | glucuronide drug linker- | |
| | | | Version 1 (V1) | |
| ADC-IgG1-ExV1 | >15 | IgG1 | Carbamic linkage-based | DAR4.0 +/− 0.4 |
| | | | glucuronide drug linker- | |
| | | | Version 1 (V1) | |
| ADC-AFI-DXd | >15 | mAb_v64 | GGFG-DXd | DAR4.0 +/− 0.4 |

### Purification Method by Zeba Spin Desalting Column (10 mL)

The Zeba Spin desalting column was pre-processed according to the following procedure:
1) Removed the column's bottom closure and centrifuged (1,000 g, 2 min) to remove the storage solution.
2) Added 5 mL of 0.2 M NaOH on top of the resin to sanitize the columns. Let the columns sit for 30 min.
3) Centrifuged (1,000 g, 2 min) and discarded flow-through.
4) Added 5 mL of formulation buffer onto the resin, centrifuged (1,000 g, 2 min) and discarded flow-through. Repeated this step two additional times until the pH of the flow through was same as formulation buffer. The centrifuge time for the last balance was 6 min.
5) Transferred the columns to new collection tubes and applied the conjugation mixture on top of the resin.
6) Centrifuged (1,000 g, 4 min) and collected flow-through that contained product.

### Concentration Determination of ADC Products

Concentrations of the products were determined by Pierce BCA protein assay.
1) Working reagent was prepared by adding 200 μL of Pierce BCA protein assay reagent B to 10 mL of BCA protein assay reagent A and mixed well.
2) 200 μL of the 1.0 mg/ml mAb solution (mAb_V64) was diluted with dH₂O down to 15.625 ug/mL by 2-fold serial dilution to generate a series of standards for standard curve from 15.625 μg/mL to 1,000 μg/mL.
3) The conjugation sample was diluted with dH₂O.
4) 25 μL each of the triplicate standards and samples were introduced to designated wells of a 96-well plate followed by adding 200 μL of BCA working reagent to each well.
5) OD 562 nm was read after incubating at 37° C. for 30 min in a microplate reader.
6) A standard curve was prepared by plotting the average blank-corrected 562 nm measurement for each antibody standard vs. its concentration in μg/mL.
7) Standard curve was used to determine the protein concentration of ADC sample.

### Aggregation Determination by SEC-HPLC

Size-exclusion chromatography was performed using an Agilent 1260 series HPLC system with the TSK gel G3000SWXL Size-exclusion chromatography column (7.8×300 mm, 5 μm) at 25° C. The mobile phase was consisted of 78 mM KH₂PO₄, 122 mM K₂HPO₄, 250 mM KCl, 15% IPA at pH 7.0±0.1. The flow rate was set at 0.75 mL/min. Sample loading was 40~50 μg per injection. Samples were detected at 280 nm with a UV detector. The retention time of the aggregation peak was recorded based on its relative molecular weight and the aggregation level was determined by the relative area of the peak.

TABLE 2
SEC-HPLC Method.

| | |
|---|---|
| Column | TSK gel G3000SWXL, 7.8 × 300 mm, 5 μm |
| Detection Wavelength | 280 nm |
| Column Oven Temp. | 25 °C |
| Sample Temp. | 10 °C |
| Sample Injection | 40~50 μg |
| amount | |
| Flow rate | 0.75 mL/min |
| Mobile Phase A | 78 mM KH2PO4, 122 mM K2HPO4, 250 mM |
| | KCl, 15% |
| | IPA, pH 7.0 ± 0.1 |
| Gradient | Isocratic, 18 min |

### DAR Determination by HIC-HPLC

Hydrophobic interaction chromatography was performed using an Agilent 1260 series HPLC system with the TSK gel Butyl-NPR hydrophobic interaction chromatography column (4.6 mm I.D.×3.5 cm, 2.5 μm) at 25° C. The mobile phase A was consisted of 1.5 M (NH₄)₂SO₄, 50 mM K₂HPO₄·3H₂O, pH7.0. The mobile phase B was consisted of 21.3 mM KH₂PO₄, 28.6 mM K₂HPO₄·3H₂O, 25% Isopropanol, pH7.0. The flow rate was set at 0.6 mL/min. Sample loading was 8 μL per injection. Samples were detected at 280 nm with a UV detector. The retention time of the DAR species was recorded based on its relative mAb and the HIC-DAR was calculated based on different DAR species with the area of the peak.

TABLE 3
HIC-HPLC Method.

| Column | TSKgel ® Butyl-NPR HIC Column, | | |
|---|---|---|---|
| | 4.6 mm × 3.5 cm, 2.5 μm | | |
| Column Oven Temp | 25 °C | | |
| Flow rate | 0.6 mL/min | | |
| Injection Amount | 8 μL | | |
| Mobile A | 50 mM K2HPO4, 1.5M Ammonium Sulfate, | | |
| | pH 7.0 | | |
| Mobile B | 50 mM K2HPO4, 25% iPrOH, pH 7.0 | | |
| Gradient | Time (min) | A % | B % |
| | 0.00 | 100.0 | 0.0 |
| | 2.00 | 100.0 | 0.0 |
| | 20.00 | 30.0 | 70.0 |
| | 21.00 | 0.0 | 100.0 |
| | 22.00 | 0.0 | 100.0 |
| | 23.00 | 100.0 | 0.0 |
| | 25.00 | 100.0 | 0.0 |

### DAR Determination by Reduced LC-MS

Sample preparation method: 6 μL of 1 M tris, pH 8.0 buffer and 3 μL of 0.1 M DTT solution was added to 30 μg of ADC sample and then dH2O was added to make the final antibody concentration at 1 mg/mL. The mixture was incubated at 37° C. for 30 min. 2 μL sample was injected.

LC-MS was performed using a combination of Agilent 1260 series HPLC system and TOF mass spectrometry with the Agilent PLRP-S 1000A, 8 μm, 50×2.1 mm at 25° C. 0.05% TFA containing dH₂O was used as mobile phase A and acetonitrile containing 0.05% TFA was used as phase B. The flow rate was set at 0.5 mL/min. Sample loading was 2~10 μg. DAR was calculated based on the peak abundance of the deconvoluted Mass.

TABLE 4
LC-MS Method.
LC conditions

| Column | Agilent PLRP-S 1000A, 8 μm, 50 × 2.1 mm | | |
|---|---|---|---|
| Column Temp. | 80 °C | | |
| Mobile phase | Phase A: 0.05% TFA in H₂O; | | |
| | Phase B: 0.05% TFA in Acetonitrile; | | |
| Flow rate | 0.5 mL/min | | |
| Injection amount | 2 μg | | |
| Detection wavelength | 280 nm, 214 nm | | |
| Gradient | Time | A (%) | B (%) |
| | 0.00 | 75 | 25 |
| | 0.70 | 75 | 25 |
| | 1.00 | 66 | 34 |
| | 5.00 | 55 | 45 |
| | 6.00 | 10 | 90 |
| | 7.00 | 10 | 90 |
| | 7.10 | 75 | 25 |
| | 10.00 | 75 | 25 |

MS parameters

| | |
|---|---|
| Gas Temp. | 350 °C |
| Drying Gas | 13 L/min |
| Nebulizer | 45 psig |
| VCap | 5000 V |
| Fragmentor | 250 V |
| Skimmer | 65 V |
| Mass Range | 200-7000 m/z |
| Acquisition Rate | 1 spectra/s |
| Equipment | Agilent Technologies 6224/6230 TOF |

### Residual Free Drug Determination by RP-HPLC

Determine Free Drug for Linker-Payload of Version 1—Carbamic Linkage-Based glucuronide Drug Linker.

The residual free drug level was determined by reverse phase HPLC. After protein precipitation, supernatant was loaded to RP-C18 HPLC column, and eluted by a gradient of increasing the organic mobile phase. The percentage of residual free drug was quantified via peak area by comparing it to external standard curve.

### 1) Solvent Preparation:

### a) Solvent I (for Protein Precipitation)

Weighed 10 g NaCl to the pre-mixed organic solvent of 30 mL MeOH and 50 mL ACN, mixed and stirred 1 hour at least, allowed the solution to stand for at least 1 hour before use. The supernatant was the saturated sodium chloride solution.

### b) Solvent II (Diluent)

Added 2 mL DMA to 8 mL formulation buffer and mixed well. Then added 10 mL solvent I and mixed well again.

### 2) Preparation of Standard Curve

The stock standard linker-drug solution was diluted with Solvent II (diluent) to 1 mM. Added 10 μL of the 1 mM linker-payload solution to 90 μL Solvent II to a final concentration of 100 μM. Then serially diluted the 100 μM standard solution according to the table below down to 0.2 μM.

TABLE 5
Linker-payload Standard Curve Preparation.

| | Volume | Volume | | |
|---|---|---|---|---|
| Standard | of | of | Total | Final |
| Concentra- | Stock | Diluent | Volume | Concentra- |
| tion (μM) | (μL) | (μL) | (μL) | tion (μM) |
| 100 | 40 | 360 | 400 | 10 |
| 10 | 180 | 180 | 360 | 5 |
| 5 | 120 | 180 | 300 | 2 |
| 2 | 180 | 180 | 360 | 1 |
| 1 | 180 | 180 | 360 | 0.5 |
| 0.5 | 120 | 180 | 300 | 0.2 |

### 3) Preparation of Samples

Added 7.5 μL of DMA to 42.5 μL of ADC sample and mixed well. Then added 100 μL solvent I and vortex the mixture for 10 minutes on a mixer at room temperature. Centrifuged the mixture for 10 minutes at 16,000 rcf at room temperature. And then pipetted 80 μL of the supernatant immediately into a glass HPLC vial for analysis.

### 4) HPLC Method

TABLE 6
RP-HPLC method for free drug test.

| Column: | Agilent Poroshell 120 C18 120A 4.6*100 mm | |
|---|---|---|
| | 2.7 μm | |
| Detection Wavelength: | 248 nm&252 nm&370 nm, 4 nm Width | |
| Column Oven Temp.: | 25 °C | |
| Sampler Temp.: | 2~8 °C | |
| Flow Rate: | 0.6 mL/min | |
| Injection Volume: | 40 μL | |
| Mobile Phases: | A: 0.05% TFA in water | |
| | B: 0.05% TFA in ACN | |
| Gradient Program | Isocratic, 20 min | |
| Gradient | Time (min) | B (%) |
| | 0.0 | 20 |
| | 1.0 | 20 |
| | 11.0 | 70 |
| | 12.0 | 95 |
| | 14.0 | 95 |
| | 14.1 | 20 |
| | 20.0 | 20 |

### 5) Data Analysis

a) Integrated the standard curve injections. Plotted the peak area (Y) as a function of concentration (X), Y=kX+b. Calculated the slope (k) and intercept (b).
b) Integrated the related drug peak in the sample. Recorded the sum peak area (Y) and interpolated against the standard curve. Calculated the concentration obtained.

$$Cfreedrug = ( \frac{Y - b}{k} ) \times dilute fold$$

$$Where :$$

$${C}_{free drug} = concentration of free drug by RP - HPLC ( μ M )$$

$$Y = sum peak areas of drug related impurities$$

$$k = slope of linear curve$$

$$Report the relative amount of residual free drug in ADC product ( \% mol / mol ) with two decimal place .$$

$$\% mol / mol = {C}_{free drug} / ( {C}_{Free drug} + {{C}_{protein}}^{*} DAR$$

$$Where :$$

$${C}_{free drug} = concentration of free drug by RP - HPLC ( μ M )$$

$${C}_{protein} = concentration of protein ( μ M )$$

$$DAR = drug to antibody ratio by reduced LC - MS$$

### Endotoxin Determination

The endotoxin level was determined by Endosafe®-PTS™ (Charles River, MCS150K). 25 μL of sample was pipetted into each of the four reservoirs of the PTS Cartridge. The reader drew and mixed the sample with the LAL reagent in the sample channels in addition to the LAL reagent plus positive product control in the spike channels. The sample was combined with the chromogenic substrate then incubated. After mixing, the optical density of the wells was measured and analyzed against an internally archived standard curve.

### Free Drug Removal by Dextran Coated Charcoal

The dextran coated charcoal can be used to remove residual free drug for some linker-payload. Weigh the required amount of charcoal and added it to an Ultrafree-CL centrifuge ultrafiltration tube equipped with a microporous filter membrane. Then added 1 mL of ultrapure water into charcoal, mixed well and centrifuged at 1000×g for 2 minutes. Discarded the flow through in the ultrafiltration tube. Repeat the wash step for 2 times. Every 300 mg of charcoal was then re-suspended in 1 mL formulation buffer. Discarded the flow through in the ultrafiltration tube. Added formulation buffer to the charcoal and mixed well. 10% sample volume of charcoal solution was added into ADC solution. The mixture of charcoal and ADC sample was placed in a 22° C. incubator with slow agitation and incubated for 2 hours. After 2 hours, the tube was centrifuged at 1000×g for 2 minutes to make the charcoal settle to the bottom of the tube. Took out the supernatant and filter through 0.22 μm membrane. The obtained product was then submitted for characterization.

### Conjugation Method ADC-AFI-EXV1

### Conjugation Buffer and Formulation Buffer

TABLE 7
Conjugation and formulation buffer.

| | Reduction/Conjugation | |
|---|---|---|
| Product Name | Buffer | Formulation Buffer |
| ADC-AFI-ExV1 | 50 mM PB, pH 7.0 | 20 mM Histidine, 7% |
| | | sucrose, pH 5.5 |

### Experimental Procedure for Pilot Conjugation

mAb in original buffer (20 mM His, 150 mM NaCl, pH 6.5) was pipetted into three 1.5 mL EP tubes, and then were reduced by 1.5, 2.5 and 3.5 molar eq. of TCEP. Reaction buffer was added to each tube to make the mAb concentration in the reaction at 4.5 mg/mL. The reaction vials were placed in an incubator-shaker under 37° C. with agitation speed at 60 rpm. After 2 hours of reduction, 10 mM Target 1 in DMA was added to each of the three samples to make the drug to mAb molar eq. at 8.0. DMA solvent was added into each sample to make the organic solvent at 10%, and were incubated under 4° C. for another 1 hour. After 1 hour, samples were purified by spin desalting column (40 K, 0.5 mL). The pilot products were characterized for SEC-HPLC, HIC-HPLC and LC-MS.

### Experimental Procedure for Confirming Conjugation

1 mg scale conjugation was performed to confirm the optimum condition from pilot conjugations for target DAR 4.0. mAb in original buffer was pipetted into 1.5 mL EP tubes, and then was reduced by the optimum molar eq. of TCEP. Reaction buffer was added to the tube to make the mAb concentration at 4.5 mg/mL. The reaction vial was placed in an incubator-shaker under 37° C. with agitation speed at 60 rpm. After 2 hours of reduction, 10 mM Target 1 in DMA was added to sample to make the drug to mAb molar eq. at 8.0. DMA was added into sample to make the organic solvent at 10%, and was incubated under 4° C. for another 1 hour. After 1 hour, the sample was purified by spin desalting column (40 K, 0.5 mL). The confirming product was characterized by SEC-HPLC and LC-MS.

### Experimental Procedure for Bulk Conjugation

mAb (15 mg) in original buffer was pipetted into 50 mL tube, and was reduced by the confirmed optimum eq. of TCEP. Reaction buffer was added to tube to make the mAb concentration at 4.5 mg/mL. The reaction vial was placed in an incubator-shaker under 37° C. with agitation speed at 60 rpm. After 2 hours of reduction, 10 mM Target 1 in DMA was added to sample to make the drug to mAb molar eq. at 8.0. DMA was added into sample to make the organic solvent at 10%, and was incubated under 4° C. for another 1 hour. After 1 hour, the sample was purified via spin desalting column (40 K, 10 mL). The product was characterized by SEC-HPLC and LC-MS. If the reduced MS-DAR meets the requirements, then another 15 mg scale conjugation will be performed with same condition. If the reduced MS-DAR does not meet the requirements, then molar eq. of TCEP/mAb will be adjusted in the 2^{nd} round of 15 mg scale conjugation. Both batch of products were pooled and dialyzed overnight. After dialysis, the product was applied to dextran-coated charcoal for free drug remove.

### Efficacy Study Using Human Gastric SNU16 Subcutaneous Xenograft Model (CDX)

This project was performed in compliance with the internal operating standards at Wuxi AppTec.

The objective of this study was to evaluate the in vivo anti-tumor efficacy of Exatecan ADC (ADC-AFI-ExV1) in the human gastric SNU-16 subcutaneous xenograft model in female BALB/c Nude mice. Expression of the target STn was performed prior to cell injections by FACS and detection using mAb_V64 together with secondary fluorescent mAb (FIG. 38). Experimental design of efficacy study relative to the inventiveness of this patent is summarized as follows:

TABLE 8
Description of experimental design.

| | | | | Dose | | |
|---|---|---|---|---|---|---|
| | | | Dose | volume | | |
| Group | N^{a} | Treatment | mg/kg | μL/g^{b} | Route | Schedule |
| 1 | 7 | Vehicle (PBS) | — | 10 | IV | PG-D 0, |
| | | | | | | D 18 |
| 2 | 7 | ADC-IgG1-ExV1 | 12 | 10 | IV | PG-D 0, |
| | | (DAR4) | | | | D 18 |
| 3 | 7 | ADC-AFI-ExV1 | 8 | 10 | IV | PG-D 0, |
| | | (DAR4) | | | | D 18 |
| 4 | 7 | ADC-AFI-DXd | 8 | 10 | IV | PG-D 0, |
| | | (DAR4) | | | | D 18 |

N: number of animals per group; Dose volume: adjust dosing volume based on body weight 10 μL/g.

### Animals

Species: Mus musculus
Strain: BALB/c Nude
Age: 6-8 weeks
Body weight: 17.14-23.47 g
Sex: female
Number of animals: 42 mice plus spare
Animal supplier: Zhejiang Vital River Laboratory Animal Technology Co., Ltd.
No. of certificate of quality: 20220711Abzz0619000293

### Housing Condition

The mice were kept in individual ventilation cages at constant temperature and humidity with ¾ animals in each cage.
Temperature: 20-26° C.
Humidity 40-70%.

Cages: Made of polycarbonate. The size is 325 mm×210 mm×180 mm. The bedding material was corn cob, which was changed twice per week.

### Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

### Tumor Measurements and Endpoints

The major endpoint was to see if the tumor growth could be delayed or mice could be cured. Tumor size was measured twice weekly in two dimensions using a caliper, and the volume was expressed in mm³ using the formula: V=0.5a×b² where a and b are the long and short diameters of the tumor, respectively.

The antitumor efficacy of the compound was evaluated by TGI (%) or T/C (%). TGI (%), reflecting the tumor growth inhibition rate. TGI was calculated for each group using the formula: TGI (%)=[1−(Tᵢ−T₀)/(Vᵢ−V₀)]×100%; Tᵢ is the average tumor volume of a treatment group on a given day, T₀ is the average tumor volume of the treatment group on day 0, Vᵢ is the average tumor volume of the vehicle control group on the same day with Tᵢ, and V₀ is the average tumor volume of the vehicle group on day 0.

The T/C value (in percent) is an indication of antitumor effectiveness; T/C was calculated for each group using the formula: T/C (%)=T_{RTV}/C_{RTV}×100%; T_{RTV} was the RTV of treatment group, while C_{RTV} is the RTV of control group on the same day. RTV for each tumor was calculated as RTV=Vₜ/V₀, Vₜ is the average tumor volume on a given day, V₀ is the average tumor volume on the day of treatment start for the same tumor.

Tumor weight was measured at the study termination. T/C_{weight} value (in percent) was calculated using the formula: T/C_{weight}%=T_{weight}/C_{weight}×100% where T_{weight} and C_{weight} were the mean tumor weights of the treated and the vehicle control groups, respectively.

In this study, the therapeutic efficacy of Exatecan ADCs as a single agent in the treatment of the human gastric SNU-16 xenograft model was evaluated. The results of tumor sizes in different groups at different time points after the start of treatment are shown in FIG. 4a-b.

Additional efficacy study reported in Sup. FIG. 25a was performed using same animal models. Treatment schedules were reported in legend of same figure.

### Biodistribution

The experiments conducted in this study complied with UK laws and were inclusive of ethics approval.

### PET Scanner

The Siemens Inveon PET scanner was used to acquire the longitudinal images. Positron emission tomography (PET) is a nuclear medical imaging technique that produces a three-dimensional image of functional processes in the body. The system detects pairs of gamma rays emitted indirectly by a positron-emitting radionuclide (tracer), which is introduced into the body on a biologically active molecule.

In preclinical studies, PET imaging can be used for non-invasive detection and investigation of diseases in small animal models.

The Siemens Inveon PET System is a state-of-the-art system for laboratory animal PET studies with its specification and capabilities noted below.
LSO with 1.6×1.6 mm detector
Sensitivity

### Gamma Counter

The Perkin Elmer Wallac Wizard Gamma counter was used to understand the ex-vivo organ biodistribution data. Its detector system consists of a thallium activated sodium iodide crystal. It offers a radionuclide library of 51 nuclides with a sample changer and storage capacity of 100 racks (1000 samples) an energy range of 15-2000 keV.

### Animal Model

Female BALB/C mice at 8 weeks old were purchased from Envigo and acclimatised for 7 days prior to cancer cells implantation in the right flank region. Animals were split into 3 groups; 2 groups received parental 4T1 cell line (WT 4T1) and 1 group received STn 4T1 cell line (STn 4T1). Following 8 days of tumour growth, mice were randomised and separated as follows: half of WT 4T1 group received i.v. injection of ⁸⁹Zr-anti-STn antibody, whereas the other half received ⁸⁹Zr-isotype control. The STn 4T1 group received ⁸⁹Zr-anti-STn antibody (Ab). All procedures were carried out under HO project licence PPL P15A1884A.

### Radiotracers

⁸⁹Zr was purchased from our commercial radiotracer supplier (Wolfson Molecular Imaging Centre, University of Manchester) and injected intravenously into the animals. Mice were imaged at 2 h, 8 h, 24 h, 48 h, 72 h and 96 h post tracer injection.

Syringe activity was measured before and after injection using the BriTec well counter and the times of measurement noted using a clock synchronised to the PET system. The injected dose was then calculated in Microsoft Excel as the difference of the pre and post injection syringe activities after decay was corrected to the time of injection.

### Animal Protocol

Mice were split into 3 groups: group 1 were implanted with WT 4T1 cells and received an injection of isotype control; group 2 were implanted with WT 4T1 cells and received anti-STn Ab; group 3 were implanted with STn 4T1 cells and received anti-STn Ab. A bolus injection of ⁸⁹Zr-Ab (~100 μl) was administered intravenously and mice imaged under static PET at 2 h, 8 h, 24 h, 48 h, 72 h, 96 h post-injection. PET scan was performed for 20 min in anaesthetised mice. Anaesthesia was induced and maintained with isoflurane delivered in 100% oxygen (~1.5% isoflurane, 3 L oxygen). Heating pads were provided throughout, respiration and body temperature of the animal were monitored through BioVet.

### Imaging Protocol

Briefly, a 20-minute PET static scan was acquired with 3D histogramming and MAP/3D reconstruction. QC measurement was carried out for the PET scanner prior to imaging commencing. Immediately after the final PET scanning time point, mice were euthanised and tissues collected for biodistribution study. Blood, muscle, lungs, liver, spleen, kidneys, heart, pancreas, ovary, large intestine, small intestine, stomach, TDLN, NDLN, tumour, and tail (injection site) were dissected from each animal for gamma counter ex-vivo analysis.

Tumours and ovary were fixed in formalin and transferred to ethanol for further histological examination. Both tumours and ovary were shipped to CellmAbs for histological evaluation. Blood samples were also collected from each animal at 2 h, 8 h, and 24 h after the PET scanning for gamma counter analysis.

### Stickiness ELISA

Biomolecules including DNA, LPS, Lysozym and Cell lysate were immobilized to ELISA plate. Further incubation of different antibodies including control IgG1 Palivizumab and Trastuzumab, together with novel mAb_v1 and mAb_v64 and detection by human Fc. Absorbance data (A450-A620) were reported as after normalization to therapeutic antibody Palivizumab.

### Antibody Stability—SEC-HPLC

Different antibodies including mAb_v1, mAb_v64 and control IgG1 (Palivizumab) were used to perform stability tests under two stress conditions as follow:
Temperature stress for 48 h at 45° C.
pH stress for 24 h at pH3

Data were the represented after stress tests as SEC-HPLC analysis showing monomeric mAb forms for all the antibodies tested as shown in FIG. 6c.

### Internalization Assay

The objective of this study was to evaluate the internalization kinetics of anti-STn antibodies in Breast Cancer cell lines overexpressing STn (MDA-MB231-STn) and its parental WT cell line (negative STn expression), together with naturally STn expressing cell lines colon cancer (COLO205) and gastric (SNU16) cell lines. To evaluate this feature, we assessed an internalization assay using parental L2A5, humanized Abs clones (mAb_v1) and affinity matured mAb clones (mAb_v57; mAb_v48; mAb_v46; mAb_v53; mAb_v25; mAb_v64) together with a negative (IgG1-Palivizumab), anti-STn positive control (mAb_PC) and benchmark mAb Trastuzumab antibodies controls as shown in FIG. 7a-d.

Antibody internalization-assay analysis using 1×10⁴ target cells per well were plated in 96-well plates. In this assay, the antibodies were labelled with a Zenon pH-rodo fluorophore (Invitrogen, Cat. Z25611) that is activated only in low pH conditions found in the early endosome. Labelled antibodies (3 μg/mL) were incubated for Oh, 4 h or 24 h with the target cells and the pHrodo fluorescence was measured by flow cytometry. Antibody internalization was indirectly evaluated by measuring pHrodo fluorescence by flow cytometry. A signal-to-background ratio (S/B) was calculated by dividing the MFI data from each antibody to the non-internalizing mAb Palivizumab) MFI. The data normalized to Palivizumab was plotted as internalization factor for each time point.

### Antibody Binding

Validation of antibody binding was performed with cell lines that naturally express STn (COLO205, SNU16, OV90). All antibodies showed higher and specific binding to the target cells. FIG. 8 shows results of the binding profile of different antibody variants (listed in FIGS. 9 to 13 Supplementary antibody sequences in cell lines with different STn expression levels. Each antibody was titrated using a 7-point concentration curve and the binding intensity was measured by flow cytometry using a secondary antibody conjugated to a fluorophore (Invitrogen Cat., A-21445).

The embodiments of the disclosure described above are intended to be merely exemplary, numerous variations and modifications will be apparent to those skilled in the art. All such variations and modifications are intended to be within the scope of the present invention as defined in any appended claims.

1. An antibody-drug conjugate (ADC) comprising an antibody which binds to sialyl Tn (STn) or a glycan terminated by an alpha 2,6-linked sialic acid, conjugated to a drug via a linker, wherein the linker comprises a linker cleavable by glucuronidase, and the drug comprises a growth inhibitory agent.

2. An antibody-drug conjugate (ADC) according to claim 1 wherein the linker comprises a β-glucuronide moiety.

3. An antibody-drug conjugate (ADC) according to claim 1 or 2 wherein the linker comprises a β-glucuronide moiety as shown in formula I′: 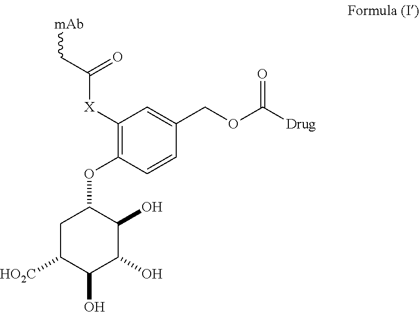
wherein X is NH, N—CH₃ or CF₂.

4. An antibody-drug conjugate (ADC) according to claim 1, 2 or 3 wherein the linker comprises a β-glucuronide moiety as shown in formula I:

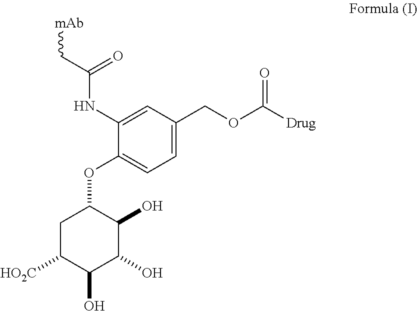

5. An antibody-drug conjugate (ADC) according to any preceding claim wherein the linker is linked to, or is modified to link to, the drug via a carbamate linkage.

6. An antibody-drug conjugate (ADC) according to any one of claims 1 to 4 wherein the linker is linked to, or is modified to link to, the drug via a quaternary ammonium salt linkage.

7. An antibody-drug conjugate (ADC) according to any one of claims 1 to 6 wherein the linker is PEGylated with a group comprising polyethylene glycol (PEG).

8. An antibody-drug conjugate (ADC) according to claim 7 wherein the linker comprises a β-glucuronide moiety PEGylated with a group comprising polyethylene glycol (PEG).

9. An antibody-drug conjugate (ADC) according to any one of claims 7 to 8 wherein the linker is PEGylated with a group comprising polyethylene glycol (PEG) based on the structure: 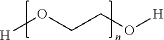
wherein n is from 1 to 5.

10. An antibody-drug conjugate (ADC) according to claim 9 wherein n is 2 to 4.

11. An antibody-drug conjugate (ADC) according to claim 10 wherein n is 3.

12. An antibody-drug conjugate (ADC) formed by conjugating a drug to an antibody as defined in claim 1 with a linker, wherein the linker is a group of formula II:

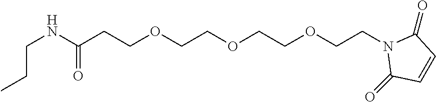 [SMILES: CCCNC(=O)CCOCCOCCOCCN1C(=O)C=CC1=O]

13. An antibody-drug conjugate (ADC) comprising an antibody as defined in claim 1 conjugated to a drug via a linker, wherein the linker comprises a group of formula IIA:

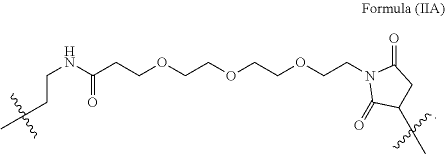 [SMILES: *CCNC(=O)CCOCCOCCOCCN1C(=O)CC(*)C1=O]

14. An antibody-drug conjugate (ADC) comprising an antibody as defined in claim 1 conjugated to a drug via a linker, wherein the linker is a group as shown in formula IIIA′: 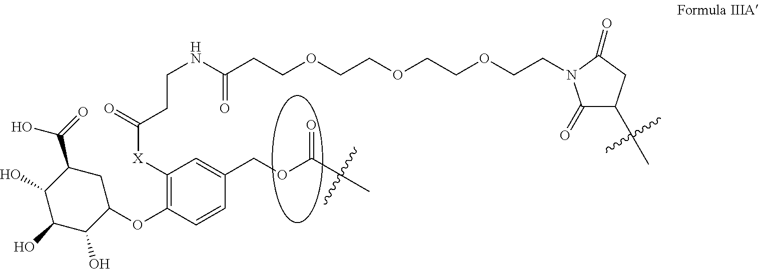
wherein X is NH, N—CH₃ or CF₂.

15. An antibody-drug conjugate (ADC) comprising an antibody as defined in claim 1 conjugated to a drug via a linker, wherein the linker is a group as shown in formula IIIA:

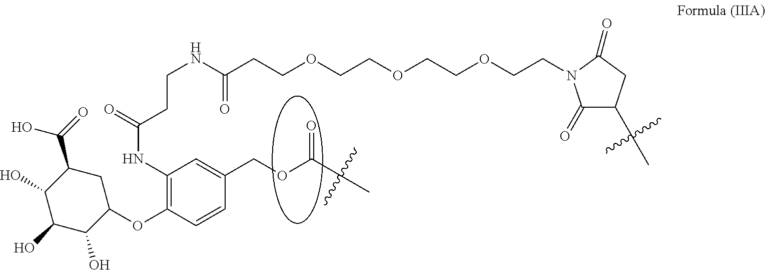 [SMILES: *C(=O)OCc1ccc(OC2C[C@H](C(=O)O)[C@@H](O)[C@H](O)[C@H]2O)c(NC(=O)CCNC(=O)CCOCCOCCOCCN2C(=O)CC(*)C2=O)c1]

16. An antibody-drug conjugate (ADC) formed by conjugating a drug to an antibody as defined in claim 1 with a linker, wherein the linker is as shown in formula III:

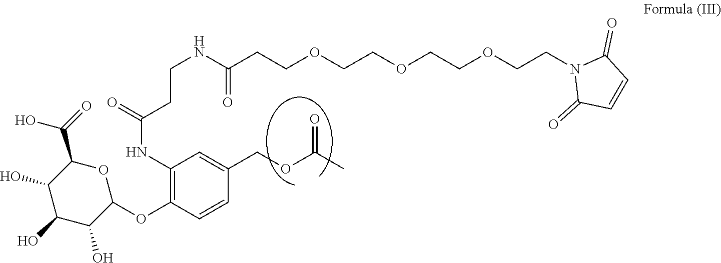 [SMILES: CC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

17. An antibody-drug conjugate (ADC) comprising an antibody as defined in claim 1 conjugated to a drug via a linker, wherein the linker is as shown in formula IVA′: 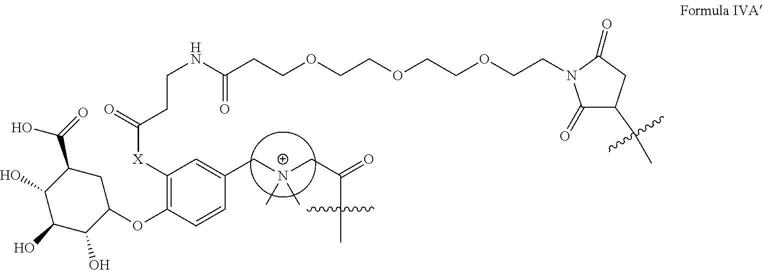
wherein X is NH, N—CH₃ or CF₂.

18. An antibody-drug conjugate (ADC) comprising an antibody as defined in claim 1 conjugated to a drug via a linker, wherein the linker is as shown in formula IVA:

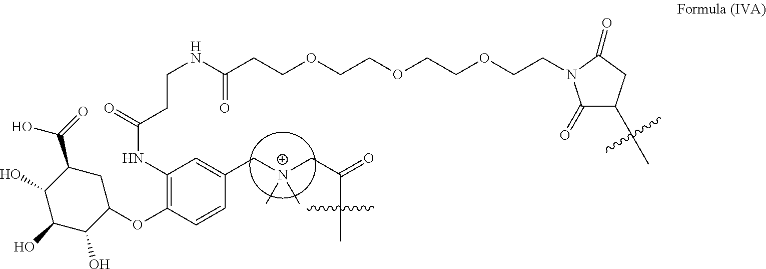 [SMILES: *C(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)CC(*)C2=O)c1]

19. An antibody-drug conjugate (ADC) formed by conjugating a drug to an antibody as defined in claim 1 with a linker, wherein the linker is as shown in formula IV:

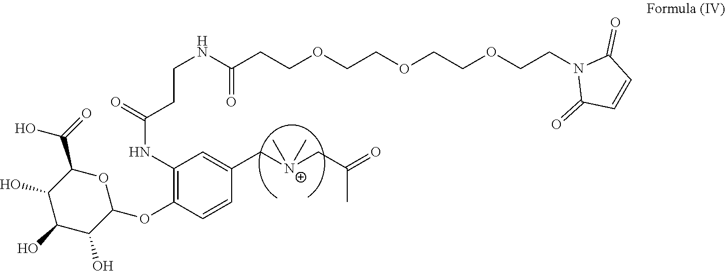 [SMILES: CC(=O)C[N+](C)(C)Cc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

20. An antibody-drug conjugate (ADC) comprising an antibody as defined in claim 1 conjugated to a drug via a linker, wherein the drug-linker moiety of the ADC is as shown in formula VA′: 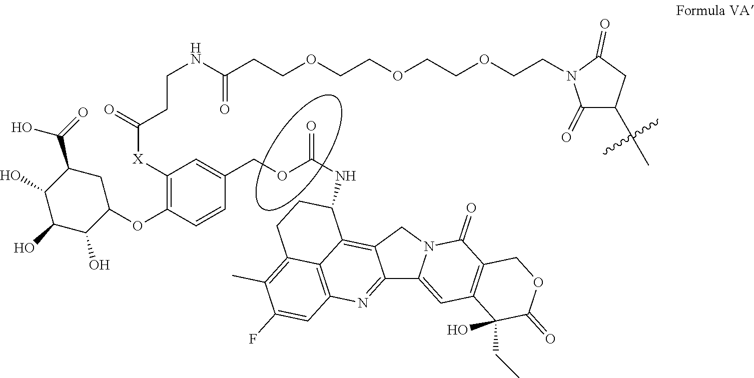
wherein X is NH, N—CH₃ or CF₂.

21. An antibody-drug conjugate (ADC) comprising an antibody as defined in claim 1 conjugated to a drug via a linker, wherein the drug-linker moiety of the ADC is as shown in formula VA:

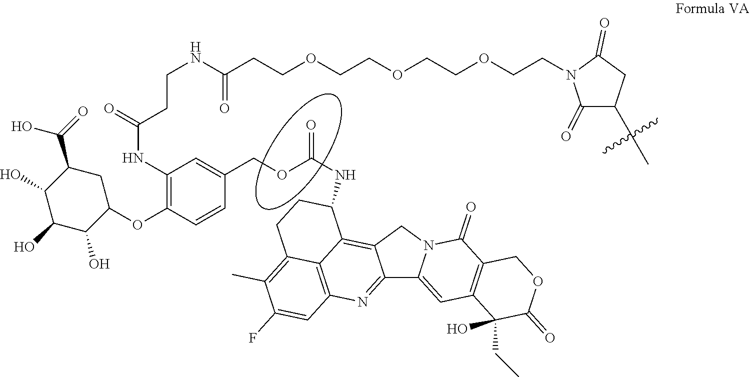 [SMILES: *C1CC(=O)N(CCOCCOCCOCCC(=O)NCCC(=O)Nc2cc(COC(=O)N[C@H]3CCc4c(C)c(F)cc5nc6c(c3c45)Cn3c-6cc4c(c3=O)COC(=O)[C@]4(O)CC)ccc2OC2C[C@H](C(=O)O)[C@@H](O)[C@H](O)[C@H]2O)C1=O]

22. An antibody-drug conjugate (ADC) formed by conjugating a drug to an antibody as defined in claim 1 with a linker, wherein the drug-linker moiety of the ADC is as shown in Formula V:

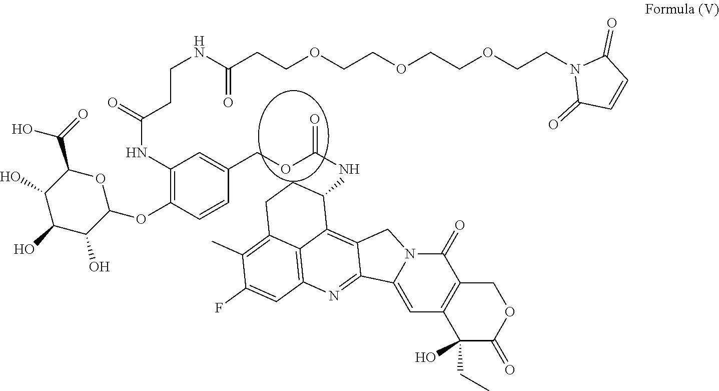 [SMILES: CC[C@@]1(O)C(=O)OCc2c1cc1n(c2=O)Cc2c-1nc1cc(F)c(C)c3c1c2[C@@H](NC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1)CC3]

23. An antibody-drug conjugate (ADC) comprising an antibody as defined in claim 1 conjugated to a drug via a linker, wherein the drug-linker moiety of the ADC is as shown in formula VIA′: 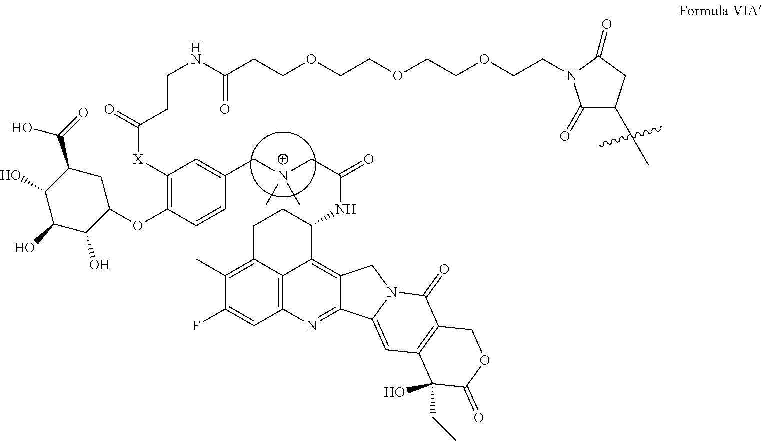
wherein X is NH, N—CH₃ or CF₂.

24. An antibody-drug conjugate (ADC) comprising an antibody as defined in claim 1 conjugated to a drug via a linker, wherein the drug-linker moiety of the ADC is as shown in formula VIA:

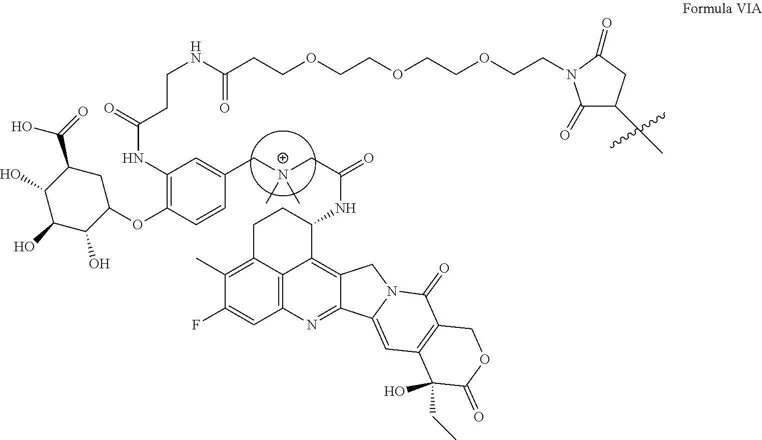 [SMILES: *C1CC(=O)N(CCOCCOCCOCCC(=O)NCCC(=O)Nc2cc(C[N+](C)(C)CC(=O)N[C@H]3CCc4c(C)c(F)cc5nc6c(c3c45)Cn3c-6cc4c(c3=O)COC(=O)[C@]4(O)CC)ccc2OC2C[C@H](C(=O)O)[C@@H](O)[C@H](O)[C@H]2O)C1=O]

25. An antibody-drug conjugate (ADC) formed by conjugating a drug to an antibody as defined in claim 1 with a linker, wherein the drug-linker moiety of the ADC is as shown in Formula VI:

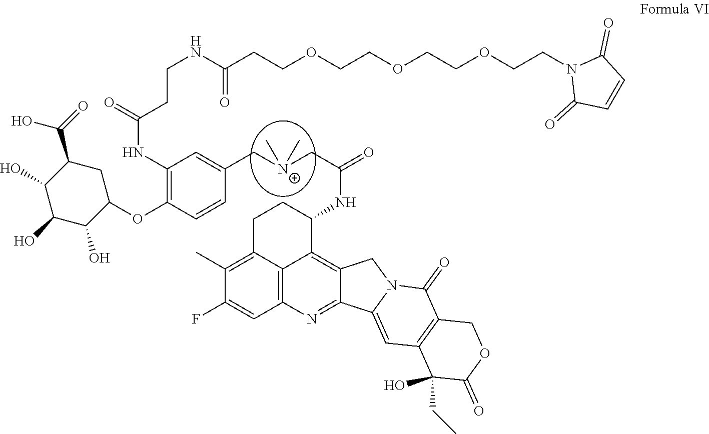 [SMILES: CC[C@@]1(O)C(=O)OCc2c1cc1n(c2=O)Cc2c-1nc1cc(F)c(C)c3c1c2[C@@H](NC(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)C=CC2=O)c1)CC3]

26. An antibody-drug conjugate (ADC) according to any preceding claim wherein the sialyl Tn (STn) or a glycan terminated by an alpha 2,6-linked sialic acid is a human or animal protein.

27. An antibody-drug conjugate (ADC) according to any preceding claim wherein the drug is exatecan, or deruxtecan, or a derivative thereof.

28. An antibody-drug conjugate (ADC) according to any preceding claim wherein the antibody is an antibody fragment.

29. An antibody-drug conjugate (ADC) according to claim 28 wherein the antibody fragment is selected from the group consisting of Fv, Fab, F(ab′)2, Fab′, dsFv, (dsFv)2, scFv, sc(Fv)2, and diabodies.

30. An antibody-drug conjugate (ADC) according to any preceding claim wherein the antibody is a human or animal antibody.

31. An antibody-drug conjugate (ADC) according to any preceding claim wherein the antibody comprises:
(a) a heavy chain variable region (VH) wherein the VH comprises complementarity determining regions (CDRs) selected from the group consisting of:
(i) H-CDR1, H-CDR2 and H-CDR3 as shown in any one SEQ ID NOs. 1 to 24 or 49 to 88 respectively
and/or,
(b) a light chain variable region (VL) wherein the VL comprises complementarity determining regions (CDRs) selected from the group consisting of:
(i) L-CDR1, L-CDR2, and L-CDR3 as shown in any one of SEQ ID NOs. 25 to 48 or 89 to 128 respectively; optionally wherein at least one CDR may comprise one or two amino acid substitutions compared to the recited sequence.

32. An antibody-drug conjugate (ADC) according to claim 31, wherein the antibody comprises a heavy chain variable region (VH) and light chain variable region (VL).

33. An antibody-drug conjugate (ADC) according to claim 31 or 32, wherein the antibody comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 2 paired with L-CDR1, L-CDR2, and L-CDR3 as shown in SEQ ID NO. 26;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 4 paired with L-CDR1, L-CDR2, and L-CDR3 as shown in SEQ ID NO. 28;
H-CDR1, H-CDR2 and H-CDR3 as shown in SEQ ID NO. 5 paired with L-CDR1, L-CDR2, and L-CDR3 as shown in SEQ ID NO. 29;
H-CDR1, H-CDR2 and H-CDR3 as shown in SEQ ID NO. 6 paired with L-CDR1, L-CDR2, and L-CDR3 as shown in SEQ ID NO. 30;
H-CDR1, H-CDR2 and H-CDR3 as shown in SEQ ID NO. 7 paired with L-CDR1, L-CDR2, and L-CDR3 as shown in SEQ ID NO. 31;
H-CDR1, H-CDR2 and H-CDR3 as shown in SEQ ID NO. 8 paired with L-CDR1, L-CDR2, and L-CDR3 as shown in SEQ ID NO. 32;
H-CDR1, H-CDR2 and H-CDR3 as shown in SEQ ID NO. 9 paired with L-CDR1, L-CDR2, and L-CDR3 as shown in SEQ ID NO. 33;
H-CDR1, H-CDR2 and H-CDR3 as shown in SEQ ID NO. 10 paired with L-CDR1, L-CDR2, and L-CDR3 as shown in SEQ ID NO. 34;
H-CDR1, H-CDR2 and H-CDR3 as shown in SEQ ID NO. 11 paired with L-CDR1, L-CDR2, and L-CDR3 as shown in SEQ ID NO. 35;
H-CDR1, H-CDR2 and H-CDR3 as shown in SEQ ID NO. 12 paired with L-CDR1, L-CDR2, and L-CDR3 as shown in SEQ ID NO. 36;
H-CDR1, H-CDR2 and H-CDR3 as shown in SEQ ID NO. 13 paired with L-CDR1, L-CDR2, and L-CDR3 as shown in SEQ ID NO. 37;
H-CDR1, H-CDR2 and H-CDR3 as shown in SEQ ID NO. 14 paired with L-CDR1, L-CDR2, and L-CDR3 as shown in SEQ ID NO. 38;
H-CDR1, H-CDR2 and H-CDR3 as shown in SEQ ID NO. 15 paired with L-CDR1, L-CDR2, and L-CDR3 as shown in SEQ ID NO. 39;
H-CDR1, H-CDR2 and H-CDR3 as shown in SEQ ID NO. 16 paired with L-CDR1, L-CDR2, and L-CDR3 as shown in SEQ ID NO. 40;
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 18 paired with L-CDR1, L-CDR2, and L-CDR3 as shown in SEQ ID NO. 42;
H-CDR1, H-CDR2 and H-CDR3 as shown in SEQ ID NO. 19 paired with L-CDR1, L-CDR2, and L-CDR3 as shown in SEQ ID NO. 43;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45;
H-CDR1, H-CDR2 and H-CDR3 as shown in SEQ ID NO. 22 paired with L-CDR1, L-CDR2, and L-CDR3 as shown in SEQ ID NO. 46;
H-CDR1, H-CDR2 and H-CDR3 as shown in SEQ ID NO. 23 paired with L-CDR1, L-CDR2, and L-CDR3 as shown in SEQ ID NO. 47; or
H-CDR1, H-CDR2 and H-CDR3 as shown in SEQ ID NO. 24 paired with L-CDR1, L-CDR2, and L-CDR3 as shown in SEQ ID NO. 48; optionally wherein at least one CDR may comprise one or two amino acid substitutions compared to the recited sequence.

34. An antibody-drug conjugate (ADC) according to claim 33, wherein the antibody comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 2 paired with L-CDR1, L-CDR2, and L-CDR3 as shown in SEQ ID NO. 26; optionally wherein at least one CDR may comprise one or two amino acid substitutions compared to the recited sequence.

35. An antibody-drug conjugate (ADC) according to claim 31 or 32, wherein the antibody comprises one of the following pairs of light chain CDRs and heavy chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 50 paired with L-CDR1, L-CDR2, and L-CDR3 as shown in SEQ ID NO. 90;
H-CDR1, H-CDR2 and H-CDR3 as shown in SEQ ID NO. 51 paired with L-CDR1, L-CDR2, and L-CDR3 as shown in SEQ ID NO. 91;
H-CDR1, H-CDR2 and H-CDR3 as shown in SEQ ID NO. 52 paired with L-CDR1, L-CDR2, and L-CDR3 as shown in SEQ ID NO. 92;
H-CDR1, H-CDR2 and H-CDR3 as shown in SEQ ID NO. 53 paired with L-CDR1, L-CDR2, and L-CDR3 as shown in SEQ ID NO. 93;
H-CDR1, H-CDR2 and H-CDR3 as shown in SEQ ID NO. 54 paired with L-CDR1, L-CDR2, and L-CDR3 as shown in SEQ ID NO. 94;
H-CDR1, H-CDR2 and H-CDR3 as shown in SEQ ID NO. 55 paired with L-CDR1, L-CDR2, and L-CDR3 as shown in SEQ ID NO. 95;
H-CDR1, H-CDR2 and H-CDR3 as shown in SEQ ID NO. 56 paired with L-CDR1, L-CDR2, and L-CDR3 as shown in SEQ ID NO. 96;
H-CDR1, H-CDR2 and H-CDR3 as shown in SEQ ID NO. 57 paired with L-CDR1, L-CDR2, and L-CDR3 as shown in SEQ ID NO. 97;
H-CDR1, H-CDR2 and H-CDR3 as shown in SEQ ID NO. 58 paired with L-CDR1, L-CDR2, and L-CDR3 as shown in SEQ ID NO. 98;
H-CDR1, H-CDR2 and H-CDR3 as shown in SEQ ID NO. 59 paired with L-CDR1, L-CDR2, and L-CDR3 as shown in SEQ ID NO. 99;
H-CDR1, H-CDR2 and H-CDR3 as shown in SEQ ID NO. 60 paired with L-CDR1, L-CDR2, and L-CDR3 as shown in SEQ ID NO. 100;
H-CDR1, H-CDR2 and H-CDR3 as shown in SEQ ID NO. 61 paired with L-CDR1, L-CDR2, and L-CDR3 as shown in SEQ ID NO. 101;
H-CDR1, H-CDR2 and H-CDR3 as shown in SEQ ID NO. 62 paired with L-CDR1, L-CDR2, and L-CDR3 as shown in SEQ ID NO. 102;
H-CDR1, H-CDR2 and H-CDR3 as shown in SEQ ID NO. 63 paired with L-CDR1, L-CDR2, and L-CDR3 as shown in SEQ ID NO. 103;
H-CDR1, H-CDR2 and H-CDR3 as shown in SEQ ID NO. 64 paired with L-CDR1, L-CDR2, and L-CDR3 as shown in SEQ ID NO. 104;
H-CDR1, H-CDR2 and H-CDR3 as shown in SEQ ID NO. 65 paired with L-CDR1, L-CDR2, and L-CDR3 as shown in SEQ ID NO. 105;
H-CDR1, H-CDR2 and H-CDR3 as shown in SEQ ID NO. 66 paired with L-CDR1, L-CDR2, and L-CDR3 as shown in SEQ ID NO. 106;
H-CDR1, H-CDR2 and H-CDR3 as shown in SEQ ID NO. 67 paired with L-CDR1, L-CDR2, and L-CDR3 as shown in SEQ ID NO. 107;
H-CDR1, H-CDR2 and H-CDR3 as shown in SEQ ID NO. 68 paired with L-CDR1, L-CDR2, and L-CDR3 as shown in SEQ ID NO. 108;
H-CDR1, H-CDR2 and H-CDR3 as shown in SEQ ID NO. 69 paired with L-CDR1, L-CDR2, and L-CDR3 as shown in SEQ ID NO. 109;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 71 paired with L-CDR1, L-CDR2, and L-CDR3 as shown in SEQ ID NO. 111;
H-CDR1, H-CDR2 and H-CDR3 as shown in SEQ ID NO. 72 paired with L-CDR1, L-CDR2, and L-CDR3 as shown in SEQ ID NO. 112;
H-CDR1, H-CDR2 and H-CDR3 as shown in SEQ ID NO. 73 paired with L-CDR1, L-CDR2, and L-CDR3 as shown in SEQ ID NO. 113;
H-CDR1, H-CDR2 and H-CDR3 as shown in SEQ ID NO. 74 paired with L-CDR1, L-CDR2, and L-CDR3 as shown in SEQ ID NO. 114;
H-CDR1, H-CDR2 and H-CDR3 as shown in SEQ ID NO. 75 paired with L-CDR1, L-CDR2, and L-CDR3 as shown in SEQ ID NO. 115;
H-CDR1, H-CDR2 and H-CDR3 as shown in SEQ ID NO. 76 paired with L-CDR1, L-CDR2, and L-CDR3 as shown in SEQ ID NO. 116;
H-CDR1, H-CDR2 and H-CDR3 as shown in SEQ ID NO. 77 paired with L-CDR1, L-CDR2, and L-CDR3 as shown in SEQ ID NO. 117;
H-CDR1, H-CDR2 and H-CDR3 as shown in SEQ ID NO. 78 paired with L-CDR1, L-CDR2, and L-CDR3 as shown in SEQ ID NO. 118;
H-CDR1, H-CDR2 and H-CDR3 as shown in SEQ ID NO. 79 paired with L-CDR1, L-CDR2, and L-CDR3 as shown in SEQ ID NO. 119;
H-CDR1, H-CDR2 and H-CDR3 as shown in SEQ ID NO. 80 paired with L-CDR1, L-CDR2, and L-CDR3 as shown in SEQ ID NO. 120;
H-CDR1, H-CDR2 and H-CDR3 as shown in SEQ ID NO. 81 paired with L-CDR1, L-CDR2, and L-CDR3 as shown in SEQ ID NO. 121;
H-CDR1, H-CDR2 and H-CDR3 as shown in SEQ ID NO. 82 paired with L-CDR1, L-CDR2, and L-CDR3 as shown in SEQ ID NO. 122;
H-CDR1, H-CDR2 and H-CDR3 as shown in SEQ ID NO. 83 paired with L-CDR1, L-CDR2, and L-CDR3 as shown in SEQ ID NO. 123;
H-CDR1, H-CDR2 and H-CDR3 as shown in SEQ ID NO. 84 paired with L-CDR1, L-CDR2, and L-CDR3 as shown in SEQ ID NO. 124;
H-CDR1, H-CDR2 and H-CDR3 as shown in SEQ ID NO. 85 paired with L-CDR1, L-CDR2, and L-CDR3 as shown in SEQ ID NO. 125;
H-CDR1, H-CDR2 and H-CDR3 as shown in SEQ ID NO. 86 paired with L-CDR1, L-CDR2, and L-CDR3 as shown in SEQ ID NO. 126;
H-CDR1, H-CDR2 and H-CDR3 as shown in SEQ ID NO. 87 paired with L-CDR1, L-CDR2, and L-CDR3 as shown in SEQ ID NO. 127; or
H-CDR1, H-CDR2 and H-CDR3 as shown in SEQ ID NO. 88 paired with L-CDR1, L-CDR2, and L-CDR3 as shown in SEQ ID NO. 128; optionally wherein at least one CDR may comprise one or two amino acid substitutions compared to the recited sequence.

36. An antibody-drug conjugate (ADC) according to claim 35, wherein the antibody comprises one of the following pairs of heavy chain CDRs and light chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 81 paired with L-CDR1, L-CDR2, and L-CDR3 as shown in SEQ ID NO. 121; or
H-CDR1, H-CDR2 and H-CDR3 as shown in SEQ ID NO. 88 paired with L-CDR1, L-CDR2, and L-CDR3 as shown in SEQ ID NO. 128; optionally wherein at least one CDR may comprise one or two amino acid substitutions compared to the recited sequence.

37. An antibody-drug conjugate (ADC) according to any one of claims 31 to 36, wherein the antibody comprises:
(a) a heavy chain variable region (VH) wherein the VH comprises a humanised heavy chain framework region
and/or,
(b) a light chain variable region (VL) wherein the VL comprises a humanised light chain framework region.

38. An antibody-drug conjugate (ADC) according to any one of claims 31 to 37, wherein in the antibody comprises:
(a) a heavy chain variable region (VH) wherein the VH comprises a humanised heavy chain framework region as shown in the heavy chain variable region (VH) sequences selected from the group consisting of:
(i) any one SEQ ID NOs. 1 to 24 or 49 to 88 respectively or a sequence with at least 80% sequence identity to the sequences recited;
and/or,
(b) a light chain variable region (VL) wherein the VL comprises a humanised light chain framework region as shown in the light chain variable region (VL) sequences selected from the group consisting of:
(ii) any one of SEQ ID NOs. 25 to 48 or 89 to 128 respectively or a sequence with at least 80% sequence identity to the sequences recited.

39. An antibody-drug conjugate (ADC) according to claim 37, wherein the antibody comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 1 paired with light chain framework region from SEQ ID NO. 25;
heavy chain framework region of SEQ ID NO. 2 paired with light chain framework region from SEQ ID NO. 26;
heavy chain framework region of SEQ ID NO. 3 paired with light chain framework region from SEQ ID NO. 27;
heavy chain framework region of SEQ ID NO. 4 paired with light chain framework region from SEQ ID NO. 28;
heavy chain framework region of SEQ ID NO. 5 paired with light chain framework region from SEQ ID NO. 29;
heavy chain framework region of SEQ ID NO. 6 paired with light chain framework region from SEQ ID NO. 30;
heavy chain framework region of SEQ ID NO. 7 paired with light chain framework region from SEQ ID NO. 31;
heavy chain framework region of SEQ ID NO. 8 paired with light chain framework region from SEQ ID NO. 32;
heavy chain framework region of SEQ ID NO. 9 paired with light chain framework region from SEQ ID NO. 33;
heavy chain framework region of SEQ ID NO. 10 paired with light chain framework region from SEQ ID NO. 34;
heavy chain framework region of SEQ ID NO. 11 paired with light chain framework region from SEQ ID NO. 35;
heavy chain framework region of SEQ ID NO. 12 paired with light chain framework region from SEQ ID NO. 36;
heavy chain framework region of SEQ ID NO. 13 paired with light chain framework region from SEQ ID NO. 37;
heavy chain framework region of SEQ ID NO. 14 paired with light chain framework region from SEQ ID NO. 38;
heavy chain framework region of SEQ ID NO. 15 paired with light chain framework region from SEQ ID NO. 39;
heavy chain framework region of SEQ ID NO. 16 paired with light chain framework region from SEQ ID NO. 40;
heavy chain framework region of SEQ ID NO. 17 paired with light chain framework region from SEQ ID NO. 41;
heavy chain framework region of SEQ ID NO. 18 paired with light chain framework region from SEQ ID NO. 42;
heavy chain framework region of SEQ ID NO. 19 paired with light chain framework region from SEQ ID NO. 43;
heavy chain framework region of SEQ ID NO. 20 paired with light chain framework region from SEQ ID NO. 44;
heavy chain framework region of SEQ ID NO. 21 paired with light chain framework region from SEQ ID NO. 45;
heavy chain framework region of SEQ ID NO. 22 paired with light chain framework region from SEQ ID NO. 46;
heavy chain framework region of SEQ ID NO. 23 paired with light chain framework region from SEQ ID NO. 47; or
heavy chain framework region of SEQ ID NO. 24 paired with light chain framework region from SEQ ID NO. 48;
optionally wherein the heavy chain framework region and/or the light chain framework region have at least 80% sequence identity to the sequences recited.

40. An antibody-drug conjugate (ADC) according to claim 37, wherein the antibody comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 49 paired with light chain framework region from SEQ ID NO. 89;
heavy chain framework region of SEQ ID NO. 50 paired with light chain framework region from SEQ ID NO. 90;
heavy chain framework region of SEQ ID NO. 51 paired with light chain framework region from SEQ ID NO. 91;
heavy chain framework region of SEQ ID NO. 52 paired with light chain framework region from SEQ ID NO. 92;
heavy chain framework region of SEQ ID NO. 53 paired with light chain framework region from SEQ ID NO. 93;
heavy chain framework region of SEQ ID NO. 54 paired with light chain framework region from SEQ ID NO. 94;
heavy chain framework region of SEQ ID NO. 55 paired with light chain framework region from SEQ ID NO. 95;
heavy chain framework region of SEQ ID NO. 56 paired with light chain framework region from SEQ ID NO. 96;
heavy chain framework region of SEQ ID NO. 57 paired with light chain framework region from SEQ ID NO. 97;
heavy chain framework region of SEQ ID NO. 58 paired with light chain framework region from SEQ ID NO. 98;
heavy chain framework region of SEQ ID NO. 59 paired with light chain framework region from SEQ ID NO. 99;
heavy chain framework region of SEQ ID NO. 60 paired with light chain framework region from SEQ ID NO. 100;
heavy chain framework region of SEQ ID NO. 61 paired with light chain framework region from SEQ ID NO. 101;
heavy chain framework region of SEQ ID NO. 62 paired with light chain framework region from SEQ ID NO. 102;
heavy chain framework region of SEQ ID NO. 63 paired with light chain framework region from SEQ ID NO. 103;
heavy chain framework region of SEQ ID NO. 64 paired with light chain framework region from SEQ ID NO. 104;
heavy chain framework region of SEQ ID NO. 65 paired with light chain framework region from SEQ ID NO. 105;
heavy chain framework region of SEQ ID NO. 66 paired with light chain framework region from SEQ ID NO. 106;
heavy chain framework region of SEQ ID NO. 67 paired with light chain framework region from SEQ ID NO. 107;
heavy chain framework region of SEQ ID NO. 68 paired with light chain framework region from SEQ ID NO. 108;
heavy chain framework region of SEQ ID NO. 69 paired with light chain framework region from SEQ ID NO. 109;
heavy chain framework region of SEQ ID NO. 70 paired with light chain framework region from SEQ ID NO. 110;
heavy chain framework region of SEQ ID NO. 71 paired with light chain framework region from SEQ ID NO. 111;
heavy chain framework region of SEQ ID NO. 72 paired with light chain framework region from SEQ ID NO. 112;
heavy chain framework region of SEQ ID NO. 73 paired with light chain framework region from SEQ ID NO. 113;
heavy chain framework region of SEQ ID NO. 74 paired with light chain framework region from SEQ ID NO. 114;
heavy chain framework region of SEQ ID NO. 75 paired with light chain framework region from SEQ ID NO. 115;
heavy chain framework region of SEQ ID NO. 76 paired with light chain framework region from SEQ ID NO. 116;
heavy chain framework region of SEQ ID NO. 77 paired with light chain framework region from SEQ ID NO. 117;
heavy chain framework region of SEQ ID NO. 78 paired with light chain framework region from SEQ ID NO. 118;
heavy chain framework region of SEQ ID NO. 79 paired with light chain framework region from SEQ ID NO. 119;
heavy chain framework region of SEQ ID NO. 80 paired with light chain framework region from SEQ ID NO. 120;
heavy chain framework region of SEQ ID NO. 81 paired with light chain framework region from SEQ ID NO. 121;
heavy chain framework region of SEQ ID NO. 82 paired with light chain framework region from SEQ ID NO. 122;
heavy chain framework region of SEQ ID NO. 83 paired with light chain framework region from SEQ ID NO. 123;
heavy chain framework region of SEQ ID NO. 84 paired with light chain framework region from SEQ ID NO. 124;
heavy chain framework region of SEQ ID NO. 85 paired with light chain framework region from SEQ ID NO. 125;
heavy chain framework region of SEQ ID NO. 86 paired with light chain framework region from SEQ ID NO. 126;
heavy chain framework region of SEQ ID NO. 87 paired with light chain framework region from SEQ ID NO. 127; or
heavy chain framework region of SEQ ID NO. 88 paired with light chain framework region from SEQ ID NO. 128;
optionally wherein the heavy chain framework region and/or the light chain framework region have at least 80% sequence identity to the sequences recited.

41. An antibody-drug conjugate (ADC) according to any one of claims 37 to 40, wherein the heavy chain framework region and/or the light chain framework region have at least 90% sequence identity to the sequences recited.

42. An antibody-drug conjugate (ADC) according to claim 41, wherein the heavy chain framework region and/or the light chain framework region have at least 95% or at least 99% sequence identity to the sequences recited.

43. An antibody-drug conjugate (ADC) according to any one of claims 31 to 42, wherein the antibody comprises:
(a) a heavy chain variable region (VH) selected from the group consisting of:
(i) any one SEQ ID NOs. 1 to 24 or 49 to 88 respectively or a sequence with at least 80% sequence identity to the sequences recited;
and/or,
(a) a light chain variable region (VL) selected from the group consisting of:
(ii) any one of SEQ ID NOs. 25 to 48 or 89 to 128 respectively or a sequence with at least 80% sequence identity to the sequences recited.

44. An antibody-drug conjugate (ADC) according to claim 43, wherein the antibody comprises one of the following pairs of heavy chain variable regions (VH) and light chain variable regions (VL):
SEQ ID NO. 1 paired with SEQ ID NO. 25;
SEQ ID NO. 2 paired with SEQ ID NO. 26;
SEQ ID NO. 3 paired with SEQ ID NO. 27;
SEQ ID NO. 4 paired with SEQ ID NO. 28;
SEQ ID NO. 5 paired with SEQ ID NO. 29;
SEQ ID NO. 6 paired with SEQ ID NO. 30;
SEQ ID NO. 7 paired with SEQ ID NO. 31;
SEQ ID NO. 8 paired with SEQ ID NO. 32;
SEQ ID NO. 9 paired with SEQ ID NO. 33;
SEQ ID NO. 10 paired with SEQ ID NO. 34;
SEQ ID NO. 11 paired with SEQ ID NO. 35;
SEQ ID NO. 12 paired with SEQ ID NO. 36;
SEQ ID NO. 13 paired with SEQ ID NO. 37;
SEQ ID NO. 14 paired with SEQ ID NO. 38;
SEQ ID NO. 15 paired with SEQ ID NO. 39;
SEQ ID NO. 16 paired with SEQ ID NO. 40;
SEQ ID NO. 17 paired with SEQ ID NO. 41;
SEQ ID NO. 18 paired with SEQ ID NO. 42;
SEQ ID NO. 19 paired with SEQ ID NO. 43;
SEQ ID NO. 20 paired with SEQ ID NO. 44;
SEQ ID NO. 21 paired with SEQ ID NO. 45;
SEQ ID NO. 22 paired with SEQ ID NO. 46;
SEQ ID NO. 23 paired with SEQ ID NO. 47; or
SEQ ID NO. 24 paired with SEQ ID NO. 48;
optionally wherein the heavy chain variable region (VH) and/or the light chain variable region (VL) have at least 80% sequence identity to the sequences recited.

45. An antibody-drug conjugate (ADC) according to claim 43, wherein the antibody comprises one of the following pairs of heavy chain variable regions (VH) and light chain variable regions (VL):
SEQ ID NO. 49 paired with SEQ ID NO. 89;
SEQ ID NO. 50 paired with SEQ ID NO. 90;
SEQ ID NO. 51 paired with SEQ ID NO. 91;
SEQ ID NO. 52 paired with SEQ ID NO. 92;
SEQ ID NO. 53 paired with SEQ ID NO. 93;
SEQ ID NO. 54 paired with SEQ ID NO. 94;
SEQ ID NO. 55 paired with SEQ ID NO. 95;
SEQ ID NO. 56 paired with SEQ ID NO. 96;
SEQ ID NO. 57 paired with SEQ ID NO. 97;
SEQ ID NO. 58 paired with SEQ ID NO. 98;
SEQ ID NO. 59 paired with SEQ ID NO. 99;
SEQ ID NO. 60 paired with SEQ ID NO. 100;
SEQ ID NO. 61 paired with SEQ ID NO. 101;
SEQ ID NO. 62 paired with SEQ ID NO. 102;
SEQ ID NO. 63 paired with SEQ ID NO. 103;
SEQ ID NO. 64 paired with SEQ ID NO. 104;
SEQ ID NO. 65 paired with SEQ ID NO. 105;
SEQ ID NO. 66 paired with SEQ ID NO. 106;
SEQ ID NO. 67 paired with SEQ ID NO. 107;
SEQ ID NO. 68 paired with SEQ ID NO. 108;
SEQ ID NO. 69 paired with SEQ ID NO. 109;
SEQ ID NO. 70 paired with SEQ ID NO. 110;
SEQ ID NO. 71 paired with SEQ ID NO. 111;
SEQ ID NO. 72 paired with SEQ ID NO. 112;
SEQ ID NO. 73 paired with SEQ ID NO. 113;
SEQ ID NO. 74 paired with SEQ ID NO. 114;
SEQ ID NO. 75 paired with SEQ ID NO. 115;
SEQ ID NO. 76 paired with SEQ ID NO. 116;
SEQ ID NO. 77 paired with SEQ ID NO. 117;
SEQ ID NO. 78 paired with SEQ ID NO. 118;
SEQ ID NO. 79 paired with SEQ ID NO. 119;
SEQ ID NO. 80 paired with SEQ ID NO. 120;
SEQ ID NO. 81 paired with SEQ ID NO. 121;
SEQ ID NO. 82 paired with SEQ ID NO. 122;
SEQ ID NO. 83 paired with SEQ ID NO. 123;
SEQ ID NO. 84 paired with SEQ ID NO. 124;
SEQ ID NO. 85 paired with SEQ ID NO. 125;
SEQ ID NO. 86 paired with SEQ ID NO. 126
SEQ ID NO. 87 paired with SEQ ID NO. 127; or
SEQ ID NO. 88 paired with SEQ ID NO. 128; optionally wherein the heavy chain variable region (VH) and/or the light chain variable region (VL) have at least 80% sequence identity to the sequences recited.

46. An antibody-drug conjugate (ADC) according to any one of claims 31 to 45 wherein L-CDR3 is further mutated to replace D (aspartic acid/aspartate) at position 93 with any one of the following amino acid residues:
A, E, F, G, H, I, K, L, N, P, Q, R, S, T, V, W, or Y.

47. An antibody-drug conjugate (ADC) according to any one of claims 31 to 46 wherein L-CDR3 is further mutated to replace P (proline) at position 94 with any one of the following amino acid residues:
A, E, F, H, I, K, L, N, Q, R, T, V, W, or Y.

48. An antibody-drug conjugate (ADC) according to claim 46 wherein any one of the mutations indicated for position 93 is paired with any one of the mutations indicated in claim 35 for position 94.

49. An antibody-drug conjugate (ADC) according to claim 46, 47 or 48, wherein L-CDR3 is further mutated to replace the DP at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY.

50. An antibody-drug conjugate (ADC) according to any one of claims 46 to 49 wherein the light chain variable sequence VL is as shown in any one of SEQUENCE ID NOS 150 to 173 or a variant having at least 80% sequence identity thereto.

51. An antibody-drug conjugate (ADC) according to any one of claims 36 to 50 wherein H-CDR2 is further mutated to replace D (aspartic acid/aspartate) at position 55 with any one of the following amino acid residues:
A, E, F, G, H, I, K, L, P, Q, R, V, W or Y.

52. An antibody-drug conjugate (ADC) according to any one of claims 36 to 51 wherein H-CDR2 is further mutated to replace G (glycine) at position 56 with any one of the following amino acid residues:
A, E, F, H, I, K, L, N, Q, R, T, V, W or Y.

53. An antibody-drug conjugate (ADC) according to claim 51 wherein any one of the mutations indicated for position 55 is paired with any one of the mutations indicated in claim 22 for position 56.

54. An antibody-drug conjugate (ADC) according to claim 51, 52 or 53 wherein H-CDR2 is further mutated to replace the DG at positions 55 and 56 with any one of the following pairs of amino acid residues:
DE, DK, DA, EG, QG, RG, AA, EE, FF, GG, HH, KK, LL, DN, PQ, QQ, RR, DT, VV, WW, or YY.

55. An antibody-drug conjugate (ADC) according to any one of claims 51 to 54 wherein the heavy chain variable sequence VH is as shown in any one of SEQUENCE ID NOs 129 to 149 or a variant having at least 80% sequence identity thereto.

56. An antibody-drug conjugate (ADC) according to any one of claims 46 to 55 wherein L-CDR3 is further mutated to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY;
and wherein H-CDR2 is further mutated to replace the DG at positions 55 and 56 with any one of the following pairs of amino acid residues:
DE, DK, DA, EG, QG, RG, AA, EE, FF, GG, HH, KK, LL, DN, PQ, QQ, RR, DT, VV, WW, or YY.

57. An antibody-drug conjugate (ADC) according to any one of claims 46 to 56 wherein L-CDR3 is further mutated to replace the “DP” at positions 93 and 94 with any one of the following pairs of amino acid residues:
DA, DK, DN, EP, KP, NP, QP, or RP;
and wherein H-CDR2 is further mutated to replace the DG at positions 55 and 56 with any one of the following pairs of amino acid residues:
DE, DK, DA, EG, QG, or RG.

58. An antibody-drug conjugate (ADC) according to any one of claims 1 to 30 comprising an antibody comprising a combination of a light chain variable region (VL) and a heavy chain variable region (VH), wherein:
the VL comprises complementarity determining regions (CDRs) L-CDR1, L-CDR2, and L-CDR3 as set forth in SEQ ID NOs. 179, 181, and 183, respectively; and,
the VH comprising CDRs H-CDR1, H-CDR2 and H-CDR3 as set forth in SEQ ID NOs. 185, 187 and 189, respectively.

59. An antibody-drug conjugate (ADC) according to claim 58 wherein the VL comprises SEQ ID NOs. 178, 180, and 182; and the VH comprises SEQ ID NOs. 184, 186 and 188; optionally wherein the heavy chain variable region (VH) and/or the light chain variable region (VL) have at least 80% sequence identity to the sequences recited.

60. An antibody-drug conjugate (ADC) according to claim 58 or 59 wherein the VL comprises SEQ ID No. 177 and the VH comprises SEQ ID No. 176; optionally wherein the heavy chain variable region (VH) and/or the light chain variable region (VL) have at least 80% sequence identity to the sequences recited.

61. An antibody-drug conjugate (ADC) according to any one of claims 47 to 59, wherein the heavy chain variable region (VH) and/or the light chain variable region (VL) have at least 90%, at least 95% or at least 99% sequence identity to the sequences recited.

62. An antibody-drug conjugate (ADC) according to any preceding claim wherein the glycans terminated by alpha 2,6-linked sialic acids comprise STn, 2,6-sialyl T, di-sialyl T, or 2,6-sialolactosamine.

63. An antibody-drug conjugate (ADC) according to any preceding claim wherein the antibody is subject to glycan changes at glycosylation sites.

64. An antibody-drug conjugate (ADC) according to any preceding claim wherein the antibody is a monoclonal antibody, chimeric antibody, or a humanized antibody.

65. An antibody-drug conjugate (ADC) according to any preceding claim wherein the antibody is a functional antibody fragment that binds STn and a group of glycans terminated by alpha 2,6-linked sialic acids.

66. A pharmaceutical composition comprising an antibody-drug conjugate (ADC) according to any one of claims 1 to 60, and a pharmaceutically acceptable carrier.

67. An antibody-drug conjugate (ADC) according to any one of claims 1 to 65 for use in medicine.

68. An antibody-drug conjugate (ADC) according to any one of claims 1 to 65 for use in treating the human or animal body.

69. An antibody-drug conjugate (ADC) according to any one of claims 1 to 65 for use in detecting and/or treating different types of cancer.

70. A linker cleavable by glucuronidase, wherein the linker is configured to be coupled to a drug via a quaternary ammonium salt linkage, and is PEGylated with a group comprising polyethylene glycol (PEG).

71. A linker cleavable by glucuronidase, wherein the linker is configured to be coupled to a drug via a carbamate linkage, and does not include an alkyne moiety.

72. A linker cleavable by glucuronidase, wherein the linker is configured to be coupled to a drug via a carbamate linkage and terminates in a maleimide moiety.

73. A linker according to claims 70 to 72 wherein the linker comprises a β-glucuronide moiety.

74. A linker according to any one of claims 70 to 73 wherein the linker comprises a β-glucuronide moiety as shown in formula I′: 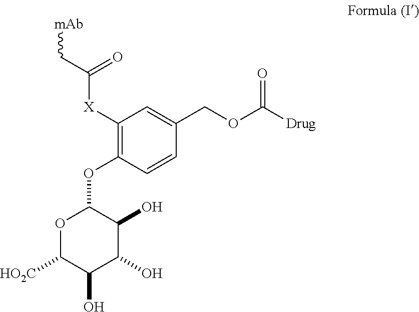
wherein X is NH, N—CH₃ or CF₂.

75. A linker according to any one of claims 70 to 74 wherein the linker comprises a β-glucuronide moiety as shown in formula I:

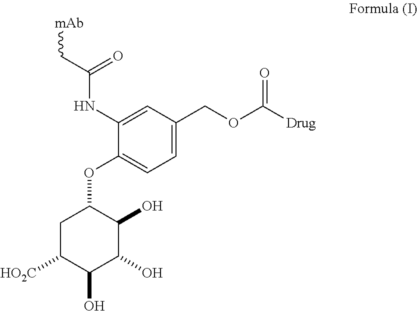

76. A linker according to any one of claim 74 or 75 wherein the linker is PEGylated with a group comprising polyethylene glycol (PEG) on the amide moiety in formula (I′) or (I).

77. A linker according to any one of claims 70 to 76 wherein the linker is PEGylated with a group comprising polyethylene glycol (PEG) based on the structure: 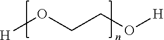
wherein n is from 1 to 5.

78. A linker according to claim 77 wherein n is 2 to 4.

79. A linker according to claim 78 wherein n is 3.

80. A linker according to any one of claims 70 to 79 wherein the linker comprises a group of formula II:

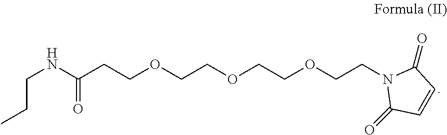 [SMILES: CCCNC(=O)CCOCCOCCOCCN1C(=O)C=CC1=O]

81. A linker according to any one of claims 70 to 80 wherein the linker is as shown in formula III′: 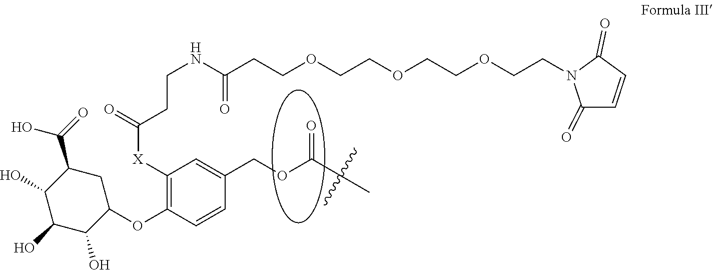
wherein X is NH, N—CH₃ or CF₂.

82. A linker according to any one of claims 70 to 81 wherein the linker is as shown in formula III:

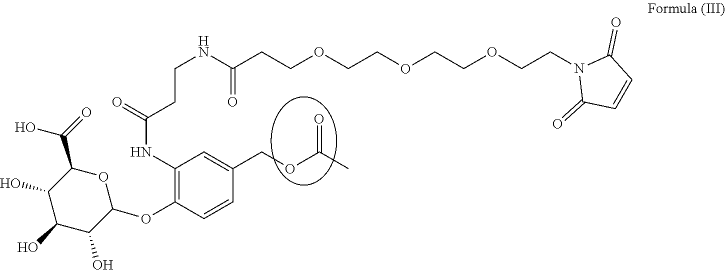 [SMILES: CC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1]

83. A linker according to any one of claims 70 or 73 to 82 wherein the linker is as shown in formula IV′: 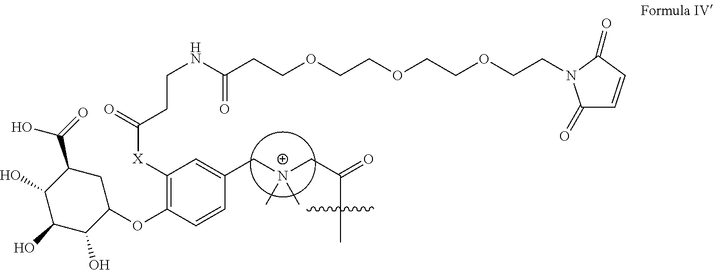
wherein X is NH, N—CH₃ or CF₂.

84. A linker according to any one of claims 70 or 73 to 83, wherein the linker is as shown in formula IV:

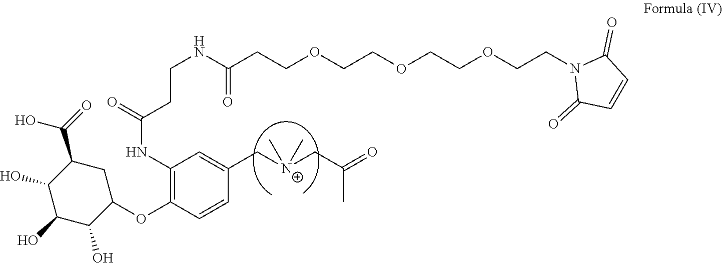 [SMILES: CC(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)C=CC2=O)c1]

85. A drug-linker payload, as shown in formula V′: 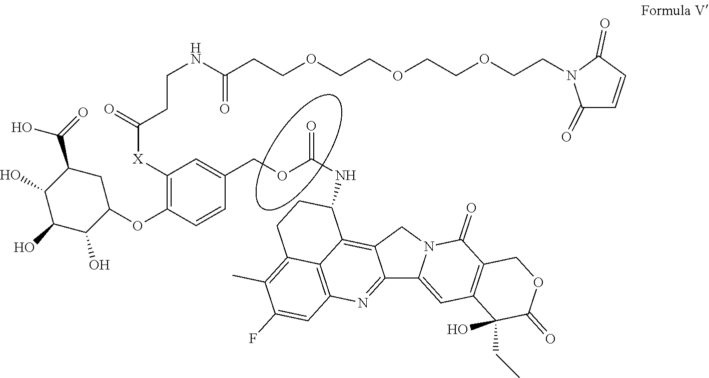
wherein X is NH, N—CH₃ or CF₂.

86. A drug-linker payload according to claim 85 as shown in formula V:

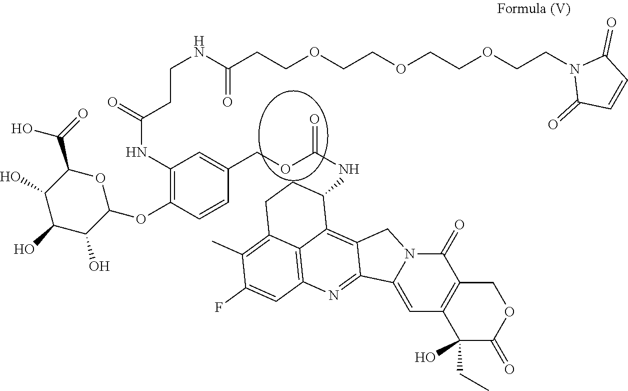 [SMILES: CC[C@@]1(O)C(=O)OCc2c1cc1n(c2=O)Cc2c-1nc1cc(F)c(C)c3c1c2[C@@H](NC(=O)OCc1ccc(OC2O[C@H](C(=O)O)[C@@H](O)[C@H](O)[C@H]2O)c(NC(=O)CCNC(=O)CCOCCOCCOCCN2C(=O)C=CC2=O)c1)CC3]

87. A drug-linker payload, as shown in formula VI′: 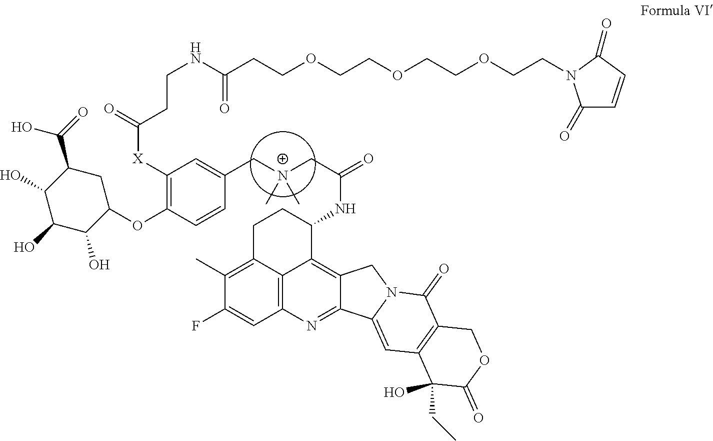
wherein X is NH, N—CH₃ or CF₂.

88. A drug-linker payload according to claim 87, of formula (VI):

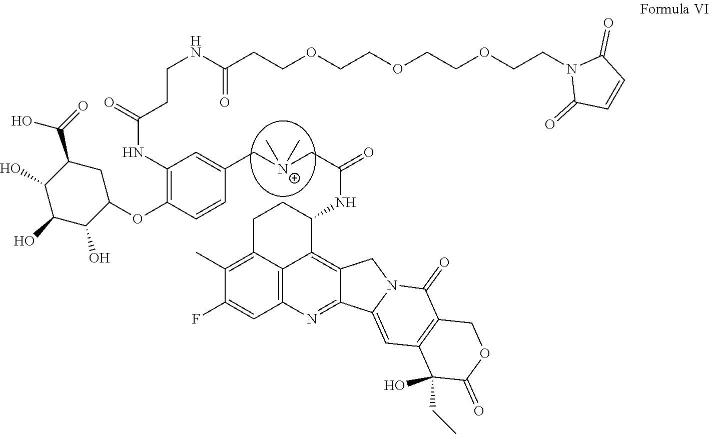 [SMILES: CC[C@@]1(O)C(=O)OCc2c1cc1n(c2=O)Cc2c-1nc1cc(F)c(C)c3c1c2[C@@H](NC(=O)C[N+](C)(C)Cc1ccc(OC2C[C@H](C(=O)O)[C@@H](O)[C@H](O)[C@H]2O)c(NC(=O)CCNC(=O)CCOCCOCCOCCN2C(=O)C=CC2=O)c1)CC3]

89. An antibody drug conjugate (ADC) comprising an antibody conjugated to a drug via a linker according to any one of claims 70 to 84.

90. An antibody-drug conjugate (ADC) according to claim 89 wherein the antibody binds to sialyl Tn (STn) or a glycan terminated by an alpha 2,6-linked sialic acid.

91. An antibody-drug conjugate (ADC) according to claim 89 or 90 wherein the antibody binds to a tumor biomarker.

92. An antibody-drug conjugate (ADC) according to claim 89, 90 or 91 wherein the drug comprises a growth inhibitory agent suitable for treating cancer.

93. An antibody-drug conjugate (ADC) according to claim 89, 90 or 91 wherein the conjugate comprises a labeling detection means for diagnostic purposes.

94. An antibody-drug conjugate (ADC) according to any one of claims 1 to 65 or 89 to 93, having an antibody-drug ratio (DAR) which is an integer from 1 to 10.

95. An antibody-drug conjugate (ADC) according to claim 94, having an antibody-drug ratio (DAR) which is an integer from 2 to 6.

96. An antibody-drug conjugate (ADC) according to claim 95, having an antibody-drug ratio (DAR) which is an integer from 3 to 5.

97. An antibody-drug conjugate (ADC) according to claim 96, having an antibody-drug ratio (DAR) which is an integer from 2 to 6.

98. An exatecan derivative compound with a handle moiety covalently bonded to said exatecan parent molecule at a position that does not significantly affect the therapeutic or pharmacokinetic properties of the parent molecule, wherein said handle moiety comprises a quaternary ammonium salt of the formula:

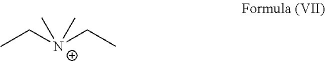

99. An exatecan derivative compound having the following general structure:

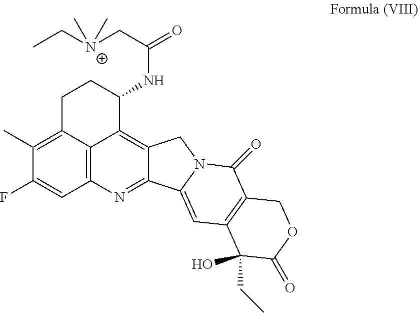 [SMILES: CC[C@@]1(O)C(=O)OCc2c1cc1n(c2=O)Cc2c-1nc1cc(F)c(C)c3c1c2[C@@H](NC(=O)C[N+](C)(C)CC)CC3]

100. An exatecan derivative compound according to claim 94 or 95 wherein the exatecan derivative compound exhibits an improved safety profile compared to the exatecan parent molecule and retains therapeutic activity against cancer cells.
